(19) United States
(12) Patent Application Publication
(10) Pub. No.: US 2024/0343824 A1
(43) Pub. Date: Oct. 17, 2024

(54) BINDING MOLECULES THAT INHIBIT CANCER GROWTH

(71) Applicants: Merus N.V., Utrecht (NL); Fundació Institut de Recerca Biomèdica (IRB Barcelona), Barcelona (ES); Institució Catalana de Recerca I Estudis Avançats, Barcelona (ES)

(72) Inventors: Mark THROSBY, Utrecht (NL); Ton LOGTENBERG, Utrecht (NL); Johannes Carolus CLEVERS, Utrecht (NL); Robert Gerhardus Jacob VRIES, Utrecht (NL); Eduard BATLLE, Barcelona (ES); Bram HERPERS, Leiden (NL)

(21) Appl. No.: 18/431,642

(22) Filed: Feb. 2, 2024

Related U.S. Application Data
(62) Division of application No. 15/770,317, filed on Apr. 23, 2018, now Pat. No. 11,939,394.

(30) Foreign Application Priority Data
Oct. 23, 2015 (EP) 15191343.1
May 6, 2016 (EP) 16168647.2

(51) Int. Cl. C07K 16/30; C07K 16/28

(52) U.S. Cl. CPC C07K 16/3046; C07K 16/28; C07K 16/30; C07K 2317/31; C07K 2317/34; C07K 2317/55; C07K 2317/76; C07K 2317/92

(57) ABSTRACT

The invention provides means and methods for inhibiting growth of a cancer. The means in some embodiments comprise proteins and antibodies that binds an extracellular part of a membrane associated member of the epidermal growth factor (EGF) receptor family and an extracellular part of a membrane associated member of a WNT signaling pathway. Further provided are various cells and assays that are helpful in the production of the proteins, antibodies and cells.

### RELATED APPLICATIONS

This application is a Division of U.S. patent application Ser. No. 15/770,317, filed Apr. 23, 2018, which claims priority to PCT/NL2016/050726, filed Oct. 21, 2016. The entire contents of these patent applications, along with all documents cited therein, are hereby incorporated by reference.

### REFERENCE TO SEQUENCE LISTING SUBMITTED ELECTRONICALLY

The content of the electronically submitted sequence listing (Name: 4096_0110003_Seglisting_ST26.xml; Size: 448,242 bytes; and Date of Creation: Jan. 8, 2024) is incorporated herein by reference in its entirety.

The invention relates to the field of binding molecules. In particular it relates to the field of therapeutic binding molecules for the treatment of diseases involving aberrant cells. More in particular it relates to binding molecules that bind an extracellular part of a membrane associated member of the epidermal growth factor (EGF) receptor family and an extra-cellular part of a membrane associated member of a WNT signaling pathway.

Cancer is still a major cause of death in the world, in spite of the many advances that have been made in the treatment of the disease and the increased knowledge of the molecular events that lead to cancer. Colorectal cancer (CRC), for instance, is the third most common cancer worldwide. In 2008, 1.23 million people were diagnosed with the disease. It is the second most common cancer in Europe, with around 447,000 new cases diagnosed in 2012 (13% of the total). Colorectal cancer is the fourth most common cause of cancer death, estimated to be responsible for 608,000 (EU 148,000) deaths per annum. While some new treatments have been advanced in CRC many have failed clinical testing; metastatic CRC is still largely incurable.

Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases, the tumors in the patients stop growing or temporarily shrink (referred to as remission) only to start proliferating again, some times more rapidly (referred to as relapse), and become increasingly more difficult to treat. More recently the focus of cancer drug development has moved away from broadly cytotoxic chemotherapy to targeted cytostatic therapies with less toxicity. Treatment of advanced cancer with targeted therapy that specifically inhibits signaling pathway components has been validated clinically in leukemia. However, in a majority of carcinomas, targeted approaches are still proving ineffective. In colorectal cancer, over 80% of patients overexpress the receptor tyrosine kinase EGFR, but treatment with EGFR blocking therapies results in response rates of 10% and, as with chemotherapy, these responses are not durable. While in some patients the poor response rate can be linked to activating mutations downstream of the blocking agent, there is accumulating scientific evidence that a special type of self-renewing cancer cell may explain the limited activity of cancer drugs in many situations.

Cancer stem cells are tumor cells that share characteristics with normal stem cells, most importantly the ability to self-renew over long periods of time and give rise to many of the cell types within a given cancer. Recent evidence suggests that while conventional chemotherapy and current targeted therapies kill differentiated and differentiating cells that form the bulk of tumors, the self-renewing cancer stem cell is less sensitive to these therapeutic approaches. Therefore, Cancer stem cells, which typically form a small subpopulation of cells within the tumor, may be responsible for the regeneration of the disease after therapy and the formation of metastases. Cancer stem cells are thought to arise from adult stem cells that have accumulated one or more mutations that initiate cancer development. In normal stem cells, proliferation is strictly controlled both spatially and temporally through signals transmitted in their immediate environment. In contrast, cancer stem cells proliferate and differentiate in a less controlled manner outside of the normal stem cell compartment where they are dependent on mutations in oncogenes and tumor suppressor genes.

Without being bound by theory it is thought that means and methods have been developed that target cancer stem cells and shut down important growth and differentiation pathways in these cells. Targeting is achieved by using proteins that have a binding arm specific for a stem cell target and another binding arm that specifically blocks a growth factor receptor pathway.

### SUMMARY OF THE INVENTION

In one aspect the invention provides a protein that binds an extracellular part of a membrane associated member of the epidermal growth factor (EGF) receptor family and an extracellular part of a membrane associated member of a WNT signaling pathway. The protein is preferably an antibody, preferably a bispecific antibody or a functional part, derivative and/or analogue thereof.

The invention also provides a bispecific antibody or a functional part, derivative and/or analogue thereof that binds an extracellular part of a membrane associated member of the epidermal growth factor (EGF) receptor family and an extra-cellular part of a membrane associated member of a WNT-signaling pathway.

Also provided is a method for the treatment of an individual that has a cancer, the method comprising administering a protein of the invention or a bispecific antibody of the invention to the individual in need thereof.

The invention further provides a protein of the invention or a bispecific antibody of the invention, for use in the treatment of an individual that has cancer.

In one embodiment the cancer is an EGF-receptor ligand responsive cancer that expresses a membrane associated member of the WNT pathway.

Further provided is a cell system comprising a protein of the invention or a bispecific antibody of the invention, and a cell that expresses a membrane associated member of the epidermal growth factor (EGF) receptor family and that expresses a membrane associated member of the WNT pathway. The cell is preferably an EGF-receptor ligand responsive cell that expresses a membrane associated member of the WNT pathway.

Also provided is a method for inhibiting growth of a cell that expresses a membrane associated member of the epidermal growth factor (EGF) receptor family and that expresses a membrane associated member of the WNT pathway in a system permissive for growth of the cell, the method providing the system with a protein of the invention or a bispecific antibody of the invention. The cell is preferably an EGF-receptor ligand responsive cell that expresses a membrane associated member of the WNT pathway.

The invention provides an antibody that comprises a variable domain that can bind an epitope on an extracellular part of LGR5 which epitope is located within amino acid residues 21-118 of SEQ ID NO: 1 depicted in FIG. 39 of which amino acid residues D43; G44, M46, F67, G90, and F91 are involved in binding of the antibody to the epitope.

The antibody is preferably an antibody wherein one or more of the amino acid residue substitutions of D43A; G44A, M46A, F67A, G90A, and F91A reduces the binding of the antibody of LGR5.

The invention further provides an antibody that comprises a variable domain that can bind an epitope on an extracellular part of LGR5 which epitope is located within amino acid residues 21-118 of SEQ ID NO: 1 depicted in FIG. 39, and wherein the binding of the antibody to LGR5 reduced by one or more of the following amino acid residue substitutions D43A; G44A, M46A, F67A, G90A, and F91A.

The antibody is preferably an antibody wherein interaction of the antibody with LGR5 on an LGR5-expressing cell does not inhibit the binding of Rspondin 1 (RSPO 1) to LGR5 by more than 20%. The inhibition of binding of the antibody to LGR5 is preferably measured when the antibody and the RSPO are present in a molar ratio of 0.1 or less; preferably in a molar ratio of between 0.1 to 0.001, (inclusive), preferably 0.1 to 0.01 (inclusive).

The invention further provides an antibody that comprises a variable domain that can bind an epitope on LGR5 that is located within amino acid residues 21-118 of SEQ ID NO: 1 depicted in FIG. 39, and wherein interaction of the antibody with LGR5 on an LGR5-expressing cell does not inhibit the binding of an RSPO to LGR5 by more than 20% when the antibody and the RSPO are present in a molar ratio of 0.1 or less; preferably in a molar ratio of between 0.1 to 0.001, (inclusive), preferably 0.1 to 0.01 (inclusive).

The epitope is preferably a conformational epitope. The epitope is preferably located within amino acid residues 40-95 of SEQ ID NO: 1 depicted in FIG. 39. The binding of the antibody to LGR5 is preferably reduced with one or more of the following amino acid residue substitutions D43A; G44A, M46A, F67A, G90A, and F91A. The antibody preferably further comprises a further variable domain that can bind a further protein. The further protein is preferably a membrane protein comprising an extracellular part. The further protein is preferably a membrane associated member of the epidermal growth factor (EGF) receptor family or cMET.

In a particularly preferred embodiment the antibody is a bispecific antibody.

The bispecific antibody preferably comprises a variable domain that can bind said epitope on an extracellular part of LGR5 and a variable domain that can bind a further protein. As mentioned herein the further protein is preferably a membrane associated member of the EGFR receptor family or cMET. The variable domain that binds this further protein preferably binds an extracellular part of said further protein.

The invention further provides a bispecific antibody comprising a variable domain that can bind an epitope on an extracellular part of LGR5 and a variable domain that can bind a further protein, wherein the LGR5 epitope is located within amino acid residues 21-118 of SEQ ID NO: 1 depicted in FIG. 39 of which amino acid residues D43; G44, M46, F67, G90, and F91 are involved in binding of the antibody. The binding of the variable domain to LGR5 is preferably reduced with one or more of the following amino acid residue substitutions D43A; G44A, M46A, F67A, G90A, and F91A. The further protein is preferably a membrane protein comprising an extracellular part. The further protein is preferably a membrane associated member of the EGF receptor family or cMET.

The binding of the (bi)specific antibody to the membrane associated member of the EGF receptor family or cMET preferably reduces ligand-induced signaling in a cell that comprises said membrane associated member of the EGF receptor family or cMET.

The invention further provides an antibody that comprises a variable domain that can bind an epitope on an extracellular part of LGR5 which epitope is located within amino acid residues 21-118 of SEQ ID NO: 1 depicted in FIG. 39 and wherein the binding of the protein to LGR5 is reduced with one or more of the following amino acid residue substitutions D43A; G44A, M46A, F67A, G90A, and F91A.

Without being bound by theory it is believed that M46, F67, G90, and F91 of LGR5 as depicted in FIG. 39, are contact residues for the variable domain, i.e. the antigen-binding site of the variable domain that can bind the LGR epitope. That amino acid residue substitution D43A and G44A reduces the binding of the antibody can be due to the fact that these are contact residues, however, it is also possible that these amino acid residue substitution induce a (slight) modification of the conformation of the part of LGR that has one or more of the other contact residues (i.e. at positions 46, 67, 90 or 91) and that conformation change is such that antibody binding is reduced. The epitope is characterized by the mentioned amino acid substitutions. Whether an antibody binds the same epitope can be determined in various ways. In the examples a preferred method is described. The method utilizes a CHO cells. The CHO cells express LGR5 on the cell membrane, or on alanine substitution mutant, preferably a mutant comprising one or more of the substitutions M46A, F67A, G90A, or F91A. The test antibody is contacted with the CHO cells and binding of the antibody to the cells compared. A test antibody binds the epitope if it binds to LGR5 and to a lesser extent to an LGR5 with a M46A, F67A, G90A, or F91A substitution. Comparing binding with a panel of mutants each comprising one alanine residue substitution is preferred. Such binding studies are well known in the art. Often the panel comprises single alanine substitution mutants covering essentially all amino acid residues. For LGR5 the panel only needs to cover the extracellular part of the protein. Expression of a particular mutant can be compromised but this is easily detected by one or more LGR5 antibodies that bind to different region(s). If expression is also reduced for these control antibodies the level or folding of the protein on the membrane is compromised for this particular mutant. Binding characteristics of the test antibody to the panel readily identifies whether the test antibodies exhibits reduced binding to mutants with a M46A, F67A, G90A, or F91A substitution and thus whether the test antibody is an antibody of the invention. Reduced binding to mutants with a M46A, F67A, G90A, or F91A substitution also identifies the epitope to be located within amino acid residues 21-118 of SEQ ID NO: 1 depicted in FIG. 39, the same applies to the location within amino acid residues 40-95 of SEQ ID NO: 1 depicted in FIG. 39. In a preferred embodiment the panel includes a D43A substitution mutant; a G44A substitution mutant of both. The antibody with the VH sequence of the VH of MF5816 exhibits reduced binding to these substitution mutants.

In a preferred embodiment the interaction of RSPO 1 with LGR5 on an LGR5-expressing cell does not inhibit the binding of the antibody to LGR5 by more than 20%. Inhibition of binding of the antibody to LGR5 is preferably measured under conditions wherein the antibody and the RSPO 1 are present in a molar ratio from of 0.1 or less; preferably in a molar ratio of between 0.1 to 0.001, (inclusive), preferably 0.1 to 0.01 (inclusive).

Inhibition of binding is preferably measured when the antibody and the RSPO 1 are present in a molar ratio from of 0.1 or less, preferably in a molar ratio of between 0.1 to 0.01 (inclusive). It was found that molar ratio's of antibody to RSPO of less than 0.001 can sometimes reduce the binding of the antibody to LGR5.

When herein molar ratio's of antibody to RSPO are mentioned it is preferred that the antibody is present in amounts that result in 40%-80% of the binding achieved when saturating amounts of the antibody are present.

The invention further provides an antibody that comprises a variable domain that can bind an epitope on an extracellular part of human EGFR of which amino acid residues 1462; G465; K489; I491; N493; and C499 are involved in binding of the antibody to the epitope. The antibody is preferably an antibody characterized in that the binding of the antibody to EGFR is reduced with an EGFR wherein one or more of the amino acid residue substitutions selected from I462A; G465A; K489A; I491A; N493A; and C499A have been introduced.

The invention further provides an antibody that comprises a variable domain that can bind an epitope on an extracellular part of human EGFR which epitope is located within amino acid residues 420-480 of SEQ ID NO: 2 depicted in FIG. 40, and wherein the binding of the antibody to LGR5 is reduced by one or more of the following amino acid residue substitutions I462A; G465A; K489A; I491A; N493A; and C499A. The binding of the antibody to human EGFR interferes with the binding of EGF to the receptor. The epitope on EGFR is a conformational epitope.

The epitope is located within amino acid residues 420-480 of SEQ ID NO: 2 depicted in FIG. 40, preferably within 430-480 of SEQ ID NO: 2 depicted in FIG. 40; preferably within 438-469 of SEQ ID NO: 2 depicted in FIG. 40.

The antibody preferably comprises a further variable domain which further variable domain can bind a further protein. The further protein is preferably a membrane protein comprising an extracellular part. The further protein is preferably a membrane associated member WNT-pathway.

The antibody is typically a bispecific antibody.

The variable domain that binds human EGFR, is preferably a variable domain with a heavy chain variable region that comprises at least the CDR3 sequence of the VH of MF3755 as depicted in FIG. 1 or a CDR3 sequence that differs in at most three, preferably in at most two, preferably in no more than one amino acid from a CDR3 sequence of the VH of MF3755 as depicted in FIG. 1.

The variable domain that binds human EGFR, is preferably a variable domain with a heavy chain variable region that comprises at least the CDR1, CDR2 and CDR3 sequences of the VH of MF3755 as depicted in FIG. 1; or the CDR1, CDR2 and CDR3 sequences of the VH of MF3755 as depicted in FIG. 1 with at most three, preferably at most two, preferably at most one amino acid substitutions.

The variable domain that binds human EGFR, is preferably a variable domain with a heavy chain variable region that comprises the sequence of the VH chain of MF3755 as depicted in FIG. 1; or the amino acid sequence of the VH chain of MF3755 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3755.

The further protein is preferably LGR4; LGR5; LGR6; RNF43 or ZNRF3, preferably LGR5.

The invention further provides a bispecific antibody comprising a variable domain that can bind an epitope on an extracellular part of EGFR and a variable domain that can bind a further protein, wherein the EGFR epitope is located within amino acid residues 420-480 of SEQ ID NO: 2 depicted in FIG. 40, which amino acid residues 1462; G465; K489; I491; N493; and C499 are involved in binding of the antibody to the epitope. The binding of the variable domain to EGFR is preferably reduced with one or more of the following amino acid residue substitutions I462A; G465A; K489A; I491A; N493A; and C499A. The further protein is preferably a membrane protein comprising an extracellular part, preferably a membrane associated member of the WNT-pathway, preferably LGR5.

Without being bound by theory it is believed that the contact residues of the epitope, i.e. where the variable domain contacts the human EGFR are likely 1462; K489; I491; and N493. The amino acid residues G465 and C499 are likely indirectly involved in the binding of the antibody to EGFR, probably because mutation by substitution into an alanine induces a (slight) conformational alteration of the epitope resulting in a reduced binding to the epitope.

It has been shown that antibodies comprising one or more variable domains that bind EGFR with the mentioned epitope have a better effectivity when used to inhibit growth of an EGFR ligand responsive cancer or cell. In the context of bispecific antibodies, an arm of the antibody comprising an EGFR binding variable domain with the mentioned epitope combines better with a variety of other arms comprising variable domains that bind extra-cellular parts of other cell surface proteins.

### DETAILED DESCRIPTION OF THE INVENTION

The invention discloses a protein that binds an extracellular part of a membrane associated member of the epidermal growth factor (EGF) receptor family and an extracellular part of a membrane associated member of a WNT signaling pathway. Such a protein is further also referred to as “a protein of the invention”.

In a preferred embodiment the protein of the invention is an antibody (or antibody part, derivative, or analogue, as described elsewhere in the application), an antibody mimetic, a polypeptide, an aptamer or a combination thereof. These proteins or aptamers typically bind to one target. The protein of the invention binds to two targets. It is to be understood that any combination of these antibodies, antibody mimetics, polypeptides and aptamers can be linked together by methods known in the art. For example, in some embodiments the protein of the invention is a conjugate or a fusion protein. For antibodies the technology of making multi-specific antibodies has progressed to also include bispecific antibodies that have the same overall structure as a normal mono-specific antibody but wherein the two arms of the antibody each bind a different target.

An antibody mimetic is a polypeptide that, like antibodies, can specifically bind an antigen, but that is not structurally related to antibodies. Antibody mimetics are usually artificial peptides or proteins with a molar mass of about 3 to 20 kDa. Common advantages over antibodies are better solubility, tissue penetration, stability towards heat and enzymes, and comparatively low production costs. Non-limiting examples of antibody mimetics are affibody molecules (typically based on the Z domain of Protein A); affilins (typically based on Gamma-B crystalline or Ubiquitin); affimers (typically based on Cystatin); affitins (typically based on Sac7d from Sulfolobus acidocaldarius); alphabodies (typically based on Triple helix coiled coil); anticalins (typically based on Lipocalins); avimers (typically based on A domains of various membrane receptors); DARPins (typically based on ankyrin repeat motif); fynomers (typically based on SH3 domain of Fyn 7); kunitz domain peptides (typically based on Kunitz domains of various protease inhibitors); and monobodies (typically based on type III domain of fibronectin).

Monobodies are synthetic binding proteins that are constructed using a fibronectin type III domain (FN3) as a molecular scaffold. Monobodies are simple and robust alternative to antibodies for creating target-binding proteins. The term “monobody” was coined in 1998 by the Koide group who published the first paper demonstrating the monobody concept using the tenth FN3 domain of human fibronectin.

Monobodies and other antibody mimetics are typically generated from combinatorial libraries in which portions of the scaffold are diversified using molecular display and directed evolution technologies such as phage display, mRNA display and yeast surface display. A large number of antibody mimetics have high affinity and high specificity to their respective targets.

Aptamers are oligonucleotide or peptide molecules that bind to a specific target molecule. Aptamers are usually created by selecting them from a large random sequence pool, but natural aptamers also exist in riboswitches. Aptamers can be used for both basic research and clinical purposes as macromolecules.

As used herein, the term “conjugate” refers to two or more molecules that have been covalently joined, optionally by a linking region. For example, in some embodiments, a conjugate is a first protein or non-protein moiety joined to a second protein or non-protein moiety by a linking region. For example, in some embodiments of a protein of the invention it comprises or consists of two or more antibodies that have been covalently joined. A conjugate is not limited to a first and second moiety but in some embodiments may also have a third, fourth or more moieties joined by further linking regions. As described elsewhere in this application, examples of protein moieties include, but are not limited to: a polypeptide, a peptidomimetic or an antibody (or antibody part, derivative, or analogue, as described elsewhere in the application). Examples of non-protein moieties include, but are not limited to aptamers. Numerous types of linker can be used, and the linker will be selected to be appropriate according to the molecule types in the conjugate and on the desired properties of the linker (length, flexibility, resistance to protease activity and other similar characteristics). Such linkers may comprise nucleotides, polypeptides, or a suitable synthetic material. For example, a linker may be a flexible peptide linker. In certain embodiments, the linker may be a cleavable linker, allowing the parts of the conjugate to be separated from each other. In other embodiments, a peptide linker might be a helical linker. Various examples and kits for linking proteins and other molecules are well known in the art. As used herein, the term “fusion protein” refers to a protein that comprises two or more polypeptides or proteins that have been joined at the DNA level by recombination and are expressed together as a single polypeptide. A fusion protein may also comprise a peptide linking region also encoded by the DNA and expressed together with the fusion protein. A peptide linker that is part of a fusion protein, may be designed to have particular characteristics such as flexibility, hydrophilicity, protease-resistance, cleavability etc. All these properties can be designed within the DNA sequence and methods for designing linkers are well known in the art. For example, antibodies can be linked together by methods well-known in the art, and as described herein, to form bispecific or multi-targeting antibodies. Furthermore, bispecific antibodies can be constructed by various methods known in the art, for example, by using technology such as BiClonics®. A bispecific monoclonal antibody (BsMAb, BsAb) typically comprises binding domains of two different monoclonal antibodies and consequently binds to two different epitopes. Biclonics® molecules, but also other full length IgG bispecific antibodies have two different antigen binding specificities encoded by two different variable regions of a full length IgG molecule of a Fab of a scFv. Biclonics® can be produced by co-transfection of individual cells with genetic constructs encoding two different common light chain (cLC) antibodies as detailed elsewhere herein. CH3 engineering ensures efficient hetero-dimerization and formation of essentially pure bispecific antibodies. In some embodiments, a protein of the invention suppresses signaling in a cell, group of cells, tissue or tumor by an amount that is useful for the intended purpose of the protein of the invention, for example, may reduce induction of any one or more of these responses by at least 5%, 10%, 15%, 20%, 25%, 30%, 35%, preferably 40%, 45%, 50%, 55%, 60%, more preferably 70%, 80%, 85%, and most preferably 90%, 95%, 99%, or 100% compared to the signaling induced by a neutral substance or negative control as measured in an assay known in the art.

A protein of the invention preferably comprises an antibody or a part, derivative or analogue thereof. A protein of the invention is preferably a bispecific antibody.

Antibodies typically bind their target via the so-called antigen binding site. An unmodified antigen-binding site is typically formed by and present in the variable domain of the antibody. The variable domain contains said antigen-binding site. A variable domain that binds an antigen is a variable domain comprising an antigen-binding site that binds the antigen.

In one embodiment an antibody variable domain comprises a heavy chain variable region (VH) and a light chain variable region (VL). The antigen-binding site can be present in the combined VH/VL variable domain, or in only the VH region or only the VL region. When the antigen-binding site is present in only one of the two regions of the variable domain, the counterpart variable region can contribute to the folding and/or stability of the binding variable region, but does not significantly contribute to the binding of the antigen itself.

As used herein, antigen-binding refers to the typical binding capacity of an antibody to its antigen. Binding of an antibody to an antigen can be assessed in various ways. One way is to incubate the antibody with the antigen (preferably cells expressing the antigen), removing unbound antibody (preferably by a wash step) and detecting bound antibody by means of a labeled antibody that binds to the bound antibody.

Antigen binding by an antibody is typically mediated through the complementarity determining regions (CDR) of the antibody and the specific three-dimensional structure of both the antigen and the variable domain allowing these two structures to bind together with precision (an interaction similar to a lock and key), as opposed to random, non-specific sticking of proteins. As an antibody typically recognizes part of an antigen called the epitope of an antigen, and as such epitope may be present in other compounds as well, antibodies according to the present invention may recognize other proteins as well, if such other compounds contain the same epitope. Hence, the term “binding” does not exclude binding of the antibodies to another protein or protein(s) that contain the same epitope. Such other protein(s) is preferably not a human protein.

A protein of the invention such as an antibody typically does not bind to other proteins than the specified target protein on the membrane of cells in a post-natal, preferably adult human.

An antibody of the invention comprising an antigen-binding site that binds to an extracellular part of one membrane associated member of the epidermal growth factor (EGF) receptor family binds to the specified member and, under otherwise identical conditions, at least 100-fold lower to the extracellular part of another member of the EGF receptor family of the same species. For instance, an antibody comprising an antigen-binding site that binds to ErbB-1, binds to ErbB-1 and, under otherwise identical conditions, at least a 100-fold lower to the homologous receptors ErbB-2 (HER2), ErbB-3 (HER3) and ErbB-4 (HER4) of the same species. An antibody comprising an antigen-binding site that binds to ErbB-2, binds to ErbB-2 and, under otherwise identical conditions, at least a 100-fold lower to the homologous receptors ErbB-1, ErbB-3 and ErbB-4 of the same species. An antibody comprising an antigen-binding site that binds to ErbB-3, binds to ErbB-3 and, under otherwise identical conditions, at least a 100-fold lower to the homologous receptors ErbB-1, ErbB-2 and ErbB-4 of the same species. An antibody comprising an antigen-binding site that binds to ErbB-4, binds to ErbB-4 and, under otherwise identical conditions, at least a 100-fold lower to the homologous receptors ErbB-1, ErbB-2 and ErbB-3 of the same species. Of course, when an antibody is designed to bind to two or more members of the family, the binding to the two or more members can be essentially the same. In the present invention it is preferred that respective antibodies each bind to only one member of a family. Considering that the ErbB-family is a family of cell surface receptors, the binding is typically assessed on cells that express the receptor(s) on their cell surface.

An antibody of the invention preferably interferes with the binding of a ligand for the member of the EGF receptor family. The term “interferes with binding” as used herein means that binding of the antibody to the member of the EGF receptor family competes with ligand for binding to the member of the EGF receptor family. The antibody may diminish ligand binding, displace ligand when this is already bound to the member of the EGF receptor family or it may, for instance through steric hindrance, at least partially prevent that ligand can bind to the member of the EGF receptor family.

An EGFR (ErbB1) or HER3 (ErbB3) binding protein of the invention, preferably antibody of the invention preferably inhibits respectively EGFR ligand or HER3 ligand-induced signaling, measured as ligand-induced growth of BxPC3 cells (ATCC CRL-1687) or BxPC3-luc2 cells (Perkin Elmer 125058) or ligand-induced cell death of A431 cells (ATCC CRL-1555). The mentioned EGFR or HER3 binding protein can reduce ligand induced signaling by at least 5%, 10%, 15%, 20%, 25%, 30%, 35%, preferably 40%, 45%, 50%, 55%, 60%, more preferably 70%, 80%, 85%, and most preferably 90%, 95%, 99%, or 100% compared to the ligand induced effect in the presence of a neutral substance or negative control as measured in an assay known in the art. EGFR and ErbB-3 each can bind a number of ligands and stimulate growth of the mentioned BxPC3 cells or BxPC3-luc2 cells. In the presence of a ligand for one or both receptors the growth of BxPC3 or BxPC3-luc2 cells is stimulated. EGFR and/or ErbB-3 ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The preferred ErbB-3 ligand for measuring ErbB-3 ligand-induced growth of BxPC3 or BxPC3-luc2 cells is NRG1. The ligand-induced growth is preferably measured using saturating amounts of ligand. In a preferred embodiment EGF is used in an amount of 100 ng/ml of culture medium. NRG1 is preferably used in 10 ng/ml of culture medium. EGF and NRG1 are preferably the EGF and NRG1 of R&D systems, cat. nr. 396-HB and 236-EG as described in the examples. Determination of whether a protein or antibody of the invention inhibits signaling in bivalent format, it is preferred that the method as described herein above is performed with a monospecific monovalent or bivalent version of the protein or antibody. In other words a protein or antibody that only has binding sites for the receptor of which signaling is to be determined. For an antibody it would be a bivalent monospecific antibody wherein the antigen binding variable domains consist of variable domains that bind the EGF-receptor family member.

An EGFR or HER3 binding protein of the invention, preferably antibody of the invention preferably inhibits respectively EGFR ligand or HER3 ligand, induced growth of BxPC3 cells (ATCC CRL-1687) or BxPC3-luc2 cells (Perkin Elmer 125058). The mentioned EGFR or HER3 binding protein can reduce ligand induced growth signaling by at least 5%, 10%, 15%, 20%, 25%, 30%, 35%, preferably 40%, 45%, 50%, 55%, 60%, more preferably 70%, 80%, 85%, and most preferably 90%, 95%, 99%, or 100% compared to the ligand induced growth induced by a neutral substance or negative control as measured in an assay known in the art. EGFR and ErbB-3 each can bind a number of ligands and stimulate growth of the mentioned BxPC3 cells or BxPC3-luc2 cells. In the presence of a ligand for one or both receptors the growth of BxPC3 or BxPC3-luc2 cells is stimulated. EGFR and/or ErbB-3 ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The preferred ErbB-3 ligand for measuring ErbB-3 ligand-induced growth of BxPC3 or BxPC3-luc2 cells is NRG1. The ligand-induced growth is preferably measured using saturating amounts of ligand. In a preferred embodiment EGF is used in an amount of 100 ng/ml of culture medium. NRG1 is preferably used in 10 ng/ml of culture medium. EGF and NRG1 are preferably the EGF and NRG1 of R&D systems, cat. nr. 396-HB and 236-EG as described in the examples.

An EGF-receptor family ligand is preferably an EGFR or HER3 receptor ligand. “HER3 or ErbB-3 ligand” as used herein refers to polypeptides which bind and activate ErbB-3. Examples of ErbB-3 ligands include, but are not limited to neuregulin 1 (NRG1) and neuregulin 2 (NRG2) (for review Olayioye M A et al.; EMBO J (2000) Vol 19: pp 3159-3167). The term preferably includes biologically active fragments and/or variants of a naturally occurring polypeptide. “EGFR or ErbB-1 ligand” as used herein refers to polypeptides which bind and activate EGFR. Examples of EGFR ligands include, but are not limited to EGF, TGF-α, HB-EGF, amphiregulin, betacellulin and epiregulin (for review Olayioye M A et al.; EMBO J (2000) Vol 19: pp 3159-3167). The term preferably includes biologically active fragments and/or variants of a naturally occurring polypeptide.

An antibody of the invention comprising an antigen-binding site that binds to extracellular part of a membrane associated member of a WNT signaling pathway binds to the specified member and, under otherwise identical conditions, at least 100-fold lower to the extracellular part of another membrane associated protein that is not a member of a WNT-signaling pathway of the same species such as insulin-like growth factor 1 (IGF-1) receptor. An antibody comprising an antigen-binding site that binds to LGR5, binds to LGR5 and, under otherwise identical conditions, at least a 100-fold lower to an extracellular part of an IGF-1 receptor of the same species. An antibody comprising an antigen-binding site that binds to LGR4, binds to LGR4 and, under otherwise identical conditions, at least a 100-fold lower to IGF-1 receptor of the same species. An antibody comprising an antigen-binding site that binds to RNF43, binds to RNF43 and, under otherwise identical conditions, at least a 100-fold lower to IGF-1 receptor, LGR4 or LGR5 of the same species. An antibody comprising an antigen-binding site that binds to ZNRF3, binds to ZNRF3 and, under otherwise identical conditions, at least a 100-fold lower to IGF-1 receptor or RNF43 of the same species. Of course, when an antibody is designed to bind to two or more members of the family, the binding to the two or more members can be essentially the same. For instance, some of the antibodies that bind LGR5 can bind LGR4 of the same species and vice versa. In a preferred embodiment an antibody of the invention that comprises an antigen-binding site that binds to extracellular part of a membrane associated member of a WNT signaling pathway binds to the specified member and, under otherwise identical conditions, at least 100-fold lower to the extracellular part of another membrane associated of the family of the same species. An antibody comprising an antigen-binding site that binds to LGR5, binds to LGR5 and, under otherwise identical conditions, at least 10-fold and preferably at least 100-fold lower to LGR4 of the same species. An antibody comprising an antigen-binding site that binds to LGR4, binds to LGR4 and, under otherwise identical conditions, at least a 10-fold and preferably at least 100-fold lower to LGR5 of the same species In the present invention it is preferred that respective antibodies each bind to only one member of a family. Preferred members of the WNT signaling pathway in the context of the present invention are LRP5, LRP6, LGR4, LGR5, LGR6, FRZ1, FRZ2, FRZ3, FRZ4, FRZ5, FRZ6, FRZ7, FRZ8, FRZ9 FRZ10, ZNRF3, RNF43, N-Cadherin, Kremen1 and Kremen2, ROR2/RYK). Considering that the members of this list are cell surface receptors, the binding is typically assessed on cells that express the receptor(s) on their cell surface.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Specific binding, is defined as binding with affinities (KD) of at least 1×10e-6 M, more preferably 1×10e-7 M, more preferably with affinities higher than 1×10e-9 M. Typically, antibodies for therapeutic applications have affinities of up to 1×10e-10 M or higher. Antibodies such as the bispecific antibodies of the present invention typically comprise the constant domains (Fc part) of a natural antibody. An antibody of the invention is typically a bispecific full length antibody, preferably of the human IgG subclass. An antibody of the present invention is preferably of the human IgG1 subclass. Such antibodies of the invention have good ADCC properties which can, if desired, be enhanced by techniques known in the art, have favorable half-life upon in vivo administration to humans and CH3 engineering technology exists that can provide for modified heavy chains that preferentially form heterodimers over homodimers upon co-expression in clonal cells.

An antibody of the invention is preferably a “full length” antibody. The term ‘full length’ according to the invention is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH for the heavy chain, and CL, VL for the light chain. An antibody binds to antigen via the variable domains contained in the Fab fragment portion. The antibody can interact with molecules and cells of the immune system through the constant domains, mostly through the Fc portion.

The terms ‘variable domain’, ‘VH/VL pair’, ‘VH/VL’ are used herein interchangeably. Full length antibodies according to the invention encompass antibodies wherein mutations may be present that provide desired characteristics or are just alternatives to the ones in the original chain. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region. For instance, ADCC activity of an antibody can be improved when the antibody itself has a low ADCC activity, by slightly modifying the constant region of the antibody (Junttila, T. T., K. et al. (2010). Cancer Research 70: 4481-4489). Changes are sometimes also made to improve storage or production or to remove C-terminal lysines (Engineered therapeutic antibodies with improved effector functions. Kubota T, Niwa R, Satoh M, Akinaga S, Shitara K, Hanai N). Another way to improve ADCC activity of an antibody is by enzymatically interfering with the glycosylation pathway resulting in a reduced fucose (von Horsten et al. (2010); Glycobiology 20:1607-1618). Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

A bispecific antibody of the invention can in one embodiment be afucosylated. A bispecific antibody of the invention preferably comprises a reduced amount of fucosylation of the N-linked carbohydrate structure in the Fc region, when compared to the same antibody produced in a normal CHO cell.

Full length IgG antibodies are preferred because of their favorable half-life and the need to stay as close to fully autologous (human) molecules for reasons of immunogenicity. An antibody of the invention is preferably a bispecific IgG antibody, preferably a bispecific full length IgG1 antibody. IgG1 is favored based on its long circulatory half-life in man. In order to prevent any immunogenicity in humans it is preferred that the bispecific IgG antibody according to the invention is a human IgG1.

The term ‘bispecific’ (bs) means that one part of the antibody (as defined above) binds to one epitope on an antigen whereas a second part binds to a different epitope on either the same antigen, or a different antigen. The different epitopes are typically present on different antigens. The different epitopes can, however, also be present on the same antigen. According to the present invention, said first and second antigens are in fact two different proteins. A preferred bispecific antibody is an antibody that comprises parts of two different monoclonal antibodies and consequently can bind to two different types of antigen. Dependent on the expression level, (sub-)cellular localization and stoichiometry of the two antigens recognized by a bispecific antibody, both Fab arms of the antibody may or may not simultaneously bind their epitope. One arm of the bispecific antibody typically contains the variable domain of one antibody and the other arm contains the variable domain of another antibody (i.e. one arm of the bispecific antibody is formed by one heavy chain paired with one light chain whereas the other arm is formed by a different heavy chain paired with a light chain). The heavy chain variable regions of the bispecific antibody of the invention are typically different from each other, whereas the light chain variable regions are preferably the same in the bispecific antibodies of the invention. A bispecific antibody wherein the different heavy chain variable regions are associated with the same or a common, light chain is also referred to as a bispecific antibody with a common light chain (cLC). Further provided is therefore a bispecific antibody according to the invention, wherein both arms comprise a common light chain.

Preferred bispecific antibodies can be obtained by co-expression of two different heavy chains and a common light chain in a single cell. When wildtype CH3 domains are used, co-expression of two different heavy chains (A and B) and a common light chain will result in three different antibody species, AA, AB and BB. AA and BB are designations for the two mono-specific, bivalent antibodies, and AB is a designation for the bispecific antibody. To increase the percentage of the desired bispecific product (AB) CH3 engineering can be employed, or in other words, one can use heavy chains with compatible hetero-dimerization domains, as defined hereunder.

The term ‘compatible hetero-dimerization domains’ as used herein refers to protein domains that are engineered such that engineered domain A′ will preferentially form heterodimers with engineered domain B′ and vice versa, whereas homo-dimerization between A′-A′ and B′-B′ is diminished.

Bispecific antibodies as described herein preferably comprise a common light chain. The term ‘common light chain’ according to the invention refers to light chains which may be identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common VL’, ‘single light chain’, ‘single VL’, with or without the addition of the term ‘rearranged’ are all used herein interchangeably. It is an aspect of the present invention to use as common light chain a human light chain that can combine with different heavy chains to form antibodies with functional antigen binding domains (WO2004/009618, WO2009/157771, Merchant et al. 1998 and Nissim et al. 1994). Preferably, the common light chain has a germline sequence. A preferred germline sequence is a light chain variable region that is frequently used in the human repertoire and has good thermodynamic stability, yield and solubility. A preferred germline light chain is O12, preferably the rearranged germline human kappa light chain IgVκ1-39*01/IGJκ1*01 (FIG. 3) or a fragment or a functional equivalent (i.e. same IgVκ1-39 gene segment but different IGJκ gene segment) thereof (nomenclature according to the IMGT database worldwide web at imgt.org). However, the same principle also works with a lambda light chain and this is therefore also provided in the context of the invention. Further provided is therefore a bispecific antibody according to the invention, wherein said common light chain is a germline light chain, preferably a rearranged germline human kappa light chain comprising the IgV_{κ}1-39 gene segment, most preferably the rearranged germline human kappa light chain IgV_{κ}1-39*01/IGJ_{κ}1*01 (FIG. 3). The terms rearranged germline human kappa light chain IgVκ1-39*01/IGJκ1*01, IGKV1-39/IG_{κ}J1, huVκ1-39 light chain or in short huVκ1-39, or simply 1-39 are used interchangeably throughout the application. Obviously, those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions. The light chain of the present invention can also be a light chain as specified herein above, having 1-20, preferably 1-5 amino acid insertions, deletions, substitutions or a combination thereof.

Also contemplated are antibodies wherein a VH is capable of specifically recognizing a first antigen and the VL, paired with the VH in an immunoglobulin variable domain, is capable of specifically recognizing a second antigen. The resulting VH/VL pair will bind either antigen 1 or antigen 2. Such so called “two-in-one antibodies”, described in for instance WO2008/027236, WO2010/108127 and Schaefer et al (2011 Cancer Cell 20, 472-486), are different from bispecific antibodies of the invention and are further referred to as “two-in-one” antibodies. A part of an antibody is an antigen binding part and typically contains the variable domains of the antibody. A part can also be a so-called single domain antibody fragment. A single-domain antibody fragment (sdAb, called Nanobody by Ablynx, the developer) is an antibody fragment with a single monomeric variable antibody domain. Like a whole antibody, it is able to bind selectively to a specific antigen. With a molecular weight of only 12-15 kDa, single-domain antibody fragments are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Single-domain antibodies by themselves are not much smaller than normal antibodies (being typically 90-100 kDa). Single-domain antibody fragments are mostly engineered from heavy-chain antibodies found in camelids; these are called VHH fragments (Nanobodies®). Some fishes also have heavy-chain only antibodies (IgNAR, ‘immunoglobulin new antigen receptor’), from which single-domain antibody fragments called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. A non-limiting example of an antibody part contains a variable domain of a heavy chain and/or a light chain of an antibody or an equivalent thereof. Non-limiting examples of such parts are VHH, Human Domain Antibodies (dAbs) and Unibodies. Preferred antibody parts or derivatives have at least a variable domain of a heavy chain and a light chain of an antibody or equivalents thereof. Non-limiting examples of such binding molecules are F(ab)-fragments and Single chain Fv fragments. A functional part of a bispecific antibody comprises the antigen binding parts of the bispecific antibody, or a derivative and/or analogue of the binding parts. As mentioned herein above, the binding part of an antibody is encompassed in the variable domain.

A derivative of an antibody is a protein that but for the CDR regions deviates from the amino acid sequence of a natural antibody in at most 20 amino acids. A derivative of an antibody as disclosed herein is an antibody that deviates from said amino acid sequence in at most 20 amino acids.

Preferred membrane associated member of the WNT pathway that a protein of the invention binds to is LRP5, LRP6, LGR4, LGR5, LGR6, FRZ1, FRZ2, FRZ3, FRZ4, FRZ5, FRZ6, FRZ7, FRZ8, FRZ9 FRZ10, ZNRF3, RNF43, N-Cadherin, Kremen1 and Kremen2, ROR2, RYK. From this list the membrane associated members of the LGR family that are active in the WNT pathway are preferred, in particular LGR4, LGR5 and LGR6. In a preferred embodiment the membrane associated member of the LGR family is LGR4 or LGR 5, in particular LGR5. Other preferred membrane associated members of the canonical WNT pathway are ZNRF3 and RNF43.

Mouse Lgr4, Lgr5, and Lgr6 have been demonstrated to be high affinity receptors for R-spondins (R-spondin 1-4) leading to increased phosphorylation of Wnt receptors Lrp5/6 and stabilization of b-catenin without the involvement of G-protein signaling (Carmon, K. S., et al. (2011). Proc Natl Acad Sci USA 108, 11452-11457; de Lau, W., et al. (2011) Nature 476, 293-297.). The R-spondins are members of a much larger family of proteins characterized by the presence of thrombospondin repeats (TSRs). R-spondins also contain N-terminal cysteine-rich Furin repeats which are required to exert the Wnt-enhancing activity as measured by b-catenin stabilization and phosphorylation of the Wnt/Frizzled coreceptor Lrp6 (Kim et al. (2005)). Gene fusions that increase expression levels of functional R-spondins have been reported in a subset of human colon cancers (Seshagiri, S., et al. (2012). Nature 488, 660-664.). While the perceived role of the Lgr-R-spondin complex is to modulate signaling through the Lrp-Frizzed-wnt receptor complex, current models suggest that the Lgr-R-spondin complex is itself subject to modulation through the highly homologous E3 ligases Rnf43 and Znrf3. Specifically, Lgr4, Lgr5, and Lgr6 receptors serve to efficiently recruit R-spondin ligands and bring these into position for interaction with Rnf43/Znrf3, which also contain R-spondin binding sites (de Lau, W., et al (2014). Genes Dev 28, 305-316). This interaction leads to membrane clearance of the Rnf43 or Znrf3, resulting in persistence of surface Frizzled receptors, and the boosting of wnt signal strength( de Lau, et al (2014). Genes Dev 28, 305-316) and enriched in colon cancer (Hao, H.-X., et al. (2012) Nature 485, 195-200).

A membrane associated member of the WNT pathway that is active in the canonical pathway, it can also be active, depending on the particular member, in the non-canonical pathway and vice versa.

The human epidermal growth factor (EGF) receptor family (HER) has four members: ErbB (Erythroblastoma)-1, ErbB-2, ErbB-3 and ErbB-4. Epidermal growth factor (EGF) receptor (EGFR, ErbB1, or HER1) is a member of a family of four receptor tyrosine kinases (RTKs), named Her- or cErbB-1, -2, -3 and -4. ErbB-1 is also known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. The major signal transduction pathway activated by EGFR is composed of the Ras-mitogen-activated protein kinase (MAPK) mitogenic signalling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. The database accession number for the human EGFR protein and the gene encoding it is (GenBank NM_005228.3). This accession number is primarily given to provide a further method of identification of EGFR protein as a target, the actual sequence of the EGFR protein bound by an antibody may vary, for instance because of a mutation in the encoding gene such as those occurring in some cancers or the like. The words cancer and tumor are used herein and typically both refer to cancer, unless otherwise specifically stated.

Where reference herein is made to EGFR, the reference refers to human EGFR unless otherwise stated. The antigen-binding site that binds EGFR, binds EGFR and a variety of variants thereof such as those expressed on some EGFR positive tumors.

The term ‘ErbB-3’ as used herein refers to the protein that in humans is encoded by the ERBB3 gene. Alternative names for the gene or protein are HER3; LCCS2; MDA-BF-1; c-ErbB-3; c-ErbB3; ErbB3-S; p180-ErbB3; p45-sErbB3; and p85-sErbB3. Where reference is made herein to ErbB-3, the reference refers to human ErbB-3. An antibody comprising an antigen-binding site that binds ErbB-3, binds human ErbB-3. The ErbB-3 antigen-binding site may, due to sequence and tertiary structure similarity between human and other mammalian orthologs, also bind such an ortholog but not necessarily so. Database accession numbers for the human ErbB-3 protein and the gene encoding it are (NP_001005915.1, NP_001973.2, NC_000012.11, NC_018923.2, NT_029419.12). The accession numbers are primarily given to provide a further method of identification of ErbB-3 as a target, the actual sequence of the ErbB-3 protein bound by an antibody may vary, for instance because of a mutation in the encoding gene such as those occurring in some cancers or the like. The ErbB-3 antigen binding site binds ErbB-3 and a variety of variants thereof, such as those expressed by some ErbB-3 positive tumor cells. The antigen-binding site that binds ErbB-3 preferably binds domain III of ErbB-3. The EGF receptor family member as mentioned herein as a target for the protein of the invention or the bispecific antibody of the invention is preferably epidermal growth factor receptor Erbb-1 (EGFR), ErbB-3 or ErbB-4. In a preferred embodiment the EGF receptor family member is ErbB-1 or ErbB-3.

The term “LGR” refers to the family of proteins known as Leucine-rich repeat-containing G-protein coupled receptors. Several members of the family are known to be involved in the WNT signaling pathway, of note LGR4; LGR5 and LGR6.

LGR4 is Leucine-Rich Repeat Containing G Protein-Coupled Receptor 4 Alternative names for the gene or protein are; GPR48; G Protein-Coupled Receptor 48; BNMD17; Leucine-Rich Repeat-Containing G Protein-Coupled Receptor 4; Leucine-Rich Repeat-Containing G-Protein Coupled Receptor 4; G-Protein Coupled Receptor 48; A protein or antibody of the invention that binds LGR4, binds human LGR4.

The LGR4 binding protein or antibody of the invention may, due to sequence and tertiary structure similarity between human and other mammalian orthologs, also bind such an ortholog but not necessarily so. Database accession numbers for the human LGR4 protein and the gene encoding it are (NC_000011.10; NC_018922.2; NT_009237.19; NP_060960.2). The accession numbers are primarily given to provide a further method of identification of LGR4 as a target, the actual sequence of the LGR4 protein bound may vary, for instance because of a mutation in the encoding gene such as those occurring in some cancers or the like. The LGR4 antigen binding site binds LGR4 and a variety of variants thereof, such as those expressed by some LGR4 positive tumor cells.

LGR5 is Leucine-Rich Repeat Containing G Protein-Coupled Receptor 5 Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX.

A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so. Database accession numbers for the human LGR5 protein and the gene encoding it are (NC_000012.12; NT_029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. The LGR5 antigen binding site binds LGR5 and a variety of variants thereof, such as those expressed by some LGR5 positive tumor cells.

ZNRF3 is Zinc And Ring Finger 3. Alternative names for the gene or protein are Zinc And Ring Finger 3; Zinc/RING Finger Protein 3; RING Finger Protein 203; KIAA1133; RNF203; Novel C3HC4 Type Zinc Finger (Ring Finger); E3 Ubiquitin-Protein Ligase ZNRF3; CTA-292E10.6; EC 6.3.2.; and BK747E2.3 3.

A protein or antibody of the invention that binds ZNRF3, binds human ZNRF3. The ZNRF3 binding protein or antibody of the invention may, due to sequence and tertiary structure similarity between human and other mammalian orthologs, also bind such an ortholog but not necessarily so. Database accession numbers for the human ZNRF3 protein and the gene encoding it are (NC_000022.11; NT_011520.13; NC_018933.2; NP_001193927.1; NP_115549.2). The accession numbers are primarily given to provide a further method of identification of ZNRF3 as a target, the actual sequence of the ZNRF3 protein bound may vary, for instance because of a mutation in the encoding gene such as those occurring in some cancers or the like. The ZNRF3 antigen binding site binds ZNRF3 and a variety of variants thereof, such as those expressed by some ZNRF3 positive tumor cells.

RNF43 is Ring Finger Protein 43. Alternative names for the gene or protein are Ring Finger Protein 43; RNF124; E3 Ubiquitin-Protein Ligase RNF43; RING Finger Protein 43; EC 6.3.2.; URCC.

A protein or antibody of the invention that binds RNF43, binds human RNF43. The RNF43 binding protein or antibody of the invention may, due to sequence and tertiary structure similarity between human and other mammalian orthologs, also bind such an ortholog but not necessarily so. Database accession numbers for the human RNF43 protein and the gene encoding it are (NC_000017.11; NT_010783.16; NC_018928.2; NP_001292473.1; NP_001292474.1; NP_060233.3). The accession numbers are primarily given to provide a further method of identification of RNF43 as a target, the actual sequence of the RNF43 protein bound may vary, for instance because of a mutation in the encoding gene such as those occurring in some cancers or the like. The RNF43 antigen binding site binds RNF43 and a variety of variants thereof, such as those expressed by some RNF43 positive tumor cells.

Insulin-like growth factor 1 (IGF-1) receptor binds insulin-like growth factor with a high affinity. The receptor has tyrosine kinase activity. The insulin-like growth factor I receptor plays a critical role in transformation events. Cleavage of the precursor generates alpha and beta subunits. It is overexpressed in most malignant tissues where it functions as an anti-apoptotic agent by enhancing cell survival. The protein is known under a number of different names such as IGF-I Receptor; EC 2.7.10.1; Insulin-Like Growth Factor I Receptor; Soluble IGF1R; Variant 1; Soluble IGF1R Variant 2; CD221 Antigen; EC 2.7.10; CD221; IGF1R; JTK13; and IGFR. External Ids for IGF1R are HGNC: 5465; Entrez Gene: 3480; Ensembl: ENSG00000140443; OMIM: 147370 and UniprotKB: P08069.

The invention further provides a method for inhibiting growth of a cell that expresses a membrane associated member of the epidermal growth factor (EGF) receptor family and that expresses a membrane associated member of the WNT pathway in a system permissive for growth of the cell, the method comprising providing the system with a protein of the invention or a bispecific antibody of the invention. The cell is preferably an EGF-receptor ligand responsive cell that expresses a membrane associated member of the WNT pathway. The system is preferably a culture system. The method preferably comprises culturing said cell in said system.

In the context of the present invention cell is said to express a membrane associated member of the WNT pathway if the cell comprises detectable RNA that codes for the membrane associated member of the WNT pathway. Expression can often also be detected by incubating the cell with an antibody that binds to the membrane associated member of the WNT pathway. However, some members are not expressed high enough for such an antibody test or for some members, there are no specific antibodies available. In such cases mRNA detection is preferred.

Where herein accession numbers or alternative names of proteins/genes are given, they are primarily given to provide a further method of identification of the mentioned protein as a target, the actual sequence of the target protein bound by an antibody of the invention may vary, for instance because of a mutation and/or alternative splicing in the encoding gene such as those occurring in some cancers or the like.

The invention also provides a method for the treatment of an individual that has a cancer, the method comprising administering a protein of the invention or a bispecific antibody of the invention to the individual in need thereof. The individual is preferably an individual that has cancer. The cancer is preferably an adenocarcinoma. Preferred cancers are Colorectal cancer; Pancreatic cancer; Lung cancer; Breast cancer; Liver cancer; Prostate cancer; Ovarian cancer; Cervical cancer; Endometrial cancer; Head and Neck cancer; Melanoma; Testis cancer; Urothelial cancer; Renal cancer; Stomach cancer; or Carcinoid cancer. In a preferred embodiment the cancer is Colorectal cancer; Pancreatic cancer; Lung cancer; Breast cancer; Liver cancer; Prostate cancer; Ovarian cancer; Cervical cancer; Endometrial cancer; Head and Neck cancer; or Melanoma. In a particularly preferred embodiment the cancer is Colorectal cancer; Pancreatic cancer; Lung cancer; Breast cancer; or Liver cancer. In a particularly preferred embodiment the cancer is a gastrointestinal cancer. In a preferred embodiment the cancer is Colorectal cancer.

The cancer is preferably, but not limited to, a cancer that exhibits a growth response when provided with an EGF receptor family member ligand. The protein of the invention is preferably a protein that binds to a EGF receptor family member for which the cancer exhibits a growth response. In a preferred embodiment the cancer exhibits a growth response in vitro in response to EGF family members in the culture medium. The in vitro is preferably a culture as detailed for organoids in the experimental section. The cancer is preferably a cancer that expresses the membrane associated member of the WNT pathway that the protein of the invention binds to.

Further provided is a cell system comprising a protein of the invention or a bispecific antibody of the invention, and a cell that expresses a membrane associated member of the epidermal growth factor (EGF) receptor family and that expresses a membrane associated member of the WNT pathway. The cell is preferably an EGF-receptor ligand responsive cell that expresses a membrane associated member of the WNT pathway.

The system or cell system as described herein is preferably an in vitro culture system. The system can be used to detect growth responses of the cell. In a preferred embodiment the EGF-receptor ligand is a ligand for a preferred member of the EGF receptor family as defined elsewhere herein. In a preferred embodiment the membrane associated member of the WNT pathway is a preferred membrane associated member of the WNT pathway as defined elsewhere herein.

A cancer or a cell that is EGF-receptor ligand responsive exhibits a growth (proliferation) response to an EGF receptor ligand. A preferred method for determining whether a cell or a cancer is EGF-receptor ligand responsive is to determine growth factor responsiveness of the cell in an organoid culture assay as described in the examples. One method is to seed the cell or cells of the cancer in the organoid cell system in the presence or absence of growth factors such as (EGF (5 ng/ml), or NRG (5 ng/ml)) and then culture for 5 days. The number of viable cells can subsequently be determined using the Cell Titer Glo cell viability assay (Promega, cat. nr. G7571). The luminescence readout using growth factor-stimulated cells can then be compared to that obtained using non-stimulated cells (absence of the growth factor(s).

The invention further provides a method for inhibiting growth of a cell that expresses a membrane associated member of the EGF receptor family and that expresses a membrane associated member of the WNT pathway in a system permissive for growth of the cell, the method comprising providing the system with a protein of the invention or a bispecific antibody of the invention. The cell is preferably an EGF-receptor ligand responsive cell that expresses a membrane associated member of the WNT pathway. The inhibition is preferably a decrease of at least 10% in cell number or a derivative measure for the number of cells such as tumor size, when compared to the number of cells resulting under otherwise similar conditions but for the presence of the protein or the bispecific antibody of the invention. The inhibition is preferably a decrease of at least 20%, 30%, 40%, 50%, 60%, 70%, 80% or 90% in the number of cells. The inhibition may also be a decrease of at least 10% in other parameters associated with tumour malignancy or dysplasia, such as the number of lumens per organoid, when compared to the number of lumens resulting under otherwise similar conditions but for the presence of the protein or the bispecific antibody of the invention. The inhibition is preferably a decrease of at least 20%, 30%, 40%, 50%, 60%, 70%, 80% or 90% in the number of lumens per organoid.

For the avoidance of doubt the reference to the growth of a cell as used herein refers to a change in the number of cells. Inhibition of growth refers to a reduction in the number of cells that would otherwise have been obtained. Increase in growth refers to an increase in the number of cells that would otherwise have been obtained. The growth of a cell typically refers to the proliferation of the cell.

The binding of the protein or bispecific antibody of the invention to a cell comprising said membrane associated member of the epidermal growth factor (EGF) receptor family or cMET; and/or said membrane associated member of a WNT signaling pathway on the membrane reduces the growth/proliferation of said cell in the presence of a growth factor for said epidermal growth factor (EGF) receptor family or cMET and/or Rspondin. The reduction is compared to the growth/proliferation of the same cell under otherwise identical conditions in the absence of the protein or bispecific antibody of the invention.

The protein or bispecific antibody of the invention, or functional part, derivative and/or analogue thereof is preferably selected from a protein or bispecific antibody of the invention, or functional part, derivative and/or analogue thereof that binds ErbB-1 and LGR4; ErbB-1 and LGR5; ErbB-1 and LGR6; ErbB-1 and ZNRF3; ErbB-1 and RNF43; ErbB-2 and LGR4; ErbB-2 and LGR5; ErbB-2 and LGR6; ErbB-2 and ZNRF3; ErbB-2 and RNF43; ErbB-3 and LGR4; ErbB-3 and LGR5; ErbB-3 and LGR6; ErbB-3 and ZNRF3; ErbB-3 and RNF43; ErbB-4 and LGR4; ErbB-4 and LGR5; ErbB-4 and LGR6; ErbB-4 and ZNRF3; and ErbB-4 and RNF43.

In a preferred embodiment it is selected from ErbB-1 and LGR4; ErbB-1 and LGR5; ErbB-1 and ZNRF3; ErbB-1 and RNF43; ErbB-3 and LGR4; ErbB-3 and LGR5; ErbB-3 and ZNRF3; ErbB-3 and RNF43; ErbB-4 and LGR4; ErbB-4 and LGR5; ErbB-4 and ZNRF3; and ErbB-4 and RNF43. In a preferred embodiment it is selected from ErbB-1 and LGR4; ErbB-1 and LGR5; ErbB-1 and ZNRF3; ErbB-1 and RNF43; ErbB-3 and LGR4; ErbB-3 and LGR5; ErbB-3 and ZNRF3; ErbB-3 and RNF43. Preferably it is selected from ErbB-1 and LGR5; ErbB-1 and ZNRF3; ErbB-1 and RNF43; ErbB-3 and LGR5; ErbB-3 and ZNRF3; and ErbB-3 and RNF43. In a particularly preferred embodiment the protein or bispecific antibody of the invention, or functional part, derivative and/or analogue thereof is selected from a protein or bispecific antibody of the invention, or functional part, derivative and/or analogue thereof that binds ErbB-1 and LGR5; and ErbB-3 and LGR5.

The antibody or bispecific antibody preferably comprises two variable domains wherein one variable domain binds an extracellular part of one protein and the other variable domain binds an extracellular part of the other protein.

In one embodiment the invention provides a bispecific antibody comprising a variable domain that binds ErbB-1, wherein a heavy chain variable region of said variable domain comprises at least the CDR3 sequence of an ErbB-1 specific heavy chain variable region selected from the group consisting of MF3370; MF3755; MF4280 or MF4289 as depicted in FIG. 1 or wherein a heavy chain variable region of said variable domain comprises a heavy chain CDR3 sequence that differs in at most three, preferably in at most two, preferably in no more than one amino acid from a CDR3 sequence of a VH selected from the group consisting of MF3370; MF3755; MF4280 or MF4289 as depicted in FIG. 1. Said variable domain preferably comprises a heavy chain variable region comprising at least the CDR3 sequence of MF3370; MF3755; MF4280 or MF4289 as depicted in FIG. 1.

Said variable domain preferably comprises a heavy chain variable region comprising at least the CDR1, CDR2 and CDR3 sequences of an ErbB-1 specific heavy chain variable region selected from the group consisting of MF3370; MF3755; MF4280 or MF4289 as depicted in FIG. 1, or heavy chain CDR1, CDR2 and CDR3 sequences that differ in at most three, preferably in at most two, preferably in at most one amino acid from the CDR1, CDR2 and CDR3 sequences of ErbB-1 specific heavy chain variable region selected from the group consisting of MF3370; MF3755; MF4280 or MF4289 as depicted in FIG. 1. Said variable domain preferably comprises a heavy chain variable region comprising at least the CDR1, CDR2 and CDR3 sequences of MF3370; MF3755; MF4280 or MF4289 as depicted in FIG. 1. A preferred heavy chain variable region is MF3755. Another preferred heavy chain variable region is MF4280.

It has been shown that the antibodies comprising one or more variable domains with a heavy chain variable region MF3755 have a better effectivity when used to inhibit growth of an EGFR ligand responsive cancer or cell. In the context of bispecific antibodies, an arm of the antibody comprising a variable domain with a heavy chain variable region MF3755 combines better with a variety of other arms comprising variable domains that bind extra-cellular parts of other cell surface proteins. Antibodies comprising a variable domain with heavy chain variable region MF4280 also work well with a variable domain that binds LGR4, LGR5, LGR6, ZNRF3 or RNF43.

In one embodiment the invention provides a bispecific antibody comprising a variable domain that binds ErbB-3, wherein a heavy chain variable region of said variable domain comprises at least the CDR3 sequence of an ErbB-3 specific heavy chain variable region selected from the group consisting of MF3125; MF3176; MF3178; or MF4863 as depicted in FIG. 1 or wherein a heavy chain variable region of said variable domain comprises a heavy chain CDR3 sequence that differs in at most three, preferably in at most two, preferably in no more than one amino acid from a CDR3 sequence of a VH selected from the group consisting of MF3125; MF3176; MF3178; or MF4863 as depicted in FIG. 1. Said variable domain preferably comprises a heavy chain variable region comprising at least the CDR3 sequence of MF3125; MF3176; MF3178; or MF4863 as depicted in FIG. 1.

Said variable domain preferably comprises a heavy chain variable region comprising at least the CDR1, CDR2 and CDR3 sequences of an ErbB-3 specific heavy chain variable region selected from the group consisting of MF3125; MF3176; MF3178; or MF4863 as depicted in FIG. 1, or heavy chain CDR1, CDR2 and CDR3 sequences that differ in at most three, preferably in at most two, preferably in at most one amino acid from the CDR1, CDR2 and CDR3 sequences of ErbB-3 specific heavy chain variable region selected from the group consisting of MF3125; MF3176; MF3178; or MF4863 as depicted in FIG. 1. Variable domain preferably comprises a heavy chain variable region comprising at least the CDR1, CDR2 and CDR3 sequences of MF3125; MF3176; MF3178; or MF4863 as depicted in FIG. 1. A preferred heavy chain variable region is MF3178.

In one embodiment the invention provides a bispecific antibody comprising a variable domain that binds LGR4, wherein a heavy chain variable region of said variable domain comprises at least the CDR3 sequence of an LGR4 specific heavy chain variable region selected from the group consisting of MF5777; or MF5781 as depicted in FIG. 1 or wherein a heavy chain variable region of said variable domain comprises a heavy chain CDR3 sequence that differs in at most three, preferably in at most two, preferably in no more than one amino acid from a CDR3 sequence of a VH selected from the group consisting of MF5777; or MF5781 as depicted in FIG. 1. Said variable domain preferably comprises a heavy chain variable region comprising at least the CDR3 sequence of MF5777; or MF5781 as depicted in FIG. 1.

Said variable domain preferably comprises a heavy chain variable region comprising at least the CDR1, CDR2 and CDR3 sequences of an LGR4 specific heavy chain variable region selected from the group consisting of MF5777; or MF5781 as depicted in FIG. 1, or heavy chain CDR1, CDR2 and CDR3 sequences that differ in at most three, preferably in at most two, preferably in at most one amino acid from the CDR1, CDR2 and CDR3 sequences of LGR4 specific heavy chain variable region selected from the group consisting of MF5777; or MF5781 as depicted in FIG. 1. Said variable domain preferably comprises a heavy chain variable region comprising at least the CDR1, CDR2 and CDR3 sequences of MF5777; or MF5781 as depicted in FIG. 1. A preferred heavy chain variable region is MF5781.

In one embodiment the invention provides a bispecific antibody comprising a variable domain that binds LGR5, wherein a heavy chain variable region of said bispecific antibody comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 1 or wherein a heavy chain variable region of variable domain comprises a heavy chain CDR3 sequence that differs in at most three, preferably in at most two, preferably in no more than one amino acid from a CDR3 sequence of a VH selected from the group consisting of MF5790; MF5803; MF5805; MF5808; MF5809; MF5814; MF5816; MF5817; or MF5818 as depicted in FIG. 1. Said variable domain preferably comprises a heavy chain variable region comprising at least the CDR3 sequence of MF5790; MF5803; MF 5805; MF5808; MF5809; MF5814; MF5816; MF5817; or MF5818 as depicted in FIG. 1.

Said variable domain preferably comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 1, or heavy chain CDR1, CDR2 and CDR3 sequences that differ in at most three, preferably in at most two, preferably in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 1. Said variable domain preferably comprises a heavy chain variable region comprising at least the CDR1, CDR2 and CDR3 sequences of MF5790; MF5803; MF5805; MF5808; MF5809; MF5814; MF5816; MF5817; or MF5818 as depicted in FIG. 1. Preferred heavy chain variable regions are MF5790; MF5803; MF5814; MF5816; MF5817; or MF5818. Particularly preferred heavy chain variable regions are MF5790; MF5814; MF5816; and MF5818; preferably MF5814, MF5818 and MF5816, heavy chain variable region MF5816 is particularly preferred. Another preferred heavy chain variable region is MF5818.

In one embodiment the invention provides a bispecific antibody comprising a variable domain that binds RNF43, wherein a heavy chain variable region of said variable domain comprises at least the CDR3 sequence of an RNF43 specific heavy chain variable region selected from the group consisting of MF5832; MF5836; or MF5839 as depicted in FIG. 1 or wherein a heavy chain variable region of said variable domain comprises a heavy chain variable region comprising a heavy chain CDR3 sequence that differs in at most three, preferably in at most two, preferably in no more than one amino acid from a CDR3 sequence of a VH selected from the group consisting of MF5832; MF5836; or MF5839 as depicted in FIG. 1. Said variable domain preferably comprises a heavy chain variable region comprising at least the CDR3 sequence of MF5832; MF5836; or MF5839 as depicted in FIG. 1.

Said variable domain preferably comprises a heavy chain variable region comprising at least the CDR1, CDR2 and CDR3 sequences of an RNF43 specific heavy chain variable region selected from the group consisting of MF5832; MF5836; or MF5839 as depicted in FIG. 1, or heavy chain CDR1, CDR2 and CDR3 sequences that differ in at most three, preferably in at most two, preferably in at most one amino acid from the CDR1, CDR2 and CDR3 sequences of RNF43 specific heavy chain variable region selected from the group consisting of MF5832; MF5836; or MF5839 as depicted in FIG. 1. Said variable domain preferably comprises a heavy chain variable region comprising at least the CDR1, CDR2 and CDR3 sequences of MF5832; MF5836; or MF5839 as depicted in FIG. 1. Preferred heavy chains are MF5832; or MF5836. A preferred heavy chain variable region is MF5836.

In one embodiment the invention provides a bispecific antibody comprising a variable domain that binds ZNRF3, wherein a heavy chain variable region of said variable domain comprises at least the CDR3 sequence of an ZNRF3 specific heavy chain variable region selected from the group consisting of MF5850; MF5853; MF5855; MF5862; MF5882; MF5884; MF5887; or MF5888 as depicted in FIG. 1 or wherein a heavy chain variable region of said variable domain comprises a heavy chain CDR3 sequence that differs in at most three, preferably in at most two, preferably in no more than one amino acid from a CDR3 sequence of a VH selected from the group consisting of MF5850; MF5853; MF5855; MF5862; MF5882; MF5884; MF5887; or MF5888 as depicted in FIG. 1. Said variable domain preferably comprises a heavy chain variable region comprising at least the CDR3 sequence of MF5850; MF5853; MF5855; MF5862; MF5882; MF5884; MF5887; or MF5888 as depicted in FIG. 1.

Said variable domain preferably comprises a heavy chain variable region comprising at least the CDR1, CDR2 and CDR3 sequences of an ZNRF3 specific heavy chain variable region selected from the group consisting of MF5850; MF5853; MF5855; MF5862; MF5882; MF5884; MF5887; or MF5888 as depicted in FIG. 1, or heavy chain CDR1, CDR2 and CDR3 sequences that differ in at most three, preferably in at most two, preferably in at most one amino acid from the CDR1, CDR2 and CDR3 sequences of ZNRF3 specific heavy chain variable region selected from the group consisting of MF5850; MF5853; MF5855; MF5862; MF5882; MF5884; MF5887; or MF5888 as depicted in FIG. 1. Said variable domain preferably comprises a heavy chain variable region comprising at least the CDR1, CDR2 and CDR3 sequences of MF5850; MF5853; MF5855; MF5862; MF5882; MF5884; MF5887; or MF5888 as depicted in FIG. 1. Preferred heavy chain variable regions are MF5850; MF5853; MF5855; MF5884; or MF5888. Preferred heavy chain variable regions are MF5888 and MF5850, preferably MF5850.

CDR sequences are for instance varied for optimization purposes, preferably in order to improve binding strength or the stability of the antibody. Optimization is for instance performed by mutagenesis procedures where after the stability and/or binding affinity of the resulting antibodies are preferably tested and an improved EGFR or ErbB 3-specific CDR sequence is preferably selected. A skilled person is well capable of generating antibody variants comprising at least one altered CDR sequence according to the invention. For instance, conservative amino acid substitution may be applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

The bispecific antibody of the invention that comprises a variable domain that binds EGFR, preferably comprises the VH chain of said variable domain comprises the amino acid sequence of VH chain MF3370; MF3755; MF4280 or MF4289 as depicted in FIG. 1; or the amino acid sequence of VH chain MF3370; MF3755; MF4280 or MF4289 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1. The bispecific antibody of the invention that comprises a variable domain that binds EGFR, preferably comprises the VH chain of said variable domain and preferably comprises the amino acid sequence of VH chain MF3755. In a preferred embodiment this bispecific antibody comprises a variable domain that binds a membrane associated member of the WNT pathway as defined elsewhere herein. In a preferred embodiment the membrane associated member of the WNT pathway is LGR4, LGR5, RNF43 or ZNRF3.

The bispecific antibody of the invention that comprises a variable domain that binds HER3, preferably comprises the VH chain of said variable domain comprises the amino acid sequence of VH chain MF3125; MF3176; MF3178; or MF4863 as depicted in FIG. 1; or the amino acid sequence of VH chain MF3125; MF3176; MF3178; or MF4863 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1. A preferred heavy chain is MF3178. In a preferred embodiment this bispecific antibody comprises a variable domain that binds a membrane associated member of the WNT pathway as defined elsewhere herein. In a preferred embodiment the membrane associated member of the WNT pathway is LGR4, LGR5, RNF43 or ZNRF3.

The bispecific antibody of the invention that comprises a variable domain that binds LGR4, preferably comprises the VH chain of said variable domain comprises the amino acid sequence of VH chain MF5777; or MF5781 as depicted in FIG. 1; or the amino acid sequence of VH chain MF5777; or MF5781 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1. In a preferred embodiment this bispecific antibody comprises a variable domain that binds an extracellular part of membrane associated member of the EGFR family as defined elsewhere herein. In a preferred embodiment the membrane associated member of the EGFR family is EGFR or HER3, preferably EGFR. A preferred heavy chain is MF3755.

The bispecific antibody of the invention that comprises a variable domain that binds LGR5, preferably comprises the VH chain of said variable domain comprises the amino acid sequence of VH chain MF5790; MF5803; MF5805; MF5808; MF5809; MF5814; MF5816; MF5817; or MF5818 as depicted in FIG. 1; or the amino acid sequence of VH chain MF5790; MF5803; MF5805; MF5808; MF5809; MF5814; MF5816; MF5817; or MF5818 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1. In a preferred embodiment this bispecific antibody comprises a variable domain that binds an extracellular part of membrane associated member of the EGFR family as defined elsewhere herein. In a preferred embodiment the membrane associated member of the EGFR family is EGFR or HER3, preferably EGFR. A preferred heavy chain is MF3755.

The bispecific antibody of the invention that comprises a variable domain that binds RNF43, preferably comprises the VH chain of said variable domain comprises the amino acid sequence of VH chain MF5832; MF5836; or MF5839 as depicted in FIG. 1; or the amino acid sequence of VH chain MF5832; MF5836; or MF5839 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1. In a preferred embodiment the bispecific antibody comprises a variable domain that binds an extracellular part of membrane associated member of the EGFR family as defined elsewhere herein. In a preferred embodiment the membrane associated member of the EGFR family is EGFR or HER3, preferably EGFR. A preferred heavy chain is MF3755.

The bispecific antibody of the invention that comprises a variable domain that binds ZNRF3, preferably comprises the VH chain of said variable domain comprises the amino acid sequence of VH chain MF5850; MF5853; MF5855; MF5862; MF5882; MF5884; MF5887; or MF5888 as depicted in FIG. 1; or the amino acid sequence of VH chain MF5850; MF5853; MF5855; MF5862; MF5882; MF5884; MF5887; or MF5888 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 1. In a preferred embodiment the bispecific antibody comprises a variable domain that binds an extracellular part of membrane associated member of the EGFR family as defined elsewhere herein. In a preferred embodiment the membrane associated member of the EGFR family is EGFR or HER3, Preferably EGFR. A preferred heavy chain is MF3755.

Preferably, the mentioned amino acid insertions, deletions and substitutions in a VH or VL as specified herein are not present in the CDR3 region. The mentioned amino acid insertions, deletions and substitutions are also preferably not present in the CDR1 and CDR2 regions. The mentioned amino acid insertions, deletions and substitutions are also preferably not present in the FR4 region.

The invention also provides an antibody comprising a variable domain that binds EGFR and a variable domain that binds LGR4 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR4 comprises
the amino acid sequence of VH chain MF5777 as depicted in FIG. 1; or
the amino acid sequence of VH chain MF5777 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR4 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR4 comprises
the amino acid sequence of VH chain MF5781 as depicted in FIG. 1; or
the amino acid sequence of VH chain MF5781 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR5 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5790 as depicted in FIG. 1; or
the amino acid sequence of VH chain MF5790 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR5 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5803 as depicted in FIG. 1; or
the amino acid sequence of VH chain MF5803 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR5 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5814 as depicted in FIG. 1; or
the amino acid sequence of VH chain MF5814 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR5 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5816 as depicted in FIG. 1; or
the amino acid sequence of VH chain MF5816 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR5 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5817 as depicted in FIG. 1; or
the amino acid sequence of VH chain MF5817 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds LGR5 wherein the VH chain of the variable domain that binds EGFR comprises
the amino acid sequence of VH chain MF3755 as depicted in FIG. 1 or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5818 as depicted in FIG. 1; or
the amino acid sequence of VH chain MF5818 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds RNF43 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds RNF43 comprises
the amino acid sequence of VH chain MF5832 as depicted in FIG. 1; or
the amino acid sequence of VH chain MF5832 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds RNF43 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds RNF43 comprises
the amino acid sequence of VH chain MF5836 as depicted in FIG. 1; or
the amino acid sequence of VH chain MF5836 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds ZNRF3 wherein the VH chain of the variable domain that binds EGFR comprises
the amino acid sequence of VH chain MF3755 as depicted in FIG. 1 or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5850 as depicted in FIG. 1; or
the amino acid sequence of VH chain MF5850 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds ZNRF3 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5853 as depicted in FIG. 1; or
the amino acid sequence of VH chain MF5853 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds ZNRF3 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5855 as depicted in FIG. 1; or
the amino acid sequence of VH chain MF5855 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds ZNRF3 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5884 as depicted in FIG. 1; or
the amino acid sequence of VH chain MF5884 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds EGFR and a variable domain that binds ZNRF3 wherein the VH chain of the variable domain that binds EGFR comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5888 as depicted in FIG. 1; or
the amino acid sequence of VH chain MF5888 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR4 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR4 comprises
the amino acid sequence of VH chain MF5777 as depicted in FIG. 1; or
the amino acid sequence of VH chain MF5777 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR4 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR4 comprises
the amino acid sequence of VH chain MF5781 as depicted in FIG. 1; or
the amino acid sequence of VH chain MF5781 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR5 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5790 as depicted in FIG. 1; or
the amino acid sequence of VH chain MF5790 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR5 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5803 as depicted in FIG. 1; or
the amino acid sequence of VH chain MF5803 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR5 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5814 as depicted in FIG. 1; or
the amino acid sequence of VH chain MF5814 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR5 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5816 as depicted in FIG. 1; or
the amino acid sequence of VH chain MF5816 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR5 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5817 as depicted in FIG. 1; or
the amino acid sequence of VH chain MF5817 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds LGR5 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds LGR5 comprises
the amino acid sequence of VH chain MF5818 as depicted in FIG. 1; or
the amino acid sequence of VH chain MF5818 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds RNF43 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds RNF43 comprises
the amino acid sequence of VH chain MF5836 as depicted in FIG. 1; or
the amino acid sequence of VH chain MF5836 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds ZNRF3 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5850 as depicted in FIG. 1; or
the amino acid sequence of VH chain MF5850 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds ZNRF3 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5853 as depicted in FIG. 1; or
the amino acid sequence of VH chain MF5853 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds ZNRF3 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5855 as depicted in FIG. 1; or
the amino acid sequence of VH chain MF5855 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds ZNRF3 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5884 as depicted in FIG. 1; or
the amino acid sequence of VH chain MF5884 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides an antibody comprising a variable domain that binds HER3 and a variable domain that binds ZNRF3 wherein the VH chain of the variable domain that binds HER3 comprises
the amino acid sequence of VH chain MF3178 as depicted in FIG. 1; or
the amino acid sequence of VH chain MF3178 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and

wherein the VH chain of the variable domain that binds ZNRF3 comprises
the amino acid sequence of VH chain MF5888 as depicted in FIG. 1; or
the amino acid sequence of VH chain MF5888 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

A protein or bispecific antibody of the present invention is preferably used in humans. To this end an antibody of the invention is preferably a human or humanized antibody. Tolerance of a human to a polypeptide is governed by many different aspects. Immunity, be it T-cell mediated, B-cell mediated or other is one of the variables that are encompassed in tolerance of the human for a polypeptide. The constant region of a bispecific antibody of the present invention is preferably a human constant region (FIG. 4). The constant region may contain one or more, preferably not more than 10, preferably not more than 5 amino-acid differences with the constant region of a naturally occurring human antibody. It is preferred that the constant part is entirely derived from a naturally occurring human antibody. Various antibodies produced herein are derived from common light chain mice immunized with the respective target as described in WO2009/157771. Various antibodies produced herein are derived from a human antibody variable domain library. As such these variable domains are human. The unique CDR regions may be derived from humans, be synthetic or derived from another organism. The variable region is considered a human variable region when it has an amino acid sequence that is identical to an amino acid sequence of the variable region of a naturally occurring human antibody, but for the CDR regions. The variable region of a VH of antibody that binds a EGFR-family member or membrane associated member of the WNT pathway, or a light chain in an antibody of the invention may contain one or more, preferably not more than 10, preferably not more than 5 amino-acid differences with the variable region of a naturally occurring human antibody, not counting possible differences in the amino acid sequence of the CDR regions. Such mutations also occur in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; de-immunization of the murine heavy chain variable region, preferably done by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

De-immunized murine heavy chain variable regions are less immunogenic in humans than the original murine heavy chain variable region. Preferably a variable region or domain of the invention is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the invention is preferably a mammal, more preferably a primate, most preferably a human.

A protein or bispecific antibody according to the invention preferably comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. In a preferred embodiment the invention provides an antibody according to the invention wherein said constant region is selected from the group of IgG, IgA, IgM, IgD, and IgE constant regions, more preferably said constant region comprises an IgG constant region, more preferably an IgG1 constant region (FIG. 4), preferably a mutated IgG1 constant region. Some variation in the constant region of IgG1 occurs in nature and/or is allowed without changing the immunological properties of the resulting antibody. Typically between about 1-10 amino acid insertions, deletions, substitutions or a combination thereof are allowed in the constant region.

Rational methods have evolved toward minimizing the content of non-human residues in the human context. Various methods are available to successfully graft the antigen-binding property of an antibody onto another antibody. The binding properties of antibodies rest predominantly in the exact sequence of the CDR3 region, often supported by the sequence of the CDR1 and CDR2 regions in the variable domain combined with the appropriate structure of the variable domain as a whole. Various methods are presently available to graft CDR regions onto a suitable variable domain of another antibody. Some of these methods are reviewed in J. C. Almagrol and J. Fransson (2008) Frontiers in Bioscience 13, 1619-1633, which is included by reference herein.

The mentioned at most 15, preferably 1, 2, 3, 4, 5, 6, 7, 8, 9 or 10 and preferably 1, 2, 3, 4 or 5 amino acid substitutions are preferably conservative amino acid substitutions, the insertions, deletions, substitutions or a combination thereof are preferably not in the CDR3 region of the VH chain, preferably not in the CDR1, CDR2 or CDR3 region of the VH chain and preferably not in the FR4 region.

The light chain of a variable domain comprising a variable heavy chain sequence as depicted in FIG. 3, is preferably a germline light chain of or based on O12, preferably the rearranged germline human kappa light chain IgVκ1-39*01/IGJκ1*01 or a fragment or a functional derivative thereof (nomenclature according to the IMGT database worldwide web at imgt.org). The terms rearranged germline human kappa light chain IgVκ1-39*01/IGJκ1*01, IGKV1-39/IGKJ1, huVκ1-39 light chain or in short huVκ1-39 are used. The light chain can have 1, 2, 3, 4 or 5 amino acid insertions, deletions, substitutions or combination thereof. The mentioned 1, 2, 3, 4 or 5 amino acid substitutions are preferably conservative amino acid substitutions, the insertions, deletions, substitutions or combination thereof are preferably not in the CDR3 region of the VL chain, preferably not in the CDR1, CDR2 or CDR3 region or FR4 region of the VL chain.

Various methods are available to produce bispecific antibodies. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection. The ratio of bispecific to other antibodies that are produced by the cell can be increased in various ways. In a preferred embodiment of the invention, the ratio is increased by expressing not two different light chains but two essentially identical light chains in the cell. This concept is in the art also referred to as the “common light chain” method. When the essentially identical light chains work together with the two different heavy chains allowing the formation of variable domains with different antigen-binding sites and concomitant different binding properties, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. The art describes various ways in which such hetero-dimerization of heavy chains can be achieved. One way is to generate ‘knob into hole’ bispecific antibodies. See US Patent Application 20030078385 (Arathoon et al.—Genentech). Another method is by using charge engineering as described in Gunasekaran (JBC 2010, vol 285, pp 19637-19646). Another and preferred method is described in U.S. provisional application 61/635,935, which has been followed up by US regular application Ser. No. 13/866,747 and PCT application No. PCT/NL2013/050294 (WO 2013/157954 A1), which are incorporated herein by reference. Methods and means are disclosed for producing bispecific antibodies (from a single cell), whereby means are provided that favor the formation of bispecific antibodies over the formation of monospecific antibodies. These methods can also be favorably employed in the present invention. Thus the invention provides a method for producing a bispecific antibody according to the invention (from a single cell), wherein said bispecific antibody comprises two CH3 domains that are capable of forming an interface, said method comprising providing in said cell a) a first nucleic acid molecule encoding a 1st CH3 domain comprising heavy chain, b) a second nucleic acid molecule encoding a 2nd CH3 domain comprising heavy chain, wherein said nucleic acid molecules are provided with means for preferential pairing of said 1st and 2nd CH3 domain comprising heavy chains, said method further comprising the step of culturing said host cell and allowing for expression of said two nucleic acid molecules and harvesting said bispecific antibody from the culture. Said first and second nucleic acid molecules may be part of the same nucleic acid molecule, vector or gene delivery vehicle and may be integrated at the same site of the host cell's genome. Alternatively, said first and second nucleic acid molecules are separately provided to said cell.

A preferred embodiment provides a method for producing a bispecific antibody according to the invention from a single cell, wherein said bispecific antibody comprises two CH3 domains that are capable of forming an interface, said method comprising providing:
a cell having a) a first nucleic acid molecule encoding a heavy chain comprising an antigen binding site that binds an extra-cellular part of a membrane associated member of the EGFR family and that contains a 1st CH3 domain, and b) a second nucleic acid molecule encoding a heavy chain comprising an antigen-binding site that binds an extra-cellular part of a membrane associated member of a WNT signaling pathway and that contains a 2nd CH3 domain, wherein said nucleic acid molecules are provided with means for preferential pairing of said 1st and 2nd CH3 domains,
   said method further comprising the step of culturing said cell and allowing for expression of the proteins encoded by said two nucleic acid molecules and harvesting said bispecific IgG antibody from the culture. In a particularly preferred embodiment, said cell also has a third nucleic acid molecule encoding a common light chain. Said first, second and third nucleic acid molecule may be part of the same nucleic acid molecule, vector or gene delivery vehicle and may be integrated at the same site of the host cell's genome. Alternatively, said first, second and third nucleic acid molecules are separately provided to said cell. A preferred common light chain is based on O12, preferably it is the rearranged germline human kappa light chain IgVκ1 39*01/IGJκ1*01, as described above. Means for preferential pairing of said 1st and said 2nd CH3 domain are preferably the corresponding mutations in the CH3 domain of the heavy chain coding regions. The preferred mutations to produce essentially only bispecific antibodies are the amino acid substitutions L351K and T366K (numbering according to EU numbering) in the first CH3 domain and the amino acid substitutions L351D and L368E in the second CH3 domain, or vice versa. Further provided is therefore a method according to the invention for producing a bispecific antibody, wherein said first CH3 domain comprises the amino acid substitutions L351K and T366K (numbering according to EU numbering) and wherein said second CH3 domain comprises the amino acid substitutions L351D and L368E, said method further comprising the step of culturing said cell and allowing for expression of proteins encoded by said nucleic acid molecules and harvesting said bispecific antibody from the culture. Also provided is a method according to the invention for producing a bispecific antibody, wherein said first CH3 domain comprises the amino acid substitutions L351D and L368E (numbering according to EU numbering) and wherein said second CH3 domain comprises the amino acid substitutions L351K and T366K, said method further comprising the step of culturing said cell and allowing for expression of said nucleic acid molecules and harvesting said bispecific antibody from the culture. Antibodies that can be produced by these methods are also part of the present invention. The CH3 hetero-dimerization domains are preferably IgG1 hetero-dimerization domains. The heavy chain constant regions comprising the CH3 hetero-dimerization domains are preferably IgG1 constant regions.

In one embodiment the invention provides a nucleic acid molecule encoding an antibody heavy chain variable region according to the invention. The nucleic acid molecule (typically an in vitro, isolated or recombinant nucleic acid molecule) preferably encodes a heavy chain variable region as depicted in FIG. 1 or a heavy chain variable region as depicted in FIG. 1 having 1, 2, 3, 4 or 5 amino acid insertions, deletions, substitutions or combination thereof. In a preferred embodiment the nucleic acid molecule comprises a sequence as depicted in FIG. 2. In another preferred embodiment the nucleic acid molecule encodes the same amino acid sequence as the nucleic acid depicted in FIG. 2 but has a different sequence because it encodes one or more different codons. The invention further provides a nucleic acid sequence encoding a heavy chain of FIG. 1.

A nucleic acid molecule as used in the invention is typically but not exclusively a ribonucleic acid (RNA) or a deoxyribonucleic acid (DNA). Alternative nucleic acids are available for a person skilled in the art. A nucleic acid according to the invention is for instance comprised in a cell. When said nucleic acid is expressed in said cell, said cell can produce an antibody according to the invention. Therefore, the invention in one embodiment provides a cell comprising an antibody according to the invention and/or a nucleic acid according to the invention. Said cell is preferably an animal cell, more preferably a mammal cell, more preferably a primate cell, most preferably a human cell. For the purposes of the invention a suitable cell is any cell capable of comprising and preferably of producing an antibody according to the invention and/or a nucleic acid according to the invention.

The invention further provides a cell comprising an antibody according to the invention. Preferably said cell (typically an in vitro, isolated or recombinant cell) produces said antibody. In a preferred embodiment said cell is a hybridoma cell, a Chinese hamster ovary (CHO) cell, an NSO cell or a PER-C6TM cell. In a particularly preferred embodiment said cell is a CHO cell. Further provided is a cell culture comprising a cell according to the invention. Various institutions and companies have developed cell lines for the large scale production of antibodies, for instance for clinical use. Non-limiting examples of such cell lines are CHO cells, NSO cells or PER.C6 cells. These cells are also used for other purposes such as the production of proteins. Cell lines developed for industrial scale production of proteins and antibodies are herein further referred to as industrial cell lines. Thus in a preferred embodiment the invention provides the use of a cell line developed for the large scale production of antibody for the production of an antibody of the invention. The invention further provides a cell for producing an antibody comprising a nucleic acid molecule that codes for a VH, a VL, and/or a heavy chain as depicted in FIG. 1. Preferably said nucleic acid molecule comprises a sequence as depicted in FIG. 2.

The invention further provides a method for producing an antibody comprising culturing a cell of the invention and harvesting said antibody from said culture. Preferably said cell is cultured in a serum free medium. Preferably said cell is adapted for suspension growth. Further provided is an antibody obtainable by a method for producing an antibody according to the invention. The antibody is preferably purified from the medium of the culture. Preferably said antibody is affinity purified.

A cell of the invention is for instance a hybridoma cell line, a CHO cell, a 293F cell, an NSO cell or another cell type known for its suitability for antibody production for clinical purposes. In a particularly preferred embodiment said cell is a human cell. Preferably a cell that is transformed by an adenovirus E1 region or a functional equivalent thereof. A preferred example of such a cell line is the PER.C6 cell line or equivalent thereof. In a particularly preferred embodiment said cell is a CHO cell or a variant thereof. Preferably a variant that makes use of a Glutamine synthetase (GS) vector system for expression of an antibody.

The invention further provides a pharmaceutical composition comprising an antibody according to the invention. The pharmaceutical composition preferably comprises a preferably pharmaceutically acceptable excipient or carrier. In a preferred embodiment the pharmaceutical composition comprises 5-50 mM Histidine, 100-300 mM Trehalose, 0.1-03 g/L PolySorbate20 or a combination thereof. The pH is preferably set at pH=5.5-6.5. In a preferred embodiment the pharmaceutical composition comprises 25 mM Histidine, 220 mM Trehalose, 0.2 g/L PolySorbate20 or a combination thereof. The pH is preferably set at pH=5.5-6.5, most preferably at pH=6.

An antibody of the invention preferably further comprises a label, preferably a label for in vivo imaging. Such a label is typically not necessary for therapeutic applications. In for instance a diagnostic setting, a label can be helpful. For instance in visualizing target cells in the body. Various labels are suited and many are well known in the art. In a preferred embodiment the label is a radioactive label for detection. In another preferred embodiment, the label is an infrared label. Preferably the infrared label is suited for in vivo imaging. Various infrared labels are available to the person skilled in the art. Preferred infrared labels are for instance, IRDye 800; IRDye 680RD; IRDye 680LT; IRDye 750; IRDye 700DX; IRDye 800RS IRDye 650; IRDye 700 phosphoramidite; IRDye 800 phosphoramidite (LI-COR USA; 4647 Superior Street; Lincoln, Nebraska).

To establish whether a cancer exhibits a growth response when provided with an EGF receptor family member ligand, the skilled person can for instance determine the EGFR amplification and/or staining immune-histochemistry (for an EGF-response). At least 10% of the tumor cells in a tumor sample should be positive. The tumor sample can also contain 20%, 30% 40% 50% 60% 70% or more positive cells. To establish whether a cancer exhibits a growth response to a HER3 ligand the skilled person can for instance determine the HER3 amplification and/or staining in immunohistochemistry. At least 10% tumor cells in a tumor sample should be positive. The sample can also contain 20%, 30% 40% 50% 60% 70% or more positive cells. To establish whether a cancer will be sensitive to Wnt (LGR5, LGR4, ZNRF3, RNF43) receptor targeting the skilled person can for instance determine the Wnt target amplification and/or staining in immunohistochemistry. At least 1% tumor cells in a tumor sample should be positive. The sample can also contain 5%, 10% 20% 30% 40% 50% or more positive cells.

The amount of antibody according to the invention to be administered to a patient is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. An antibody according to the invention exerting sufficient therapeutic effects at low dosage is, therefore, preferred. The dosage can be in range of the dosing regimen of cetuximab. The dosage can also be lower.

A bispecific antibody according to the invention preferably induces less skin toxicity as compared to cetuximab under otherwise similar conditions of course. A bispecific antibody according to the invention preferably produces less proinflammatory chemokines, preferably of CXCL14 as compared to cetuximab under otherwise similar conditions of course. A bispecific antibody according to the invention preferably induces less impairment of antimicrobial RNAses, preferably Rnase 7, as compared to cetuximab under otherwise similar conditions of course.

The present invention describes among others antibodies that target the an extra-cellular part of a membrane associated member of the EGF receptor family and an extra-cellular part of a membrane associated member of the WNT signaling pathway and result in potent proliferation inhibition of cancer cell lines in vitro and tumor growth inhibition in vivo. The antibodies were produced as bispecific antibodies by cloning them into complementary expression vectors that contain mutations in the CH3 region that drives hetero-dimerization of heavy chains. Many bispecific antibodies were produced at small scale and tested in binding and functional assays on cancer cell lines. An antibody of the invention, particularly a bispecific antibody of the invention can combine low toxicity profiles with high efficacy. An antibody of the invention can be useful in various types and lines of EGFR family member targeted therapies. An antibody of the invention can have an increased therapeutic window when compared to an antibody that binds the same antigen(s) with both arms. A bispecific antibody of the invention can exhibit better growth inhibitory effects in vitro, in vivo or a combination thereof when compared to the MEHD7945A antibody.

Also provided is a method for counteracting the formation of a metastasis in a subject having a cancer that expresses a member of the EGF-receptor family and that expresses a membrane associated member of the WNT pathway. The cancer is preferably an EGF-receptor ligand responsive cancer. High Her ligand (e.g. EGF, Heregulin) levels are typically present during the formation of metastases (i.e. the migration, invasion, growth and/or differentiation of tumor cells or tumor initiating cells). Typically, tumor initiating cells are identified based on stem cell markers such as CD44. These processes can therefore barely be counteracted with currently known therapies like trastuzumab and pertuzumab. Since an antibody according to the invention is capable of counteracting growth and/or differentiation of tumor cells or tumor initiating cells such as cancer stem cells, such antibody according to the invention is also particularly suitable for counteracting the formation of metastases. Further provided is therefore a method for counteracting the formation of a metastasis in a subject having a EGFR, ErbB-3 or EGFR/ErbB-3 positive tumor, wherein said EGFR, ErbB-3 or EGFR/ErbB-3 positive tumor cell and/or surrounding stromal tissue cells (e.g. fibroblasts, Leukocytes such as macrophages and monocytes, endothelium, etc.) has a Her ligand expression level that is at least 60%, preferably at least 70%, more preferably at least 80%, more preferably at least 85%, more preferably at least 90% or 95% of the her ligand expression level of BXPC3 or MCF7 cells, comprising administering to the subject a bispecific antibody or a functional part, derivative and/or analogue thereof that binds an extra-cellular part of EGFR or HER3, an extra-cellular part of a membrane associated member of the WNT signaling pathway. Also provided is a bispecific antibody or a functional part, derivative and/or analogue thereof that binds an extra-cellular part of EGFR or HER3, and an extra-cellular part of a membrane associated member of the WNT signaling pathway, for use in the treatment or prevention of the formation of metastases. The tumor from which said metastases originate is preferably an ErbB-3 or EGFR/ErbB-3 positive tumor. The tumor and/or surrounding stromal tissue cells preferably has a her ligand expression level that is at least 60%, preferably at least 70%, more preferably at least 80%, more preferably at least 85%, more preferably at least 90% or 95% of the heregulin expression level of BXPC3 or MCF7 cells.

Further provided is a use of a bispecific antibody according to the invention or a functional part, derivative and/or analogue thereof, for the preparation of a medicament for the treatment or prevention of the formation of metastases. The tumor from which said metastases originate is preferably an ErbB-3 or EGFR/ErbB-3 positive tumor. The tumor and/or surrounding stromal tissue cells preferably has a her ligand expression level that is at least 60%, preferably at least 70%, more preferably at least 80%, more preferably at least 85%, more preferably at least 90% or 95% of the heregulin expression level of BXPC3 or MCF7 cells.

Antibodies of the invention can be produced at levels >50 mg/L after transient transfection in suspension 293F cells. The bispecific antibodies can be purified to greater than 98% purity with yields >70%. Analytical characterization studies show bispecific lgG1 antibody profiles that are comparable to bivalent monospecific lgG1.

For the purpose of clarity and a concise description features are described herein as part of the same or separate embodiments, however, it will be appreciated that the scope of the invention may include embodiments having combinations of all or some of the features described.

In mathematics, the Euclidean distance or Euclidean metric is the “ordinary” (i.e. straight-line) distance between two points in Euclidean space. With this distance, Euclidean space becomes a metric space. The associated norm is called the Euclidean norm.

Receptor tyrosine kinases (RTK)s are the high-affinity cell surface receptors for many polypeptide growth factors, cytokines, and hormones. Of the presently known 90 unique tyrosine kinase genes identified in the human genome, 58 encode receptor tyrosine kinase proteins. Receptor tyrosine kinases have been shown to be relevant for normal cellular processes but also to have a role in the development and progression of cancer. Receptor tyrosine kinases contain at least one extracellular domain.

In one aspect the invention provides a protein that binds an extracellular part of a membrane associated member of the RTK receptor family and an extracellular part of a membrane associated member of a WNT signaling pathway. The protein is preferably an antibody, preferably a bispecific antibody or a functional part, derivative and/or analogue thereof.

The invention also provides a bispecific antibody or a functional part, derivative and/or analogue thereof that binds an extracellular part of a membrane associated member of the RTK receptor family and an extra-cellular part of a membrane associated member of a WNT-signaling pathway.

Also provided is a method for the treatment of an individual that has a cancer, the method comprising administering a protein of the invention or a bispecific antibody of the invention to the individual in need thereof.

The invention further provides a protein of the invention or a bispecific antibody of the invention, for use in the treatment of an individual that has cancer.

In one embodiment the cancer is a cancer that is responsive to the ligand of the respective member of the RTK receptor family and a cancer that expresses a membrane associated member of the WNT pathway.

Further provided is a cell system comprising a protein of the invention or a bispecific antibody of the invention, and a cell that expresses a membrane associated member of the RTK receptor family and that expresses a membrane associated member of the WNT pathway. The cell system is preferably is a cell system comprising a protein of the invention or a bispecific antibody of the invention, and a cell that expresses a membrane associated member of the RTK receptor family and that expresses a membrane associated member of the WNT pathway.

Also provided is a system permissive for inhibiting growth/proliferation of the cell, the method comprising providing the system with a protein of the invention or a bispecific antibody of the invention. The cell is preferably is an RTK-receptor ligand responsive cell that expresses a membrane associated member of the WNT pathway in a system permissive for growth/proliferation of the cell, the method comprising providing the system with a protein of the invention or a bispecific antibody of the invention. The cell is preferably is an RTK-receptor ligand responsive cell that expresses a membrane associated member of the WNT pathway.

Preferred members of the RTK receptor family are the mentioned EGF-receptor family and c-MET; Axl and MST1R.

MET or C-MET is a single pass tyrosine kinase receptor relevant for embryonic development, organogenesis and wound healing. Hepatocyte growth factor/Scatter Factor (HGF/SF) and its splicing isoform (NK1, NK2) are the presently known ligands of the MET receptor. MET is normally expressed by cells of epithelial origin, while expression of HGF/SF is observed in cells of mesenchymal origin. When HGF/SF binds its cognate receptor MET it induces activation. Met is also known as MET Proto-Oncogene, Receptor Tyrosine Kinase; Hepatocyte Growth Factor Receptor; Tyrosine-Protein Kinase Met; Scatter Factor Receptor; Proto-Oncogene C-Met; HGF/SF Receptor; HGF Receptor; SF Receptor; EC 2.7.10.1; Met Proto-Oncogene Tyrosine Kinase; Met Proto-Oncogene; EC 2.7.10; AUTS9; RCCP2; C-Met; and HGFR 3. Accession numbers for the gene and protein are NC_000007.14; NT_007933.16; NC_018918.2; NP_000236.2; NP_001120972.1. The accession numbers are primarily given to provide a further method of identification of C-MET as a target, the actual sequence of the C-MET protein bound by an antibody may vary, for instance because of a mutation in the encoding gene such as those occurring in some cancers or the like. The C-MET antigen binding site binds C-MET and a variety of variants thereof, such as those expressed by some C-MET positive tumor cells.

AXL or AXL Receptor Tyrosine Kinase codes for Tyrosine-protein kinase receptor UFO which is an enzyme that in humans is encoded by the AXL gene. Other names for AXL are AXL Receptor Tyrosine Kinase; AXL Oncogene; EC 2.7.10.1; UFO; Tyrosine-Protein Kinase Receptor UFO; AXL Transforming Sequence/Gene; EC 2.7.10; JTK11; Tyro7; and ARK 3. Accession numbers for the gene and protein are NC_000019.10; NC_018930.2; NT_011109.17; NP_001265528.1; NP_001690.2; NP_068713.2). The accession numbers are primarily given to provide a further method of identification of AXL as a target, the actual sequence of the AXL protein bound by an antibody may vary, for instance because of a mutation in the encoding gene such as those occurring in some cancers or the like. The AXL antigen binding site binds AXL and a variety of variants thereof, such as those expressed by some AXL positive tumor cells.

MST1R or Macrophage stimulating 1 receptor or RON. Other names for MST1R are Macrophage Stimulating 1 Receptor; RON; PTK8 Protein Tyrosine Kinase 8; C-Met-Related Tyrosine Kinase; MSP Receptor; EC 2.7.10.1; P185-Ron; CDw136; PTK8; Macrophage Stimulating 1 Receptor (C-Met-Related Tyrosine Kinase) 3; Macrophage-Stimulating Protein Receptor; Protein-Tyrosine Kinase 8; MST1R Variant RON30; MST1R Variant RON62; RON Variant E2E3; RON Variant 21; CD136 Antigen; EC 2.7.10; CD136, all in membrane bound form. Accession numbers for the gene and protein are NC_000003.12; NT_022517.19; NC 018914.2; NP 001231866.1; NP 002438.2. The accession numbers are primarily given to provide a further method of identification of MST1R as a target, the actual sequence of the MST1R protein bound by an antibody may vary, for instance because of a mutation in the encoding gene such as those occurring in some cancers or the like. The MST1R antigen binding site binds MST1R and a variety of variants thereof, such as those expressed by some MST1R positive tumor cells.

The invention also provides a monospecific antibody comprising variable domains that bind LGR4 wherein the VH chain of the variable domains comprises
the amino acid sequence of VH chain MF5777 as depicted in FIG. 1; or
the amino acid sequence of VH chain MF5777 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR4 wherein the VH chain of the variable domains comprises
the amino acid sequence of VH chain MF5781 as depicted in FIG. 1; or
the amino acid sequence of VH chain MF5781 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR5 wherein the VH chain of the variable domains comprises
the amino acid sequence of VH chain MF5790 as depicted in FIG. 1; or
the amino acid sequence of VH chain MF5790 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR5 wherein the VH chain of the variable domains comprises
the amino acid sequence of VH chain MF5803 as depicted in FIG. 1; or
the amino acid sequence of VH chain MF5803 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR5 wherein the VH chain of the variable domains comprises
the amino acid sequence of VH chain MF5814 as depicted in FIG. 1; or
the amino acid sequence of VH chain MF5814 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR5 wherein the VH chain of the variable domains comprises
the amino acid sequence of VH chain MF5816 as depicted in FIG. 1; or
the amino acid sequence of VH chain MF5816 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR5 wherein the VH chain of the variable domains comprises
the amino acid sequence of VH chain MF5817 as depicted in FIG. 1; or
the amino acid sequence of VH chain MF5817 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind LGR5 wherein the VH chain of the variable domains comprises
the amino acid sequence of VH chain MF5818 as depicted in FIG. 1; or
the amino acid sequence of VH chain MF5818 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind RNF43 wherein the VH chain of the variable domains comprises
the amino acid sequence of VH chain MF5832 as depicted in FIG. 1; or
the amino acid sequence of VH chain MF5832 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind RNF43 wherein the VH chain of the variable domains comprises
the amino acid sequence of VH chain MF5836 as depicted in FIG. 1; or
the amino acid sequence of VH chain MF5836 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind ZNRF3 wherein the VH chain of the variable domains comprises
the amino acid sequence of VH chain MF5850 as depicted in FIG. 1; or
the amino acid sequence of VH chain MF5850 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind ZNRF3 wherein the VH chain of the variable domains comprises
the amino acid sequence of VH chain MF5853 as depicted in FIG. 1; or
the amino acid sequence of VH chain MF5853 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind ZNRF3 wherein the VH chain of the variable domains comprises
the amino acid sequence of VH chain MF5855 as depicted in FIG. 1; or
the amino acid sequence of VH chain MF5855 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind ZNRF3 wherein the VH chain of the variable domains comprises
the amino acid sequence of VH chain MF5884 as depicted in FIG. 1; or
the amino acid sequence of VH chain MF5884 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention also provides a monospecific antibody comprising variable domains that bind ZNRF3 wherein the VH chain of the variable domains comprises
the amino acid sequence of VH chain MF5888 as depicted in FIG. 1; or
the amino acid sequence of VH chain MF5888 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

In one embodiment the invention a monospecific antibody comprising variable domains that bind EGFR wherein the VH chain of the variable domains comprises
the amino acid sequence of VH chain MF3755 as depicted in FIG. 1; or
the amino acid sequence of VH chain MF3755 as depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention relates to an antibody that comprises a variable domain that can bind an epitope on an extracellular part of LGR5 which epitope is located within amino acid residues 21-118 of SEQ ID NO: 1 depicted in FIG. 39. It was shown that amino acid residues D43; G44, M46, F67, G90, and F91 are involved in binding of the antibody. Involved in does not necessarily mean that all residues are indeed contacted by the antibody. Without being bound by theory it is believed that the substitution D43A or G44A results in a (slight) conformational change in the epitope and that the conformation change results in reduced binding of the antibody to the epitope.

The interaction of the antibody with LGR5 does not inhibit the binding of RSPO 1 to LGR5 expressed on the membrane of an LGR5-expressing cell. The interaction of the antibody with RSPO 1 does not inhibit the binding of the antibody to LGR5 expressed on the membrane of an LGR5-expressing cell. This means that the antibody and RSPO1 do not compete with each other for binding to LGR5. At least not in the range of molar ratio's indicated herein. The molar ratio of antibody to RSPO 1 it typically from 0.1 to 0.001 (inclusive) preferably in a molar ratio of between 0.1 to 0.01 (inclusive). In this range the binding of the antibody to LGR5 does not inhibit (block) the binding of RSPO 1 to LGR5.

When herein molar ratio's of antibody to RSPO are mentioned it is preferred that the antibody is present in amounts that result in 40%-80% of the binding achieved when saturating amounts of the antibody are present.

Various antibodies that bind to LGR5 have been described. Binding of an antibody to LGR5 can block the binding of an Rspondin to LGR5 or not. It has been found that antibodies that block the binding of Rspondin 1 ligand to LGR5 bind to a region encompassing the N-terminal region, Leucine-rich repeat (LRR) region 1 and LRR2 of the protein (Lau et al 2011; Nature vol 476; pp 293-297 and supplemental information described therein). The elements are contained in amino acids 21-118 (inclusive) of an LGR5 protein sequence as depicted in SEQ ID NO: 1 depicted in FIG. 39; wherein amino acids 21-70 constitute the N-terminal region, amino acids 71-94 (inclusive) constitute LRR1 and 95-118 constitute LRR2. Only few antibodies that do not block the interaction of LGR5 with Rspondin 1 have been reported in the art and to the best of the inventor's knowledge none of these bind to the N-terminal region of LGR5.

The invention now provides a protein that binds to an epitope within amino acid residues 21-118 of an LGR5 sequence of SEQ ID NO: 1 depicted in FIG. 39 and which protein does not block the binding of Rspondin 1 to LGR5.

The test for determining whether an antibody blocks or does not block the binding of an Rspondin to LGR5 preferably incudes CHO cells that express LGR5 on the cell membrane. The antibody and the Rspondin to be analyzed are mixed together and added to the cells whereupon the binding of the antibody to the cells is determined. The amount of binding of the antibody to the LGR5 expressing cells in the presence of an excess of Rspondin indicates that the antibody does not block the binding of Rspondin to LGR5. The test preferably further includes a control antibody that binds LGR5 and that blocks the binding of the Rspondin to LGR5. The control antibody is preferably antibody PB10261 or OMP88R20 as described in as described in the examples A protein does not block the binding of Rspondin 1 to LGR5 if, under otherwise the same conditions, more protein when compared to control antibody can bind to LGR5 expressing cells. This is preferably assessed under conditions wherein the molar ratio of protein or control antibody to Rspondin is 1:10 or less, i.e. 0.1 or less; preferably in a molar ratio of between 0.1 to 0.001, (inclusive), preferably 0.1 to 0.01 (inclusive). The protein preferably also binds an extracellular part of another membrane associated protein. In a preferred embodiment the other membrane associated protein is a membrane associated member of the EGF-receptor family or cMET.

The invention also provides a protein that can bind an epitope on an extracellular part of LGR5 which epitope is located within amino acid residues 21-118 of SEQ ID NO: 1 depicted in FIG. 39 and wherein the binding of the protein to LGR5 is reduced with one or more of the following amino acid residue substitutions D43A; G44A, M46A, F67A, G90A, and F91A. In a preferred embodiment the interaction of Rspondin (RSPO) 1 with LGR5 on an LGR5-expressing cell does not inhibit the binding of the protein to LGR5 by more than 20%. The inhibition of binding of the protein to LGR5 is preferably measured when the protein and the RSPO 1, 2, 3 or 4 are present in a molar ratio of 0.1 or less; preferably in a molar ratio of between 0.1 to 0.001, (inclusive), preferably 0.1 to 0.01 (inclusive) (protein:RSPO).

The invention further provides a protein that can bind an epitope on LGR5 that is located within amino acid residues 21-118 of SEQ ID NO: 1 depicted in FIG. 39, and wherein interaction of RSPO 1 with LGR5 on an LGR5-expressing cell does not inhibit the binding of the protein to LGR5 by more than 20% when the protein and the RSPO 1 are present in a molar ratio from 0.1 or less; preferably in a molar ratio of between 0.1 to 0.001, (inclusive), preferably 0.1 to 0.01 (inclusive). The epitope is preferably located within amino acid residues 40-95 of SEQ ID NO: 1 depicted in FIG. 39. The binding of the protein to LGR5 is preferably reduced with one or more of the following amino acid residue substitutions D43A; G44A, M46A, F67A, G90A, and F91A. This typically indicates that the protein interacts with these mentioned amino acids. Changing these amino acids these alters the binding of the protein to LGR5. These amino acids are therefore likely to be the contact residues in LGR5 for the binding protein. In the present case the inventors consider residues M46; F67, G90A and F91 to be contact residues. D43 and G44 can also be contact residues but can also be residues that alter the specific conformation of other residues such that they are no longer able to be bound by the protein. The protein preferably can bind a further protein. The further protein is preferably a membrane protein comprising an extracellular part. The further protein is preferably a membrane associated member of the epidermal growth factor (EGF) receptor family or cMET. The LGR5 binding protein as discussed herein is preferably an antibody preferably a bispecific antibody.

The invention further provides a bispecific antibody comprising a variable domain that can bind an epitope on an extracellular part of LGR5 which epitope is located within amino acid residues 21-118 of SEQ ID NO: 1 depicted in FIG. 39 and wherein the binding of the variable domain to LGR5 is reduced with one or more of the following amino acid residue substitutions D43A; G44A, M46A, F67A, G90A, and F91A; the bispecific antibody further comprises a variable domain that can bind a further protein. The further protein is preferably a membrane protein comprising an extracellular part. The further protein is a membrane associated member of the EGF receptor family or cMET.

The binding of the protein or bispecific antibody as mentioned herein to the membrane associated member of the EGF receptor family or cMET preferably reduces ligand-induced signaling in a cell that comprises said membrane associated member of the EGF receptor family or cMET.

The invention provides an antibody that comprises a variable domain that can bind an epitope on an extracellular part of LGR5 which epitope is located within amino acid residues 21-118 of SEQ ID NO: 1 depicted in FIG. 39.

The invention further provides an antibody that binds an epitope on an extracellular part of LGR5, wherein amino acid residues D43; G44, M46, F67, G90, and F91 are involved in binding of the antibody to the epitope.

The invention further provides an antibody that binds an epitope on an extracellular part of LGR5, wherein one or more of the amino acid residue substitutions of D43A; G44A, M46A, F67A, G90A, and F91A reduces the binding of the antibody to the epitope.

The invention further provides an antibody that comprises a variable domain that can bind an epitope on an extracellular part of LGR5 which epitope is located within amino acid residues 21-118 of SEQ ID NO: 1 depicted in FIG. 39, and wherein the binding of the antibody to the epitope is reduced by one or more of the following amino acid residue substitutions D43A; G44A, M46A, F67A, G90A, and F91A.

A membrane protein as used herein is a cell membrane protein, i.e. a protein that is in the outer membrane of a cell, the membrane that separates the cell from the outside world. The membrane protein has an extracellular part. A membrane protein is at least on a cell if it contains a transmembrane region that is in the cell membrane of the cell.

The antibody preferably further comprises a further variable domain can bind a further protein. The further protein is preferably a membrane protein comprising an extracellular part. The further protein is preferably a membrane associated member of the epidermal growth factor (EGF) receptor family or cMET. The antibody is a bispecific antibody.

For the purpose of clarity and a concise description features are described herein as part of the same or separate embodiments, however, it will be appreciated that the scope of the invention may include embodiments having combinations of all or some of the features described.

### BRIEF DESCRIPTION OF THE DRAWINGS

FIG. 1: amino acid sequences of VH fragments described in the current application. CDR1, CDR2 and CDR3 regions, as well as FR1, FR2, FR3 and FR4 regions are indicated.

FIG. 2: nucleotide sequences of selected VH-encoding cDNA's described in the current application.

FIG. 3: Annotated sequence of the common light chain (cLC).

FIG. 4: Annotated Fc (CH1-CH3) region amino acid sequence of antibodies tested in the present application.

FIG. 5: The effect of growth factors EGF and HRG on the tumoroid morphology in tumoroids P14T and P18T.

Example of the effect of growth factors EGF and HRG on the tumoroid morphology in tumoroids P14T and P18T. Growth factors induce a simultaneous change in tumoroid size, the lumen size and the distribution of the lumen. The combination of these three morphological parameters locates the observed organoids in a unique feature space. The Euclidian distance can be calculated based on the combination of these three features with the no growth factor (no GE) as a reference point. The Euclidian distance is then the change in X, Y and Z direction induced by the treatments.

FIG. 6: FACS analysis of sera of ZNRF3-immunised animals binding to Freestyle 293F cells that stably over-expressed the antigen, taken at day 35.

Sera were tested compared to the day 0 (pre-immune) sera for binding to the 293F Freestyle cell line stably expressing the ECD of ZNRF3. Every panel shows an overlay of control (non-stained) cells (filled grey) and the cells stained with the respective sera (no fill). Mice are numbered L19-L24; control stainings are indicated on the right side of the figure n.c.: negative control.

FIG. 7: Example of FACS staining of a mix of DiD-labelled ZNRF3 over-expressing cells and non-labelled (parental) 293F freestyle cells using preparations of selected monoclonal phage clones.

Every dot-plot is an overlay of non-stained cells and cells stained with a selected Fab expressed on phage. The upper half of every panel shows the DiD-labelled antigen-positive cells and the lower half of the panel the non-labelled antigen-negative 293F freestyle cells. Of the six clones shown in the figure (labelled 1-6), four are antigen-specific (1, 3, 4 and 6) and two (2 and 5) are non-reactive. Control for expression of the protein was staining with the antibody directed to the cMyc-derived epitope tag and negative control was staining with the secondary antibodies (anti-M13 and Strep-PE) only.

FIG. 8: Affinity ranking of monovalent IgG directed to hLGR5 (all combined with the Tetanus toxoid Fab Fragment) on the cellular surface of Freestyle 293F cells overexpressing hLGR5.

A representative example of the entire panel is shown. A two-fold dilution series of IgG (5 μg/ml-0.08 μg/ml) was tested on a fixed number of cells (5×105 cells/well) stable expressing hLGR5. The mean fluorescence intensity (MFI) was measured for each data point and was plotted against the increasing amount of IgG used for staining. From each IgG the area under the curve (AUC) was calculated based on the individual curves and used for ranking. The table (right panel) shows the AUC values of bispecific IgG shown in the graph (left panel). Based on the AUC values the IgG were ranked for binding affinity to their respective target.

FIG. 9: rhR-Spondin3 blocking capacity of hLGR5 monovalent binding IgG (all combined with a TT Fab fragment) was tested in an R-Spondin blocking ELISA.

A representative example is shown of the LGR5 panel. Maximum binding (normalized to 0%) was established by uninhibited binding to 2 μg/ml coated rhR-Spondin3 using 0,031 μg/ml rhLGR5-Fc, and detection with an anti-Fc antibody. The OD450 nm value for maximum binding was set at 100%. R-Spondin3 surplus indicates the addition of excess R-Spondin3 (15 μg/ml) to serve as a positive control for competition. The bispecific IgG, tested at 15 μg/ml, the OD450 nm values were normalized based on the maximum signal and are indicated by the blue bars. Clones were considered partially blocking when the percentage was below 80% and considered blocking when the percentage was below 50%. This example shows a representative panel of LGR5 targeting LGR5/TT IgG fragments.

FIG. 10: Growth inhibiting effects of the reference antibody PG3755 (EGFR bivalent, mono-specific) and PB10651 (EGFR/LGR5 bispecific) on the tumoroid line P18T in the presence of 5 ng/ml EGF in the culture medium.

The antibodies revert the EGF-induced tumoroid phenotype to a no growth factor stimulated phenotype (first column). PB10651 is active as a growth inhibitor at lower doses than the EGFR bispecific reference antibody PG3755.

FIG. 11: Example of the validation screen results for one colon tumoroid exposed to EGFR/WNT targeting bispecific antibodies and control treatments.

The lower the Euclidian distance, the more potent the treatment effect; 1 equals normal growth. PB10651 (EGFR/LGR5 antibody with MF3755×MF5816) shows complete growth inhibition at the dose tested (2 μg/ml) and outperforms Cetuximab.

FIG. 12: PB10651 retains immune-reactivity after labelling with ¹²⁵I. Immuno-reactivity of radio-active labelled protein. A: Lindmo assay for the anti-EGFR Fab arm in PB10651; B: Lindmo assay for the anti-LGR5 Fab arm of PB10651. There was very little loss of immune-reactivity after labelling of the protein with ¹²⁵I.

FIG. 13: PB10651 binds with sub-nanomolar affinity to both EGFR and LGR5. Affinity analysis of PB10651 binding to LGR5 and EGFR. A: analysis of PB10651 binding to CHO-LGR5 cells; B: analysis of PB10651 binding to CHO-EGFR cells and C: affinity analysis of PB10651 binding to DLD-1 cells.

FIG. 14: The anti-EGFR Fab arm in PB10651 binds to residues in domain III of EGFR as determined by shotgun mutagenesis analysis.

The epitope on EGFR recognised by the anti-EGFR Fab arm present in PB10651 is depicted, modelled onto the structure of EGFR (Li et al., 2005; pdb reference 1YY9). Residues that were found to be relevant for the binding of PB10651 are highlighted in the structure.

FIG. 15: The anti-LGR5 Fab arm in PB10651 binds to residues present in the N-CAP domain and first leucine-rich repeat of LGR5, as determined by shotgun mutagenesis analysis.

The figure shows the epitope on LGR5 recognised by PB10651, modelled onto the structure of LGR5 in complex with RSPO1 (Peng et al., 2013: pdb reference 4BSR). The residues that are recognised are indicated in black in the left (light grey) LGR5 molecule of the dimer. RSPO1 is indicated in ball and stick.

FIG. 16: The copied version of the OMP88R20 anti-LGR5 antibody is dose-dependently inhibited for binding LGR5 by the ligand R-spondin1.

The copied version of OMP88R20 (PG7711) was tested for binding at the EC50 (50 ng/ml) for binding to LGR5 over-expressing cells in the presence of increasing amounts of R-spondin1. The MFI value obtained was normalised to the control (no R-spondin1) value.

FIG. 17: The anti-LGR5 Fab MF5816 present in PB10651 is not inhibited for binding LGR5 in a large molar excess of the ligand R-spondin1.

The copied version of OMP88R20 (PG7711) was tested for binding at 50 ng/ml and bispecifics were tested at 100 ng/ml for binding to LGR5 over-expressing cells in the presence of increasing concentrations of R-spondin1, or of the rat antibody 1D9. Binding of the indicated antibodies was detected with a PE-labeled anti-human IgG secondary antibody. For every point in the curve, the MFI value obtained was normalised to (divided by) the value found in the control (no R-spondin1) situation.

FIG. 18: Bivalent, mono-specific PG3755 potently inhibits EGFR-mediated signalling.

The proliferation (measured as fluorescence counts after alamar blue addition to the cells: Y-axis) of A431 cells was measured in the presence of 62.5 ng/ml of human EGF and increasing amounts of the indicated anti-EGFR antibodies. PG1337 served as non-binding (negative) control. An increasing number of counts is indicative of an increased number of cells and thereby of blocking EGFR-mediated signalling.

FIG. 19: Afucosylated PB10651 binds with comparable affinity to the cynomolgus orthologues of both EGFR and LGR5.

MFI values obtained after staining with the indicated antibody at the indicated concentration in FACS are depicted as a function of the concentration of antibody used. For EGFR, cetuximab was used as positive control for cynomolgus EGFR binding. For LGR5, the copied version of hu8E11v2 (PG7543) from Genentech was used as positive control for cynomolgus LGR5 binding.

FIG. 20: Staining of patient-derived organoids in FACS using lead anti-LGR5/EGFR bispecific antibodies visualises LGR5 expression.

An organoid sample derived from a colorectal cancer patient (P18) was stained using two different anti-LGR5 antibodies (MF5816 and MF5814) and a negative control that recognizes Tetanus Toxin (TT). The staining of the TT antibody was used to set the threshold for the staining of the two anti-LGR5 antibodies. With the TT staining set at 0.8%, the MF5816 and MF5814 antibodies showed comparable staining levels with 53.6% and 52.7% of the cells scored positive for LGR5 expression.

FIG. 21: Staining and sorting of patient-derived organoids using lead bispecific anti-LGR5/EGFR bispecific antibodies enriches for LGR5-expressing (cancer stem) cells.

A colorectal cancer organoid line (P18T) was used for FACS staining and sorting of the top (positive) and bottom (negative) 15% of stained cells identified by the anti-LGR5 antibodies MF5814 and MF5816. 2000 single cells were sorted for each population and used to generate cDNA for quantitative PCR. The expression of B2M was used as an endogenous control, and the expression of the LGR5 mRNA in the positive fraction was represented relative to the negative fraction using the 2-ΔΔCT method and the StepOne 2.2 plus software. The experiment was ran on two separate occasions (Exp.1 and Exp. 2), with each antibody, and experimental repeats, displaying an increase in LGR5 mRNA in the positive fraction relative to the negative. Error bars represent the relative quantification max, which was produced as part of the analysis using the StepOne 2.2 plus software.

FIG. 22: Sorting of patient-derived organoid for LGR5-expressing cells using lead anti-LGR5/EGFR bispecific antibody enriches for tumour-initiating cells.

Left: pictures of the organoids depict one of the BME drops and were captured using an Olympus MVX10 MacroView microscope. The inset on the MF5816 picture displays a closer view of one of the organoids in the drop. Right: bar graph depicting the number of organoids counted after two weeks of growth found in the LGR5-positive and -negative cell population.

FIG. 23: Treatment of patient-derived organoids with lead anti-LGR5/EGFR bispecific antibody potently inhibits organoid outgrowth.

Left: bar graphs depicting the average organoid number (top) and organoid size (bottom) found after the different treatments with the indicated antibodies (indicated below the graphs). Right: representative examples of images obtained from the organoids after the different treatments. Single cells were seeded and left to establish organoids for three days. On day three the media was removed and replaced with media containing the antibodies at a concentration of 2 μg/mL. The organoids were further cultured for another 7 days. On day 7 the organoids were scanned using an Olympus ScanR using the x4 light objective, and the number of organoids quantified using ImageJ running an in-house designed macro. The data is presented relative to the TT control, and a two-tailed, paired sample T-Test was run to assess for significant differences between treatments. Error bars represent standard deviation. *=p<0.05 relative to TT, **=p<0.001 relative to TT. #=p<0.05 relative to EGFR-TT, ##=p<0.001 relative to EGFR-TT.

FIG. 24: LGR5×EGFR bispecifics potentiate the reduction of the non-differentiated cell population

Single cells were seeded and grown for three days before being treated with the antibodies. After seven days, total RNA was extracted and used for quantitative real-time PCR analysis to detect the mRNA levels of LGR5 and CK20 (a marker of differentiation). Differences in expression were detected using the 2-ΔΔCT method. The data is presented relative to the negative control (TT-TT). The experiment was carried out on two separate occasions, and the average of the two experiments shown. Error bars represent the standard deviation of the two experiments.

FIG. 25: Examples of superclusters indicating amino acid changes that are tolerated in a VH and/or CDR therein of the present invention without losing binding specificity. (FIG. 25A, FIG. 25B, and FIG. 25C).

FIG. 26: Comparison of the potency of PB10651 versus Cetuximab in inhibiting growth of patient-derived tumoroids and organoids derived from normal tissue in vitro

Examples of the effects of treatments with concentrations ranges of PB10651 and Cetuximab on organoid cultures from organoids derived from normal tissue and from cancerous tissue (FIG. 26A and FIG. 26B). FIG. 26C: IC50 values for growth inhibition obtained using patient-derived tumoroids and organoids derived from normal tissue. The ratio in the third column depicts the ratio IC50(Cetuximab)/IC50(PB10651).

FIG. 27: PB10651 is significantly more potent than monospecific anti-EGFR and anti-LGR5 antibodies in inhibiting organoid growth in vitro.

In vitro treatment of p18T (A) and C1M (B) organoid cultures in 3D with the indicated antibodies. Organoid size is plotted as a function of the concentration of antibody used. Anti-LGR5 antibodies do not show any effects on organoid growth;

FIG. 28: PB10651 is more potent in reducing organoid growth in vitro than a mix of the parental bivalent, mono-specific antibodies.

The figure shows growth inhibiting effects of the reference antibodies PG3755 (EGFR bivalent, mono-specific), PG5816 (LGR5 bivalent, mono-specific) or an equimolar combination thereof and PB10651 (EGFR/LGR5 bispecific) on the tumoroid line P18T in the presence of EGF. FIG. 28A. P18T organoid size is depicted as a function of antibody concentration used for treatment. Organoids treated with PG5816 showed no growth inhibition; PG3755 significantly reduced growth, but not to the same extent as PB10651. The combination of PG3755 and PG5816 was also significantly less potent than PB10651 in inhibiting growth of P18T tumoroids. FIG. 28B. A comparison of the IC50 values for growth inhibition found for treatment of the indicated organoids (top) with the indicated antibodies (left panel). The IC50 values found for PB10651 are always lower than those found for the mix of the parental antibodies.

FIG. 29: In vitro organoid treatment with PB10651 causes intra-cellular localisation of antibody that is not observed with monospecific, bivalent antibodies directed to LGR5 or EGFR.

After 24 hours of antibody treatment, organoids were fixed, permeabilised and stained for human IgG to reveal (sub-) cellular localisation of antibody. Only in the case of PB10651, a punctate intra-cellular staining was observed that was absent in the organoids treated with bivalent, mono-specific antibodies. In the latter case, antibody was concentrated and localised onto the cell membrane.

FIG. 30: Organoids that are responsive to treatment with PB10651 show intra-cellular localisation of antibody that is not observed after treatment of organoids that are not responsive to the treatment.

After 24 hours of antibody treatment, organoids were fixed, permeabilised and stained for human IgG to reveal (sub-) cellular localisation of antibody. The different organoids that were responsive to treatment with PB10651 (COM, C55T, P18T, C31M) showed intra-cellular staining of antibody, whereas the organoids that were non-responsive showed cell surface localisation of PB10651 (C28T, P19Tb, P8T, C51N).

FIG. 31: Vector maps of MV1453 and MV1626 plasmids.

FIG. 32: Vector map of MV1622 (RMD expression vector).

FIG. 33: CEX-HPLC profile of afucosylated PB10651.

FIG. 34: Results of the ADCC reporter assay of PB10651(ADCC-enhanced) compared to PB10651(non-enhanced) and PG1337 (control).

FIG. 35: Expression of EGFR and LGR5 in patient-derived xenograft (PDX) models.

Expression of EGFR and LGR5 genes in live study and frozen stock tumors was measured by TaqMan real time PCR. Expression is represented as the log-transformed value obtained with the 2-ΔΔCT method, using GAPDH as housekeeping gene. Tumors extracted from live animals presented gene expression values similar to the reference stock tumors.

FIG. 36: PB10651 inhibits the in vivo growth of PDX.

The colorectal PDX models were treated weekly (arrows) with PB10651 or Cetuximab or PBS. Tumor growth (indicated in mm3) was followed during and after cessation of the treatment. Models responsive to cetuximab (one WT and one KRAS G13D mutant) also responded to PB10651. One out of three KRAS G12V/D mutant models that did not respond to Cetuximab displayed significant tumor growth inhibition with PB10651.

FIG. 37: Treatment with PB10651 causes a significant reduction in the number of proliferating cells in the tumour.

Immunohistochemical Staining of P18 Tumoroids After 48 Hours of Treatment (2 μg/mL). The top panel depicts a marker of proliferation (ki67), and the lower panel a marker of apoptosis (cleaved caspase-3). Images were taken using a ×20 objective, brown staining indicates expression, with nuclei counter stained using haematoxylin

FIG. 38: PB10651 is significantly more potent than cetuximab in inhibiting organoid growth in vivo.

FIG. 38A. The Effect of Weekly Antibody Dosing on P18 Xenograft Growth. Once tumours reached an average size of 50 mm³ antibody treatments began (day 0) and the relative change in volume is plotted. Mice were treated once a week (indicated by arrows). All tumour volumes within a treatment group were averaged and plotted; the number of xenografts at each time point is represented in the table. Error bars represent the standard error of the mean. *=P<0.01 compared to PBS, #=P<0.01 compared to cetuximab as determined by paired t-test analysis.

FIG. 38B. The Effect of Weekly Antibody Dosing on C31M Xenograft Growth.

Once tumours reached an average size of 30 mm³ antibody treatments began (day 0) and the relative change in volume is plotted. Mice were treated once a week (indicated by arrows). All tumour volumes within a treatment group were averaged and plotted; the number of xenografts at each time point is represented in the table. Error bars represent the standard error of the mean. *=P<0.01 compared to PBS, #=P<0.05 compared to cetuximab as determined by paired t-test analysis

FIG. 39: Annotated amino acid sequence of human LGR5.

Leader indicates the amino acid sequence that is cut from the mature protein, the N-region spans amino acids 21-70 inclusive; LRR stands for leucine-rich repeat region, LLR1 is leucine-rich repeat region 1; LRR2 is leucine-rich repeat region 2 etc. LLR1 starts at amino acid 71 and ends at amino acid 94 (inclusive); LLR2 starts at 95 and ends at 118 inclusive. CRL is the cysteine rich linker region. TM indicates the various transmembrane regions and C-TERM the c-terminal end of the protein. The annotated parts together form one consecutive sequence designated SEQ ID NO: 1. This complete sequence is SEQ ID NO: 1 this numbering prevails even if for some reason another sequence is also listed as SEQ ID NO: 1.

FIG. 40: Annotated amino acid sequence of wild type, human EGFR (GenBank NM_005228).

The signal peptide, extra-cellular part, predicted trans-membrane helix and intracellular tyrosine kinase including the C-terminal tail are all indicated. The annotated parts together form one consecutive sequence designated SEQ ID NO: 2. This complete sequence is SEQ ID NO: 2 this numbering prevails even if for some reason another sequence is also listed as SEQ ID NO: 2.

### EXAMPLES

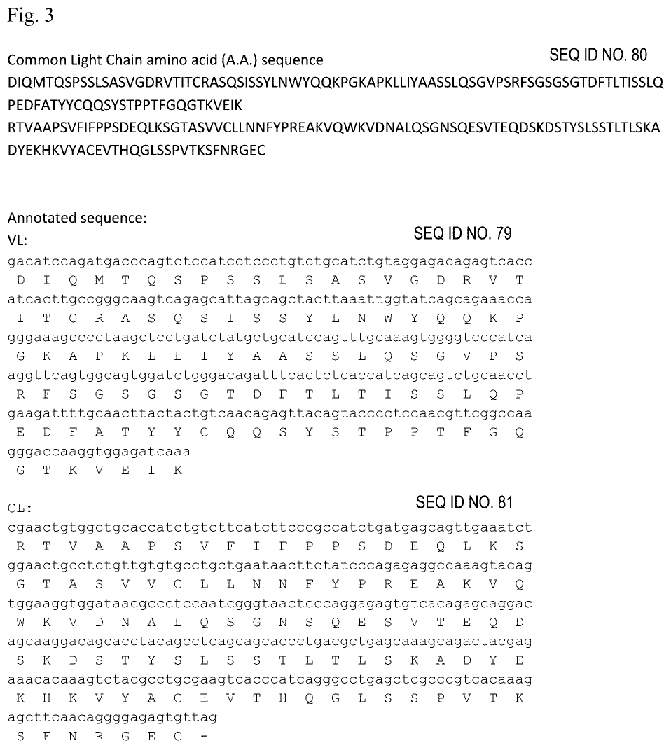

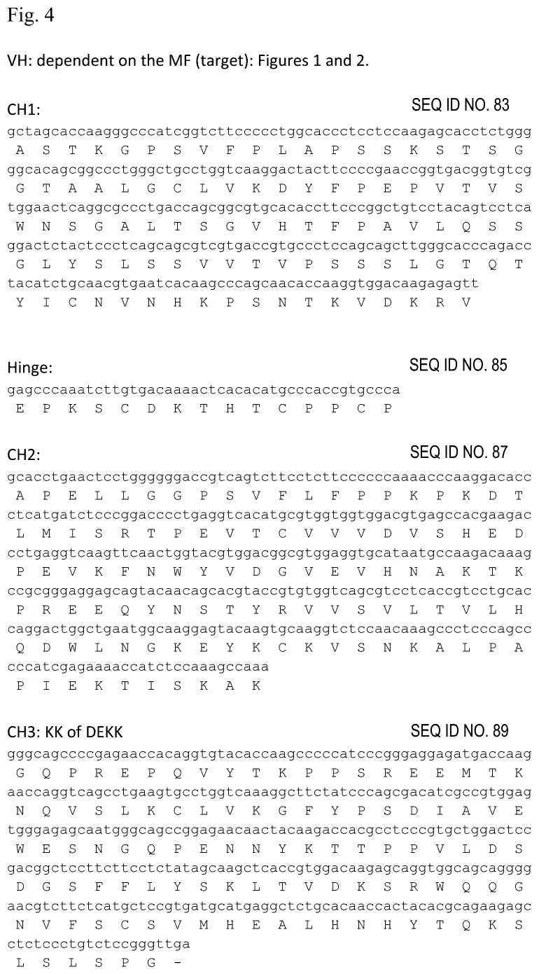

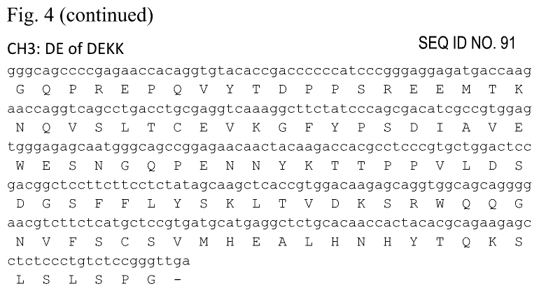

As used herein “MFXXXX” wherein X is independently a numeral 0-9, refers to a Fab comprising a variable domain wherein the VH has the amino acid sequence identified by the 4 digits. Unless otherwise indicated the light chain variable region of the variable domain typically has a sequence of FIG. 3. The light chain has a sequence as depicted in FIG. 3. “MFXXXX VH” refers to the amino acid sequence of the VH identified by the 4 digits. The MF further comprises a constant region of a light chain and a constant region of a heavy chain that normally interacts with a constant region of a light chain. PG refers to a monospecific antibody comprising identical heavy and light chains. PB refers to a bispecific antibody with two different heavy chains. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954). In PB, PG and other codes the numerical indication is sometimes followed by an indication of the production batch. For instance PB10651p06 refers to PB10651 batch 6.

### Example: 1

### Methods, Materials and Screening of Antibodies

### Cell Lines:

Freestyle 293F cells (cat. nr. p/n51-0029) were obtained from Invitrogen and routinely maintained in 293 FreeStyle medium. HEK293T (ATCC-CRL-11268) and CHO-K1 (DSMZ ACC110) cell lines were purchased from ATCC and routinely maintained in DMEM/F12 (Gibco) supplemented with L-Glutamine (Gibco) and FBS (Lonza).

### Generation of Recombinant Human and Mouse LGR4, LGR5, ZNRF3 and RNF43 Expression Vectors

Human LGR4 Full length human (h)LGR4 was present in vector pEF1_Myc/His (Invitrogen), containing 2 FLAG tags and 2 HA tags (pEF1_hLGR4-FLAG-HA), and was cloned into pVax1 (Invitrogen), resulting in pVax1_hLGR4-FLAG-HA. The insert sequence was verified by comparison with the NCBI Reference sequence NM_018490. The sequence contained the following deviations on amino acid level: P2A in the signal peptide. The insert from construct pEF1_hLGR4-FLAG-HA was recloned into pVax1. Alternative the full length hLGR4 in pVax1 was replaced with a truncated version of hLGR4; hLGR4(ECD)-GPA33(TM)-FLAG resulting in pVax1_hLGR4(ECD)-GPA33(TM)-FLAG.

Amino acid sequence full length hLGR4-FLAG-HA
insert (both in pEF1_Myc/His and pVax1) for
expression on cell surface
SEQ ID NO: 3
MAGPLGLLCFLALGLLGSAGPSGAAPPLCAAPCSCDGDRRVDCSGKGLTA
VPEGLSAFTQALDISMNNITQLPEDAFKNFPFLEELQLAGNDLSFIHPKA
LSGLKELKVLTLQNNQLKTVPSEAIRGLSALQSLRLDANHITSVPEDSFE
GLVQLRHLWLDDNSLTEVPVHPLSNLPTLQALTLALNKISSIPDFAFTNL
SSLVVLHLHNNKIRSLSQHCFDGLDNLETLDLNYNNLGEFPQAIKALPSL
KELGFHSNSISVIPDGAFDGNPLLRTIHLYDNPLSFVGNSAFHNLSDLHS
LVIRGASMVQQFPNLTGTVHLESLTLTGTKISSIPNNLCQEQKMLRTLDL
SYNNIRDLPSFNGCHALEEISLQRNQIYQIKEGTFQGLISLRILDLSRNL
IHEIHSRAFATLGPITNLDVSFNELTSFPTEGLNGLNQLKLVGNFKLKEA
LAAKDFVNLRSLSVPYAYQCCAFWGCDSYANLNTEDNSLQDHSVAQEKGT
ADAANVTSTLENEEHSQIIIHCTPSTGAFKPCEYLLGSWMIRLTVWFIFL
VALFFNLLVILTTFASCTSLPSSKLFIGLISVSNLFMGIYTGILTFLDAV
SWGRFAEFGIWWETGSGCKVAGFLAVFSSESAIFLLMLATVERSLSAKDI
MKNGKSNHLKQFRVAALLAFLGATVAGCFPLFHRGEYSASPLCLPFPTGE
TPSLGFTVTLVLLNSLAFLLMAVIYTKLYCNLEKEDLSENSQSSMIKHVA
WLIFTNCIFFCPVAFFSFAPLITAISISPEIMKSVTLIFFPLPACLNPVL
YVFFNPKFKEDWKLLKRRVTKKSGSVSVSISSQGGCLEQDFYYDCGMYSH
LQGNLTVCDCCESFLLTKPVSCKHLIKSHSCPALAVASCQRPEGYWSDCG
TQSAHSDYADEEDSFVSDSSDQVQACGRACFYQSRGFPLVRYAYNLPRVK
DSRDYKDDDDKAGADYKDDDDKLDGGYPYDVPDYAAGAYPYDVPDYA

Of which:

signal peptide
SEQ ID NO: 267
MAGPLGLLCFLALGLLGSAGPSGA:
hLGR4 (FL)
SEQ ID NO: 268
APPLCAAPCSCDGDRRVDCSGKGLTAVPEGLSAFTQALDISMNNITQLPE
DAFKNFPFLEELQLAGNDLSFIHPKALSGLKELKVLTLQNNQLKTVPSEA
IRGLSALQSLRLDANHITSVPEDSFEGLVQLRHLWLDDNSLTEVPVHPLS
NLPTLQALTLALNKISSIPDFAFTNLSSLVVLHLHNNKIRSLSQHCFDGL
DNLETLDLNYNNLGEFPQAIKALPSLKELGFHSNSISVIPDGAFDGNPLL
RTIHLYDNPLSFVGNSAFHNLSDLHSLVIRGASMVQQFPNLTGTVHLESL
TLTGTKISSIPNNLCQEQKMLRTLDLSYNNIRDLPSFNGCHALEEISLQR
NQIYQIKEGTFQGLISLRILDLSRNLIHEIHSRAFATLGPITNLDVSFNE
LTSFPTEGLNGLNQLKLVGNFKLKEALAAKDFVNLRSLSVPYAYQCCAFW
GCDSYANLNTEDNSLQDHSVAQEKGTADAANVTSTLENEEHSQIIIHCTP
STGAFKPCEYLLGSWMIRLTVWFIFLVALFFNLLVILTTFASCTSLPSSK
LFIGLISVSNLFMGIYTGILTFLDAVSWGRFAEFGIWWETGSGCKVAGFL
AVFSSESAIFLLMLATVERSLSAKDIMKNGKSNHLKQFRVAALLAFLGAT
VAGCFPLFHRGEYSASPLCLPFPTGETPSLGFTVTLVLLNSLAFLLMAVI
YTKLYCNLEKEDLSENSQSSMIKHVAWLIFTNCIFFCPVAFFSFAPLITA
ISISPEIMKSVTLIFFPLPACLNPVLYVFFNPKFKEDWKLLKRRVTKKSG
SVSVSISSQGGCLEQDFYYDCGMYSHLQGNLTVCDCCESFLLTKPVSCKH
LIKSHSCPALAVASCQRPEGYWSDCGTQSAHSDYADEEDSFVSDSSDQVQ
ACGRACFYQSRGFPLVRYAYNLPRVKD:
SR: linker region
FLAG tag
SEQ ID NO: 269
DYKDDDDK:
AGA: linker region
FLAG tag
SEQ ID NO: 269
DYKDDDDK:
Linker region
SEQ ID NO: 270
LDGG:
IIA tag
SEQ ID NO: 271
YPYDVPDYA:
AGA: Linker region
HA tag
SEQ ID NO: 271
YPYDVPDYA:
Amino acid sequence hLGR4(ECD)-GPA33-FLAG
insert in pVax1 for expression on cell
surface:
SEQ ID NO: 4
MPGPLGLLCFLALGLLGSAGPSGAAPPLCAAPCSCDGDRRVDCSGKGLTA
VPEGLSAFTQALDISMNNITQLPEDAFKNFPFLEELQLAGNDLSFIHPKA
LSGLKELKVLTLQNNQLKTVPSEAIRGLSALQSLRLDANHITSVPEDSFE
GLVQLRHLWLDDNSLTEVPVHPLSNLPTLQALTLALNKISSIPDFAFTNL
SSLVVLHLHNNKIRSLSQHCFDGLDNLETLDLNYNNLGEFPQAIKALPSL
KELGFHSNSISVIPDGAFDGNPLLRTIHLYDNPLSFVGNSAFHNLSDLHS
LVIRGASMVQQFPNLTGTVHLESLTLTGTKISSIPNNLCQEQKMLRTLDL
SYNNIRDLPSENGCHALEEISLQRNQIYQIKEGTFQGLISLRILDLSRNL
IHEIHSRAFATLGPITNLDVSFNELTSFPTEGLNGLNQLKLVGNFKLKEA
LAAKDFVNLRSLSVPYAYQCCAFWGCDSYANLNTEDNSLQDHSVAQEKGT
ADAANVTSTLENEEHSQIIIHCTPSTGAFKPCEYLLGSWMIRVALYVGIA
VGVVAALIIIGIIIYCCCCRGKDDNTEDKEDARPNREAYEEPPEQLRELS
REREEEDDYRQEEQRSTGRESPDHLDQDYKDDDDK

Of which:

signal peptide
SEQ ID NO: 272
MPGPLGLLCFLALGLLGSAGPSGA:
hLGR4(ECD)
SEQ ID NO: 273
APPLCAAPCSCDGDRRVDCSGKGLTAVPEGLSAFTQALDISMNNITQLPE
DAFKNFPFLEELQLAGNDLSFIHPKALSGLKELKVLTLQNNQLKTVPSEA
IRGLSALQSLRLDANHITSVPEDSFEGLVQLRHLWLDDNSLTEVPVHPLS
NLPTLQALTLALNKISSIPDFAFTNLSSLVVLHLHNNKIRSLSQHCFDGL
DNLETLDLNYNNLGEFPQAIKALPSLKELGFHSNSISVIPDGAFDGNPLL
RTIHLYDNPLSFVGNSAFHNLSDLHSLVIRGASMVQQFPNLTGTVHLESL
TLTGTKISSIPNNLCQEQKMLRTLDLSYNNIRDLPSFNGCHALEEISLQR
NQIYQIKEGTFQGLISLRILDLSRNLIHEIHSRAFATLGPITNLDVSFNE
LTSFPTEGLNGLNQLKLVGNFKLKEALAAKDFVNLRSLSVPYAYQCCAFW
GCDSYANLNTEDNSLQDHSVAQEKGTADAANVTSTLENEEHSQIIIHCTP
STGAFKPCEYLLGSWMIR:
GPA33 sequence containing the TM region
SEQ ID NO: 274
VALYVGIAVGVVAALIIIGIIIYCCCCRGKDDNTEDKEDARPNREAYEEP
PEQLRELSREREEEDDYRQEEQRSTGRESPDHLDQ:
FLAG
SEQ ID NO: 269
DYKDDDDK:

### Human LGR5

Full length human (h)LGR5 was present in vector pEF1_Myc/His (pEF1_hLGR5-FLAG-HA), containing 2 FLAG tags and 2 HA tags, and was cloned into pVax1 (Invitrogen), resulting in pVax1_hLGR5-FLAG-HA. The sequence was verified by comparison with the NCBI Reference sequence NM_003667.3. The insert from construct pEF1_hLGR5-FLAG-HA was recloned into pVax1, resulting in pVax1_hLGR5-FLAG-HA. Alternative the full length hLGR5 in pVax1 was replaced with a truncated version of hLGR5; hLGR5(ECD)-GPA33(TM)-FLAG resulting in pVax1_hLGR5(ECD)-GPA33(TM)-FLAG.

Amino acid sequence full length hLGR5-FLAG-HA
insert (both in pEF1_Myc/His and pVax1) for
expression on cell surface
SEQ ID NO: 5
MDTSRLGVLLSLPVLLQLATGGSSPRSGVLLRGCPTHCHCEPDGRMLLRV
DCSDLGLSELPSNLSVFTSYLDLSMNNISQLLPNPLPSLRFLEELRLAGN
ALTYIPKGAFTGLYSLKVLMLQNNQLRHVPTEALQNLRSLQSLRLDANHI
SYVPPSCFSGLHSLRHLWLDDNALTEIPVQAFRSLSALQAMTLALNKIHH
IPDYAFGNLSSLVVLHLHNNRIHSLGKKCFDGLHSLETLDLNYNNLDEFP
TAIRTLSNLKELGFHSNNIRSIPEKAFVGNPSLITIHFYDNPIQFVGRSA
FQHLPELRTLTLNGASQITEFPDLTGTANLESLTLTGAQISSLPQTVCNQ
LPNLQVLDLSYNLLEDLPSFSVCQKLQKIDLRHNEIYEIKVDTFQQLLSL
RSLNLAWNKIAIIHPNAFSTLPSLIKLDLSSNLLSSFPITGLHGLTHLKL
TGNHALQSLISSENFPELKVIEMPYAYQCCAFGVCENAYKISNQWNKGDN
SSMDDLHKKDAGMFQAQDERDLEDFLLDFEEDLKALHSVQCSPSPGPFKP
CEHLLDGWLIRIGVWTIAVLALTCNALVTSTVFRSPLYISPIKLLIGVIA
AVNMLTGVSSAVLAGVDAFTFGSFARHGAWWENGVGCHVIGFLSIFASES
SVFLLTLAALERGFSAKYSAKFETKAPFSSLKVIILLCALLALTMAAVPL
LGGSKYGASPLCLPLPFGEPSTMGYMVALILLNSLCFLMMTIAYTKLYCN
LDKGDLENIWDCSMVKHIALLLFTNCILNCPVAFLSFSSLINLTFISPEV
IKFILLVVVPLPACLNPLLYILFNPHFKEDLVSLRKQTYVWTRSKHPSLM
SINSDDVEKQSCDSTQALVTFTSSSITYDLPPSSVPSPAYPVTESCHLSS
VAFVPCLARDYKDDDDKAGADYKDDDDKLDGGYPYDVPDYAAGAYPYDVP
DYA

Of which:

signal peptide
SEQ ID NO: 275
MDTSRLGVLLSLPVLLQLATG:
hLGR5
SEQ ID NO: 276
GSSPRSGVLLRGCPTHCHCEPDGRMLLRVDCSDLGLSELPSNLSVFTSYL
DLSMNNISQLLPNPLPSLRFLEELRLAGNALTYIPKGAFTGLYSLKVLML
QNNQLRHVPTEALQNLRSLQSLRLDANHISYVPPSCFSGLHSLRHLWLDD
NALTEIPVQAFRSLSALQAMTLALNKIHHIPDYAFGNLSSLVVLHLHNNR
IHSLGKKCFDGLHSLETLDLNYNNLDEFPTAIRTLSNLKELGFHSNNIRS
IPEKAFVGNPSLITIHEYDNPIQFVGRSAFQHLPELRTLTLNGASQITEF
PDLTGTANLESLTLTGAQISSLPQTVCNQLPNLQVLDLSYNLLEDLPSFS
VCQKLQKIDLRHNEIYEIKVDTFQQLLSLRSLNLAWNKIAIIHPNAFSTL
PSLIKLDLSSNLLSSFPITGLHGLTHLKLTGNHALQSLISSENFPELKVI
EMPYAYQCCAFGVCENAYKISNQWNKGDNSSMDDLHKKDAGMFQAQDERD
LEDFLLDFEEDLKALHSVQCSPSPGPFKPCEHLLDGWLIRIGVWTIAVLA
LTCNALVTSTVFRSPLYISPIKLLIGVIAAVNMLTGVSSAVLAGVDAFTF
GSFARHGAWWENGVGCHVIGFLSIFASESSVFLLTLAALERGFSAKYSAK
FETKAPFSSLKVIILLCALLALTMAAVPLLGGSKYGASPLCLPLPFGEPS
TMGYMVALILLNSLCFLMMTIAYTKLYCNLDKGDLENIWDCSMVKHIALL
LFTNCILNCPVAFLSFSSLINLTFISPEVIKFILLVVVPLPACLNPLLYI
LFNPHFKEDLVSLRKQTYVWTRSKHPSLMSINSDDVEKQSCDSTQALVTF
TSSSITYDLPPSSVPSPAYPVTESCHLSSVAFVPCL:
AR: linker region
FLAG tag
SEQ ID NO: 269
DYKDDDDK:
AGA: linker region
FLAG tag
SEQ ID NO: 269
DYKDDDDK:
linker region
SEQ ID NO: 270
LDGG:
HIA tag
SEQ ID NO: 271
YPYDVPDYA:
AGA: linker region
HA tag
SEQ ID NO: 271
YPYDVPDYA:
Amino acid sequence hLGR5(ECD)-GPA33(TM)-
FLAG insert in pVaxl for expression on
cell surface:
SEQ ID NO: 6
MDTSRLGVLLSLPVLLQLATGGSSPRSGVLLRGCPTHCHCEPDGRMLLRV
DCSDLGLSELPSNLSVFTSYLDLSMNNISQLLPNPLPSLRFLEELRLAGN
ALTYIPKGAFTGLYSLKVLMLQNNQLRHVPTEALQNLRSLQSLRLDANHI
SYVPPSCFSGLHSLRHLWLDDNALTEIPVQAFRSLSALQAMTLALNKIHH
IPDYAFGNLSSLVVLHLHNNRIHSLGKKCFDGLHSLETLDLNYNNLDEFP
TAIRTLSNLKELGFHSNNIRSIPEKAFVGNPSLITIHFYDNPIQFVGRSA
FQHLPELRTLTLNGASQITEFPDLTGTANLESLTLTGAQISSLPQTVCNQ
LPNLQVLDLSYNLLEDLPSFSVCQKLQKIDLRHNEIYEIKVDTFQQLLSL
RSLNLAWNKIAIIHPNAFSTLPSLIKLDLSSNLLSSFPITGLHGLTHLKL
TGNHALQSLISSENFPELKVIEMPYAYQCCAFGVCENAYKISNQWNKGDN
SSMDDLHKKDAGMFQAQDERDLEDFLLDFEEDLKALHSVQCSPSPGPFKP
CEHLLDGWLIRVALYVGIAVGVVAALIIIGIIIYCCCCRGKDDNTEDKED
ARPNREAYEEPPEQLRELSREREEEDDYRQEEQRSTGRESPDHLDQDYKD
DDDK

Of which:

signal peptide
SEQ ID NO: 275
MDTSRLGVLLSLPVLLQLATG:
hLGR5 (CD)
SEQ ID NO: 276
GSSPRSGVLLRGCPTHCHCEPDGRMLLRVDCSDLGLSELPSNLSVFTSYL
DLSMNNISQLLPNPLPSLRFLEELRLAGNALTYIPKGAFTGLYSLKVLML
QNNQLRHVPTEALQNLRSLQSLRLDANHISYVPPSCFSGLHSLRHLWLDD
NALTEIPVQAFRSLSALQAMTLALNKIHHIPDYAFGNLSSLVVLHLHNNR
IHSLGKKCFDGLHSLETLDLNYNNLDEFPTAIRTLSNLKELGFHSNNIRS
IPEKAFVGNPSLITIHFYDNPIQFVGRSAFQHLPELRTLTLNGASQITEF
PDLTGTANLESLTLTGAQISSLPQTVCNQLPNLQVLDLSYNLLEDLPSFS
VCQKLQKIDLRHNEIYEIKVDTFQQLLSLRSLNLAWNKIAIIHPNAFSTL
PSLIKLDLSSNLLSSFPITGLHGLTHLKLTGNHALQSLISSENFPELKVI
EMPYAYQCCAFGVCENAYKISNQWNKGDNSSMDDLHKKDAGMFQAQDERD
LEDFLLDFEEDLKALHSVQCSPSPGPFKPCEHLLDGWLIR:
GPA33 sequence containing the TM region
SEQ ID NO: 277
VALYVGIAVGVVAALIIIGIIIYCCCCRGKDDNTEDKEDARPNREAYEEP
PEQLRELSREREEEDDYRQEEQRSTGRESPDHLDQ:
FLAG
SEQ ID NO: 269
DYKDDDDK:

### Mouse LGR5

The cDNA encoding full length (FL) mouse (m)LGR5 was present in pEF1_Myc/His (Invitrogen), containing a cMyc and a HIS tag (pEF1_mLgr5-Myc-HIS). The mLgr5 cDNA sequence was verified by sequencing and comparison with the NCBI Reference sequence NM_010195.2.

Amino acid sequence full length mouse (m)
Lgr5-Myc-HIS insert in pEF1_Myc/His for
expression on cell surface
SEQ ID NO: 7
MDTSCVHMLLSLLALLQLVAAGSSPGPDAIPRGCPSHCHCELDGRMLLRV
DCSDLGLSELPSNLSVFTSYLDLSMNNISQLPASLLHRLCFLEELRLAGN
ALTHIPKGAFTGLHSLKVLMLQNNQLRQVPEEALQNLRSLQSLRLDANHI
SYVPPSCFSGLHSLRHLWLDDNALTDVPVQAFRSLSALQAMTLALNKIHH
IADYAFGNLSSLVVLHLHNNRIHSLGKKCFDGLHSLETLDLNYNNLDEFP
TAIKTLSNLKELGFHSNNIRSIPERAFVGNPSLITIHFYDNPIQFVGVSA
FQHLPELRTLTLNGASHITEFPHLTGTATLESLTLTGAKISSLPQAVCDQ
LPNLQVLDLSYNLLEDLPSLSGCQKLQKIDLRHNEIYEIKGSTFQQLFNL
RSLNLAWNKIAIIHPNAFSTLPSLIKLDLSSNLLSSFPVTGLHGLTHLKL
TGNRALQSLIPSANFPELKIIEMPSAYQCCAFGGCENVYKISNQWNKDDG
NSVDDLHKKDAGLFQVQDERDLEDFLLDFEEDLKALHSVQCSPSPGPFKP
CEHLFGSWLIRIGVWTTAVLALSCNALVALTVFRTPLYISSIKLLIGVIA
VVDILMGVSSAVLAAVDAFTFGRFAQHGAWWEDGIGCQIVGFLSIFASES
SIFLLTLAALERGFSVKCSSKFEVKAPLFSLRAIVLLCVLLALTIATIPL
LGGSKYNASPLCLPLPFGEPSTTGYMVALVLLNSLCFLIMTIAYTKLYCS
LEKGELENLWDCSMVKHIALLLFANCILYCPVAFLSFSSLLNLTFISPDV
IKFILLVIVPLPSCLNPLLYIVFNPHFKEDMGSLGKHTRFWMRSKHASLL
SINSDDVEKRSCESTQALVSFTHASIAYDLPSTSGASPAYPMTESCHLSS
VAFVPCLAAARGHPFEQKLISEEDLNMHTGHHHHHH

Of which:

signal peptide
SEQ ID NO: 278
MDTSCVHMLLSLLALLQLVAA
SEQ ID NO: 279
GSSPGPDAIPRGCPSHCHCELDGRMLLRVDCSDLGLSELPSNLSVFTSY
LDLSMNNISQLPASLLHRLCFLEELRLAGNALTHIPKGAFTGLHSLKVL
MLQNNQLRQVPEEALQNLRSLQSLRLDANHISYVPPSCFSGLHSLRHLW
LDDNALTDVPVQAFRSLSALQAMTLALNKIHHIADYAFGNLSSLVVLHL
HNNRIHSLGKKCFDGLHSLETLDLNYNNLDEFPTAIKTLSNLKELGFHS
NNIRSIPERAFVGNPSLITIHFYDNPIQFVGVSAFQHLPELRTLTLNGA
SHITEFPHLTGTATLESLTLTGAKISSLPQAVCDQLPNLQVLDLSYNLL
EDLPSLSGCQKLQKIDLRHNEIYEIKGSTFQQLFNLRSLNLAWNKIAII
HPNAFSTLPSLIKLDLSSNLLSSFPVTGLHGLTHLKLTGNRALQSLIPS
ANFPELKIIEMPSAYQCCAFGGCENVYKISNQWNKDDGNSVDDLHKKDA
GLFQVQDERDLEDFLLDFEEDLKALHSVQCSPSPGPFKPCEHLFGSWLI
RIGVWTTAVLALSCNALVALTVFRTPLYISSIKLLIGVIAVVDILMGVS
SAVLAAVDAFTFGRFAQHGAWWEDGIGCQIVGFLSIFASESSIFLLTLA
ALERGFSVKCSSKFEVKAPLFSLRAIVLLCVLLALTIATIPLLGGSKYN
ASPLCLPLPFGEPSTTGYMVALVLLNSLCFLIMTIAYTKLYCSLEKGEL
ENLWDCSMVKHIALLLFANCILYCPVAFLSFSSLLNLTFISPDVIKFIL
LVIVPLPSCLNPLLYIVENPHFKEDMGSLGKHTRFWMRSKHASLLSINS
DDVEKRSCESTQALVSFTHASIAYDLPSTSGASPAYPMTESCHLSSVAF
VPCL: mLGR5 (FL)
SEQ ID NO: 280
AAARGHPF: linker
SEQ ID NO: 281
EQKLISEEDL: cMyc derived epitope tag
SEQ ID NO: 282
NMHTG: linker
SEQ ID NO: 283
HHHHHH: Hexa His tag

Cloning of the cDNA Encoding Human ZNRF3

cDNA encoding human (h)ZNRF3 (Genbank NM_001206998.1) was used as template to amplify specific parts of the cDNA (the extra-cellular domain (ECD): amino acids 1-219, or the ECD-TM part (truncation mutant): amino acids 1-256). To be able to express the target protein on the surface of antigen-negative cells and to be able to generate stable cell clones, the cDNA encoding the ECD of hZNRF3 was cloned into pDisplay (pDisplay_hZNRF3(ECD)-cMyc-PDGFR(TM) and a truncation mutant (ECD with the autologous TM region) was cloned in pcDNA3.1 (pcDNA3.1_hZNRF3(ECD-TM)). The pDisplay construct allows confirmation of protein surface expression via an extra-cellular cMyc-derived epitope tag. Specific primers were designed, synthesized and used to amplify the specific parts of hZNRF3. These were then cloned in pDisplay (only the ECD), resulting in pDisplay_hZNRF3(ECD)-cMyc-PDGFR(TM) and in pcDNA3.1 (ECD with TM region), resulting in pcDNA3.1_hZNRF3(ECD-TM) for expression in (antigen-negative) cells.

Cloning of the cDNA Encoding Mouse ZNRF3 ECD and Human RNF43 ECD and Production of Recombinant Protein

The cDNAs encoding the ECD of mouse (m)ZNRF3 (Genbank NM_001080924.2, amino acid 53-205, the signal peptide used was derived from cystatin) and the ECD of human RNF43 (GenBank NM_017763, amino acid 24-109, containing the cystatin leader sequence) were used to generate recombinant purified, his-tagged proteins consisting of the ECDs of the targets, in vector pUPE (U-Protein Express BV). In brief: the constructs (Peng et al. 2013 Plos ONE 8:2-10) were transfected in Hek293E cells (293 c18 ATCC, CRL-10852) and after a week of production, the protein-containing supernatant was harvested. The hexa HIS-tagged proteins were purified by means of IMAC (immobilized metal-ion affinity chromatography) and gelfiltration. Another cDNA encoding the (codon-optimized) ECD of mouse ZNRF3 was made synthetically and cloned in frame with a cMyc-derived epitope tag and the transmembrane region of the Platelet-derived growth factor receptor (PDGFR) in pDisplay for expression on the cell surface (pDisplay_mZnrf3-cMyc-PDGFR(TM).

Cloning of the cDNA Encoding Human RNF43

The cDNA encoding human (h)RNF43 (GenBank NM_017763) was used as template to amplify specific parts of the cDNA (the extra-cellular domain (ECD): amino acid 1-199, or the ECD-TM part (truncation mutant): amino acid 1-222). To be able to express the target protein on the surface of antigen-negative cells and to be able to generate stable cell clones, the cDNA encoding the ECD of hRNF43 was cloned into pDisplay (pDisplay_hRNF43(ECD)-cMyc-PDGFR(TM) and a truncation mutant of hRNF43 (ECD with the autologous TM region) was cloned in pcDNA3.1 (pcDNA3.1_hRNF43(ECD-TM)). The pDisplay construct allows confirmation of protein surface expression via an extra-cellular cMyc-derived epitope tag. Specific primers were designed, synthesized and used to amplify the specific parts of hZNRF3. These were then cloned in pDisplay (only the ECD, for the purpose of immunization) and in pCDNA3.1 (ECD with TM region) for expression in (antigen-negative) cells and the generation of cell clones stably expressing the target.

Cloning of the cDNA Encoding Mouse RNF43

The cDNA encoding the ECD of mouse (m)RNF43 (Genbank NM_172448.3, amino acid 1-199) was ordered as a synthetic gene and was cloned in frame with a cMyc-derived epitope tag and the transmembrane region of the Platelet-derived growth factor receptor (PDGFR) in pDisplay for expression on the cell surface pDisplay_mRnf43-cMyc-PDGFR(TM).

Human ZNRF3(ECD) in pDisplay amino acid sequence
for expression on cell surface:
SEQ ID NO: 8
MRPRSGGRPGATGRRRRRLRRRPRGLRCSRLPPPPPLPLLLGLLLAAAG
PGAARAKETAFVEVVLFESSPSGDYTTYTTGLTGRFSRAGATLSAEGEI
VQMHPLGLCNNNDEEDLYEYGWVGVVKLEQPELDPKPCLTVLGKAKRAV
QRGATAVIFDVSENPEAIDQLNQGSEDPLKRPVVYVKGADAIKLMNIVN
KQKVARARIQHRPPRQPTEYFDMVDEQKLISEEDLNAVGQDTQEVIVVP
HSLPFKVVVISAILALVVLTIISLIILIMLWQKKPR

Of which:

SEQ ID NO: 284
MRPRSGGRPGATGRRRRRLRRRPRGLRCSRLPPPPPLPLLLGLLLAAAG
PGAARA: signal peptide
SEQ ID NO: 285
KETAFVEVVLFESSPSGDYTTYTTGLTGRFSRAGATLSAEGEIVQMHPL
GLCNNNDEEDLYEYGWVGVVKLEQPELDPKPCLTVLGKAKRAVQRGATA
VIFDVSENPEAIDQLNQGSEDPLKRPVVYVKGADAIKLMNIVNKQKVAR
ARIQHRPPRQPTEYFDM:
the ECD of human ZNRF3 VD: amino acids that are
encoded by cloning sites
SEQ ID NO: 281
EQKLISEEDL: cMyc-derived epitope tag
SEQ ID NO: 286
NAVGQDTQEVIVVPHSLPFKVVVISAILALVVLTIISLIILIMLWQKKP
R: PDGFR sequence containing the TM region
Human ZNRF3 truncation mutant amino acid sequence
for expression on cell surface:
SEQ ID NO: 9
MRPRSGGRPGATGRRRRRLRRRPRGLRCSRLPPPPPLPLLLGLLLAAAG
PGAARAKETAFVEVVLFESSPSGDYTTYTTGLTGRFSRAGATLSAEGEI
VQMHPLGLCNNNDEEDLYEYGWVGVVKLEQPELDPKPCLTVLGKAKRAV
QRGATAVIFDVSENPEAIDQLNQGSEDPLKRPVVYVKGADAIKLMNIVN
KQKVARARIQHRPPRQPTEYFDMGIFLAFFVVVSLVCLILLVKIKLKQR
RSQNSMNRPAV

Of which:

SEQ ID NO: 284
MRPRSGGRPGATGRRRRRLRRRPRGLRCSRLPPPPPLPLLLGLLLAAAG
PGAARA: signal peptide
SEQ ID NO: 288
KETAFVEVVLFESSPSGDYTTYTTGLTGRFSRAGATLSAEGEIVQMHPL
GLCNNNDEEDLYEYGWVGVVKLEQPELDPKPCLTVLGKAKRAVQRGATA
VIFDVSENPEAIDQLNQGSEDPLKRPVVYVKGADAIKLMNIVNKQKVAR
ARIQHRPPRQPTEYFD: ECD of human ZNRF3
SEQ ID NO: 289
MGIFLAFFVVVSLV: Predicted TM region
SEQ ID NO: 290
CLILLVKIKLKQRRSQNSMNRPAV: Intra cellular tail

Mouse ZNRF3 ECD-his in pUPE Soluble Amino Acid Sequence for Expression Soluble Protein:

SEQ ID NO: 10
GSKETAFVEVVLFESSPSGDYTTHTTGLTGRFSRAGAMLSAEGEIVQMH
PLGLCNNNDEEDLYEYGWVGVVKLEQPELDPKPCLTVLGKAKRAVQRGA
TAVIFDVSENPEAIDQLNQGSEDPLKRPVVYVKGADAIKLMNIVNKQKV
ARARIQHLAAAHHHHHH

Of which:

GS: amino acids encoded by cloning sites
SEQ ID NO: 291
KETAFVEVVLFESSPSGDYTTHTTGLTGRFSRAGAMLSAEGEIVQMHP
LGLCNNNDEEDLYEYGWVGVVKLEQPELDPKPCLTVLGKAKRAVQRGA
TAVIFDVSENPEAIDQLNQGSEDPLKRPVVYVKGADAIKLMNIVNKQK
VARARIQHL: ECD of mouse ZNRF3
AAA: amino acids encoded by cloning sites
tagSEQ ID NO: 283
HHHHHH: Hexa His

Mouse (m)ZNRF3 Sequence in pDisplay Amino Acid Sequence Expressed on Cell Surface:

SEQ ID NO: 11
MRPRSGGRPGAPGRRRRRLRRGPRGRRLPPPPPLPLLLGLLLAAAGPGA
ARAKETAFVEVVLFESSPSGDYTTHTTGLTGRFSRAGAMLSAEGEIVQM
HPLGLCNNNDEEDLYEYGWVGVVKLEQPELDPKPCLTVLGKAKRAVQRG
ATAVIFDVSENPEAIDQLNQGSEDPLKRPVVYVKGADAIKLMNIVNKQK
VARARIQHLPPRQPTEYFDMVDEQKLISEEDLNAVGQDTQEVIVVPHSL
PFKVVVISAILALVVLTIISLIILIMLWQKKPR

Of which:

SEQ ID NO: 292
MRPRSGGRPGAPGRRRRRLRRGPRGRRLPPPPPLPLLLGLLLAAAGPG
AARA: signal peptide
SEQ ID NO: 293
KETAFVEVVLFESSPSGDYTTHTTGLTGRFSRAGAMLSAEGEIVQMHP
LGLCNNNDEEDLYEYGWVGVVKLEQPELDPKPCLTVLGKAKRAVQRGA
TAVIFDVSENPEAIDQLNQGSEDPLKRPVVYVKGADAIKLMNIVNKQK
VARARIQHLPPRQPTEYFDM: ECD mZNRF3
VD: amino acids that are encoded by cloning
sites
SEQ ID NO: 281
EQKLISEEDL: cMyc derived epitope tag
SEQ ID NO: 287
NAVGQDTQEVIVVPHSLPFKVVVISAILALVVLTIISLIILIMLWQKK
PR: PDGFR sequence containing the TM region

Human RNF43 in pDisplay Amino Acid Sequence for Expression on Cell Surface:

SEQ ID NO: 12
MSGGHQLQLAALWPWLLMATLQAGFGRTGLVLAAAVESERSAEQKAIIR
VIPLKMDPTGKLNLTLEGVFAGVAEITPAEGKLMQSHPLYLCNASDDDN
LEPGFISIVKLESPRRAPRPCLSLASKARMAGERGASAVLFDITEDRAA
AEQLQQPLGLTWPVVLIWGNDAEKLMEFVYKNQKAHVRIELKEPPAWPD
YDVVDEQKLISEEDLNAVGQDTQEVIVVPHSLPFKVVVISAILALVVLT
IISLIILIMLWQKKPR

Of which:

SEQ ID NO: 294
MSGGHQLQLAALWPWLLMATLQAGFGRTGLVLAAAVESERSA:
signal peptide
SEQ ID NO: 295
EQKAIIRVIPLKMDPTGKLNLTLEGVFAGVAEITPAEGKLMQSHPLYL
CNASDDDNLEPGFISIVKLESPRRAPRPCLSLASKARMAGERGASAVL
FDITEDRAAAEQLQQPLGLTWPVVLIWGNDAEKLMEFVYKNQKAHVRI
ELKEPPAWPDYDV: ECD of human RNF43
VD: amino acids encoded by cloning sites
SEQ ID NO: 281
EQKLISEEDL: Mye derived epitope tag
SEQ ID NO: 287
NAVGQDTQEVIVVPHSLPFKVVVISAILALVVLTIISLIILIMLWQKK
PR: PDGFR sequence containing the TM region

Mouse RNF43 in pDisplay Amino Acid Sequence for Expression on Cell Surface

SEQ ID NO: 13
MSGGHQLQLAVLWPWLLMATLHAGFGHTGRVLAAAVESERSAEQKAVIR
VIPLKMDPTGKLNLTLEGVFAGVAEVTPAEGKLMQSHPLYLCNASDDDN
LEPGFISIVKLESPRRAPRPCLSLASKARMAGERGANAVLFDITEDRSA
AEQLQQPLGLTKPVVLIWGSDAAKLMEFVYKNRKAYVWIELKEPPAGAN
YDVVDEQKLISEEDLNAVGQDTQEVIVVPHSLPFKVVVISAILALVVLT
IISLIILIMLWQKKPR

Of which:

SEQ ID NO: 296
MSGGHQLQLAVLWPWLLMATLHAGFGHTGRVLAAAVESERSA:
signal peptide
SEQ ID NO: 297
EQKAVIRVIPLKMDPTGKLNLTLEGVFAGVAEVTPAEGKLMQSHPLYL
CNASDDDNLEPGFISIVKLESPRRAPRPCLSLASKARMAGERGANAVL
FDITEDRSAAEQLQQPLGLTKPVVLIWGSDAAKLMEFVYKNRKAYVWI
ELKEPPAGANYDV: ECD of mouse RNF43
VD: amino acids encoded by cloning site
SEQ ID NO: 281
EQKLISEEDL: Myc derived epitope tag
SEQ ID NO: 287
NAVGQDTQEVIVVPHSLPFKVVVISAILALVVLTIISLIILIMLWQKK
PR: PDGFR sequence containing TM region

Cloning of the cynomolgus and rat orthologues of LGR5 and EGFR

The cDNA encoding cynomolgus LGR5 was amplified from cynomolgus colon- and from liver cDNA using human-specific primers (Cyno-LGR5-FOR: 5′-CCAGCAGGATCCGCCGCCACCATGGACACCTCCCGGCTCGGTG-3′ SEQ ID NO: 14 and Cyno-LGR5-REV: 5′-CCAGCAGCGGCCGCTTAGAGACATGGGACAAATGCCAC-3′ SEQ ID NO: 15). DMSO was added to the reaction to possibly improve the yield and specificity of the PCR reactions. Both liver and colon cDNA allowed for the amplification of the expected 2.7 kb band and the PCR product obtained from colon cDNA was then used for cloning. The obtained cDNA was cloned into pcDNA3.1 (BamH1-Not1) and sequenced. The sequence encoding a chimeric EGF receptor composed of the cynomolgus extra-cellular domain (also described in WO2010/022736-A2) coupled to the human trans-membrane and intra-cellular part was obtained from patent US2010/0183615-A1 (page 79) and the encoding cDNA was codon-optimised for expression in human cells, ordered and cloned by GeneArt. This cDNA was then re-cloned into pcDNA3.1 using Nhe1 and Not1. The cDNA's encoding rat EGFR and rat LGR5 are available through Sino Biological (cDNA encoding rat EGFR cat. nr. RG80100-UT; GenBank reference HM801041.1) and Origene (cDNA encoding rat LGR5: cat. Nr. RN202738; GenBank reference NM_001106784). Both cDNA's were re-cloned into pcDNA3.1 using Kpn1 and Nota. All cDNA's were sequenced and shown to be correct.

Full length wt cynomolgus LGR5 amino acid sequence
SEQ ID NO: 16
MDTSRLGVLLSLPVLLQLAAGSSSPRSGALLRGCPTHCHCEPDGRMLLR
VDCSDLGLSELPSNLSVFTSYLDLSMNNISQLLPNPLPSLRFLEELRLA
GNALTYIPKGAFTGLYSLKVLMLQNNQLRQVPTEALQNLRSLQSLRLDA
NHISYVPPSCFSGLHSLRHLWLDDNALTEIPVQAFRSLSALQAMTLALN
KIHHIPDYAFGNLSSLVVLHLHNNRIHSLGKKCFDGLHSLETLDLNYNN
LDEFPTAIRTLSNLKELGFHSNNIRSIPEKAFVGNPSLITIHFYDNPIQ
FVGRSAFQHLPELRTLTLNGASQITEFPDLTGTANLESLTLTGAQISSL
PQTVCNQLPNLQVLDLSYNLLEDLPSFSVCQKLQKIDLRHNEIYEIKVD
TFQQLLSLRSLNLAWNKIAIIHPNAFSTLPSLIKLDLSSNLLSSFPVTG
LHGLTHLKLTGNHALQSLISSENFPELKIIEMPYAYQCCAFGVCENAYK
ISNQWNKGDNSSMDDLHKKDAGMFQVQDERDLEDFLLDFEEDLKALHSV
QCSPSPGPFKPCEHLLDGWLIRIGVWTIAVLALTCNALVTSTVFRSPLY
ISPIKLLIGVIAVVNMLTGVSSAVLAGVDAFTFGSFARHGAWWENGVGC
QVIGFLSIFASESSVFLLTLAALERGFSVKCSAKFETKAPFSSLKVIIL
LCALLALTMAAVPLLGGSEYGASPLCLPLPFGEPSTTGYMVALILLNSL
CFLMMTIAYTKLYCNLDKGDLENIWDCSMVKHIALLLFTNCILYCPVAF
LSFSSLLNLTFISPEVIKFILLVIVPLPACLNPLLYILFNPHFKEDLVS
LGKQTYFWTRSKHPSLMSINSDDVEKQSCDSTQALVTFTSSSIAYDLPP
SSVPSPAYPVTESCHLSSVAFVPCL

Of which

SEQ ID NO: 298
MDTSRLGVLLSLPVLLQLAAG Signal peptide
SEQ ID NO: 286
SSSPRSGALLRGCPTHCHCEPDGRMLLRVDCSDLGLSELPSNLSVFTSY
LDLSMNNISQLLPNPLPSLRFLEELRLAGNALTYIPKGAFTGLYSLKVL
MLQNNQLRQVPTEALQNLRSLQSLRLDANHISYVPPSCFSGLHSLRHLW
LDDNALTEIPVQAFRSLSALQAMTLALNKIHHIPDYAFGNLSSLVVLHL
HNNRIHSLGKKCFDGLHSLETLDLNYNNLDEFPTAIRTLSNLKELGFHS
NNIRSIPEKAFVGNPSLITIHFYDNPIQFVGRSAFQHLPELRTLTLNGA
SQITEFPDLTGTANLESLTLTGAQISSLPQTVCNQLPNLQVLDLSYNLL
EDLPSFSVCQKLQKIDLRHNEIYEIKVDTFQQLLSLRSLNLAWNKIAII
HPNAFSTLPSLIKLDLSSNLLSSFPVTGLHGLTHLKLTGNHALQSLISS
ENFPELKIIEMPYAYQCCAFGVCENAYKISNQWNKGDNSSMDDLHKKDA
GMFQVQDERDLEDFLLDFEEDLKALHSVQCSPSPGPFKPCEHLLDGWLI
RIGVWTIAVLALTCNALVTSTVFRSPLYISPIKLLIGVIAVVNMLTGVS
SAVLAGVDAFTFGSFARHGAWWENGVGCQVIGFLSIFASESSVFLLTLA
ALERGFSVKCSAKFETKAPFSSLKVIILLCALLALTMAAVPLLGGSEYG
ASPLCLPLPFGEPSTTGYMVALILLNSLCFLMMTIAYTKLYCNLDKGDL
ENIWDCSMVKHIALLLFTNCILYCPVAFLSFSSLLNLTFISPEVIKFIL
LVIVPLPACLNPLLYILFNPHFKEDLVSLGKQTYFWTRSKHPSLMSINS
DDVEKQSCDSTQALVTFTSSSIAYDLPPSSVPSPAYPVTESCHLSSVAF
VPCL Cynomolgus LGR5

### Generation of LGR4, LGR5, ZNRF3 and RNF43 Over-Expressing Cell Lines

Constructs expressing hLGR4 (pEF1_hLGR4-FLAG-HA), hLGR5 (pEF1_hLGR5-FLAG-HA), hZRNF3(ECD) (pcDNA3.1_hZNRF3(ECD) and pDisplay_hZNRF3(ECD)-Myc-PDGFR(TM) and hRNF43(ECD) (pDisplay_hRNF43-(ECD)-Myc-PDGFR(TM) were used to generate Freestyle 293F and CHO-K1 clones stably expressing the respective proteins. Constructs were transiently transfected in Freestyle 293F and CHO-K1 cells using PEI (Freestyle 293F cells) or lipofectamine (CHO cells) transfection and screened by FACS using antibodies reacting with the respective targets. After successful transfection both Freestyle 293F and CHO-K1 cells were seeded in limiting dilution and cultured under 0.5 mg/ml G418 selection pressure to obtain stable cell clones. After 2-3 weeks of selection, clones were screened by FACS. The selected clones were expanded by serial passage, retested in FACS and frozen to −150° C.

### Mice Used for Immunization

For generation of human antibodies binding to LGR4, LGR5, ZNRF3 and RNF43 mice transgenic for the human VK1-39 light chain (common light chain mice, see WO2009/157771) and for a human heavy chain (HC) minilocus (comprising a selection of human V gene segments, all human Ds and all human Js) were immunized with either DNA encoding the proteins or recombinant DNA as briefly described below. These mice are referred to as ‘MeMo®’ mice.

### Immunizations

hLGR4/hLGR5 DNA Immunizations

18 mice were immunized with 20 μg plasmid DNA (pVax1_hLGR4-FLAG-HA and pVax1_hLGR5-FLAG-HA) expressing full length hLGR4 and hLGR5 each. Additionally 12 mice were immunized with 20 μg plasmid DNA of the extracellular domain of hLGR4 and hLGR5 fused to the transmembrane domain of GPA33 and a Flag tag (pVax1_hLGR4(ECD)-GPA33-FLAG and pVax1_hLGR5(ECD)-GPA33-FLAG). Mice were vaccinated at day 0, 3, 6, 14, 17, 28, 31, 49, 63, 70, 84 and 91.

LGR4, LGR5, ZNRF3, RNF43 protein Immunizations

Six mice were immunized with 40 pig recombinant human (rh)LGR4-Fc (RND systems Cat. Nr. 7750-GP), rhLGR5-Fc (RND systems Cat. Nr. 8078-GP), rhZNRF3-Fc (RND systems Cat. Nr. 7994-RF) or rhRNF43-Fc (RND systems Cat. nr. 7964-RN) protein dissolved in PBS, and supplemented with 40 μl Gerbu adjuvant MM (Gerbu Biotechnik). Subsequently mice were boosted with 20 μg recombinant protein (+20 μl Gerbu adjuvant MM) on day 14 and day 28 followed by further boosts of 20 μg recombinant protein until a serum titer was observed or the immunization period was greater than three months.

### Determination of Antibody Titers

Anti-hLGR4, hLGR5, hZNRF3 or hRNF43 titers in the serum from immunized mice were determined by FACS on Freestyle 293F cells over-expressing the respective target.

### Recovery of Lymphoid Tissue

Spleen and draining lymph nodes were removed from all mice that were successfully immunized (see table 1). Single cell suspensions were generated from both spleen and inguinal lymph nodes and subsequently these tissues were lysed in Trizol reagent and stored at −80° C. until use.

### Generation of ‘Immune’ Phage Antibody Repertoires by RT-PCR Cloning of VH Genes

From successfully immunized mice, the inguinal lymph nodes were used for the construction of ‘immune’ phage antibody repertoires. RNA was extracted from the lymphoid tissue using Trizol and 1 μg of total RNA was used in a RT reaction using an IgG-CH1 specific primer. The resulting cDNA was then used to amplify the polyclonal pool of VH-encoding cDNA using in-house developed VH-specific primers essentially as described in Marks et al. (J Mol Biol. 1991 Dec. 5; 222(3):581-97). The resulting PCR product was then cloned in a phagemid vector for the display of Fab fragments on phage, as described in de Haard et al. (J Biol Chem. 1999 Jun. 25; 274(26):18218-30) with the exception that the light chain was the same for every antibody and was encoded by the vector. After ligation, the phagemids were used to transform E. coli TG1 bacteria and transformed bacteria were plated onto LB-agar plates containing ampicillin and glucose. All phage libraries contained >10⁶ transformants and had an insert frequency of >80%. Bacteria were harvested after overnight growth and used to prepare phage according to established protocols (de Haard et al., J Biol Chem. 1999 Jun. 25; 274(26):18218-30).

Selection of Phage Carrying Fab Fragments Specifically Binding to LGR4, LGR5, ZNRF3 and RNF43 from Synthetic or ‘Immune’ Phage Antibody Repertoires

Phage libraries were rescued according to standardized procedures (J Mol Biol. 1991 Dec. 5; 222(3):581-97; J Biol Chem. 1999 Jun. 25; 274(26):18218-30) and phage were selected for two rounds of selection in case of in-house generated synthetic repertoires and a single round in case of the immune phage antibody repertoires. In the first round, recombinant protein was coated onto the wells of a MAXISORP™ ELISA plate or to a NUNC immuno-tube, whereas in the second round, either recombinant protein or cells over-expressing the target were used. The MAXISORP™ ELISA plates or immuno-tubes were blocked with 4% ELK. Phage antibody libraries were also blocked with 4% ELK and excess of human IgG to deplete for Fc region binders prior to the addition of the phage library to the coated antigen.

Incubation with the phage library with the coated protein was performed for 2 hrs at room temperature under shaking conditions. Plates or tubes were then washed five to ten times with 0.05% Tween-20 in PBS followed by 5 to 10 times washing with PBS. Bound phage were eluted using 50 mM glycine (pH 2.2) and added to E. coli TG-1 and incubated at 37° C. for phage infection. Subsequently infected bacteria were plated on agar plates containing Ampicillin, and glucose and incubated at 37° C. overnight. After the first round of selection, colonies were scraped off the plates and combined and thereafter rescued and amplified to prepare an enriched first round phage pool for the synthetic repertoires. For the ‘immune’ repertoires, single clones were screened for target binding after the first round of phage selection.

For the synthetic repertoires, the enriched library was then selected on either the recombinant human (rh) protein (rhLGR4-Fc, rmLgr4-Fc, rhLGR5-Fc, rhZNRF3-Fc or rhRNF43-Fc, RND systems, soluble rhRNF-43-HIS, rmZnrf3-HIS (produced in-house)) using the protocol described above or on Freestyle 293F cells stably over-expressing hLGR4(FL), hLGR5(FL), hZNRF3(ECD) or hRNF43(ECD). For the cell selections, rescued phage and cells were blocked with 4% ELK. After blocking the rescued phage were incubated with 5\hat{ }10⁶ parental Freestyle 293F cells for subtraction for 1 hr. After subtraction, cells were spun down and the phage supernatant was transferred to Freestyle 293F cells expressing hLGR4, hLGR5, hZNRF3 or hRNF43. Cells plus phage were incubated for 2 hrs at 2-8° C. Washing the cells (5-10 times) was performed using 5 ml of 0.5% BSA in PBS. Bound phage were eluted using 200 mM TEA, the eluate was neutralized with Tris (pH8) and added to E. coli TG-1 and incubated at 37° C. for phage infection. Subsequently, phage-infected bacteria were plated on agar plates containing ampicillin, and glucose and incubated at 30° C. or 37° C. overnight.

After the second round selection, individual clones were picked and tested for target-reactivity. Positive phage clones binding hLGR4, hLGR5, hZNRF3, or hRNF43 were then identified in FACS for binding to the Freestyle 293F stable over-expressing the respective target (see below).

### DiD Labeling of Cells and FACS Staining of a Mix of DiD-Positive and -Negative Cells

Freestyle 293F cell clones stably over-expressing the respective targets were generated in house. These cells were cultured and harvested using standardized procedures. Approximately 20 million antigen-expressing cells were then labeled with 1 μl of DiD (Invitrogen, cat. nr. V22889) in a volume of 1 ml of cell culture medium, for 20 minutes at 37° C. DiD labeling was checked on a small aliquot of cells in FACS and consistently found to be more than 90%. Cells were then washed twice with 20 ml of cell culture medium and used subsequently for FACS staining. To this aim, a 1:1 mix of antigen-negative (parental) Freestyle 293F cells and DiD-labeled antigen-positive cells was made and aliquoted at 200,000 cells per well into round bottom 96 wells FACS microtiter plates. Staining using monoclonal phage was performed as described below.

### FACS Staining of a Mix of Target Positive and -Negative Cols Using Monoclonal Phage

Monoclonal phage of single clones selected for binding the respective targets were prepared as described (J Mol Biol. 1991 Dec. 5; 222(3):581-97; J Biol Chem. 1999 Jun. 25; 274(26):18218-30). These were tested for binding in FACS to a mix of (DiD-labeled) antigen-positive and (non-labeled)-negative cell lines by incubation with phage (30 μl) in a total of 100 μl FACS buffer (PBS, containing 0.5% BSA and 0.5 mM EDTA) containing 4% milk. After three washes, bound phage were detected by staining with a biotinylated anti-M13 antibody (Fitzgerald, cat. nr. 61R-M101ABTB62-FEZ, 1:125 in FACS buffer, 30 minutes on ice) and PE-labeled streptavidin (Invitrogen, cat. nr. SA1004-4; 1:400 in FACS buffer for 15 minutes on ice). Stained cells were analysed using a FACS Canto (Becton and Dickinson).

### RTK-Targeting Antibodies

EGFR- and HER3-targeting cLC antibodies were obtained using previously described methods (J Mol Biol. 1991 Dec. 5; 222(3):581-97; J Biol Chem. 1999 Jun. 25; 274(26):18218-30) from in-house generated large synthetic phage antibody repertoires (HER3), or from phage antibody repertoires generated from successfully target-immunised MeMo mice (EGFR). Methods to generate antibodies to EGFR and HER3 and antibody variable domain VH chains for the respective EGFR and HER3 antibodies have been described in pending applications that are incorporated herein by reference: WO 2015/130173 A1 and WO 2015/130172 A1.

Re-Cloning of VH-Encoding cDNA's from the Phagemid Vector to IgG-Expression Vectors

The VH-encoding cDNA's of all target-specific clones were sequenced. A selection of unique clones based on sequence identity and cluster analysis (FIG. 25) was then re-cloned to different IgG expression vectors using Sfi1-BstEII or a SfiI/XhoI digestion and ligation of the pool of digested cDNA's into the IgG expression plasmid according to standardised molecular biological techniques.

### Generation of Bispecific Antibodies

Bispecific antibodies were generated by transient co-transfection of two plasmids encoding IgG with different VH domains, using a proprietary CH3 engineering technology to ensure efficient hetero-dimerisation and formation of bispecific antibodies. The common light chain is also co-transfected in the same cell, either on the same plasmid or on another plasmid. In our co-pending applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa. It was previously demonstrated in our co-pending applications that the negatively charged DE-variant heavy chain and positively charged KK-variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains.

VH genes encoding the antibodies binding LGR4, LGR5, ZNRF3 and RNF43 described above were cloned into the vector encoding the positively charged CH3 domain and RTK (EGFR- or HER3-) targeting antibodies as previously obtained and disclosed in WO 2015/130172 (incorporated herein by reference) were cloned into vector encoding the negatively charged CH3 domain. Suspension growth-adapted 293F Freestyle cells were cultivated in T125 flasks on a shaker plateau until a density of 3.0×10⁶ cells/ml. Cells were seeded at a density of 0.3-0.5×10⁶ viable cells/ml in each well of a 24-deep well plate. The cells were transiently transfected with a mix of two plasmids encoding different antibodies, cloned into the proprietary vector system. Seven days after transfection, the cellular supernatant was harvested and filtered through a 0.22 μM filter (Sartorius). The sterile supernatant was stored at 4° C. until purification of the antibodies.

### Generation of Monoclonal Antibodies

A selection of VH sequences were also re-cloned into an IgG1 expression vector encoding mono-specific, bivalent IgG. Suspension adapted 293F Freestyle cells were cultivated in T125 flasks at a shaker plateau until a density of 3.0×10⁶ cells/ml. Cells were seeded at a density of 0.3-0.5×10⁶ viable cells/ml in each well of a 24-deep well plate. The cells were transiently transfected with individual sterile DNA: PE1 mixtures according to standardized procedures and further cultivated. Seven days after transfection, supernatant was harvested and filtered through a 0.22 μM (Sartorius) filter. The sterile supernatant was stored at 4° C. until antibody was purified by means of protein-A affinity chromatography.

### Cloning and Expression of Comparator Antibodies Targeting LGR5, FZD7 and R-Spondin3.

Antibodies that specifically recognise EGFR, FZD, RSPO3 or LGR5 are known in the art. Comparator antibodies were constructed according to published information: cDNA sequences encoding the VH- and VL were derived from published patents and cloned into an expression vector encoding a full length human IgG1 molecule. Antibodies were subsequently expressed in 293F Freestyle cells by transient transfection and purified from the culture supernatant using protein-A affinity chromatography according to standard procedures. The hu8E11v2 (anti-LGR5) antibody was copied from patent US 2013/0336885 A1 (Genentech). Sequence information for the BNC101 antibody (anti-LGR5) was obtained from patent US2016/0031984 A1 (Bionomics Inc.). The sequence encoding the OMP18R5 antibody (anti-FZD) was copied from patent AU2014/212081 A1 (Oncomed Pharmaceuticals Inc.). The OMP18R20 and OMP18R21 antibody sequences (anti-LGR5) were found in and copied from U.S. Pat. No. 8,628,774 B2 (Oncomed Pharmaceuticals Inc.). The sequence encoding OMP131R10 (anti-RSPO3) was copied from patent WO 2016/090024 A2 (Oncomed Pharmaceuticals Inc.). A clinical batch of cetuximab (Erbitux®) was used. A list of the copied antibodies and the internal number given to these versions is given in Table 7.

### IgG Purification for Functional Screening

Purification of IgG was performed on a small scale (<500 μg), medium scale (<10 mg) and large scale (>10 mg) using protein-A affinity chromatography. Small scale purifications were performed under sterile conditions in 24 well filter plates using filtration. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration. Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH7.4. The IgG concentration was measured using Octet. Protein samples were stored at 4° C.

### IgG Quantification Using Octet

To determine the amount of IgG purified, the concentration of antibody was determined by means of Octet analysis using protein-A biosensors (Forte-Bio, according to the supplier's recommendations) using total human IgG (Sigma Aldrich, cat. nr. 14506) as standard.

### Characterization of Binding of LGR4/LGR5/ZNRF3/RNF43 Specific IgG

Antibodies targeting LGR4, LGR5, ZNRF3 or RNF43 were first expressed as bispecific common light chain antibodies with a second Fab arm recognizing a non-relevant (control) antigen: Tetanus Toxoid. The antibodies were tested for binding in FACS to Freestyle 293F cells overexpressing hLGR4, hLGR5, hZNR3 or hRNF43. Therefore cells were harvested and diluted to 10⁶ cells/ml in FACS buffer (PBS/0.5% BSA/0.5 mM EDTA). 1-2×10⁵ cells were added to each well in a U-bottom 96 well plate. Cells were centrifuged for 2 minutes at 300 g at 4° C. Supernatant was discarded by inverting the plate(s). 50 μl of each IgG sample was added (for screening diluted to a concentration of 5 μg/ml in FACS buffer) and incubated for 1H on ice. Cells were centrifuged once, supernatant was removed and cells were washed twice with 150 μl of FACS buffer. 50 μl diluted 1:400 goat anti human IgG PE (Invitrogen) was added and incubated for 30 minutes on ice in the dark. After adding FACS buffer, cells were centrifuged once, supernatant was removed and cells were washed twice with FACS buffer. Cells were analyzed on a FACSCanto Flow cytometer (Becton and Dickinson) in a HTS setting. Binding of the antibodies to cells was assessed by measuring the mean fluorescence intensity (MFI) of the stained cell population. Antibodies were considered to bind their target when the MFI was at least five-fold that of the same cell population stained with a (negative control) non-binding antibody (directed to tetanus toxoid).

To test for binding reactivity, ELISA assays were also used. LGR4 and LGR5 targeting bispecific IgG were tested for reactivity against the antigens rhLGR4-Fc, rhLGR5-Fc and Tetanus Toxoid. ZNRF3 and RNF43 antibodies were tested for reactivity against the antigens rhZNRF3-Fc, rhRNF43-Fc and Tetanus Toxoid. Antigens were coated overnight to MAXISORP™ ELISA plates. Wells of the ELISA plates were blocked with PBS (pH 7.2) containing 5% BSA for 1 hour. Selected antibodies were tested at a concentration of 5 μg/ml diluted in PBS-5% BSA and allowed to bind for 1 hour at 25° C. Alternatively when titrating the bispecific IgG, a seven step two-fold dilution series was prepared starting at 5 μg/ml or 8 μg/ml. As a control, the procedure was performed simultaneously with a commercially-available antibody specific for the coated antigens and an anti-Tetanus Toxoid control antibody. The ELISA plates were washed 3 times with PBS-T (PBS-0.05% v/v Tween 20). Bound IgG was detected with 1:2000 diluted HRP-conjugate (Goat anti-human IgG Becton Dickinson) and was allowed to bind for 1 hour at 25° C. The ELISA plates were washed 3 times with PBS-T (PBS-0.05% Tween 20) and bound IgG was visualized by TMB/H2O2 staining and staining was quantified by means of OD450 nm measurement.

### Affinity Ranking of LGR4/LGR5/ZNRF3/RNF43 Specific IgG in FACS

Cells were harvested and diluted to 10⁶ cells/ml in FACS buffer (PBS/0.5% BSA/0.5 mM EDTA). 1-2×10⁵ cells were added to each well in a U-bottom 96 well FACS plate. Cells were centrifuged for 2 minutes at 300 g at 4° C. Supernatant was discarded by inverting plate(s). 50 μl of each IgG sample was added in a seven step two-fold serial dilution starting at 5 μg/ml and incubated for 1 hr on ice. Cells were centrifuged once, supernatant was removed and cells were washed twice with 150 μl of FACS buffer. 50 μl 1:400 diluted mouse anti human IgG PE (Invitrogen) was added and incubated for 30 minutes on ice in the dark. After adding FACS buffer, cells were centrifuged once, supernatant was removed and cells were washed twice with FACS buffer. Cells were analyzed on a FACSCanto Flow cytometer in a HTS setting. Binding of the antibodies to cells was quantified by measuring the mean fluorescence intensity (MFI) and calculating the area under the curve (AUC) resulting from the resulting plots of the MFI as a function of the antibody concentration used for staining.

### IgG Purification for Functional Studies

Medium scale purifications were performed on an ÄKTAexplorer 100 system using HiTrap MabSelect Sure columns and HiTrap desalting columns. Samples were loaded at 5 ml/min. The column was washed with 2 column volumes of PBS. IgG was eluted at pH 3.0 with 0.1 M citrate buffer. Next the sample was desalted and ended up in a final buffer of PBS pH 7.4. IgG were filtered through a 0.45 μM filter (Sartorius). The IgG concentration was measured using OD280. Protein samples were stored at −80° C.

Testing Antibodies for their Reactivity with the Mouse Orthologue of the Target

To test for mLgr4 binding reactivity, an ELISA assay was used. Both LGR4- and LGR5-targeting antibodies were tested for reactivity against the recombinant mLgr4-Fc (RND systems, Cat. nr. 8077-GP) protein. mLgr4-Fc was coated overnight to MAXISORP™ ELISA plates. Wells of the ELISA plates were blocked with PBS (pH 7.2) containing 5% BSA for 1 hour. Selected antibodies were tested at a single concentration of 5 μg/ml diluted in PBS-5% BSA and allowed to bind for 1 hour at 25° C. As a control, the procedure was performed simultaneously with a commercially-available antibody specific for the coated antigens and a control (anti-TT cLC) antibody. The ELISA plates were washed 3 times with PBS-T (PBS-0.05% v/v Tween 20). Bound IgG was detected with 1:2000 diluted HRP-conjugate (Goat anti-human IgG; Becton Dickinson) and was allowed to bind for 1 hour at 25° C. The ELISA plates were washed 3 times with PBS-T (PBS-0.05% Tween 20) and bound IgG was visualized by TMB/H₂O₂ staining; staining was quantified by means of OD450 nm measurement.

Bispecific IgG were also tested for binding in FACS to Freestyle 293F cells transiently expressing mLgr5, mZnr3 or mRnf43. Therefore cells were transiently transfected with mLgr5, mZnrf3 or mRnf43-encoding constructs (pEF1_mLgr5-Myc-HIS, pDisplay_mZnrf3-Myc-PDGFR(TM), pDisplay_mRnf43-Myc-PDGFR(TM) using lipofectamine and were harvested and diluted to 10⁶ cells/ml in FACS buffer (PBS/0.5% BSA/0.5 mM EDTA). 1-2×10⁵ cells were added to each well in a U-bottom 96 well plate. Cells were centrifuged for 2 minutes at 300 g at 4° C. Supernatant was discarded by inverting plate(s). 50 μl of each IgG sample was added at a concentration of 5 μg/ml and incubated for 1 hour on ice. Cells were centrifuged once, supernatant was removed and cells were washed twice with FACS buffer. 50 μl diluted 1:400 mouse anti human IgG PE (Invitrogen) was added and incubated for 30-60 minutes on ice in the dark. After adding FACS buffer, cells were centrifuged once, supernatant was removed and cells were washed twice with FACS buffer. Cells were analyzed on a FACSCanto Flow cytometer in a HTS setting. Binding of the antibodies to cells was quantified by measuring the mean fluorescence intensity (MFI) of the transfected cell population.

### R-Spondin3 Blocking ELISA Assay

All four WNT targets have R-Spondin as their ligand (Prog Biophys Mol Biol. 2015 September; 118(3):112-8; J Struct Biol. 2015 August; 191(2):149-55). For each target, a ligand-blocking assay was developed to test the ability of target (LGR4, LGR5, ZNRF3 or RNF43) specific antibodies to interfere with R-Spondin3 binding to the target. Binding of the Fc fusion proteins of the ECD of the respective target to coated R-Spondin3 was tested in the presence of an excess of cLC IgG directed to LGR4, LGR5, ZNRF3 or RNF43. In case the cLC IgG binds to the R-Spondin3 binding site, the Fc fusion protein would no longer be able to bind to the coating and the ELISA signal would be lost. Recombinant R-Spondin3 (RND systems) was coated onto the wells of a MAXISORP™ ELISA plate. Wells of the ELISA plates were blocked with PBS (pH 7.2) containing 5% BSA for 1 hour. Selected antibodies were tested for blocking at a concentration of 15 μg/ml diluted in PBS-5% BSA in the presence of recombinant human target protein (rhLGR4-Fc, rhLGR5-Fc, rhZNRF3-Fc or rhRNF43-Fc). The IgG/recombinant human target protein complex was pre-incubated for 10 minutes before addition to the R-Spondin3 coated plate for 10 minutes-2 hours. As a control for blocking, the procedure was performed simultaneously by addition of a surplus (15 μg/ml) of rhR-Spondin3 instead of IgG. The ELISA plates were washed 3 times with PBS-T (PBS-0.05% v/v Tween 20). Bound Fc-protein was detected with 1:2000 diluted anti-human IgG HRP-conjugate (Goat anti-human IgG Bethyl labs) and was allowed to bind for 1 hour at 25° C. The ELISA plates were washed 3 times with PBS-T (PBS-0.05% Tween 20) and bound complex was detected by staining with TMB/H₂O₂; staining was quantified by means of OD450 nm measurement.

### Affinity Measurement of Bispecific Antibody Binding to Cell-Surface Expressed Antigen.

Afucosylated PB10651 was radio-labelled with ¹²⁵I using IODO-GEN according to the protocol described by van Uhm et al. The immuno-reactivity of the antibody after radiolabeling was investigated with the method described by Lindmo et al. Steady state cell affinity measurements of ¹²⁵I-PB10651 were performed with CHO cells expressing either EGFR or LRG5 to investigate the affinity towards EGFR and LGR5 respectively. In addition, the affinity towards the DLD-1 cell line endogenously expressing EGFR but no detectable LGR5 was also determined. The assay used a constant concentration of target (cells) and the amount of radio ligand was titrated without violating the assumptions behind affinity measurements at steady state conditions. Non-specific binding (NSB) was assessed by the presence of 100-fold molar excess unlabeled PB10651 in a parallel series. The assay was repeated twice and the estimated K_{D} value is reported as the mean of three independent experiments. Estimation of the K_{D} values were performed using GraphPad Prism v. 6.0 h non-linear regression, One-Site—Total and Non-specific binding with the constraint that K_{D} values must be greater than 0.

### REFERENCES

Lindmo T, Boven E, Cuttitta F, Fedorko J, Bunn P A. Determination of the immunoreactive fraction of radiolabeled monoclonal antibodies by linear extrapolation to binding at infinite antigen excess. J Immunol Methods. 1984; 72: 77-89.
van Uhm J I, Visser G W, van der Schans M J, Geldof A A, Meuleman E J, Nieuwenhuijzen J A. The ultimate radiochemical nightmare: upon radio-iodination of Botulinum neurotoxin A, the introduced iodine atom itself seems to be fatal for the bioactivity of this macromolecule. EJNMMI Res. 2015; 5: 5.

### Epitope Mapping Through Shotgun Mutagenesis Analysis

The epitopes on EGFR and LGR5 recognised by PB10651 were determined by shotgun mutagenesis analysis according to the method described by Davidson et al., (2014). Two mutation libraries were made from the two antigens: one library encompassed a.a. 300-520 (ligand binding domain L2 or domain III) of human EGFR (GenBank reference sequence NP_005219.2) and the other a.a. 22-560 (being the N-terminal domain until the first transmembrane helix) of human LGR5 (GenBank reference sequence AAH96324.1). The LGR5 expression construct was truncated at a.a. 834 to increase cell surface expression of the receptor. In-house developed antibodies targeting a different epitope were used as control antibodies for the expression of the mutants.

### REFERENCE

Davidson, E. and Doranz, B. J. (2014) A high-throughput shotgun mutagenesis approach to mapping B-cell antibody epitope. Immunology 143, 13-20.

### Competition for LGR5 Binding Between the LGR5 Fab Arm in PB10651 and the Ligand R-Spondin1 Using a Cell-Based Assay.

The literature-copied version of antibody OMP88R20 (called PG7711) described in U.S. Pat. No. 8,628,774B2) was used as a ligand-blocking control to set up an assay to demonstrate ligand-blocking capacity of the anti-LGR5 Fab arms. A CHO-K1-derived cell clone was generated that over-expressed human LGR5 by transient transfection of CHO-K1 cells with the expression construct encoding LGR5, followed by selection of antibiotic (G418) resistant clones by limiting dilution. The binding of the copied version of OMP88R20 to this cell line was then tested in a dilution series of antibody and the EC50 for binding was determined. Next, binding of the antibody to the LGR5 expressing CHO cell clone at the EC50 concentration was measured in FACS in the presence of increasing amounts of the ligand R-spondin1 (R&D Systems, cat. nr. 4645-RF/CF). To test whether the binding of the anti-LGR5 Fab (MF5816) of PB10651 to LGR5 could be inhibited by binding of the ligand, the antibody was tested for LGR5 binding at 100 ng/ml in the presence of increasing amounts of the ligand R-spondin1. A similar assay was performed using the rat anti-LGR5 antibody 1D9 that has been reported to bind to the juxta-membrane region of LGR5 (de Lau et al., 2011) to show the specificity of the interaction.

### Ligand Blocking Capacity of the Anti-EGFR Fab Arm in PB10651.

To test anti-EGFR IgG for their effects on EGF-induced signalling, they were tested for their ability to prevent the EGF-induced apoptotic cell death of A431 cells. In brief, high (10 nM) concentrations of EGF induce (apoptotic) cell death in A431 cells (Gulli et al., 1996). This effect can be dose-dependently reverted by the addition of ligand-blocking anti-EGFR antibodies, such as cetuximab (the murine 225 antibody is the mouse equivalent of cetuximab). A431 cells were plated at 1500 cells/well in 96 wells tissue culture plates and grown overnight. The next day, antibody was added at the indicated concentrations together with 62.5 ng/ml (10 nM) of recombinant, human EGF (R&D Systems, cat. nr. 236-EG) and cells were grown for three days. After three days, the number of metabolically active cells was determined by alamar blue (Invitrogen, cat. nr. DAL1100) addition and measurement of the fluorescence at 590 nm emission with 560 nm excitation. All anti-EGFR IgG were tested for their EGF blocking effects in this assay compared with cetuximab.

### REFERENCE

Gulli, L. F., et al., Epidermal growth factor-induced apoptosis in A431 cells can be reversed by reducing the tyrosine kinase activity. Cell Growth Differ, 1996. 7(2): p. 173-178.

### Heat Stability Assay

In order to get an indication on the stability of the bispecific IgG, all IgG were incubated at 40° C. for a week in serum containing medium (DMEM, high glucose (Gibco, cat. nr. 41966-029), supplemented with 9% Fetal bovine serum (Thermo Scientific Cat. nr. SV30180)) and were then tested for binding in a binding ELISA (essentially as described above). This ELISA consisted of coating 2 μg/ml antigen (rhLGR4-Fc, rhLGR5-Fc, rhZNRF3-Fc or rhRNF43-Fc), using 100 μl at 5 μg/ml IgG and blocking in 5% BSA in PBS. In this assay the binding of the same IgG (in serum containing medium) kept at 2-8° C. (refrigerator) to antigen was compared to that of IgG kept at 40° C. The percentage loss of binding after 1 week incubation at 40° C. was calculated.

### Colorectal Cancer (CRC) Organoid Cultures

CRC organoids were produced as described (Van de Wetering et al. 2015 Cell 161:933-45) as working cell banks and shipped for screening assays on dry ice as frozen vials at a density of 1,500,000 cells per 1 ml freezing medium (Fetal Bovine Serum containing 10% DMSO as a cryoprotectant). The cryo-tubes are stored at −80° C. for use within 3 months or at −150° C. for long-term storage.

### Culture Media

Tumoroid Expansion Medium was produced by enriching 500 ml Advanced DMEM (Thermo Fisher 12491-023) with 5 ml 100× Penicillin-Streptomycin (Thermo Fisher 15140-122), 5 ml 100× Glutamax (Thermo Fisher 35050-038) and 5 ml 1M Hepes (Thermo Fisher 15630-056). To 70 ml of the enriched Advanced DMEM, 20 ml R-spondin 1 conditioned medium and 10 ml of Noggin-conditioned medium (Van de Wetering et al. 2015 Cell 161:933-45) was added to prepare 100 ml Tumoroid Expansion Medium which was additionally supplemented with 2 ml 50× B27 supplement (Thermo Fisher 17504-044), 1 ml 1M Nicotinamide (Sigma-Aldrich N0636), 250 μl 500 mM N-Acetyl Cysteine (Sigma-Aldrich A9165), 10 μl 5 mM A83-01 (TGFβ inhibitor, Tocris 2939) and 33 μl 30 mM SB202190 (p38 inhibitor, Sigma Aldrich S7067). The Tumoroid Expansion Medium was further supplemented with 100 μl 10 mM Y27632 (Rho kinase inhibitor, Abmole Y-27632 dihydrochloride) upon fresh seeding of colon tumoroids in the presence of human EGF (Peprotech AF-100-15) or human NRG-1/HRG (ImmunoTools 11343047). EGF and HRG are growth factors that stimulate the organoid expansion and their dose determines the sensitivity of the screening assay. EGF was added at doses of 2.5 ng/ml, 5 ng/ml or 10 ng/ml during screening or 10 ng/ml or 50 ng/ml during expansion. HRG was used solely at 5 ng/ml.

Wild type organoid Expansion Medium is equal to the Tumoroid Expansion Medium, with one replacement: the 70 ml enriched Advanced DMEM was replaced by a combination of 20 ml enriched Advanced DMEM and 50 ml WNT3A-conditioned medium (Van de Wetering et al. 2015 Cell 161:933-45). Normal tissue-derived colon organoids and colon tumoroids with wild type APC are WNT-dependent and therefore require the presence of WNT for expansion.

### Gel Composition

Frozen colon tumoroids were thawed rapidly in a water bath at 37° C. and collected in 5 ml enriched Advanced DMEM. The organoids were pelleted by 5 minute centrifugation at 1000 rpm at 4° C. The supernatant was removed and the organoids taken up in Tumoroid Expansion Medium without growth factors. This organoid suspension was mixed with Cultrex Reduced Growth Factor Basement Membrane Extract, Type 2, PathClear (Amsbio 3533-010-02). The final Cultrex gel percentage was 60% and the number of cells per ml was 100,000.

### Determination of Growth Factor-Responsiveness of Patient-Derived Organoids

In order to determine whether patient-derived organoids were responsive to growth factor treatment, they were seeded as described above in the presence or absence of growth factors (EGF (5 ng/ml), or NRG (5 ng/ml)) and then cultured for 5 days. The number of viable cells was then determined using the Cell Titer Glo cell viability assay (Promega, cat. nr. G7571). The luminescence readout using growth factor-stimulated cells was then compared to that obtained using non-stimulated cells.

### Preparation of Culture Plates

The gelation was more rapid in pre-warmed culture plates. Therefore all culture plates were placed at 37° C. in a humidified CO₂ incubator (Eppendorf) the day before cell seeding.

To expand colon tumoroids, 24-wells or 6-wells plates (Greiner Bio-One) were used and per well, either 3 or 10 drops of 15 μl gel/organoid suspension was spotted at regular distances. Tumoroid Expansion Medium (0.5 ml or 2 ml) containing between 10 ng/ml to 50 ng/ml EGF was added after a 30 minute gelation period at 37° C. At day 1 of seeding the Tumoroid Expansion Medium contained 10 μM Y27632. Medium replacements during expansion were with EGF-containing Tumoroid Expansion Medium devoid of Y27632.

To perform a screen, 384-wells pelear plates (Greiner Bio-One 781091) were used. Per well, 15 μl of the gel/organoid mix was dispensed using automated liquid handling. Upon 30 minutes gelation at 37° C., 45 μl Tumoroid Expansion Medium (or Organoid Expansion Medium where applicable) was added on top of the gel in each well. The media were supplemented with or without growth factors and (reference) antibodies and compounds were mixed with the medium in v-bottom 96-wells plates before applying to the 384-wells plates with solidified gel.

### Preparation of Antibody Master Plates

Reference antibodies (Cetuximab (4° C.), negative control antibodies, single arm HER3 or EGFR targeting antibodies and HER3/EGFR antibodies (on dry ice)) were shipped and stored at 4° C. for screening. Bispecific and monospecific antibodies were delivered in deep-well 96-wells plates that were sealed and shipped at 4° C. In general, the notation to monospecific cLC antibodies throughout the examples is recognized by the prefix ‘PG’ whereas the notation to bispecific cLC antibodies is recognized by the prefix ‘PB’. For the avoidance of doubt, the notation to Fab fragments of cLC antibodies are recognized by the prefix ‘MF’. The antibodies were manually transferred to four v-bottom 96-wells plates (Greiner Bio-One 736-0118) in randomized locations ranging from well B02 to well G11 (inner 60 wells). Reference antibodies were added to the plates at random locations as well, at equal concentration. These antibody master plates were used to prepare 1:10 dilution plates in PBS. The master plates and dilution plates were stored sealed at 4° C. For validation of screening results a lead panel of bispecific IgG (46 antibodies at a concentration of 0.5 mg/ml) were shipped in screw-capped microvials, to allow easy randomization of the antibodies in screening plates and to prevent cross-contamination and evaporation. Prior to each experimental run, the bispecific IgG were placed in one v-bottom 96-wells plate (inner 60 wells) along with reference antibodies at 0.1 mg/ml by diluting them in PBS. This plate was diluted once more 1:5 in PBS to achieve a second master plate containing antibodies at 20 μg/ml. Dilutions and plate exposures were performed using the Felix liquid handler.

### Exposure Regimes

The high dose and low dose v-bottom 96-well antibody master plates were diluted 1:10 in culture medium before exposure. The antibody concentrations applied in primary screen were 10 μg/ml and 1 μg/ml or 40 μg/ml and 4 μg/ml; and in the validation screen the antibody doses were 10 μg/ml and 2 μg/ml. The antibodies were added to the organoids 30 minutes after seeding. The antibody exposure before plate fixation was minimally 7 days and maximally 9 days.

### Fixation and Imaging

To prepare the exposed tumoroid plates for imaging, the organoids were fixed and stained to visualize the nuclei and the actin cytoskeleton respectively as previously described (Di et al PlosOne 2014, PMID 25289886). Imaging of the plates was performed using a Molecular Devices ImageXpress Micro XLS connected to a Twister II robotic arm as previously described (Sandercock et al, Molecular Cancer 2015, PMID 26227951). Briefly, z-stacks of each well in a 384-wells plate was captured using a 4× lens, with a z-step size of 50 μm. The number of sections per well ranged from 20 sections to 24 sections, to cover the entire depth range of the gel in each well.

### Image Analysis

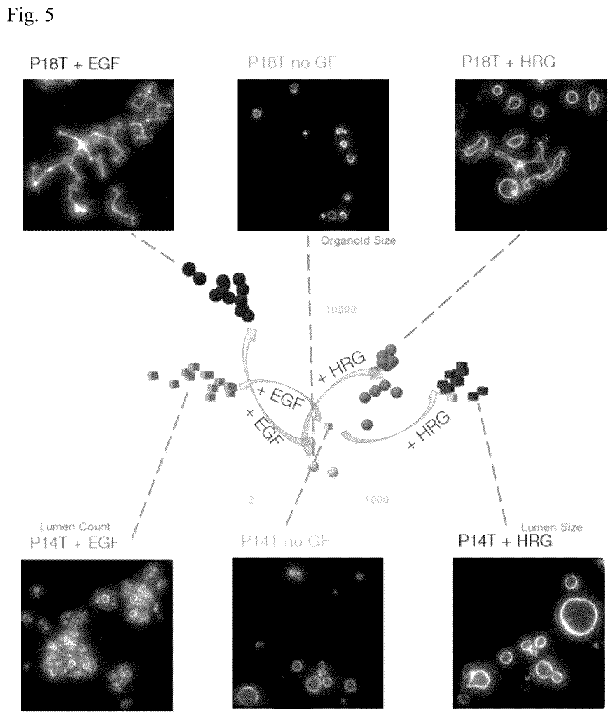

Captured images were stored on a central data server, accessible by the OcellO Ominer™ 3D image analysis platform which allows direct parallel analysis of the 3D image stacks by its distributed computational design. The software analyses the structure of the objects (nuclei and cytoskeleton) detected in each well, and their relative positions. Upon analysis, the output was checked to detect the quality of the raw images and the analysis method. The per object measurements the software produced (for the nuclei, the organoids, nuclei within organoids, the lumens, the relative positioning of lumens within organoids and the entire structure (organoids, nuclei and lumens)) were subsequently aggregated per well and the data coupled to the plate layout information (cell line, growth factor condition, treatment, etc.). Upon data aggregation, the data were checked for consistency within control treatments, absence of edge effects, consistency between replicates and the z′-factor between positive and negative controls. Next, the data were z-score normalized and inspected through loading the data into TIBCO Spotfire® to purge additional outliers. 500 different morphological features were collected; The data was then run through a series of statistics that made a sub-selection of 3 to 20 of the ˜500 features gathered from the z-stack images, based on the ability of this set of features to distinguish the reference treatment effect from the negative control morphology. The distance between the reference and the negative controls was calculated as a Euclidian distance measurement (FIG. 5) and scaled between zero and one. This unified score of morphology change was used to discriminate hits in the compound screens. The individual selected feature measurements, together with corroboration with the images was used to substantiate and verify the effects of the hit compound treatments on the organoids.

Z′-factor: The Z′-factor is a statistical measure for quality of a high throughput screening assay and indicates the separation window between positive and negative controls. The Z′-factor is defined as 1 minus 3 times the sum of the standard deviations of the positive and negative controls divided by the absolute difference between the means of the positive and negative controls. Z′-values smaller than 0 indicate that there is too much overlap between negative and positive controls, values between 0 and 0.5 indicate a useful but marginal screening window and values between 0.5 and 1.0 indicate an excellent assay with a strong separation between positive and negative controls.

FACS Staining of Cells Obtained from Organoid P18T Using Selected Anti-LGR5 cLC Antibodies

Organoids derived from a colorectal cancer sample were cultured in 100% Basement Membrane Extracts (BME, Amsbio), at 37° C. and 5% CO2, with media composed of Advanced DMEM/F12 (Invitrogen) supplemented with: 2 mM GlutaMax (Invitrogen), 10 mM HEPES (Invitrogen), 1× B27 retinoic acid free (Invitrogen), 50 ng/mL EGF (Peprotech), 0.1 μg/mL Noggin (Peprotech), Rock-inhibiter Y-27632 (Sigma-Aldrich), 10 nM PGE2 (Sigma-Aldrich), 3 μm SB202190 (Sigma-Aldrich), 10 nM Gastrin (Tocris), 1p g/ml R-SPO1 (home-made), 10 mM Nicotinomide (Sigma-Aldrich), 1.25 mM N-Acetyl-cysteine (Sigma-Aldrich), 0.5 μM A83-01 (Tocris). The day prior to analysis, the organoids were disaggregated into single cells. To this aim, the organoids were first liberated from the BME by removing the culturing media, and re-suspending the BME in cell recovery solution (BD Biosciences), and incubating for 1 hour on ice. Subsequently, the organoids were centrifuged (all centrifuge steps were for 5 minutes, 200 g at 4° C.). The pellet was re-suspended in 1 mL of 50% Trypsin/EDTA Solution (TE); 50% PBS, and pipetted up and down, and regularly visually assessed until a single cell suspension was achieved. The TE was diluted in 10 mL of PBS and centrifuged. The cells were washed twice in 10 mL of PBS before re-suspending in BME and aliquoting into 50 μL drops on to pre-warmed plates (37° C.). The BME drops were left to set for 15 minutes before 500 μL of media were added per drop. After 12 hours the cells were isolated from the BME using cell recovery solution. After 1 hour on ice, the cells were centrifuged, and washed once in 10 mL of PBS containing 0.5% BSA and 0.5 mM EDTA (staining buffer). The pellet was then re-suspended in staining buffer and counted. A maximum of 100,000 cells/100 μL of antibody was used. After counting the appropriate number of cells, they were centrifuged and re-suspended in 100 μL of the staining buffer containing the primary antibody (monoclonal and bispecific IgG) diluted to 10 μg/mL. The cells were incubated on ice for 45 minutes, with regular inversion of the tubes to ensure homogeneous staining. After the incubation the cells were washed in 1 mL of staining buffer and centrifuged. The cells were washed again in 1 mL of staining buffer before incubating with 100 μL staining buffer containing an anti-human IgG antibody conjugated to R-PE (Invitrogen, H10104) diluted 1:400. The cells were incubated for 20 minutes on ice, protected from light. After incubating, the cells were washed twice in 1 mL of staining buffer before re-suspending them in staining buffer containing 0.1 μM DAPI (Sigma-Aldrich). The cells were maintained on ice, protected from light, and analyzed immediately. Doublets were excluded using SSC-W vs SSC-A, and DAPI was used to exclude dead cells. Gating of the LGR5 antibodies was set based upon the staining of negative control antibody which was raised against Tetanus Toxin (MF1337). Fluorescence was detected using a BD FACS Aria Fusion using the UV laser and 450/50 filter set for DAPI, and the green laser with the 582/15 filter to detect the R-PE fluorescence.

LGR5 mRNA Enrichment after Sorting

To assess whether the LGR5 FACS staining enriched for cells expressing LGR5, the cells were sorted with the sorting gate set so that the highest and lowest 15% of stained cells were isolated. A total of 2000 cells were sorted into picroprofiling buffer (provided by the IRB genomics facility), and the sorted cells were then processed by the IRB genomics facility for RNA extraction and cDNA synthesis. LGR5 expression was assessed using quantitative PCR using TaqMan probes and TaqMan Universal PCR Master Mix (both from Applied Biosystems). The StepOnePlus real-time PCR machine (Applied Biosystems) was used to run the reactions in a clear optical 96-well reaction plates with optical covers, following the manufacturer's instructions. Expression between the negative and positive LGR5 fractions were assessed using an LGR5 probe (Hs00173664_ml) and normalized using the expression of the endogenous control gene B2M (Hs99999907_ml). Differences in target gene expression were determined using the StepOne 2.2 plus software.

### P18 Staining for LGR5 and Sorting of LGR5 Positive and Negative Population and the Differences in Growth Between Those Two

Cells were stained as described previously and the sorting gates were set so that the highest and lowest 15% of stained cells were sorted into 200 μL of culture media containing Primocin (Invitrogen). The number of sorted cells was determined based upon the number of FACS sorted events. After sorting, the cells were centrifuged and plated at a density of 2000 cells/25 μL of BME. The number of organoids formed after two weeks was manually counted using an inverted light microscope.

Treatment of P18T Organoid with EGFRxLGR5 Bispecifics

For the antibody treatment experiments the culture media was modified, and did not contain Gastrin or PGE2, and had a reduced concentration of EGF (2.5 ng/mL). After disaggregation of organoids, Tryphan Blue staining (Sigma-Aldrich) was used to determine the number of live cells, and 5000 cells were plated/25 μL of BME in a 48-well tissue culture plate. Each treatment had 8 technical replicates, and was cultured in 250 μL of media/well. Three days after seeding single cells, the media was removed and replaced with media containing the antibody treatments (2 μg/mL). After 7 days from the addition of the antibodies, the plate was scanned using an Olympus ScanR using the x4 light objective, and the number of organoids quantified using ImageJ running a macro designed by the IRB microscopy facility. The experiment was repeated on three separate occasions and a two-tailed, paired sample T-Test was run to assess for significant differences between treatments.

### RNA Isolation and Q-PCR Analysis of LGR5 and CK20mRNA Levels

After the tissue culture plates of treated tumoroids were scanned for enumeration, the tumoroids were isolated using cell recovery solution and incubated on ice for 1 hour. Tumoroids were then centrifuged and re-suspended in 1 mL of TRIzol® Plus RNA Purification Kit (Life Technologies), and total RNA was extracted. After phase separation with chloroform, the upper aqueous phase was mixed with 70% ethanol and washed through RNA columns (PureLink™ RNA Mini kit, Life Technologies) according to the protocol provided by the manufacturer. RNA was quantified using a Nanodrop spectrophotometer. RNA was then reverse transcribed into cDNA using the High Capacity cDNA Kit (Applied Biosystems). Quantitative real-time PCR using TaqMan probes and TaqMan Universal PCR Master Mix (both from Applied Biosystems) was used to quantify LGR5 (Hs00173664_ml) and CK20 (Hs00300643_ml) levels of mRNA. Differences in expression were detected using the 2-ΔΔCT method and the StepOne 2.2 plus software.

Ex Vivo Measurement of mRNA and Protein Levels of LGR5 and EGFR in PDX Models of Different Indications

mRNA extracted from in vivo grown PDX tumours was used to analyse the expression levels of LGR5 and EGFR genes by RNA sequencing (RNAseq). Data from Crown Bioscience database (http://hubase2.crounbio.com) are expressed as log 2 conversion of fragments per kilobase million (FKPM): Table 9. In addition, tumour cells extracted from in vivo grown different PDX tumours were stained with 15 μg/ml of PG5816 (anti-LGR5), PG3755 (anti-EGFR) and (control) PG1337 and bound antibody was then subsequently detected with PE-labeled anti-human IgG. Table 10 shows mean fluorescence intensities for all antibody staining in several cancer indications.

### Example 2: Generation of Antibody Panels Directed to WNT Pathway Targets Using Phage Antibody Selections

Immunization of MeMo® Mice with the Four Different WNT Targets.

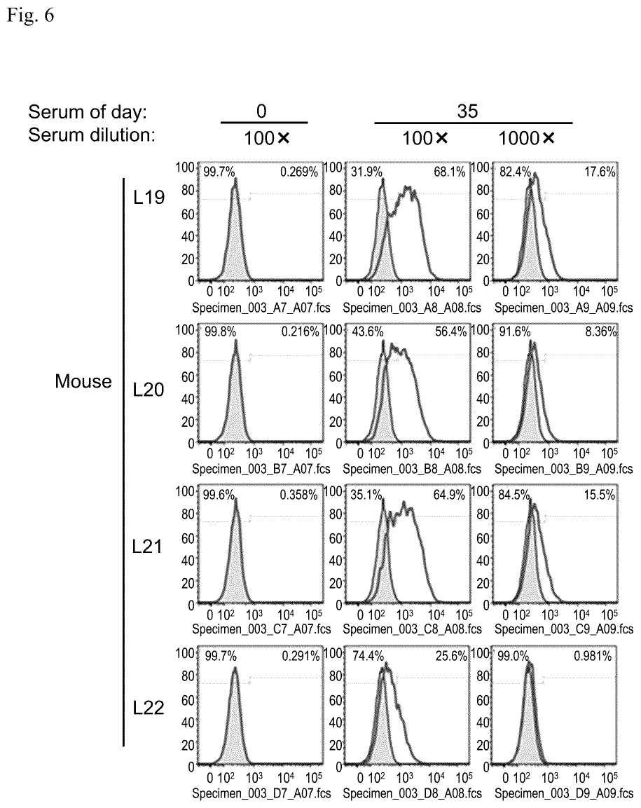

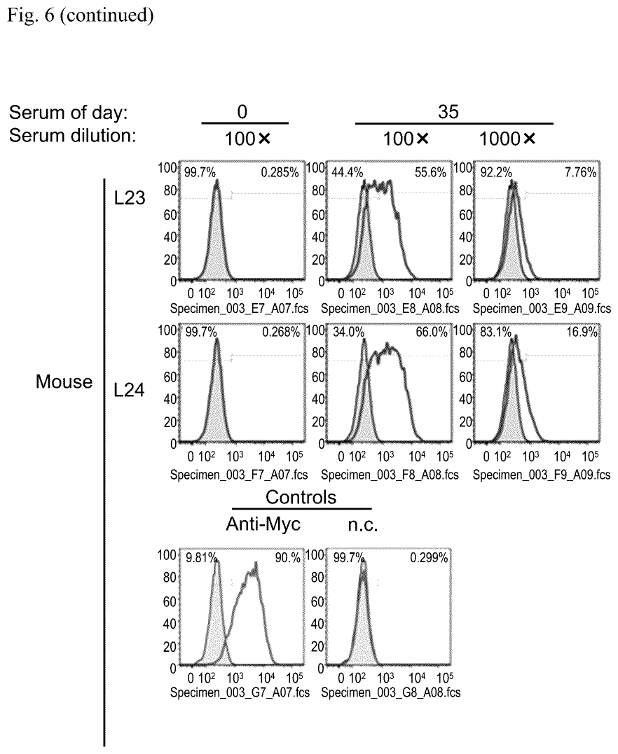

MeMo® mice were immunized with either expression constructs encoding full length human LGR4 and LGR5 (pVax1_hLGR4-FLAG-HA and pVax1_hLGR5-FLAG-HA), with the extracellular domain of LGR4 or LGR5 (pVax1_hLGR4(ECD)-GPA33-FLAG and pVax1_hLGR5(ECD)-GPA33-FLAG) or with recombinant proteins rhLGR4-Fc, rhLGR5-Fc, rhZNRF3-Fc or rhRNF43-Fc (RND systems). Mouse sera were screened for the evidence of a humoral immune response directed towards the target a FACS based assay using in-house generated Freestyle 293F cell lines that stably over-expressed the respective antigen. FIG. 6 shows an example of the data. The serum IgG titers (defined as the highest serum dilution giving a staining of the Freestyle 293F cell line stably expressing the target of at least three times the MFI of serum collected before immunization) of mice successfully immunized with the four WNT targets are depicted in Table 1. Mice that were shown to have mounted a significant and specific immune response towards the respective antigen were then taken out of the study and lymphoid tissues of these mice (inguinal lymph nodes and spleens) were harvested. From the obtained lymph nodes, ‘immune’ phage antibody repertoires were generated by RT-PCR using IgG- and VH-specific primer pairs and cloning of the polyclonal pool of VH-encoding cDNA's in a phagemid for the expression of Fab fragments on the surface of non-lytic phage. All libraries generated had a size of more than 10\hat{ }6 clones (individual transformants) and an insert frequency of more than 80%.

### Phage Selections to Generate Target-Specific cLC Antibodies

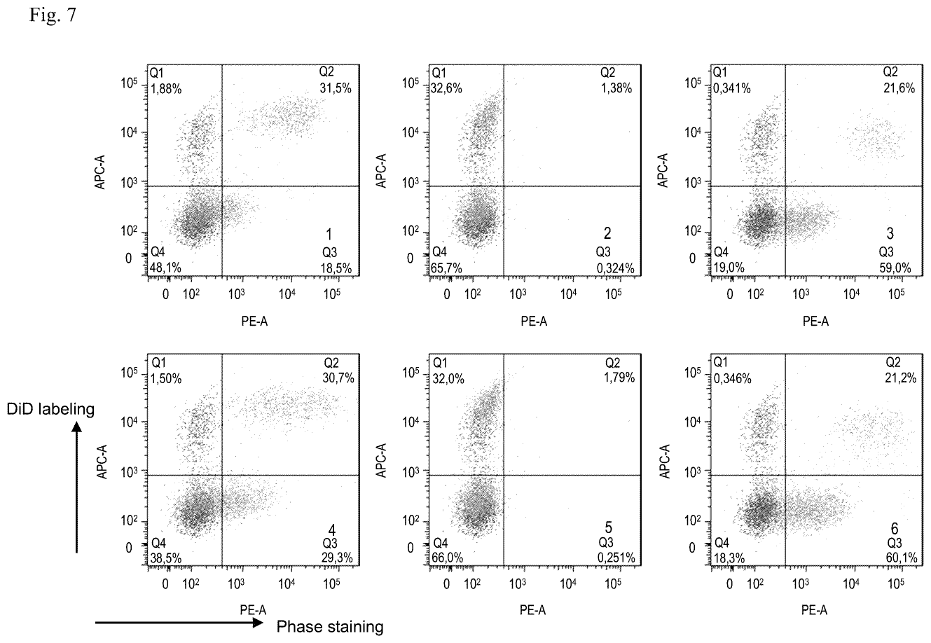

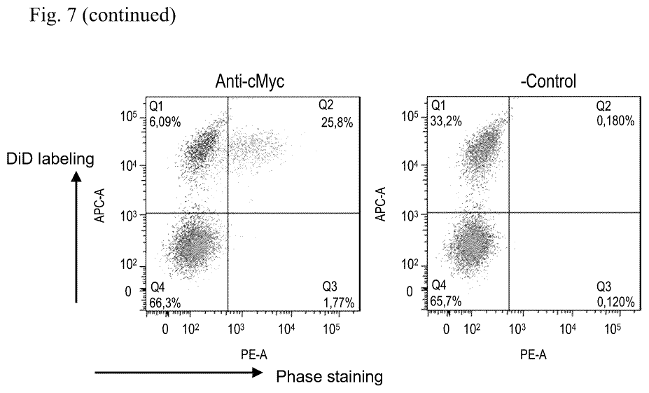

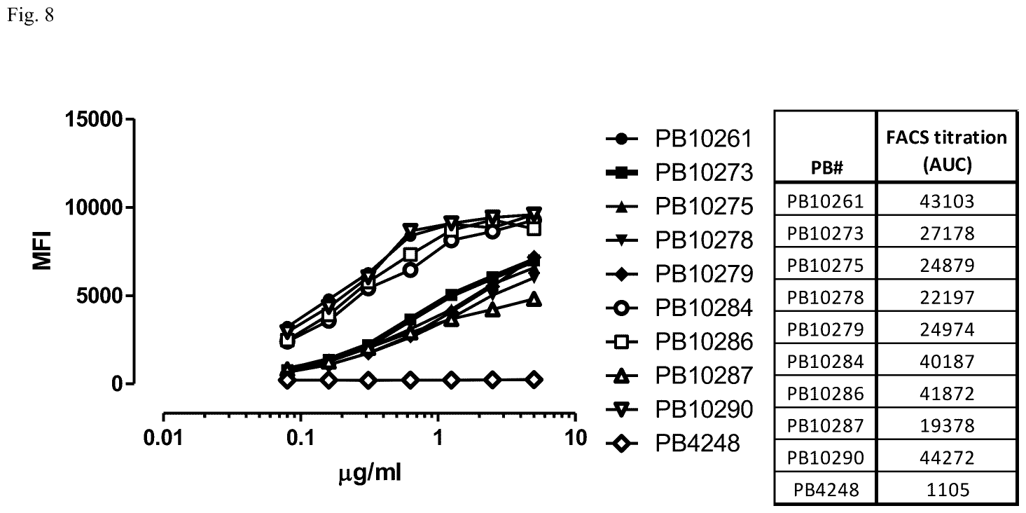

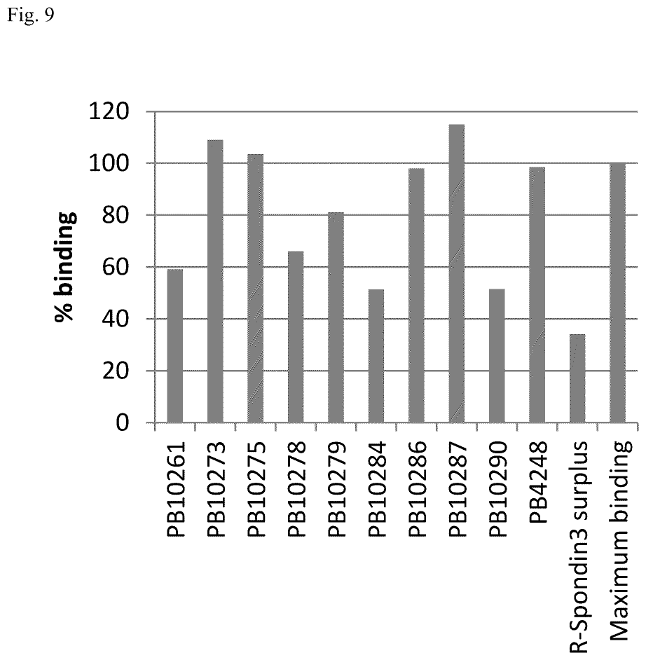

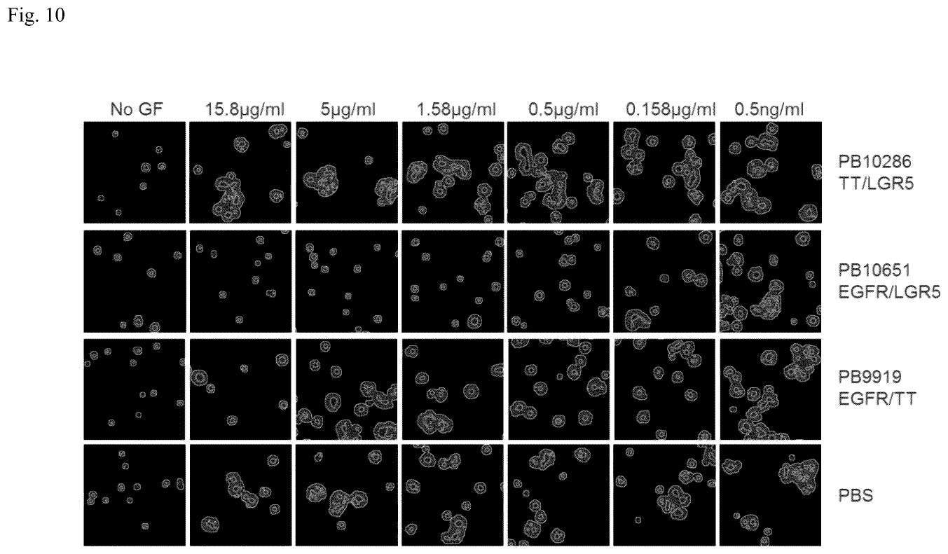

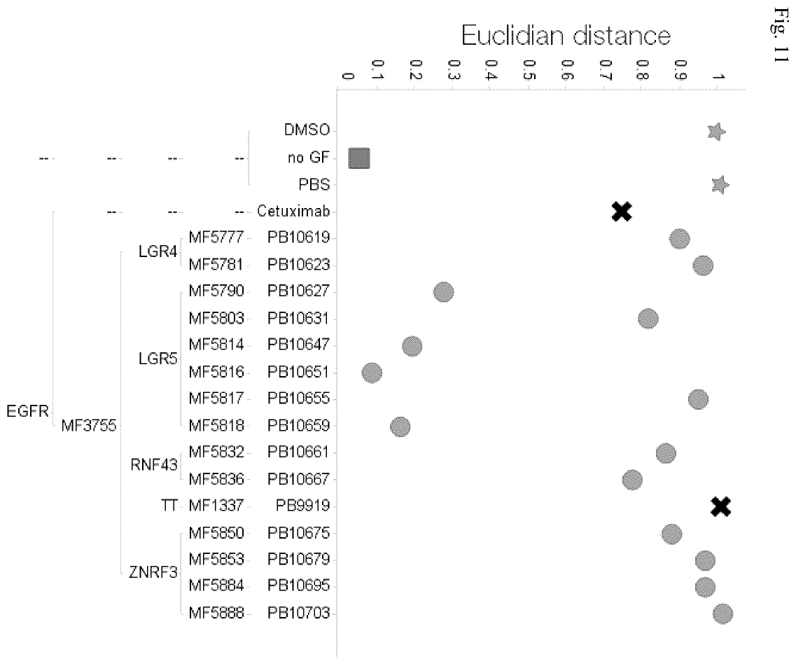

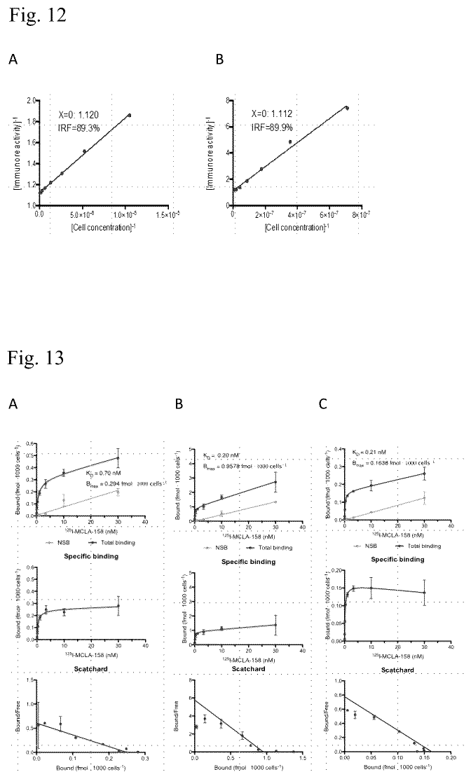

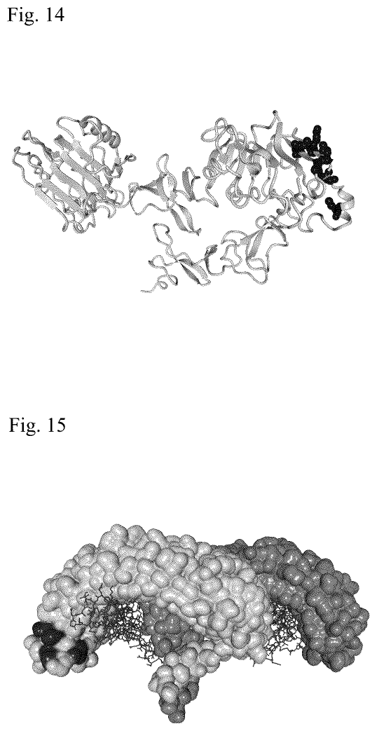

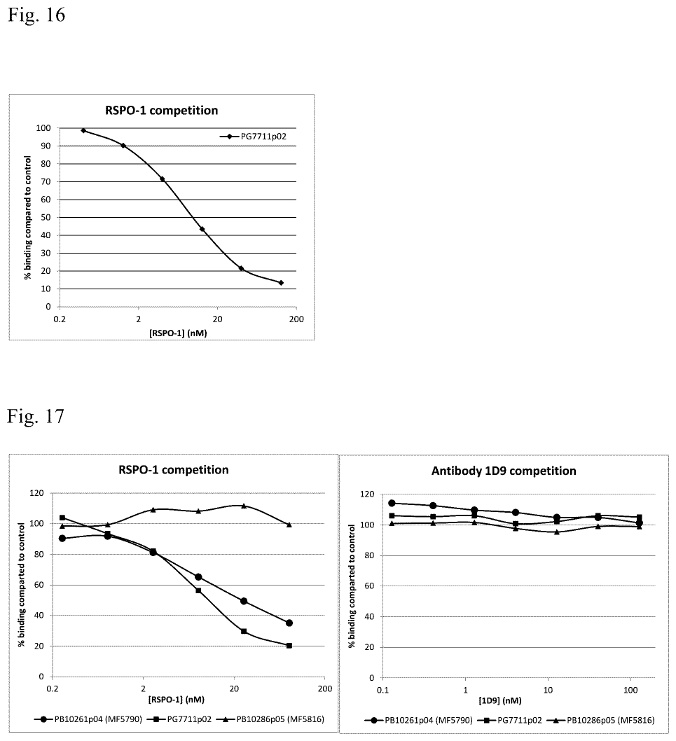

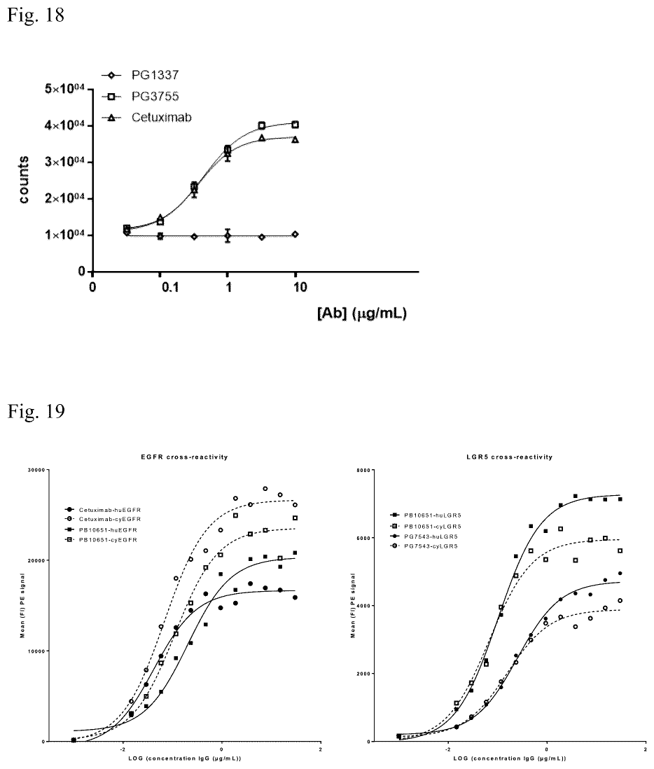

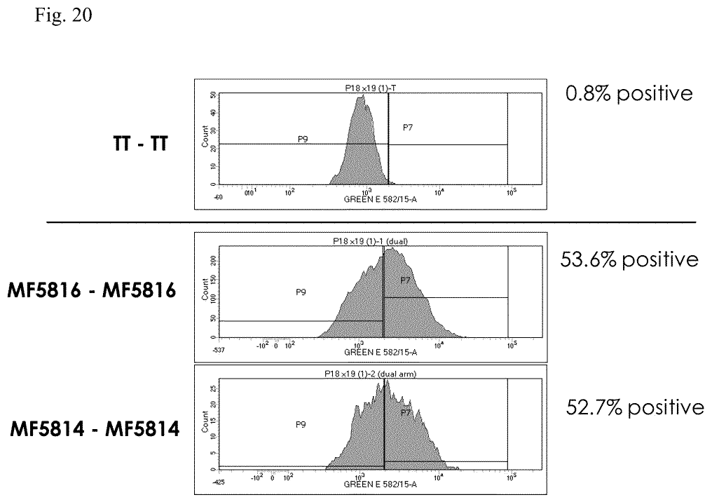

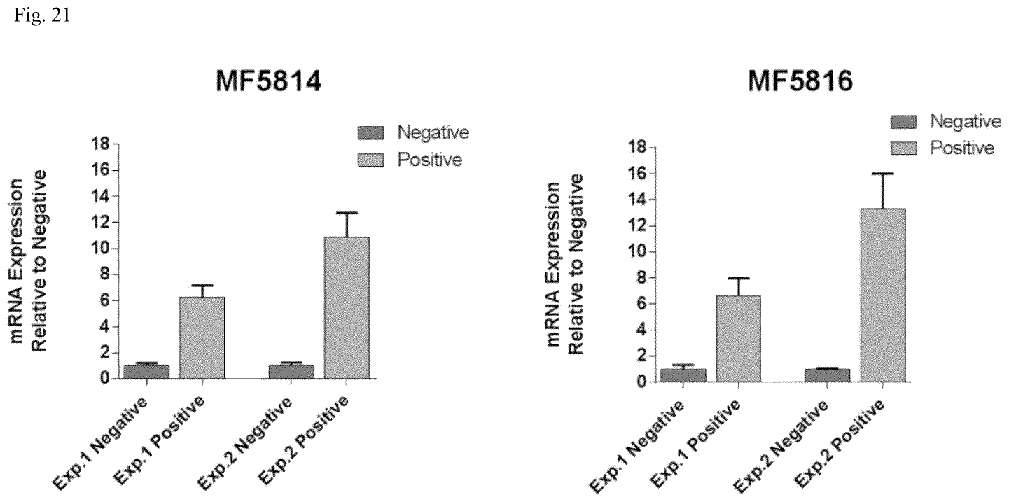

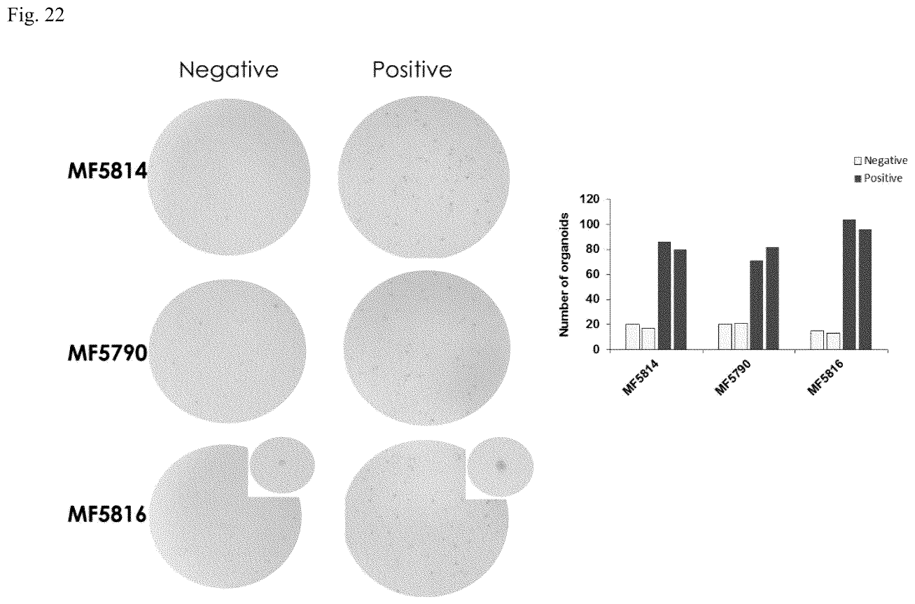

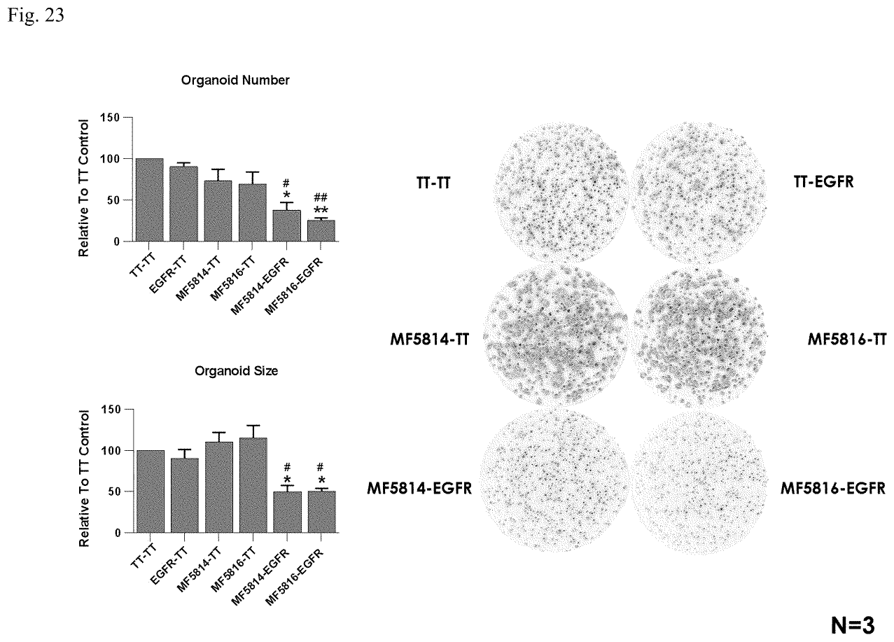

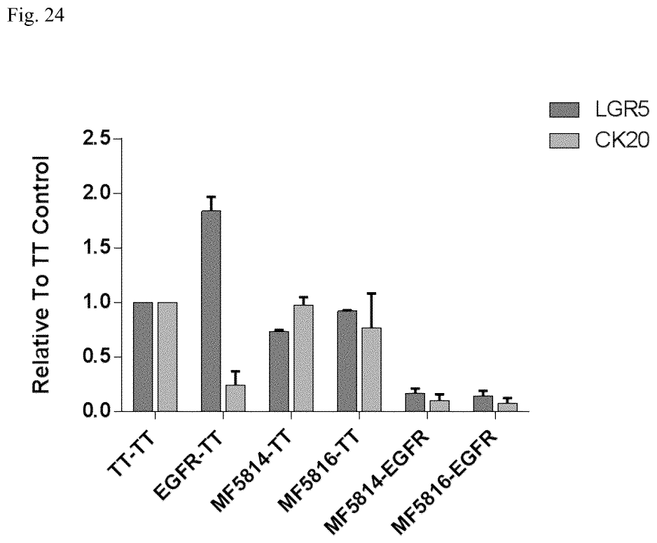

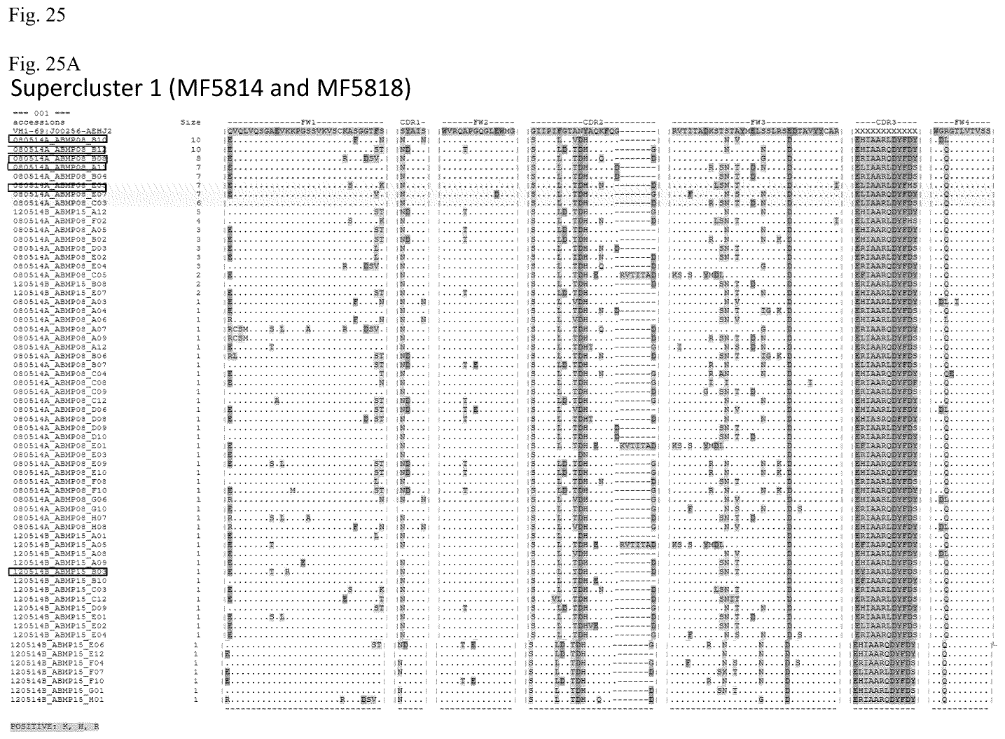

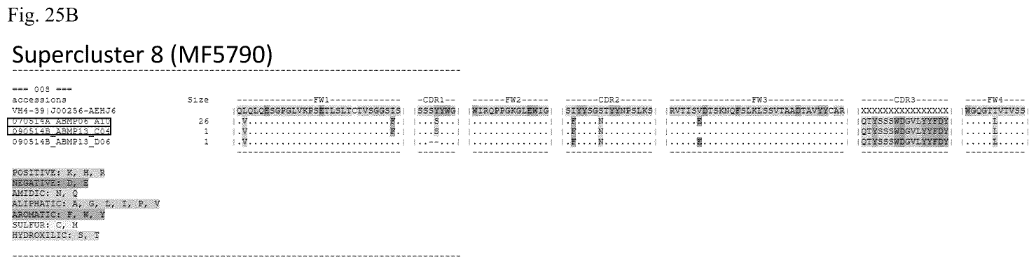

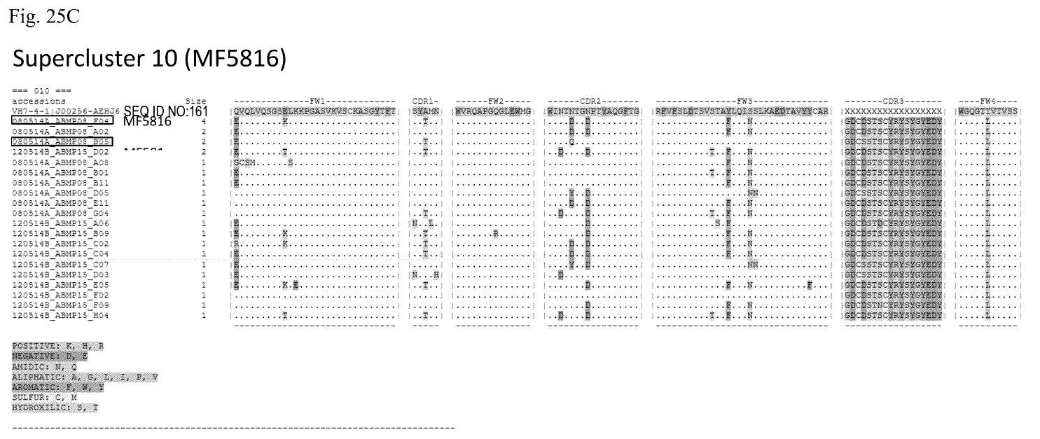

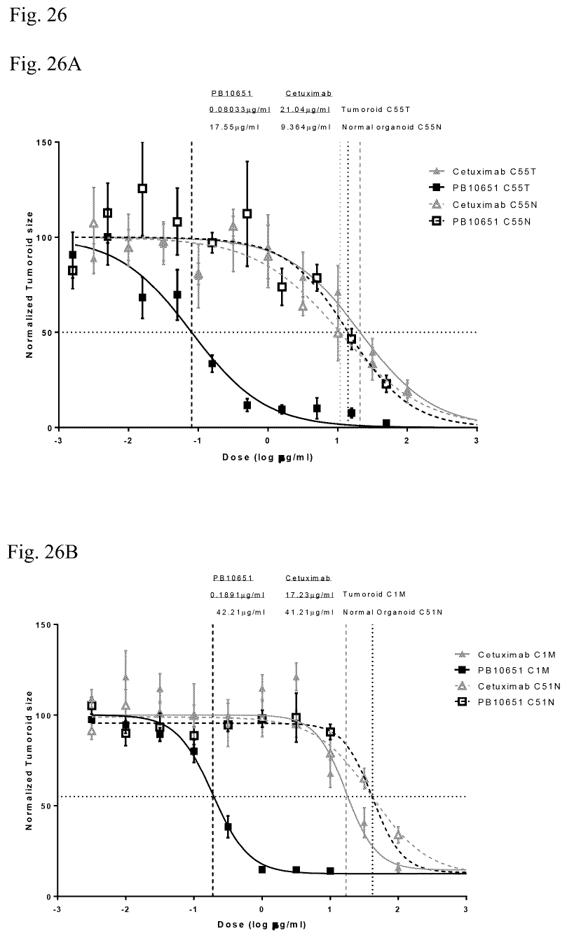

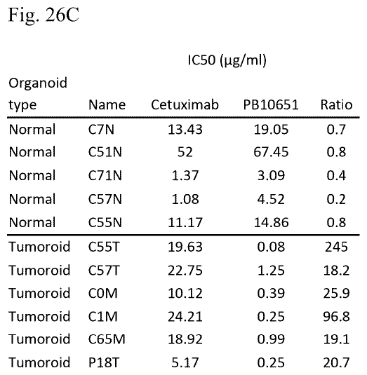

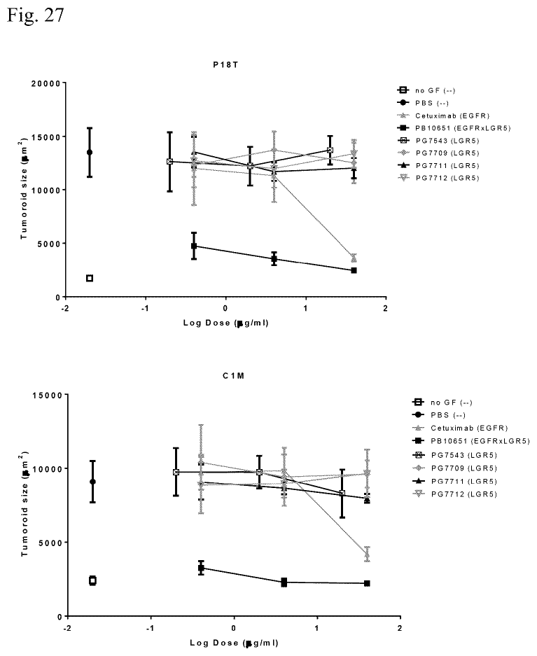

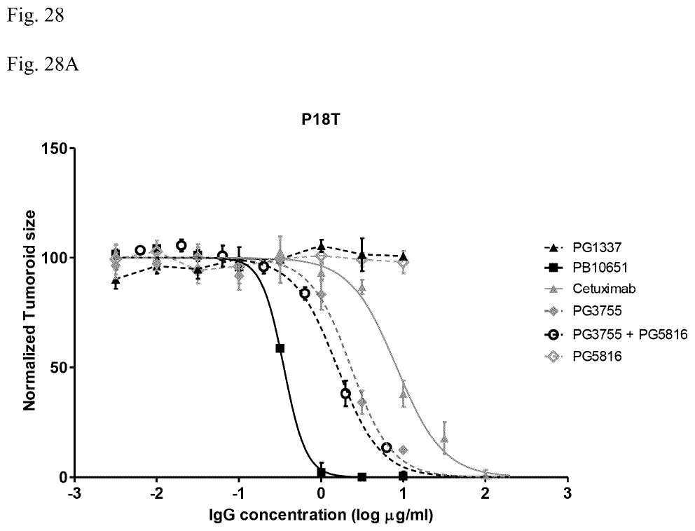

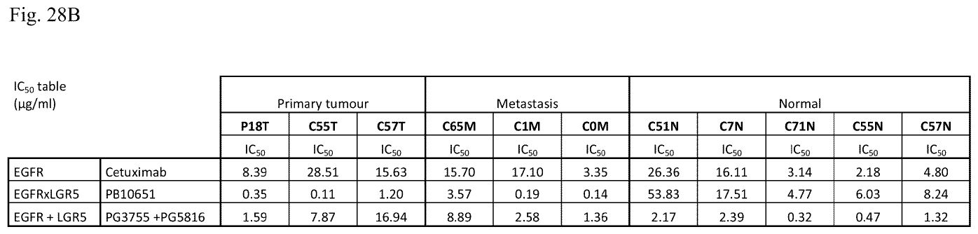

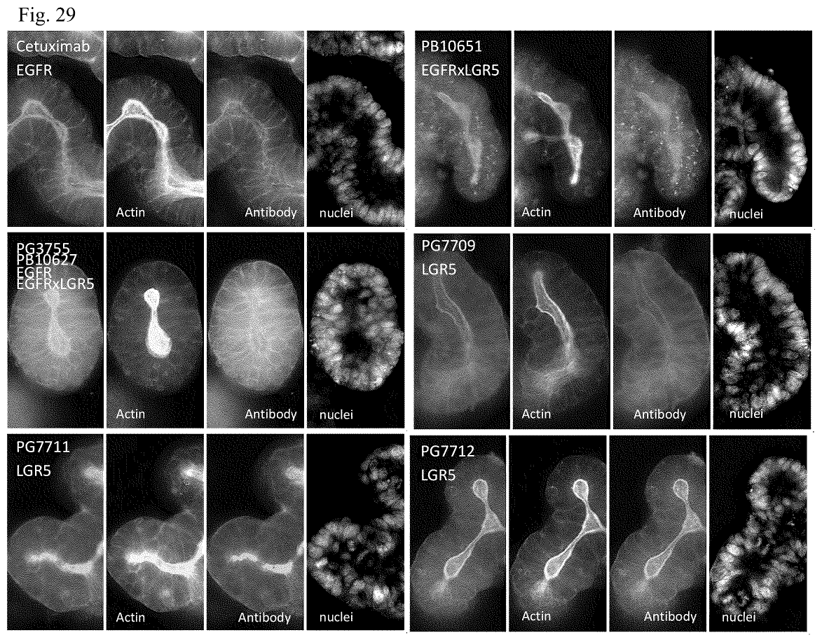

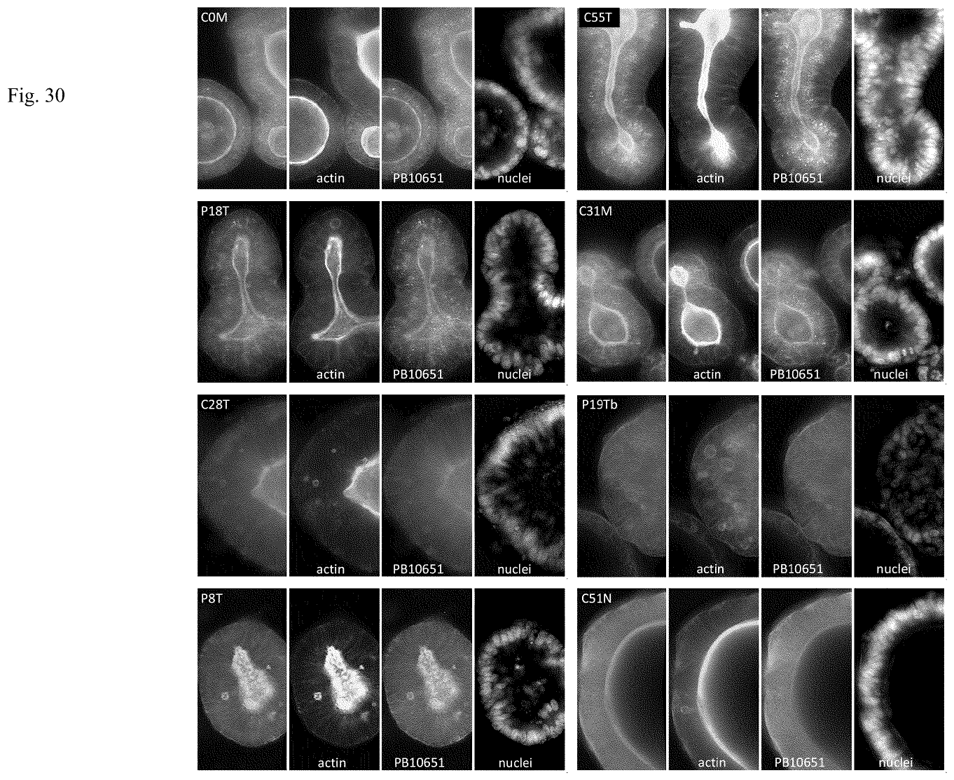

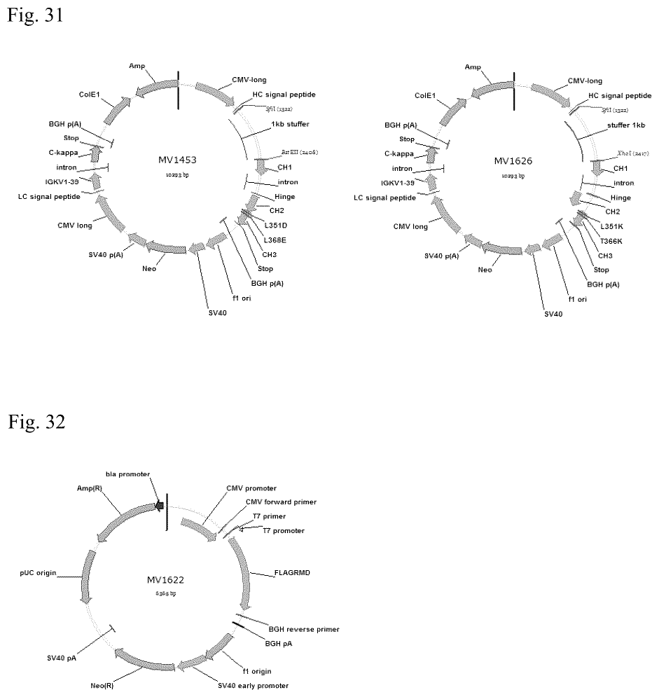

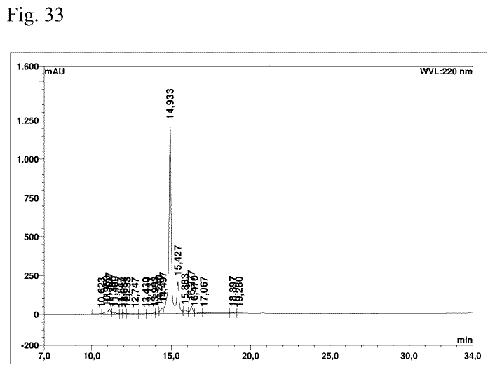

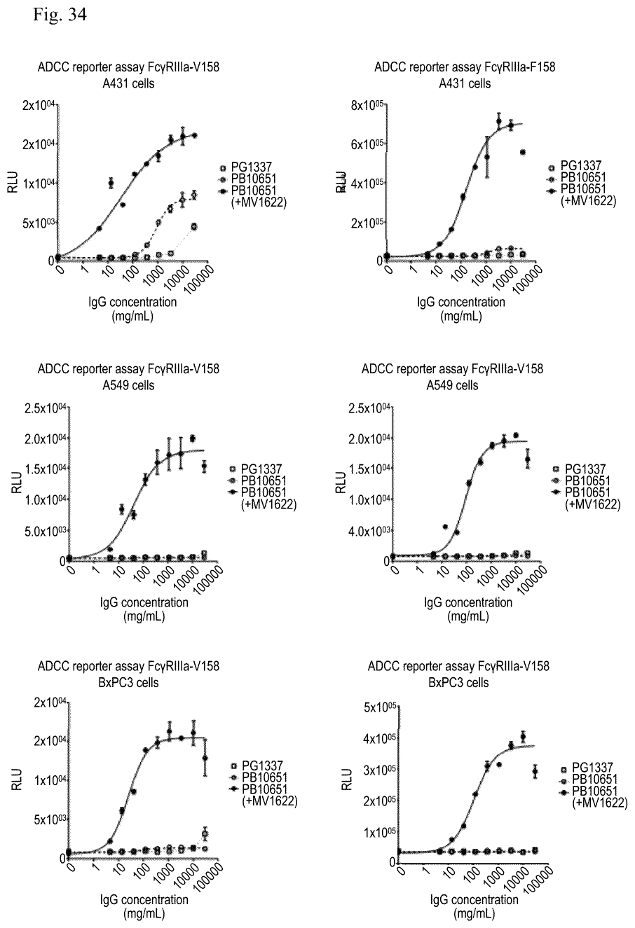

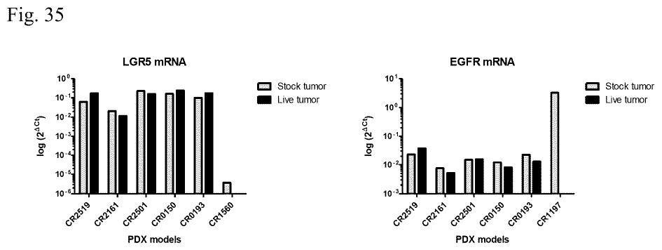

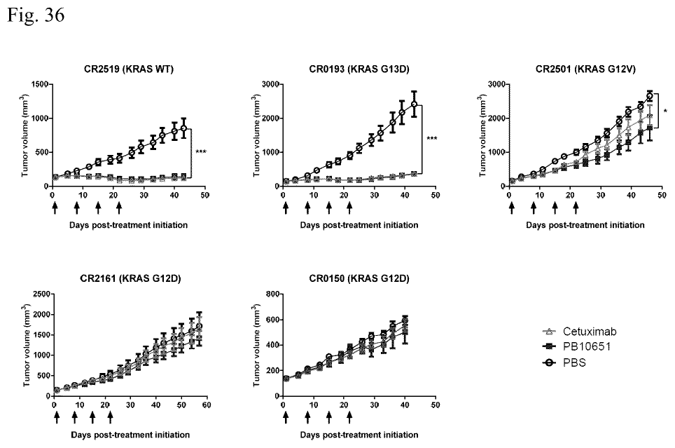

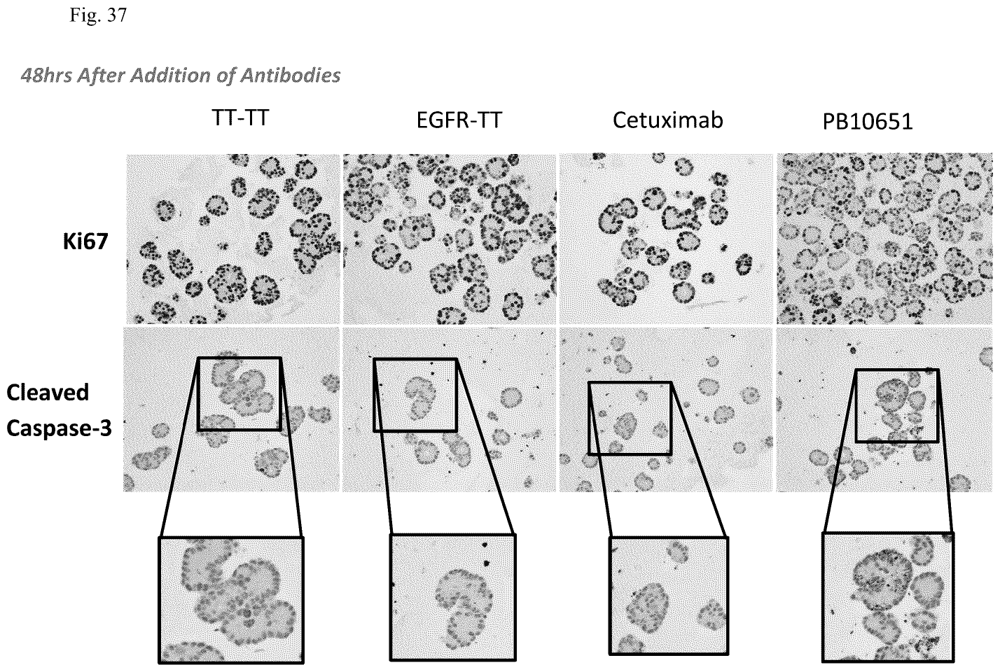

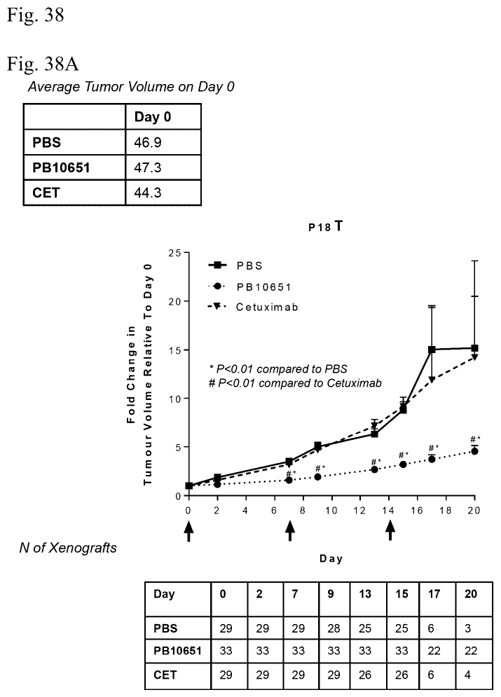

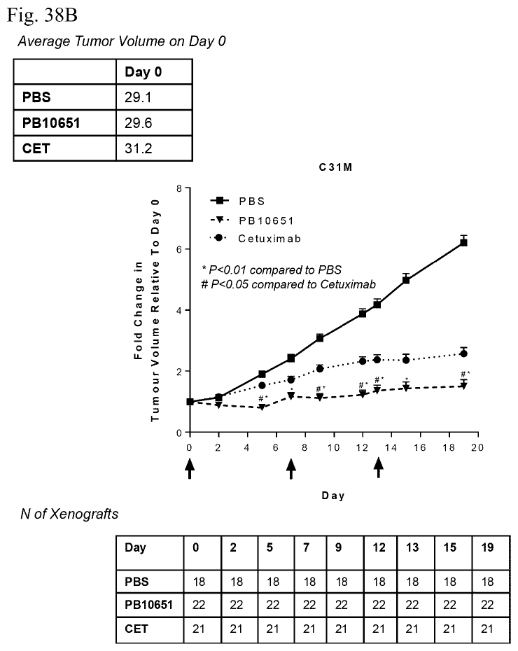

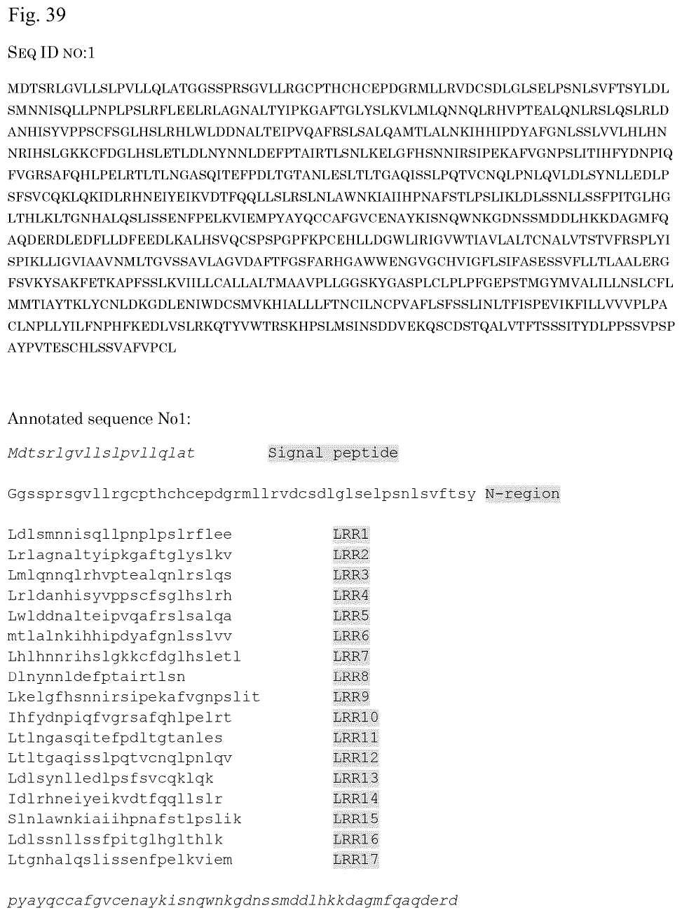

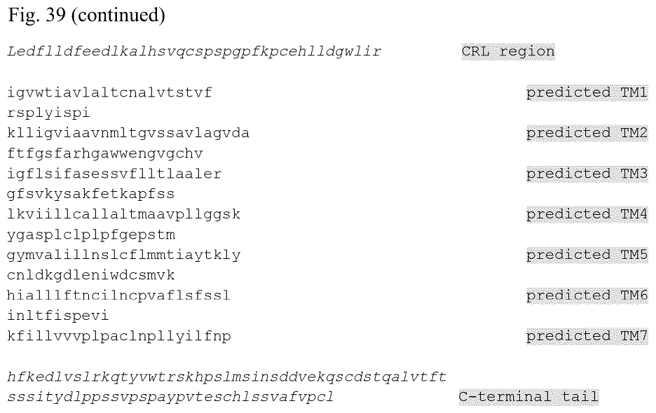

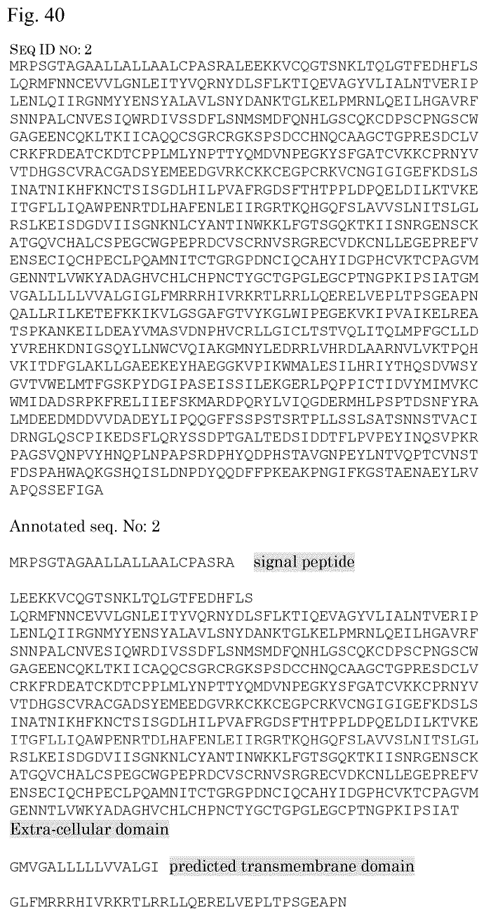

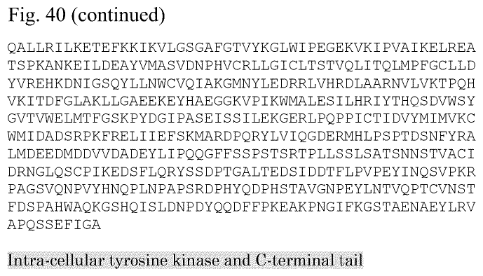

In order to generate cLC antibody panels directed to the targets hLGR4, hLGR5, hZNRF3 and hRNF43, the rhLGR4-Fc, rhLGR5-Fc, rhZNRF3-Fc, rhRNF43-Fc (RND systems) or the soluble ECD of hRNF43 or mZNRF3 (prepared in house) were coated to a solid support and in-house generated synthetic cLC phage antibody repertoires were panned for binding to the coated antigens in the presence of an excess of human IgG (to prevent the selection of Fc-binding phage) essentially as described by Marks et al. (J. Mol. Biol. 1991 Dec. 5; 222(3):581-97). In parallel, ‘immune’ phage antibody repertoires made from successfully target-immunized animals were also used for selections. Selections on coated protein were performed in microtiter plates (NUNC, maxisorp) and selections on Freestyle 293F cells that over-expressed the respective target were performed in solution; elution of bound phage was performed by a pH shock using 100 mM glycine (pH2) or 100 mM TEA (pH12). After a first round of selection using synthetic phage antibody libraries, the polyclonal pool of selected clones was used to prepare phage again and phage were either panned for binding to the same antigen, or were selected on in-house generated Freestyle 293F cells stably over-expressing the respective antigen. After a single round (immune libraries), or two rounds (synthetic libraries) of selection, single clones were picked and used to prepare monoclonal phage that were tested for binding their respective targets in FACS using a mix of two cell lines: the parental (antigen-negative) Freestyle 293F cell line and DiD-labelled 293F Freestyle cells that stably over-expressed the WNT target. Phage clones that recognized the (DiD-labelled) antigen-positive cell population and not the antigen-negative cells were considered antigen-specific and therefore characterized further. FIG. 7 shows an experiment to test selected clones for antigen specific binding to target expressing cells by FACS.

Clones that were shown to be target-specific were then sequenced and grouped on the basis of their sequence identity: a ‘cluster’ of antibody clones was defined as a group of antibodies sharing the same VH V-gene usage and having an identical HCDR3 sequence and HCDR3 length (FIG. 17). These clones are all derived from a single ancestral clone that diversified during the in vivo immune response in the MeMo mice. A ‘super-cluster’ was defined as a group of clones sharing the same VH V-gene usage and having at least 70% sequence identity in HCDR3 and the same HCDR3 length. Although this definition is arbitrary, it is probable (but not proven) that these clones also arose from a single B-cell precursor that was selected, activated and diversified during the in vivo humoral immune response. Practically, the clones in a supercluster are expected to bind the same epitope though with different affinities and/or different location on the epitope. Per super-cluster (defined as described above), at least two clones were then selected to be entered into the clone validation process, during which specific binding to the target was confirmed and the sequence of the clone was verified. These numbers are based on Fab clones of which the VH has a different germline gene usage and/or HCDR3 sequence. Clones that passed the clone validation process (i.e. of which the binding to—and specificity for the target was confirmed and for which the sequence was validated) were then used to re-clone the VH gene into an expression vector to produce and characterize the corresponding IgG. In total, 667 different antibodies were identified that specifically recognized one of the four targets LGR4, LGR5, ZNRF3 or RNF43, of which 288 were characterized further (Table 2). All these clones were then subsequently re-cloned into bispecific cLC IgG format with a ‘dummy’ (i.e. non-relevant, anti-TT) Fab arm to be able to characterize the mon-valent interaction of the Fab arm directed to the WNT target with its target.

### Example 3: Antibody Panel Characterization

The selected Fab fragments (designated with the code MFnnnn) isolated from phage display were re-cloned into an IgG1 expression vector containing DEKK mutations. To this aim, the VH-encoding cDNA was excised from the phagemid vector that was used to select the antibody fragment and re-cloned into the IgG expression vector containing (KK) DEKK mutations. By co-transfection with an expression construct containing the complementary (DE) DEKK mutations and encoding a Fab fragment directed at the non-binding control antigen Tetanus Toxoid (TT: MF1337), bispecific anti-WNTxTT IgG were obtained (WNT referring to LGR4, LGR5, ZNRF3 or RNF43). The bispecific IgG1 panel with monovalent binding activity to the WNT targets was then produced, purified and characterized with regard to their productivity and specificity.

Bispecific IgG were produced on a small scale by transient co-transfection of the plasmids encoding both Fab fragments in Freestyle 293F cells by combining different Fab fragments binding the WNT targeting arm (hLGR4, hLGR5, hZNRF3 or hRNF43) in the positively charged KK vector with the control Fab fragment directed to Tetanus Toxoid in the negatively charged DE bispecific vector. After production, bispecific IgG were purified by protein-A affinity chromatography and the buffer was exchanged to PBS. Successful productions resulted in an IgG1 full length antibody, with a minimal concentration of 0.1 mg/ml, which were assigned a unique code (PBnnnnn; where nnnnn represents a randomly generated number) to identify the specific combination of 2 different target binding Fab fragments.

Successfully produced bispecific IgG were tested for binding to their respective targets in both ELISA and FACS. The ELISA was performed using rhLGR4-Fc, rhLGR5-Fc, rhZNRF3-Fc or rhRNF43-Fc at 2 μg/ml or Tetanus Toxoid at 2.5 μg/ml as coating. IgG were tested at a single concentration of 5 μg/ml. IgG were considered binding to rhLGR4-Fc, rhLGFR5-Fc, rhZNRF3-Fc or rhRNF43-Fc when the OD_{450 nm} signal observed is five times above background (OD_{450 nm} signal of negative control antibody). Additionally, a FACS analysis of the bispecific IgG directed to TT and one of the four WNT targets (hLGR4, hLGR5, hZNRF3 or hRNF43) was performed to determine specific binding to their respective WNT target. FACS analysis was performed by using a DiD staining, mixing unlabeled Freestyle 239F cells (DiD-) with labeled antigen positive (hLGR4, hLGR5, hZRNF3 or hRNF43 expressing) Freestyle 293F cells (DiD+). Bispecific IgG were always tested in parallel on two mixes of cells for specificity. The bispecific IgG containing a Fab fragment directed to LGR4 or LGR5 were tested on both hLGR4 and hLGR5 overexpressing Freestyle 293F cells (mixed with antigen-negative Freestyle 293F cells), and the ZNRF3 and RNF43 binding antibodies were tested on both hZNRF3 and hRNF43 overexpressing Freestyle 293F cells (mixed with antigen-negative Freestyle 293F cells). Bispecific IgG were considered binding specifically to hLGR4, hLGFR5, hZNRF3 or hRNF43 when the MFI of the antigen positive population (Freestyle 293F cells stable expressing hLGR4, hLGR5, hZRNF3 or hRNF43) is five times higher than the MFI of the antigen negative population (Freestyle 293F cells). 41 out of 66 LGR4 Fab fragments, 69 out of 84 LGR5 Fab fragments, 92 out of 105 ZNRF3 Fab fragments and 29 out of 33 RNF43 Fab fragments were found to bind specifically to their respective targets (hLGR4, hLGR5, hZNRF3 or hRNF43) in bispecific (Biclonics®) format in both FACS and ELISA, or in a few cases FACS only (hLGR4 binding Fab fragments).

### Characterization of the Bispecific Antibody Panel

Bispecific monovalent IgG confirmed to bind specifically to human LGR4, LGR5, ZNRF3 or RNF43 were further characterized for affinity, stability, R-Spondin3 blocking capacity and mouse orthologue cross-reactivity after which a further sub-selection was made.

### Affinity Titration FACS Analysis

The binding antibodies were titrated in a limited antibody FACS, to rank them with regard to their affinity. Monovalent IgG directed to hLGR4, hLGR5, hZNRF3 or hRNF43 (all combined with the Tetanus toxoid Fab fragment) on the cellular surface of Freestyle 293F cells overexpressing hLGR4, hLGR5, hZNRF3 or hRNF43. Each IgG was tested on its corresponding overexpressing Freestyle 293F cell line. A two-fold dilution series of IgG (5 μg/ml-0.08 μg/ml) was tested on a fixed number of cells (5×10⁵ cells/well) stable expressing the WNT targets. The mean fluorescence intensity (MFI) from each individual measurement was determined by FlowJo (FACS analysis software BD). For each IgG the MFI values were plotted against the concentration of antibody, and from these curves the area under the curves (AUC) were calculated. Based on the AUC values a ranking of the bispecific IgG was made per target. An example of the data used for affinity ranking is given in FIG. 8.

### Mouse Orthologue Cross Reactivity of Selected Antibodies

In order to define further the binding characteristics of the bispecific IgG, mouse orthologue cross reactivity was determined. Constructs expressing the mouse orthologues of mLgr5, mZNRF3 and mRNF43 (pEF1_mLgr5-Myc-HIS, pDisplay_mZnrf3-Myc-PDGFR(TM), pDisplay_mRnf43-Myc-PDGFR(TM) were transiently transfected in HEK293T cells. Mono-valent IgG at a concentration of 5 μg/ml were used for staining and binding of IgG was analyzed by FACS. Bispecific IgG were considered mouse cross-reactive if the mean fluorescent intensity (MFI) increased two-fold compared to the non-transfected Freestyle 293F cells. 92 out of 92 anti-ZNRF3 IgG were cross reactive with mZnrf3; 9 out of 29 anti-RNF43 IgG were cross reactive with mRnf43 and 18 out of 69 anti-LGR5 IgG were cross reactive with the mouse Lgr5 orthologue. mLgr4 cross reactivity was tested in a ELISA on rmLgr4-Fc (RND systems) protein. Bispecific IgG were considered mLgr4 cross reactive in the OD_{450 nm} was 5× above background (OD_{450 nm} signal of negative control antibody). IgG were tested for binding at a single concentration of 5 μg/ml to rmLgr4-Fc and checked for cross reactivity. 36 out of 41 anti-LGR4 IgG tested positive for mLgr4 cross reactivity.

### R-Spondin Blocking

All bispecific IgG were tested in a ligand blocking ELISA to be able to test whether LGR4, LGR5, ZNRF3 or RNF43 targeting antibodies can interfere with R-Spondin3 binding. Binding of the Fc fusion proteins of the extracellular domain of the four WNT targets to coated R-Spondin3 was tested in the presence of an excess of bispecific IgG directed to LGR4, LGR5, ZNRF3 or RNF43. To test bispecific IgG directed to the different targets for their ability to block the interaction of the respective Fc fusion protein with rhR-Spondin3, they were tested in a blocking ELISA. IgG were tested using a single IgG concentration of 15 μg/ml.

Clones were considered blocking when in two independent assays at least 50% of the OD_{450 nm} signal was reduced. When only 20-50% of the OD_{450 nm} signal was reduced these clones were considered partially blocking. A subset of IgG targeting ZNRF3 (48) and RNF43 (10) and the complete LGR4 (41) and Lgr5 (69) panel, were tested for R-Spondin blocking capacity. For LGR4 3 were blocking and 5 partially blocking. For ZNRF3, 1 blocker and 1 partial blocker was identified. For RNF43 3 blockers and 2 partial blockers were identified, all belonging to 2 different superclusters. For the LGR5 panel 10 IgG that partially blocked binding of R-Spondin3 were identified. An example of the data generated in the R-Spondin3 blocking assay is given in FIG. 9.

### Measuring Antibody Stability at 40° C.

In order to gain an indication of the stability of the bispecific IgG, all IgG were incubated in serum-containing medium at 40° C. for 1 week and their binding in ELISA was then compared to that of bispecifics incubated in the same medium at 4° C. After 1 week, the IgG were used in an ELISA screening to determine whether they retained binding to their target. Binding was determined by the percentage of OD_{450 nm} signal left after one week incubation at 40° C. compared to one week incubation at 4° C. Most IgG were tested twice; when an IgG retained at least 50% of its binding in two independent assays, it was considered stable. When in the two experiments the stability varied; these IgG were considered to be partially stable, and when IgG retained two times less than 50% binding, these were considered unstable. For LGR4 12 out of 41 IgG retained binding (7 partially stable, 2 undetermined; did not bind in ELISA), for LGR5 38 out of 69 IgG retained binding (7 partially stable), for ZNRF3 28 out of 92 IgG retained binding (10 partially stable) and for RNF43 10 out of 29 IgG retained binding (9 partially stable).

### Ranking and Selection of Bispecific IgG

After characterization was finished, the data were collected and a ranking was made based on all data obtained. Selection of Wnt targeting Fab fragments for follow up functional screening was performed in the following way: Bispecific IgG which retained binding of at least 50% at 40° C. were selected. The highest affinity binders were then selected. This selection contained binders with different characteristics e.g. mouse orthologue cross reactivity or R-Spondin blocking capacity. In total, 54 bispecific Fab fragments directed at the four different WNT targets were selected for functional screening; for LGR4 10 out of 41 Fab fragments, for LGR5 17 out of 69 Fab fragments, for ZNRF3 18 out of 92 Fab fragments and for RNF43 9 out of 29 Fab fragments were selected (Table 3).

### Panel Generation for Functional Screening

The selected WNT targeting Fab fragments against hLGR4, hLGR5, hZNRF3 or hRNF43 were used for the generation of a large panel of bispecific antibodies (>500 bispecifics). The selected WNT-targeting Fab fragments were combined with receptor tyrosine kinase (RTK) binding Fab fragments into a panel of bispecific IgG and produced and purified. The Fab fragments specific for EGFR and HER3 that were selected block receptor activation in cell-based assays do not interfere with functionality of other Fab fragment when combined in bispecific format. Four EGFR Fab fragments MF3755, MF4280, MF3370 and MF4289 as well as four HER3 Fab fragments MF3178, MF3176, MF3125 and MF4863 were used in combination with the selected WNT-targeting Fab fragments (see Table 5 for a subselection of the produced panel).

Bispecifics were produced by coexpression of IgG vectors in which the WNT arm was expressed on the heavy chain containing the positively charged KK mutation and the RTK arm was expressed on the heavy chain containing the negatively charged DE mutation, purified and validated for their target binding, specificity and stability. The panels of bispecifics that passed binding and stability quality control (QC) were used for functional screening (53 out of 54 WNT targeting Fab fragments passed QC). The panel of >500 purified and validated bispecific antibodies targeting one of the four WNT targets (hLGR4, hLGR5, hZNRF3 or hRNF43) and an EGFR or HER3 Fab fragment or Tetanus Toxoid as a mock negative control Fab fragment control were screened for their activity towards patient-derived organoids in the functional screening assays described below.

### Example 4: Functional Screening of Bispecific Antibodies in Colon Organoids

Colon organoids are derived from LGR5-positive (cancer) stem cells grown in growth factor-containing expansion medium that allows the formation of epithelial colon structures that form a functional lumen (Sato et al. 2011 Gastroenterology 141:1762-1772). The organoid growth, development and lumen formation depends on the genetic background of the colorectal cancer cells and on the response to growth-stimulating growth factors, epidermal growth factor (EGF) or Neuregulin-1 (NRG)/Heregulin (HRG). Although the morphology of CRC tumoroids from different patients differs widely, the morphological profile is consistent between the tumoroids of the same origin and they are genetically almost identical to the original tumour they were derived from. High content quantitative analysis of images of cultured CRC organoids can therefore discriminate CRC organoids from different patients and can also be used to measure the morphological changes associated with activation of signaling pathways—e.g. driven by the ligands for HER receptors e.g. EGF and HRG. Similarly, inhibition of these responses by function blocking antibodies or other therapeutic molecules can be measured. Image-based analysis allows changes in morphology to be measured that may be independent of cell proliferation, providing additional information on compound activity to that which is obtained from conventional proliferation assays. Compound-induced phenotypic changes therefore form the basis for the functional screening of antibodies in CRC organoids.

The bispecific antibodies that have been developed target either EGFR or HER3 in combination with targeting the WNT-signaling related cell surface expressed proteins LGR4, LGR5, RNF43 or ZNRF3. The screening system is designed to monitor the inhibition of tumoroid outgrowth due to the activity of bispecific antibodies. This can be used for both the selection of organoids (and associated molecular profile) that are sensitive to targeted inhibition of specific pathways and also for the selection of novel molecules that are active in the organoids.

### Selection of Colon Tumoroid Models

Colon tumoroid models used for the screens were selected based on the demonstration of morphological changes in response to EGF, HRG or WNT3A. Morphological responses to these factors were investigated in a panel of 20 tumoroids of different patient origin (Van de Wetering et al. 2015 Cell 161:933-45). First, the tumoroids were expanded, split and re-seeded in 384-wells plates to analyze and document the basic morphology after 1 week of growth. Next, the same 20 tumoroids were tested for their responsiveness to growth factors, being EGF or HRG, or no growth factor present in the expansion medium. To do this, all 500 morphological features were analyzed and the changes induced by the soluble factors were measured. A set of robust features was selected for each organoid that enabled measurement of the growth factor response and discrimination between growth-factor dependent tumoroid cultures and growth-factor independent tumoroid cultures (see Table 4). This demonstrated that no single feature was sufficient to optimally quantify responses in different CRC organoids. Based on these data the following tumoroid lines were selected: P18T (APC^{mut}) which is heavily EGFR signaling dependent for growth and the formation of branched lumen structures inside the tumoroids; P14T (APC^{mut}, SMAD4^{mut}) that shows clear morphology altering effects upon EGFR and HER3 signaling and inhibition; P19Tb (APC^{wt}, but PIK3CA, TP53, BRAF, ARID1A, ARID2, ERBB3, POLE and RNF43 mutant) as a tumoroid model that lacks APC mutations and hence depends on WNT3A for expansion; finally, P26T (APC^{mut}, KRAS^{mut}, TP53^{mut} and CTNNB1^{mut}) as a model that exhibits no dependency on the presence of growth factors for expansion. In the initial screening of bispecific antibodies P26T did not show any growth factor dependency and no sensitivity towards growth factor receptor targeting antibodies, and was dropped from further antibody screening.

### Identification of Functional Fab Fragments in Bispecific Antibodies

To identify functional bispecific antibodies capable of altering the morphological characteristics of colon tumoroids, they were compared to growth conditions without EGF stimulation and to the effect of EGFR/MAPK signaling inhibiting compounds (e.g. Cetuximab and Trametinib) in EGF growth conditions. Functional bispecific antibodies were defined by their ability to limit tumoroid expansion in EGF conditions in the direction of the morphological profile obtained by EGFR/MAPK signaling inhibitors and/or omitting EGF from the culture medium. WNT-targeting Fab fragments that potentiated the effect of the EGFR-targeting arm are those that showed significantly enhanced growth inhibiting capacity compared to the equivalent monovalent (bispecific EGFRxTT)-targeting Fab fragment.

Bispecific antibodies that only targeted LGR4, LGR5, ZNRF3 or RNF43 (in combination with the non-targeting Tetanus Toxoid (TT)-Fab fragment) were compared to the negative control morphology in either no growth factor containing medium (P19Tb) or EGF or HRG containing medium (P18T and P14T) to seek potential effects of targeting WNT-signaling receptors alone.

Bispecific antibodies carrying HER3-targeting Fab fragments were functionally compared to growth conditions without HRG stimulation or the effect of HER3 targeting antibodies and the PI3K inhibitor in HRG conditions; Functional HER3-inhibiting bispecific antibodies were defined as those that inhibited the HRG-stimulated growth response into the same direction in Euclidean space as the MEK kinase inhibitor, Trametinib, no HRG culture conditions and/or HER3 targeting reference antibodies. WNT-targeting Fab fragments that potentiated the HER3-targeting arm effect in terms of growth inhibiting effect compared to the equivalent monovalent HER3 bispecific were considered as candidate targeting Fab fragments.

### Selection of Bispecific Antibody Candidates in the Primary Screen

Bispecific antibodies that, in the presence of EGF, induced a change in the morphology of P14T and P18T colon tumoroids approaching, matching or exceeding the phenotype in the absence of growth factors or presence of reference EGFR/MAPK inhibitors, these antibodies were identified as potential candidates. Similarly to the EGF conditions, the antibodies tested in HRG culture conditions were marked for follow-up if the effect approached, matched or exceeded the negative control distance of the no growth factor conditions and/or reference HER3/PI3K inhibitors. For P19Tb, which was relatively insensitive to growth factor treatment, the functional antibody selection was based on calculating the distance from the negative controls marked by the morphological profile induced by Trametinib and the PI3K-inhibitor CH5132799.

The bispecific panels were screened in 3 different colon tumoroids (P18T, P14T and P19Tb). The screened antibody panel tested the combinations of four different EGFR-targeting Fab fragments, four different HER3-targeting Fab fragments and one anti-Tetanus Toxoid (negative control) Fab fragment with 53 different WNT-targeting Fab fragments and one TT targeting Fab fragment. Each bispecific antibody was scored for its capacity to inhibit tumoroid outgrowth. None of the TT/WNT fragment combinations inhibited tumoroid outgrowth (0/53). Within the EGFR-targeting Fab fragment group, the MF3755 Fab fragment was most potent: 22/53 bispecific MF3755/WNT Fab fragment combinations inhibited tumoroid development. The runner up EGFR-targeting fragment was MF4280 with 4/53 active inhibitory EGFR/WNT Fab fragment combinations. One (1/53) EGFR-targeting bispecific MF3370/WNT Fab fragment combination showed inhibitory activity and none (0/53) of the EGFR-targeting bispecific MF4289/WNT Fab fragment combinations significantly limited the tumoroid outgrowth. Within the HER3-targeting Fab fragment containing bispecific antibody group, the MF3178 Fab fragment in combination with the WNT-targeting Fab fragments was most potent: 19/52 MF3178/WNT combinations inhibited tumoroid outgrowth. Runner up HER3 targeting Fab fragment was MF4863 with 6/53 active MF4863/WNT bispecific antibodies. Bispecific antibodies containing the HER3 targeting Fab fragments MF3125 (0/53) and MF3176 (0/52) showed no significant tumoroid outgrowth inhibition.

In ranking, the RTK-targeting Fab fragments that most potently combined in the bispecific IgG format with the WNT targeting Fab fragments were MF3755 as the EGFR targeting Fab fragment and MF3178 as the HER3 targeting Fab fragment. The LGR4 targeting Fab fragments MF5777 and MF5781, the LGR5 targeting Fab fragments MF5790, MF5803, MF5814, MF5816, MF5817 and MF5818, the RNF43 Fab fragments MF5832 and MF5836 and the ZNRF3 targeting Fab fragments MF5850, MF5853, MF5855, MF5884 and MF5888 were identified as potential candidates to progress to the validation screen based on their functional effect on P18T, P14T and P19Tb tumoroid morphology when combined with RTK targeting Fab fragments in the bispecific IgG format. The LGR4, LGR5, RNF43 and ZNRF3-targeting Fab fragments were combined with either the EGFR targeting Fab fragments MF3755, the HER3 targeting Fab fragments MF3178 or the non-targeting TT Fab fragments MF1337 and produced in new batches to confirm their activity in the follow-up validation screen.

### Bispecific RTK/WNT Antibody Validation Screen

Because the RTK/WNT bispecific antibody candidates had shown potentially therapeutically relevant activity in both EGF and in HRG culture conditions, the validation screen of the bispecific candidate antibodies was also performed in two growth factor conditions: 5 ng/ml EGF or 5 ng/ml HRG. Controls included wells that received culture medium without either EGF or HRG. Other controls used in growth factor conditions: Cetuximab (EGFR), PG3755 (EGFR/EGFR), Trametinib (MEK), CH5132799 (PI3K), PG3178 (HER3/HER3), PG2863 (HER3/HER3), PG3794 (HER3/EGFR) and PB4522 (HER3/EGFR). PG1337 (TT/TT) served as a negative control antibody, and was referred to as negative control, along with wells receiving equal volumes of DMSO or PBS as the compound- or antibody-treated wells respectively.

The antibody validation screen of 46 bispecifics was performed in 24 colon tumoroids of different patient origin, in duplicate or in quadruplicate. The set of tested colon tumoroid lines consisted of 3 growth factor-dependent patient samples described in Van de Wetering et al. (2015 Cell 161:933-45): P18T, P14T and P8T and 2 growth factor independent patient samples, P19Tb and P28N. A novel set of 19 colon tumoroids were screened: 10 growth factor dependent primary colon tumoroid lines, 5 growth factor independent primary colon tumoroid lines and 4 (growth factor dependent) metastatic colon tumoroid lines. The screening of the bispecific antibody candidates in the panel of 24 different patient-derived colon tumoroid lines revealed that growth-factor dependency for tumoroid outgrowth is able to identify RTK-targeting Fab fragment mediated tumor inhibition.

The cut-off to identify functional colon tumor inhibitory antibodies in the bispecific antibody validation screen was calculated by normalizing the morphological profile on a scale of 0 (phenotype equal to a fully growth inhibited profile) and 1 (phenotype similar to the negative controls). Every time a single antibody, at a certain dose, in a certain tumoroid line scored less than 0.5, this antibody treatment was marked as showing inhibition of tumor development. The number of times an antibody, at a certain dose in either EGF or HRG culture conditions showed inhibition was scored and expressed as a percentage of the number of treated wells for that condition. This percentage was calculated in all 24 screened colon tumoroid lines.

The validation screen in 24 colon tumoroids identified four LGR5 targeting Fab fragments and 2 RNF43 targeting Fab fragments that potentiated the RTK-Fab fragments mediated tumor development inhibition: MF5816, MF5814, MF5818 and MF5790 as LGR5 targeting antibody Fab fragments and MF5832 and MF5836 as RNF43 targeting Fab fragments. The top ranking antibody was the EGFR/LGR5 bispecific antibody PB10651 composed of the targeting Fab fragments MF3755 and MF5816: FIG. 10. In HRG conditions, the top ranking antibody was the HER3/LGR5 bispecific antibody PB10748 composed of the targeting Fab fragments MF3178 and MF5816. This showed, in two independent growth factor conditions and in combination with two different RTK targeting Fab fragments, that the LGR5-targeting Fab fragments MF5816 is the most potent WNT-targeting Fab fragments that enhances RTK targeting. MF5816 combined with MF3755 in PB10651 adds another level of tumor inhibition by potently reducing tumoroid growth and development, observable by further loss of lumen formation, cell shrinkage and rounding up of the nuclei. These morphological findings indicate that the EGFR/LGR5 antibody PB10651 actively blocks epidermal growth factor signaling and induces a cell death response in the growth-impaired tumoroid cells: FIG. 11.

This morphological phenotype was not observed with EGFR targeting antibody Cetuximab nor when MF3755 was formatted as a conventional IgG.

### Selection of the Lead Candidate Antibody

PB10651 (EGFR/LGR5 bispecific antibody (composed of the Fab fragments MF3755 and MF5816)) was selected as the primary lead candidate antibody in 5 ng/ml EGF conditions for the following characteristics: PB10651 showed potent inhibitory effects at 10 μg/ml test dose in 75% of the 24 different colon tumoroid models; 2) 67% of the wells treated with PB10651 at 10 μg/ml (40/60) showed more than 50% growth reduction; 3) 52% (31 out of 59 wells) showed more than 50% growth reduction upon PB10651 treatment at 2 μg/ml; 4) PB10651 outperformed Cetuximab (47% (56/119) at 10 μg/ml and 29% (35/119) at 2 μg/ml) and the EGFR/TT reference antibody PB9919 (27% (16/60) at 10 μg/ml and 5% (3/60) at 2 μg/ml).

PB10647 (EGFR/LGR5 MF3755xMF5814) is the second candidate antibody that inhibited 54% of the 24 different tumoroid models (13/24) at 10 μg/ml, and more than 50% growth reduction was found in 52% of the exposed wells (31/60) at 10 μg/ml and in 37% (22/60) at 2 μg/ml.

Other EGFR/LGR5 antibodies are PB10659 (MF3755xMF5818), effective in 50% of the 24 tumoroid models (12/24), 50% of the wells exposed with PB10659 at 10 μg/ml showed inhibition (30/60) and 32% (19/60) at 2 μg/ml. PB10627 (MF3755xMF5790) was active in 42% of the tumoroids (10/24) and showed reduction at 10 μg/ml in 39% of the wells (23/59) and at 2 μg/ml in 33% of the wells (20/60). PB10631 (MF3755xMF5803) was active in 33% of the tumoroids (8/24) and inhibitory at 10 μg/ml in 34% of the wells (20/59) and in 12% (7/59) at 2 μg/ml. PB10655 (MF3755xMF5817) was active in 20% (5) of the 24 tumoroid models and showed inhibition in 25% of the wells (15/60) at 10 μg/ml and 6/60 (10%) at 2 μg/ml.

EGFR/LGR4 antibody PB10619 (MF3755xMF5777) was active in 29% (7/24) tumoroid models and led to tumoroid inhibition in 28% (17/60) wells at 10 μg/ml and 15% (9/60) at 2 μg/ml. EGFR/LGR4 antibody PB10623 (MF3755xMF5781) was active in 8 of the 24 tumoroid models (33%) and inhibited at 10 μg/ml in 30% of the wells (18/60) and 10% of the wells at 2 μg/ml (6/60).

EGFR/RNF43 antibody PB10661 (MF3755xMF5832) was active in 13 of the 24 tumoroid models (54%) and showed inhibition in 42% of the wells (25/60) at 10 μg/ml and 22% (13/60) at 2 μg/ml test dose. EGFR/RNF43 antibody PB10667 (MF3755xMF5836) was active in 13/24 tumoroid models (54%) and showed inhibition at 10 μg/ml in 35% of the wells (21/60) and at 2 μg/ml in 20% of the wells (12/60).

EGFR/ZNRF3 antibodies are: PB10675 (MF3755xMF5850), active in 8/24 tumoroid models (33%) with 35% of the tested wells (21/60) showing more than 50% inhibition at 10 μg/ml and 15% (9/60) at 2 μg/ml; PB10695 (MF3755xMF5884) was active in 37% of the tumoroid models (9/24) and showed inhibition in 34% (20/59) at 10 μg/ml and 12% (7/59) at 2 μg/ml; PB10679 (MF3755xMF5853) inhibited the tumoroid development in 21% of the tumoroid models (5/24), 20% of the wells at 10 μg/ml (12/60); PB10703 (MF3755xMF5888) was active in 5 of the 24 tumoroid models (21%) and inhibited at 10 μg/ml in 22% of the wells (13/60) and at 2 μg/ml in 12% of the wells (7/59).

PB10748, the HER3/LGR5 antibody composed of the Fab fragments MF3178 and MF5816, is active as a HRG-stimulated tumoroid inhibiting antibody in 62% of the tested colon tumoroid models (15/24), showing inhibition in 56% of the exposed wells (67/120). PB10748 outperformed the HER3/TT reference antibody PB9215 (MF3178xMF1337) which inhibited more than 50% of the HRG-stimulated tumoroid development in 34% (41/120) exposed wells.

Other HER3/LGR5 antibodies are: PB10735 (MF3178xMF5814), active in 11/24 tumoroid models (46%) and inhibitory in 43% of the exposed wells (51/118); PB10756 (MF3178xMF5818) was active in 8/24 (33%) of the tumoroid models and inhibitory in 37% of the exposed wells (44/119); PB10715 (MF3178xMF5790) was active in 12/24 (50%) of the tumoroid models and inhibitory in 42% of the wells (49/117); PB10719 (MF3178xMF5803) was active in 7/24 (29%) of the tumoroid models and inhibitory in 34% of the exposed wells (40/119); PB10752 (MF3178xMF5817) was active in 12/24 (50%) of the tumoroid models and inhibitory in 28% of the wells (33/120).

Two HER3/RNF43 antibodies are: PB10764 (MF3178xMF5836), active in 17/24 tumoroid models (71%) and inhibitory in 38% of the exposed wells (46/120) and PB12336 (MF3178xMF5832), active in 11/24 tumoroid models (46%) and inhibitory in 41% of the exposed wells (49/120).

Two HER3/LGR4 antibodies are: PB10711 (MF3178xMF5781, active in 62% of the tumoroid models (15/24) and inhibitory in 50% of the exposed wells (59/119) and PB10707 (MF3178xMF5777), active in 13 of the 24 tested tumoroid models (54%) and inhibitory in 41 of the 120 exposed wells (34%).

Bispecific antibodies targeting both HER3 and ZNRF3 are: PB10776 (MF3178xMF5853), active in 11/24 (46%) of the tumoroid models exposed to HRG and inhibitory in 40% of the exposed wells (48/120); PB10772 (MF3178xMF5850), active in 12/24 models (50%) and inhibitory in 39% of the exposed wells (46/119); PB10780 (MF3178xMF5855), active in 8/24 (33%) colon tumoroid models and 31% of the exposed wells (37/119); PB10800 (MF3178xMF5888) was active in 12/24 tumoroid models (50%) and inhibitory in 29% (34/119) of the exposed wells.

### Example 5: Characterization of the Lead Candidate Antibody

### Affinity Measurement of Bispecific Antibody Binding Using a Cell Based Assay.

After optimization of the iodination protocol, a labelled PB10651 protein with a specific radioactivity of 40 GBq/μmol was obtained. The radiochemical purity was >99% as analyzed by protein precipitation. In the Lindmo assay, only a minor reduction of the immunoreactivity was observed. The immunoreactivity of ¹²⁵I-PB10651 towards EGFR and LGR5 was estimated to be >89% using CHO cells expressing either EGFR or LRG5. Results of the Lindmo assay are depicted in FIG. 12. Using steady-state affinity measurements, the K_{D} of ¹²⁵I-PB10651 towards CHO-LGR5 cells was measured to be 0.86 f 0.13 nM, and the K_{D} of ¹²⁵I-PB10651 towards CHO-EGFR cells was found to be 0.22 f 0.086 nM, The K_{D} of ¹²⁵I-PB10651 towards DLD-1 cells was estimated to 0.18 f 0.024 nM. An example of the data is depicted in FIG. 13.

### Epitope Mapping of PB10651 Through Shotgun Mutagenesis Analysis

To map the epitopes on EGFR and LGR5 respectively recognised by both Fab arms present in PB10651, the shotgun mutagenesis approach was used. Residues that were relevant for the binding of both Fab arms to their respective antigens could unequivocally be determined. Mutations that abrogated the binding of PB10651 to either EGFR or LGR5 and that did not inhibit the binding of the control antibodies (identifying relevant residues) are depicted in Table 8. These data show the anti-EGFR Fab arm to recognise domain III on a region that partially overlaps with the site that normally contacts the ligand EGF (Ogiso et al., 2002). This indicates that PB10651 may directly block the ligand-EGFR interaction. The epitope is indicated in the structure of EGFR (pdb reference 1YY9) in FIG. 14. The Fab arm recognising LGR5 was shown to recognise residues that are located in the N-CAP domain and first Leucine Rich Repeat (LRR). The residues are indicated in FIG. 15 in the structure of LGR5 in complex with RSPO1 (pdb reference 4BSR, Peng et al., 2013).

### REFERENCES

Li S, Schmitz K R, et al., (2005) Structural basis for inhibition of the epidermal growth factor receptor by cetuximab. Cancer Cell. April; 7(4): 301-11.
Ogiso, H. Ishitani, R. et al., (2002) Crystal structure of the complex of human epidermal growth factor and receptor extracellular domains. Cell, Vol. 110, 775-787.
Peng, W. C. de Lau W. et al., (2013) Structure of Stem Cell Growth Factor R-spondin 1 in Complex with the Ectodomain of Its Receptor LGR5. Cell Rep 27; 3(6): 1885-1892.

### Competition for Binding of the Anti-LGR Fab Arm in PB10651 by the Ligand R-Spondin1 Using a Cell Based Assay

To be able to determine the ability of the anti-LGR5 Fab arm present in PB10651 to bind LGR5 in the presence of the ligand R-spondin1, a cell-based assay was set up. The EC50 for binding of the ligand-blocking anti-LGR5 antibody OMP88R20 (PG7711) to the LGR5-expressing cell clone was determined to be 50 ng/ml (333 pM) and this concentration was then used to determine the competition with the ligand R-spondin1 for binding to LGR5. FIG. 16 shows the MFI signal (normalised to the MFI signal obtained in the absence of R-spondin1) of OMP88R20 binding to LGR5-expressing CHO-K1 cells measured in FACS as a function of the concentration of added R-spondin1. Bispecific anti-(TTxLGR5) antibodies were then tested for their ability to bind the LGR5 expressing CHO cell clone in the presence of increasing concentrations of R-spondin1. PG7711 was included as positive control at 50 ng/ml. The bispecifics were first tested for binding to the CHO cell clone expressing LGR5 in a concentration range to determine the EC50 for binding in FACS. The EC50 was found to be 156 ng/ml for PB10286 containing the lead Fab MF5816 and 200 ng/ml for PB10261 containing MF5790. However, bispecifics were tested at 100 ng/ml to compare the same concentrations of antigen-specific Fabs in the assay as used or PG7711 and to possibly even increase the sensitivity of the assay. FIG. 17 shows that the lead anti-LGR5 Fab MF5816 was not inhibited from binding LGR5, even in the presence of a large (120-fold) molar excess of the ligand, whereas in the same assay, binding of the copied version of OMP88R20 was fully inhibited. In addition, reduced binding of PB10261 (containing the LGR5 targeting arm MF5790) was observed, demonstrating that the assay is able to discriminate between ligand-blocking, as well as ligand non-blocking anti-LGR5 Fabs. No differences and no competition were observed when using the 1D9 rat anti-LGR5 antibody in the blocking assay (FIG. 17), demonstrating specificity of the interaction.

### REFERENCE

de Lau W, Barker N, et al., Lgr5 homologues associate with Wnt receptors and mediate R-spondin signalling. Nature. 2011 Jul. 4; 476 (7360): 293-7.

### Ligand-Blocking Capacity of the Anti-EGFR Fab Arm of PB10651 Using a Cell Based Assay.

In order to test the capacity of the anti-EGFR Fab of PB10651 to block EGF-mediated signalling, a cell-based assay was used. FIG. 18 shows the potency of PG3755 to block the EGF-mediated cell death in A431 cells (tested according to the method described by Gulli et al.,) compared to that of cetuximab. The antibody blocks EGF-mediated signalling with a potency that is at least equal to that of cetuximab.

### REFERENCE

Gulli, L. F., et al., Epidermal growth factor-induced apoptosis in A431 cells can be reversed by reducing the tyrosine kinase activity. Cell Growth Differ, 1996. 7(2): p. 173-178.

### Species Cross-Reactivity of PB10651.

PB10651 was tested for its cross-reactivity with the rat—and cynomolgus orthologues of both targets: EGFR and LGR5. The cDNA sequence of cynomolgus LGR5 that was obtained by RT-PCR from cynomolgus cDNA matched the predicted sequence from GenBank (reference XM_005571542). Both the human—as well as the rat—and cynomolgus-encoding constructs were used for a transient transfection of CHO-K1 cells. Antibody binding to cells transiently expressing either the human—rat—or cynomolgus orthologue was then tested in FACS. FIG. 19 shows the graphs of the mean fluorescence intensity (MFI) obtained in FACS after staining as a function of the antibody concentration used for staining. Both the Fab arm recognising EGFR, as well as the Fab arm recognising LGR5 were found to be fully cross-reactive to the cynomolgus orthologue of the target: the EC50 values for binding were not significantly different for binding to the human or cynomolgus orthologue. In a similar experiment, the cross-reactivity of both arms with the rat orthologue was assessed. PB10651 was found to bind well to the rat orthologue of LGR5, although the EC50 was shifted with about a log compared to the value found for binding human LGR5. However, the anti-EGFR Fab arm was hardly cross-reactive (two log difference in EC50) to the rat orthologue of EGFR, making the antibody unsuited for rat in vivo models or toxicity studies in rats. As positive control for cynomolgus LGR5 cross-reactivity, the copied version of hu8E11v2 (PG7543) was used. Cetuximab was used as control for cynomolgus EGFR cross-reactivity.

### Specific LGR5 Targeting on a Patient-Derived Organoid

In order to demonstrate that Fab fragments MF5816 and MF5814 bind to LGR5 expressed on the surface of patient-derived organoids, PG5816 and PG5814 (bivalent, monoclonal IgG) containing Fab fragments MF5816 or MF5814 were used to stain the individual cells derived from the organoid P18T. Using PG5816 resulted in staining of 53.6% of the P18T tumoroid derived cells and PG5814 resulted in staining 52.5% of the P18T tumoroid derived cells, compared to staining using the negative control antibody directed to TT (FIG. 20).

### LGR5-Sorting Enriches for LGR5-Expressing Cells and for Tumour-Initiating Cells

In addition, to show that the LGR5 targeting Fab fragments in a bispecific format actually bind LGR5 on the P18T tumoroid cells, cells were stained with bispecific antibodies PB10284 and PB10286 containing Fab fragments MF5814 or MF5816 combined with the anti-TT Fab fragment. After staining, cells were sorted using FACS, and the stained and non-stained cell populations were analyzed by Q-PCR for LGR5 mRNA levels. Staining P18T derived tumoroid cells using the bispecifics containing LGR5 Fab arm MF5816 or MF5814 resulted in a 6-14 fold enrichment of LGR5 mRNA expression levels in the sorted LGR5-positive cell fraction compared to the LGR5-negative cell fraction: FIG. 21.

Furthermore these enriched LGR5 expressing P18T tumoroid cell populations allowed for a 4-fold increase in colony forming capabilities: P18T was used for FACS staining and sorting of the top (positive) and bottom (negative) 15% of stained cells identified by the anti-LGR5 antibodies: MF5814-TT, MF5816-TT and MF5790-TT. 2000 cells were plated in 25 μL of BME in duplicate (technical replicates). After two weeks of growth, the organoids were manually counted using an inverted light microscope. The results demonstrate that on average the MF5814-TT, MF5790-TT and MF5816-TT antibodies enrich for organoid growth, with the positive fractions forming 4.5, 3.7 and 7.1 times more organoids than the negative fractions, respectively: FIG. 22. These data demonstrate a clear enrichment for tumour initiating cells after sorting for LGR5-positive cells from a patient-derived organoid.

### LGR5xEGFR Bispecifics Inhibit Patient-Derived Organoid Outgrowth

As an independent way of measuring the therapeutic efficacy of the lead bispecific anti-LGR5xEGFR antibodies, identified in the 3-D screening, organoids were treated with the different bispecifics and organoid growth was assessed in a standard colony formation assay after 7 days: FIG. 23. The images shown on the right of the figure are an example of the images produced by the macro's analysis, and depicts one drop containing organoids (black circles). The experiment was repeated on three separate occasions and the number and size of organoids was averaged between the experiments. These data corroborate the data obtained using the antibody validation screen and show that lead bispecific LGR5xEGFR antibodies are potent inhibitors of patient-derived organoid growth.

Treatment of Patient-Derived Organoids with LGR5xEGFR Bispecific Antibodies Strongly Reduces the Non-Differentiated Cell Population.

After tumoroids were treated for seven days with the lead bispecific LGR5/EGFR bispecific, quantitative real-time PCR analysis was used to assess the antibodies effects upon expression of LGR5 and CK20 (a marker of differentiation) (FIG. 24). The results demonstrate that treatment with the EGFRxTT (MF3755xMF1337; PB9919) antibody causes a 0.5-fold increase in LGR5 mRNA levels whilst reducing the levels of CK20 4-fold relative to TTxTT. The LGR5 antibodies MF5814xTT (MF5814xMF1337; PB10284) and MF5816-TT (MF5816xMF1337; PB10286) show little effects. However, when the EGFR arm is substituted for the TT arm, the LGR5 levels are reduced by 5.9 and 7.2-fold after treatment with the MF5814xEGFR (MF5814xMF3755; LGR5xEGFR; PB10647) and MF5816-EGFR (MF5816xMF3755; LGR5xEGFR; PB10651) antibodies respectively. The CK20 expression is also reduced by both the PB10647 (EGFR/LGR5, MF5814xMF3755) and PB10651 (EGFR/LGR5, MF5816xMF3755) antibodies by 10.4- and 13.7-fold. These data suggest that in this tumoroid line, through addition of an LGR5 targeting arm, the relative increase in LGR5 mRNA levels caused by EGFR inhibition can be abrogated, whilst also enhancing the original effects of the EGFR inhibition (decrease in CK20 mRNA).

Treatment of Organoids from Tumours (Tumoroids) and Organoids from Normal Tissue with PB10651.

In order to demonstrate that PB10651 is selectively targeting colon cancer-derived tumoroids and not normal colon tissue-derived organoids, organoid cultures were incubated with afucosylated PB10651 or Cetuximab. FIG. 26 shows the organoid (tumoroid) size at the respective dosage of the indicated antibody. C51N is an organoid from normal colon tissue. C1M is an organoid (tumoroid) from cancerous tissue (FIG. 26B). FIG. 26A depicts the results on an organoid from normal tissue (C55N) and cancerous tissue (C55T) from the same patient. FIG. 26C shows the IC₅₀ table for Cetuximab and PB10651 in 5 normal tissue organoids, 3 primary colon cancer tumoroids and 3 metastatic colon cancer tumoroids. The Cetuximab IC₅₀ PB10651 IC₅₀ ratio is given in the last column of FIG. 26C. This shows that PB10651 is 20-200× more potent than Cetuximab in tumoroids, while the effect of PB10651 in normal organoids is weaker than Cetuximab. Additional tests demonstrated that the presence of WNT or R-Spondin in the culture medium had no influence on the efficacy of Cetuximab or PB10651 in inhibiting outgrowth of the tumoroids (not shown).

### LGR5 Targeting Antibodies do not Inhibit Colon Tumoroid Outgrowth

To verify that the bispecific aspect of PB10651 is required to achieve the tumour-inhibiting capacity, PB10651 was compared to other WNT-targeting antibodies in production by Genentech, Bionomics or OncoMed (Table 7). In FIG. 27, the effects of LGR5 targeting antibodies (PG7709, PG7711, PG7712 and PG7543) on organoid growth are compared to those mediated by PB10651 and Cetuximab in the tumoroid models P18T and C1M. The results show that none of the comparator antibodies inhibit colon tumoroid outgrowth.

Bispecific PB10651 is More Potent than the Mix of the Bivalent, Mono-Specific Antibodies in Inhibiting Tumoroid Growth The bispecific antibody PB10651 is composed of an EGFR targeting arm (MF3755) and an LGR5-targeting arm (MF5816). To show that the actual physical interaction between both Fab arms is required to achieve the potent inhibition effect on colon tumoroids, these were incubated with increasing doses of PB10651 (MF3755xMF5816; EGFRxLGR5), Cetuximab, PG3755 (MF3755xMF3755; EGFRxEGFR), PG5816 (MF5816xMF5816; LGR5xLGR5), PG1337 (MF1337xMF1337; TTxTT) and a 1:1 mix of PG3755 with PG5816. FIG. 28A shows that treatment with a mixture of anti-LGR5 and anti-EGFR antibodies results in a less potent growth inhibition of tumoroids compared to treatment with the bispecific antibody PB10651. Interestingly, the growth of normal organoids was more potently inhibited by the mixture of anti-EGFR and anti-LGR5 antibodies than by the bispecific antibody. These effects are summarized in the IC₅₀ table for other tumoroid and normal organoid models in FIG. 28B.

### Localization Studies of PB10651 in Colon Tumoroids Reveal a Specific Intracellular Staining Pattern

In order to show that the antibodies reach the tumoroids while seeded in the BME2 RGF hydrogel, 7-day old P18T tumoroids were treated for 24 hours with the indicated antibodies (2 μg/ml) and then fixed (15 minutes, 4% paraformaldehyde) and permeabilised (0.1% Triton-X100 and 0.5% BSA in PBS). Organoids were subsequently counter-stained with goat-anti-human-FITC (Thermo Scientific, 1:3000). Nuclei and actin were stained as described in (Di et al PlosOne 2014, PMID 25289886). FIG. 29 show that all antibodies are able to penetrate the gel and bind all the cells in a tumoroid. The EGFR-targeting antibodies Cetuximab and PG3755 localize to the plasma membrane, while PB10651 shows an intracellular speckled staining pattern, which is not observed for any of the comparator LGR5 antibodies (PG7709, PG7711 or PG7712).

Localization Studies of PB10651 in Colon Tumoroids Reveal a Juxtanuclear Intracellular Punctate Staining Pattern that Correlates with Tumoroid Sensitivity

More colon tumoroid and organoid models were included in the PB10651 counterstaining and imaging assay (see above). FIG. 30 shows that the intracellular staining pattern of PB10651 is observed in P18T, COM, C55T (highly sensitive to PB10651, IC₅₀<1 μg/ml), in some tumoroids of C31M (moderately sensitive to PB10651), but not in the PB10651 insensitive (IC₅₀>10 μg/ml) C28T, P8T or P19Tb tumoroids or C51N normal colon organoids. The appearance of juxtanuclear intracellular speckles with PB10651 is consistently found, independent of the antibody incubation-time (24 hours or 7 days), at low antibody concentrations (50 ng/ml PB10651) and in Alexa-488-conjugated form.

### Example 6: Production and Biochemical Characterization of PB10651

### Plasmid Generation

The plasmids for transfection were generated from plasmids MV1453 and MV1626, see FIG. 31. These plasmids were digested with SfiI and BstEII restriction enzymes (MV1453) and SfiI and XhoI restriction enzymes (MV1626) after which the VHs MF3755 (digested SfiI and BstEII) and MF5816 (digested SfiI and XhoI), were ligated to form constructs MG3755C453 and MG5816C626. MV1622 encodes the Flag-tagged RMD enzyme (FIG. 32).

### Antibody Production

Protein production was performed by transient transfection of Freestyle 293-F suspension cells (Invitrogen cat. no. R79007) cultured in FreeStyle 293 Expression Medium (Gibco, cat. no. 12338-018) supplemented with 2 mM L-Glutamine (Gibco, cat. no. 25030-024) that were inhibited in attaching fucose residues to the tail of the antibodies [Henning von Horsten et. al., Glycobiology, vol. 20, no. 12, pp. 1607-1618, 2010]. One day before transfection, cells were seeded at a density of 5.0×10⁵ cells/mL and incubated o/n at 37° C., 8% CO₂ at an orbital shaking speed of 155 rpm. For transfection, a mixture of plasmid DNA and polyethyleneimine (PEI, MW 25,000 Da, Polysciences Inc., cat. no. 23966) in culture medium was prepared. For a 25 mL transfection volume, 25 μg endotoxin-free plasmid DNA was mixed with 62.5 μg PEI and 2.5 mL culture medium. The mixture was then vortexed, incubated for 20 minutes at RT and added to the cells. The cells were incubated at 37° C., 8% CO₂ at an orbital shaking speed of 155 rpm for 6 days. The cell suspension was collected, centrifuged at 1000 g for 10 min and the supernatant was collected and centrifuged at 4000 g for 10 min.

### Antibody Purification

Antibody purification is performed by binding the antibodies batch-wise to MabSelectSure LX (GE Healthcare) for several hours at room temperature. The MabSelectSure LX sepharose containing bound antibody was then transferred to a gravity flow column. The column was washed with PBS, antibodies were eluted using 100 mM citrate buffer pH 3.0 and pH was neutralized to 7.0 using 1M Tris pH 8.0. Samples were concentrated using Vivaspin20 (Sartorius) 10 kDa spin filters and further purified by gel filtration using a Superdex200 26/600 column (GE Healthcare) pre-equilibrated with PBS buffer.

### Cation Exchange HPLC

Cation exchange chromatography (CEX-HPLC) was used to address the charge heterogeneity of the antibody samples as well as to determine the presence and amount of product-related impurities (homodimers and halfbodies). The experiments were performed at ambient temperature on a Dionex HPLC system equipped with an SP STAT 7 μm column (Tosoh Biosciences) and a UV-vis detector. 10 μg of sample was injected in each run. A gradient of 25 mM phosphate buffer pH 6.0 with NaCl concentrations increasing from 0 to 1 M was applied to separate the antibodies. The data were analyzed using Chromeleon software.

### ADCC Reporter Assay

The activity of the afucosylated antibody PB10651 (MV1622) was tested on ADCC reporter cells containing either the V158 (high affinity) FcγRIIIa receptor variant or the F158 (low affinity) FcγRIIIa receptor variant (Promega). A serial titration of antibody, i.e. afucosylated PB10651 (PB10651-MV1622), non-afucosylated PB10651 and a non-targeting IgG1 (PG1337) was added in combination with ADCC reporter cells harboring high and low affinity FϵyRIIIa variants [Cartron et. al., Blood, vol. 99, no. 3, pp. 754-758, 2002;] Musolino et. al., Journal of Clinical Oncology, vol. 26, no. 11, pp. 1789-1796] to A431 cells, A549 cells and BxPC3 cells. ADCC activity was detected by measuring luciferase activity.

### Experimental

Freestyle 293-F cells were transfected with MG3755C453, MG5816C626 and MV1622, resulting in IgG PB10651p10. After purification, the yield of PB10651 (MV1622) was determined to be 29 mg per liter production volume by OD280 measurement. CEX-HPLC analysis was performed on the purified protein (FIG. 33), showing a main peak with some acidic charge variants at a retention time of 15 minutes representing the bispecific PB10651(MV1622) molecule. A small peak (<5%) representing MF3755xMF3755 DEDE homodimers is visible at ca. 11 minutes.

Additionally, an ADCC reporter assay was performed. The data show that afucosylated PB10651(MV1622) displays significantly increased ADCC activity for all cell lines in combination with the high (V158) and low (F158) FcγRIIIa receptor variant. The control, non-afucosylated PB10651 showed some ADCC activity in A431 cells combined with high affinity FcγRIIIa receptor variant effector cells, but the signal was significantly lower than the PB10651(MV1622) signal. For the other cell line/effector cell combinations the PB10651 was negative while PB10651(MV1622) showed strong ADCC activity. PG1337 (anti-tetanus toxoid) is a non-binding control IgG which was negative in all experiments (FIG. 34).

### Example 7 In Vivo Effectivity of the Lead Antibody

### Animal Model Selection

Therapeutic efficacy of afucosylated PB10651(MV1622) was assessed in immunocompromised mice bearing tumors from colorectal patients—known as patient-derived xenograft (PDX) models. PDX models CR2519, CR2161, CR2501, CR0150 and CR0193 (Crown Bioscience database, http://hubase2.crownbio.com) were selected based on the expression of both LGR5 and EGFR genes as analyzed by RNA sequencing (RNAseq). The models presented different status of KRAS gene. Namely, CR2519 and CR2161 were wild type for KRAS while CR2501, CR0150 were KRAS G12V and CR0193 were KRAS were G13D mutants. CR0231 and CR2501 were sensitive to Cetuximab, while CR0150 was low responsive to Cetuximab.

### Gene Expression Analysis

To verify that the PDX models had retained the indicated LGR5 and EGFR mRNA expression levels in animals used in the efficacy study, expression in live tumors was compared to that in PDX stock tumors. RNA was extracted from stock tumors (frozen material) and live tumors (study animals), homogenized with TRIzol (Ambiom 15596-018) and Tissue Lyser II (Qiagen 85300). RNA was then purified with RNeasy Mini Kit (Qiagen 74106) and RNase-Free DNase Set (Qiagen 76254). RNA quality was confirmed by NanoDrop™ spectrophotometer. cDNA was prepared by reverse transcription (ABI 4374966). Expression of LGR5 and EGFR was measured by real time RT-PCR reaction using TaqMan probes Hs00969422_ml and Hs01076090_ml, respectively, and normalized against glyceraldehyde-3-phosphate dehydrogenase (GAPDH) expression using TaqMan probe Hs02758991_g1. Data were analyzed by calculating the difference in Ct value between the gene of interest and GAPDH, and converting it to the power of 2. FIG. 35 shows that all six PDX models used in the efficacy study presented an expression of both EGFR and LGR5 comparable to the original frozen PDX tumor stocks. LGR5 expression in these six models was over a thousand-fold higher than in the PDX model presenting the lowest LGR5 expression (CR01560) from the available collection of 137 colorectal cancer PDX models. EGFR mRNA expression in the selected six models was lower than CR1197, which presented the highest EGFR expression in the whole CRC PDX collection.

### Efficacy Study in PDX

BALB/c nude mice were inoculated subcutaneously at the right flank with a PDX tumor fragment (2-3 mm in diameter) originating from one of the five colorectal cancer PDX tumor models (CR2519, CR2161, CR2501, CR0150 and CR0193). Tumors were allowed to grow to a volume of 100-200 mm³. Mice were then treated with four weekly intraperitoneal (i.p.) doses of PBS (200 μl) or afucosylated PB10651 (0.5 mg per animal in PBS). Tumors were measured biweekly by caliper and tumor volume (TV) was calculated using the formula TV=0.5×a×b² where a and b were the long and short diameters of the tumor, respectively. Afucosylated PB10651 presented strong tumoristatic activity in CR2519 and CR0193 PDX models, and this activity was similar to that of Cetuximab (FIG. 36). Afucosylated PB10651 presented limited but significant anti-tumor activity in CR2501 PDX model, while Cetuximab failed to significantly reduce tumor growth. PDX models CR2161 and CR0150 did not strongly respond to Cetuximab or afucosylated PB10651 and only showed a trend in anti-tumor activity for afucosylated PB10651 (FIG. 36).

### Example 8 P18T and C31M Xenograft Studies

### Materials and Methods

### Immunohistochemistry of P18 Tumoroids

After 48 hours from the addition of the antibodies, culture media was removed, and BME drops on a 48-well plate fixed in 300 μL of formalin for 2 hours at room temperature. BME drops were then manually broken using a pipette, pelleted, and placed in fresh formalin without disrupting the pellet. Pellets were left at room temperature overnight before washing in PBS three times. The pellet was then gently re-suspended in PBS and placed into a microcassette for processing by the IRB (Institute for Research in Biomedicine) histology facility for generation of paraffin embedded sections.

Ki67 and cleaved caspase-3 staining were performed by the IRB histology facility using an Autostainer Plus (Dako—Agilent). Prior to staining, sections were dewaxed as part of the antigen retrieval process using the low pH EnVision™ FLEX Target Retrieval Solutions (Dako, Burlington) for 20 mins at 97° C. using a PT Link (Dako—Agilent). Endogenous peroxidase was quenched for 10 mins using peroxidase-blocking solution (Dako REAL S2023). Rabbit polyclonal anti-Ki67 (ab15580, Abcam) and rabbit polyclonal anti-caspase 3 (Cell signaling, 9661S) were diluted 1:1000 and 1:500 and incubated for 60 and 120 min at room temperature respectively. BrightVision Poly-HRP-Anti Rabbit IgG Biotin-free was used for the secondary antibody (Immunologic, DPVR-110HRP), and was incubated for 30 mins at room temperature. Staining was revealed using 3-3′-diaminobenzidine (K3468, Dako), for 5 mins. Sections were counterstained with hematoxylin (Dako, S202084) and mounted with toluene-free mounting medium (CS705, Dako) using a Dako CoverStainer. Specificity of staining was confirmed by omission of the primary antibody. Images were captured using a Nikon Eclipse E600 attached to a Nikon DS-Ri1 camera.

### Treatment of P18T and C31M Organoid Xenografts

All mouse experiments were approved by the Animal Care and Use Committee of Barcelona Science Park (CEEA-PCB) and the Catalan government under protocol number DAAM7329. Tumoroids were grown for seven days before disaggregating into a single cells suspension for injection. Culture conditions and the method of creating single cells are described in section “FACS staining of cells obtained from organoid P18T using selected 5 anti-LGR5 cLC Antibodies”. For all mouse studies female NOD.CB17/AlhnRj-Prkdcscid/Rj mice (Janvier Labs) aged between 6-8 weeks were used. Xenografts were initiated by subcutaneously injecting 100 μL of BME:PBS (50:50) solution containing 200,000 P18T cells or 1,000,000 C31M cells. Once the tumor volume reached 300 mm3 mice were sacrificed and tumors harvested. One tumor was manually cut in to small pieces approximately 0.5 mm×0.5 mm×0.5 mm (width×length×height). The pieces were then placed into four flanks of recipient NOD-SCID mice, using one piece/flank. A trocker was used to implant the pieces into the mice, a device which pushes a tumour piece underneath the skin. A total of 30 mice were used for engraftment. Once the tumour volume on a mouse reached an average of 50 mm3 the mice were randomly assigned to a treatment group. Mice were injected once a week for four weeks with 200 μL of PBS (PH 7.4 Gibco Ref 10010-015), Cetuximab (clinical batch, 5 mg/ml), or afucosylated PB10651 (2.5 mg/ml). Cetuximab and afucosylated PB10651 were injected at a dose of 0.5 mg/mouse, regardless of mouse weight. Tumor volume was calculated using manual callipers with measurements taken thrice a week, and using the formula: (length×width×height)/2. Mice were sacrificed when either the tumor volume exceeded 300 mm3, the tumor ulcerated, or the end point of the study had been reached; 28 days since the first antibody injection. If only a single tumor on a mouse needed to be taken due to size constraints/ulceration then it was resected leaving the remaining in place for further study. If an additional resections or multiple tumors needed to be harvested then the mouse was culled and all the samples taken simultaneously. The data is expressed relative to day 0 (the first day of treatment) and a paired sample t-tests analysis was performed between each treatment group using GraphPad Prism.

### Results

Ki67 staining is an indicator of cellular proliferation, and was used to determine whether the effects on growth caused by the antibody treatments were due to a reduction in proliferation. Tumoroids were treated in vitro for 48 hours (2 μg/mL) before fixation. Treatment of tumoroids with afucosylated PB10651 caused a reduction in the number of positive cells, and in the intensity of staining, compared to the other treatment groups (FIG. 37). Cetuximab and the EGFR-TT antibodies caused a small, modest, reduction in the number of positive cells relative to the TT-TT control treatment. Cleaved caspase-3 staining was also performed to assess whether the antibodies promoted apoptosis. Low levels of apoptosis were observed in the TT control, EGFR-TT and cetuximab treatment groups, with comparable staining's seen between the three groups. The number of positive cells increased in the afucosylated PB10651-treated group. These results suggest the afucosylated PB10651 bispecific antibody is superior at reducing proliferation, and inducing apoptosis than cetuximab and the single anti-EGFR arm.

Xenografts were established in NOD-SICD mice and treated once a week for four weeks with afucosylated PB10651 (0.5 mg/mouse/week), Cetuximab (0.5 mg/mouse/week) or PBS. To assess antibody responses the tumour volumes were tracked and compared between groups. Treatment with afucosylated PB10651 caused a significant reduction in the average tumour volume increase compared to both PBS and cetuximab for both the P18T and C31M xenograft models from day 2 onwards (P=<0.01 for all time points, FIG. 38). In the P18T xenograft model, Cetuximab treated mice showed similar growth kinetics to the PBS treated mice and due to large tumour volumes the majority of the mice in the PBS and cetuximab group needed to be removed from the study on day 16. By day 20 the average fold change in tumour volume was 15.2 (±5.3) and 14.2 (±9.9) for PBS and cetuximab respectively, whereas for the afucosylated PB10651 group the value was 4.5 (±0.59). The tumour doubling times, determined by nonlinear curve fitting of an exponential growth equation, were calculated as 9 days for the PBS and cetuximab groups, and 6 days for the afucosylated PB10651 treatment group, a 30% reduction in tumour doubling time. Similarly, in the C31M xenograft model, PB1065 significantly reduced tumor growth compared to both PBS and cetuximab treatment arms (FIG. 38).

### Example 8: Non-GLP Cynomolgus Toxicity Study Using Repeated Doses of PB10651

To gauge the possible toxicity of PB10651, a non-GLP repeated dose toxicity study in cynomolgus monkey was performed. In view of the EGFR Fab arm present in PB10651, skin and gastro-intestinal (GI) tract toxicity may be expected. In the case of the anti-EGFR antibody Cetuximab (Erbitux), deaths were observed in monkeys at a weekly dose of 75 mg/kg due to severe skin toxicity, with a dose of 24 mg/kg/week being the highest tolerated dose in chronic monkey toxicity studies. In the case of Panitumumab (Vectibix), mortality was seen in monkeys at a dose of 30 mg/kg/week after only three weeks of treatment in a 4 week study, with occasional deaths at lower dosages (7.5 and 15 mg/kg/week) in a 13 week IV toxicity study. Based on this information, a dose level of 25 mg/kg was selected for PB10651 as the highest dose in the present study. One male and one female animal were used per group and four groups were designated: control (vehicle only), 2.5 mg/kg/week, 7.5 mg/kg/week and 25 mg/kg/week and four weekly doses were given to each group. Antibody was administered via intravenous infusion over an hour to the animals.

### Results

Toxicity studies in cynomolgus monkeys with Cetuximab and Panitumumab revealed dermal toxicity (erythema, dry/flaking skin/hair loss) and gastro-intestinal (GI) tract effects (soft faeces/diarrhoea, dehydration) as the dose-limiting toxicities (EMA-EPAR 2004 cetuximab, EMA-EPAR 2007 panitumumab), consistent with the anti-EGFR activity of the test items. For PB10651, however, even at the highest dose administered in this study, neither skin toxicity nor GI tract toxicity was observed after repeated administration. Clinical signs, such as fur thinning, bruising and incidence of loose faeces were equally observed in active and control groups and these clinical signs were therefore not considered to be drug-related. There were no organ weight and/or organ weight ratio changes considered to be related to administration of PB10651. In addition, there were no macroscopic or microscopic findings considered to be related to administration of PB10651. In conclusion, once weekly intravenous (infusion) administration of PB10651 to the cynomolgus monkey for 4 weeks did not result in organ weight or organ weight ratio changes, macroscopic or microscopic findings considered to be related to administration of PB10651.

The work leading to this invention has received funding from the [European Union][European Atomic Energy Community] Seventh Framework Programme ([FP7/2007-2013] [FP7/2007-2011]) under grant agreement no [601876].14

TABLE 1
Titers of successfully immunized MeMo ® mice.

| | | Reciprocal | | |
|---|---|---|---|---|
| | Immunized | serum titer | | Library |
| Mouse # | antigen | FACS | Library | size |
| L1 | pVax1 hLGR4- | hLGR4 100 | ML1246 | 2.8 × 10\hat{ }7 |
| | 2xFLAG-2xHA | | | |
| | pVax1 hLGR5- | hLGR5 0 | | |
| | 2xFLAG-2xHA | | | |
| H1 | rhLGR5-Fc | hLGR5 < 100 | ML1247 | 2.5 × 10\hat{ }7 |
| H2 | rhLGR5-Fc | hLGR5 < 100 | ML1248 | 2.2 × 10\hat{ }7 |
| H3 | rhLGR5-Fc | hLGR5 100 | ML1249 | 2.6 × 10\hat{ }6 |
| H4 | rhLGR5-Fc | hLGR5 100 | ML1250 | 7.7 × 10\hat{ }6 |
| H5 | rhLGR5-Fc | hLGR5 100 | | |
| H6 | rhLGR5-Fc | hLGR5 100 | ML1251 | 1.6 × 10\hat{ }7 |
| L19 | rhZNRF3-Fc | hZNRF3 100 | ML1223 | 1.0 × 10\hat{ }7 |
| L20 | rhZNRF3-Fc | hZNRF3 100 | ML1224 | 7.8 × 10\hat{ }6 |
| L21 | rhZNRF3-Fc | hZNRF3 100 | ML1225 | 1.2 × 10\hat{ }7 |
| L22 | rhZNRF3-Fc | hZNRF3 100 | ML1226 | 8.1 × 10\hat{ }6 |
| L23 | rhZNRF3-Fc | hZNRF3 100 | ML1227 | 6.1 × 10\hat{ }6 |
| L24 | rhZNRF3-Fc | hZNRF3 100 | ML1228 | 5.0 × 10\hat{ }6 |
| K14 | rhRNF43-Fc | hRNF43 100 | ML1229 | 9.1 × 10\hat{ }6 |
| K15 | rhRNF43-Fc | hRNF43 100 | ML1230 | 2.0 × 10\hat{ }6 |
| K17 | rhRNF43-Fc | hRNF43 100 | ML1231 | 5.4 × 10\hat{ }6 |

TABLE 2
Overview of the antibody panels generated from phage
antibody repertoires against the different WNT targets.

| | Final antibody panel size |
|---|---|
| Target | (number of different clones characterized) |
| LGR4 | 66 |
| LGR5 | 84 |
| ZNRF3 | 105 |
| RNF43 | 33 |

TABLE 3
Characteristics of bispecific IgG containing a selected panel of Wnt targeting Fab fragments
against LGR4, LGR5, ZNRF3 and RNF43 combined with the Tetanus toxoid Fab Fragment.

| | Target | FACS | | | 40 C. stability | | | |
|---|---|---|---|---|---|---|---|---|
| | Fab | titration | R-spondin blocking | | OD₄₅₀ | OD₄₅₀ | % (OD₄₅₀ 40° C./ | Stable |
| PB# | fragment | AUC | % binding | Blocking | 4° C. | 40° C. | OD₄₅₀ 4° C.) | at 40° C. |
| PB10251 | LGR4 | 28154 | 102 | No | 0.452 | 0.248 | 55 | Yes |
| PB10252 | LGR4 | 12888 | 103 | No | 0.099 | 0.064 | 65 | NA |
| PB10261 | LGR5 | 43103 | 59 | Partial | 1.005 | 0.876 | 87 | Yes |
| PB10273 | LGR5 | 27178 | 92 | No | 1.793 | 1.819 | 101 | Yes |
| PB10275 | LGR5 | 24879 | 94 | No | 1.677 | 1.664 | 99 | Yes |
| PB10278 | LGR5 | 22197 | 80 | Partial | 0.655 | 0.401 | 61 | Yes |
| PB10279 | LGR5 | 24974 | 82 | No | 0.839 | 0.597 | 71 | Yes |
| PB10284 | LGR5 | 40187 | 59 | Partial | 0.985 | 0.867 | 88 | Yes |
| PB10286 | LGR5 | 41872 | 95 | No | 1.050 | 0.964 | 92 | Yes |
| PB10287 | LGR5 | 19378 | 107 | No | 0.810 | 0.609 | 75 | Yes |
| PB10290 | LGR5 | 44272 | 67 | Partial | 1.059 | 1.043 | 98 | Yes |
| PB10300 | ZNRF3 | 32012 | 90 | No | 1.693 | 1.264 | 75 | Partial |
| PB10302 | ZNRF3 | 29235 | 102 | No | 1.783 | 1.722 | 97 | Yes |
| PB10304 | ZNRF3 | 38321 | 75 | Partial | 1.747 | 1.336 | 76 | Partial |
| PB10309 | ZNRF3 | 19446 | 93 | No | 1.688 | 1.087 | 64 | Yes |
| PB10328 | ZNRF3 | 26933 | 86 | No | 1.714 | 1.776 | 104 | Yes |
| PB10330 | ZNRF3 | 46341 | 50 | Yes | 1.793 | 1.357 | 76 | Yes |
| PB10332 | ZNRF3 | 24318 | 93 | No | 1.602 | 1.415 | 88 | Yes |
| PB10333 | ZNRF3 | 36923 | 82 | No | 1.434 | 1.137 | 79 | Yes |
| PB10346 | RNF43 | 82605 | 29 | Yes | 1.397 | 1.377 | 99 | Yes |
| PB10349 | RNF43 | 92972 | 71 | Partial | 1.523 | 1.529 | 100 | Yes |
| PB10350 | RNF43 | 93258 | 33 | Yes | 1.430 | 1.325 | 93 | Yes |

Shown are the FAGS affinity titration, the R-Spondin3 blocking ELISA and the 40° C. stability ELISA. For the FAGS affinity titration the area under the curve (AUC) values are indicated. For the R-Spondin blocking ELISA the percentage binding remaining per IgG compared to the maximum binding value (set at 100% binding) was indicated, as well as the final conclusion from 2 independent experiments. The 40° C. stability ELISA is shown. OD_{450 nm} values after one week at V° C. and at 40° C. are indicated in the table. The ratios (in percentage) of the OD_{450 nm} from V° C. versus 40° C. are indicated in the table. The final conclusion is inserted in the right column, whether an IgG is considered stable, partially stable or NA when GD signals were below 0.1

TABLE 4
Z′ factor scores for different features and a
multi-parametric score in different organoids, comparing
growth with and without EGF in EGF dependent tumoroids.
The multi-parametric Z′ factor score was either similar or higher than
individual feature scores, demonstrating that using such a score is both
sensitive and can be applied across multiple different organoids which
undergo different phenotypic responses after growth factor treatment.

| Z′ factor | P18T | P14T | P8T |
|---|---|---|---|
| Nucleus count | 0.58 | −0.34 | −0.68 |
| Tumoroid size | −0.01 | 0.18 | 0.05 |
| Lumen count | −0.56 | 0.56 | 0.27 |
| Lumen roundness | 0.22 | 0.13 | 0.30 |
| Multiparametric | 0.80 | 0.53 | 0.50 |

TABLE 5
Preferred heavy chain combinations for bispecific antibodies
that bind LGR4/EGFR; LGR4/HER3; LGR5/EGFR; LGR5/HER3; RNF43/EGFR;
RNF43/HER3; and ZNRF3/EGFR; ZNRF3/HER3. TT is a heavy chain
for tetanus toxoid and for reference only.

| | | TT | EGFR | | | |
|---|---|---|---|---|---|---|
| | | MF1337 | MF3755 | MF4280 | MF3370 | MF4289 |
| TT | MF1337 | PB4248 | PB9919 | PB9647 | PB9920 | PB10104 |
| LGR4 | MF5777 | PB10251 | PB10619 | PB10620 | PB10621 | PB10622 |
| | MF5781 | PB10252 | PB10623 | PB10624 | PB10625 | PB10626 |
| LGR5 | MF5790 | PB10261 | PB10627 | PB10628 | PB10629 | PB10630 |
| | MF5803 | PB10273 | PB10631 | PB10632 | PB10633 | PB10634 |
| | MF5805 | PB10275 | PB10635 | PB10636 | PB10637 | PB10638 |
| | MF5808 | PB10278 | PB10639 | PB10640 | PB10641 | PB10642 |
| | MF5809 | PB10279 | PB10643 | PB10644 | PB10645 | PB10646 |
| | MF5814 | PB10284 | PB10647 | PB10648 | PB10649 | PB10650 |
| | MF5816 | PB10286 | PB10651 | PB10652 | PB10653 | PB10654 |
| | MF5817 | PB10287 | PB10655 | PB10656 | PB10657 | PB10658 |
| | MF5818 | PB10290 | PB10659 | PB10660 | PB10663 | PB10664 |
| RNF43 | MF5832 | PB10346 | PB10661 | PB10662 | PB10665 | PB10666 |
| | MF5836 | PB10349 | PB10667 | PB10668 | PB10669 | PB10670 |
| | MF5839 | PB10350 | PB10671 | PB10672 | PB10673 | PB10674 |
| ZNRF3 | MF5850 | PB10300 | PB10675 | PB10676 | PB10677 | PB10678 |
| | MF5853 | PB10302 | PB10679 | PB10680 | PB10681 | PB10682 |
| | MF5855 | PB10304 | PB10683 | PB10684 | PB10685 | PB10686 |
| | MF5862 | PB10309 | PB10687 | PB10688 | PB10689 | PB10690 |
| | MF5882 | PB10328 | PB10691 | PB10692 | PB10693 | PB10694 |
| | MF5884 | PB10330 | PB10695 | PB10696 | PB10697 | PB10698 |
| | MF5887 | PB10332 | PB10699 | PB10700 | PB10701 | PB10702 |
| | MF5888 | PB10333 | PB10703 | PB10704 | PB10705 | PB10706 |
| TT | MF1337 | PB4248 | PB9215 | PB9921 | PB9918 | PB10111 |
| LGR4 | MF5777 | PB10251 | PB10707 | PB10708 | PB10709 | PB10710 |
| | MF5781 | PB10252 | PB10711 | PB10712 | PB10713 | PB10714 |
| LGR5 | MF5790 | PB10261 | PB10715 | PB10716 | PB10717 | PB10718 |
| | MF5803 | PB10273 | PB10719 | PB10720 | PB10721 | PB10722 |
| | MF5805 | PB10275 | PB10723 | PB10724 | PB10725 | PB10726 |
| | MF5808 | PB10278 | PB10727 | PB10728 | PB10729 | PB10730 |
| | MF5809 | PB10279 | PB10731 | PB10732 | PB10733 | PB10734 |
| | MF5814 | PB10284 | PB10735 | PB10736 | PB10737 | PB10738 |
| | MF5816 | PB10286 | PB10748 | PB10749 | PB10750 | PB10751 |
| | MF5817 | PB10287 | PB10752 | PB10753 | PB10754 | PB10755 |
| | MF5818 | PB10290 | PB10756 | PB10757 | Not made | Not made |
| RNF43 | MF5832 | PB10346 | Not made | Not made | PB10758 | PB10759 |
| | MF5836 | PB10349 | PB10764 | PB10765 | PB10766 | PB10767 |
| | MF5839 | PB10350 | PB10768 | PB10769 | PB10770 | PB10771 |
| ZNRF3 | MF5850 | PB10300 | PB10772 | PB10773 | PB10774 | PB10775 |
| | MF5853 | PB10302 | PB10776 | PB10777 | PB10778 | PB10779 |
| | MF5855 | PB10304 | PB10780 | PB10781 | PB10782 | PB10783 |
| | MF5862 | PB10309 | PB10784 | PB10785 | PB10786 | PB10787 |
| | MF5882 | PB10328 | PB10788 | PB10789 | PB10790 | PB10791 |
| | MF5884 | PB10330 | PB10792 | PB10793 | PB10794 | PB10795 |
| | MF5887 | PB10332 | PB10796 | PB10797 | PB10798 | PB10799 |
| | MF5888 | PB10333 | PB10800 | PB10801 | PB10802 | PB10803 |

TABLE 6
Preferred heavy chain combinations for bispecific
antibodies that bind LGR4/EGFR; LGR4/HER3; LGR5/EGFR;
LGR5/HER3; RNF43/EGFR; RNF43/HER3; and ZNRF3/EGFR;
ZNRF3/HER3. TT is a heavy chain for tetanus
toxoid and for reference only.

| | | TT | EGFR | HER3 |
|---|---|---|---|---|
| | | MF1337 | MF3755 | MF3178 |
| TT | MF1337 | PB4248 | PB9919 | PB9215 |
| LGR4 | MF5777 | PB10251 | PB10619 | PB10707 |
| | MF5781 | PB10252 | PB10623 | PB10711 |
| LGR5 | MF5790 | PB10261 | PB10627 | PB10715 |
| | MF5803 | PB10273 | PB10631 | PB10719 |
| | MF5814 | PB10284 | PB10647 | PB10735 |
| | MF5816 | PB10286 | PB10651 | PB10748 |
| | MF5817 | PB10287 | PB10655 | PB10752 |
| | MF5818 | PB10290 | PB10659 | PB10756 |
| RNF43 | MF5832 | PB10346 | PB10661 | PB12336 |
| | MF5836 | PB10349 | PB10667 | PB10764 |
| ZNRF3 | MF5850 | PB10300 | PB10675 | PB10772 |
| | MF5853 | PB10302 | PB10679 | PB10776 |
| | MF5855 | PB10304 | Not made | PB10780 |
| | MF5884 | PB10330 | PB10695 | Not made |
| | MF5888 | PB10333 | PB10703 | PB10800 |

TABLE 7
List of comparator antibodies copied
from literature with their internal number.
Antibody VH- and VL- sequences were copied from patents,
made as synthetic cDNA's and then cloned into an expression
vector for the expression of human IgG1. Antibodies were
expressed and purified using standardised procedures.

| | | | | Internal |
|---|---|---|---|---|
| Antibody | Target | Firm | Species | number |
| hu8E11v2 | LGR5 | Genentech | Humanised | PG7543 |
| BNC101 | LGR5 | Bionomics | Humanised | PG7709 |
| OMP18RS | FZD7 | OncoMed | Human | PG7710 |
| OMP88R20 | LGR5 | OncoMed | Human | PG7711 |
| OMP88R21 | LGR5 | OncoMed | Human | PG7712 |
| OMP131R10 | RSPO3 | OncoMed | Humanised | PG7713 |

TABLE 8
Residues in EGFR and LGR5 found to be relevant for the binding of
PB10651 to the respective target in shotgun mutagenesis analysis.

| LGR5 relevant | EGFR relevant |
|---|---|
| residues | residues |
| D43A | I462A |
| G44A | G465A |
| M46A | K489A |
| F67A | I491A |
| G90A | N493A |
| F91A | C499A |

TABLE 9
EGFR and LGR5 mRNA expression levels in PDX
tumors used for ex vivo FACS staining.

| | | EGFR | LGR5 |
|---|---|---|---|
| Cell name | Cancer type | log2(FPKM) | log2(FPKM) |
| EX-CR2394 | Colorectal Cancer | Not done yet | Not done yet |
| EX-OV3077 | Ovarian Cancer | 2.178 | 6.1989 |
| EX-GA6239 | Gastric Cancer | 2.4679 | 5.3676 |
| EX-HN2195 | Head and Neck Cancer | 8.9258 | 5.3171 |
| EX-LI0574 | Liver Cancer | 3.6252 | 5.0135 |
| EX-GA6210 | Gastric Cancer | 1.0841 | 4.6505 |
| EX-OV1286 | Ovarian Cancer | 2.3905 | 4.6434 |
| EX-CC6638 | Cholangiocarcinoma | 3.3147 | 3.5631 |
| EX-LU2529 | Lung Cancer | 4.0328 | 3.2662 |
| EX-OV0273 | Ovarian Cancer | −2 | 3.0802 |
| EX-LU6429 | Lung Cancer | 4.0896 | 2.897 |
| EX-ES0201 | Esophageal Cancer | 5.6923 | 1.8035 |

TABLE 10
Mean fluorescence intensity (MFI) values obtained after ex vivo
staining of patient derived xenografts of different indications.

| | | Mean fluorescence intensity | | |
|---|---|---|---|---|
| | | (MFI) value | | |
| | | PG5816 | PG3755 | PG1337 |
| | Crown | (anti- | (anti- | (anti-TT |
| Cancer type | model | LGR5) | EGFR) | control) |
| Ovarian Cancer | OV1286 | 7482 | 2120 | 1575 |
| Ovarian Cancer | OV0273 | 5960 | 2152 | 1728 |
| Esophageal Cancer | ES0201 | 5772 | 5967 | 1642 |
| Colorectal Cancer | CR2394 | 3661 | 2602 | 1879 |
| Gastric Cancer | GA6210 | 3240 | 1003 | 1105 |
| Liver Cancer | LI0574 | 2543 | 6110 | 1070 |
| Gastric Cancer | GA6239 | 2265 | 2104 | 1731 |
| Head and Neck Cancer | HN2195 | 1846 | 11814 | 1134 |
| Lung Cancer | LU2529 | 1705 | 6863 | 1685 |
| Lung Cancer | LU6429 | 1532 | 6081 | 1006 |
| Cholangiocarcinoma | CC6638 | 1370 | 9366 | 1104 |

The invention provides the following aspects (among others).

Aspect 1. An antibody that comprises a variable domain that can bind an epitope on an extracellular part of human EGFR of which amino acid residues 1462; G465; K489; I491; N493; and C499 are involved in binding of the antibody to the epitope.

Aspect 2. The antibody of aspect 1, wherein one or more of the amino acid residue substitutions selected from I462A; G465A; K489A; I491A; N493A; and C499A reduce the binding of the antibody to human EGFR.

Aspect 3. An antibody that comprises a variable domain that can bind an epitope on an extracellular part of human EGFR which epitope is located within amino acid residues 420-480 of SEQ ID NO: 2 depicted in FIG. 40, and wherein the binding of the antibody to LGR5 is reduced by one or more of the following amino acid residue substitutions I462A; G465A; K489A; I491A; N493A; and C499A.

Aspect 4. The antibody of any one of aspects 1-3, wherein the binding of the antibody to human EGFR interferes with the binding of EGF to the receptor.

Aspect 5. The antibody of any one of aspects 1-4, wherein the epitope is a conformational epitope.

Aspect 6. The antibody of any one of aspects 1-5, wherein the epitope is located within amino acid residues 420-480 of SEQ ID NO: 2 depicted in FIG. 40, preferably within 430-480 of SEQ ID NO: 2 depicted in FIG. 40; preferably within 438-469 of SEQ ID NO: 2 depicted in FIG. 40.

Aspect 7. The antibody of any one of aspects 1-6, wherein the antibody comprises a further variable domain which further variable domain can bind a further protein.

Aspect 8. The antibody of aspect 7, wherein the further protein is a membrane protein comprising an extracellular part.

Aspect 9. The antibody of aspect 7 or aspect 8, wherein the further protein is a membrane associated member WNT-pathway.

Aspect 10. The antibody of any one of aspects 1-9, wherein the antibody is a bispecific antibody comprising a variable domain that binds human EGFR and a variable domain that binds a further protein.

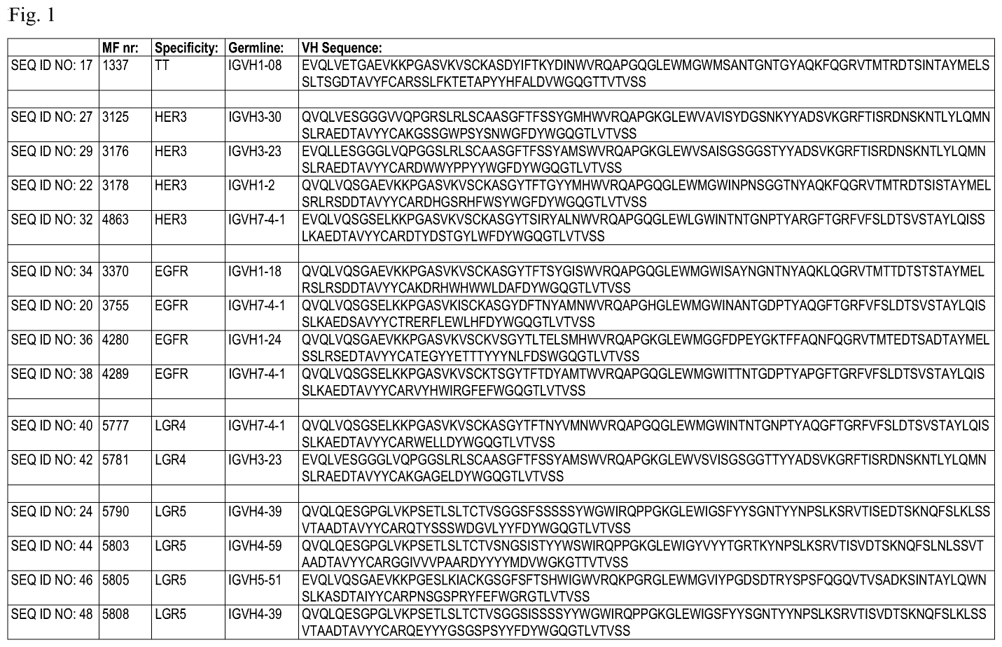

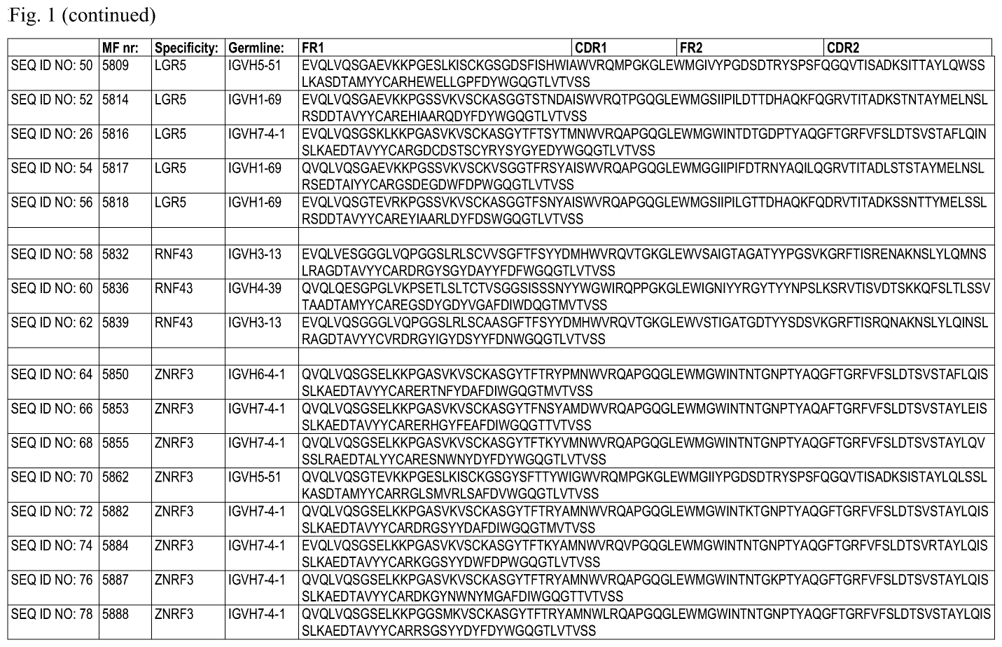

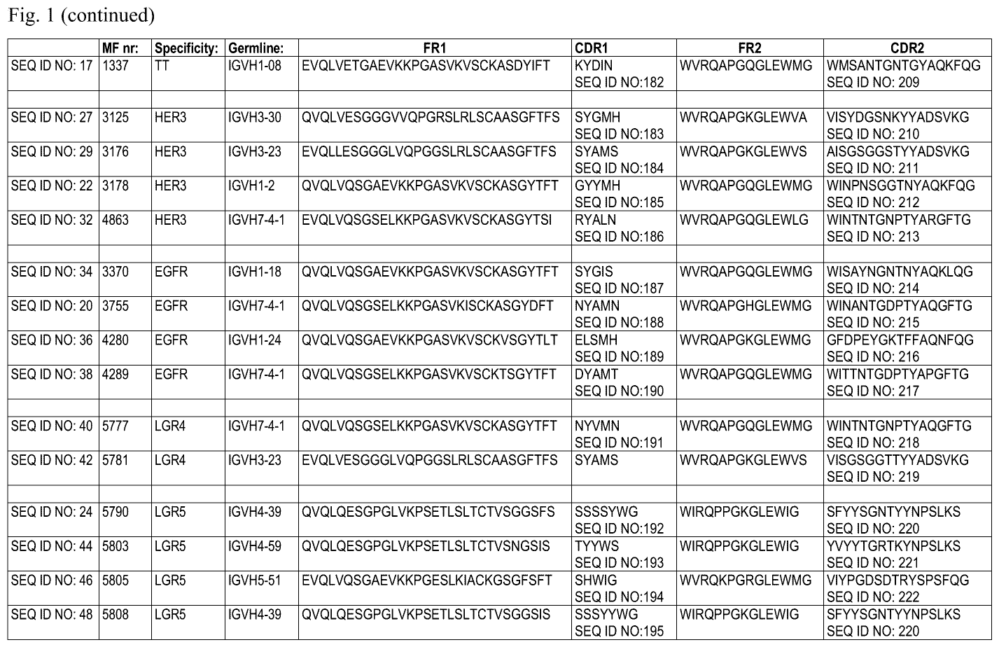

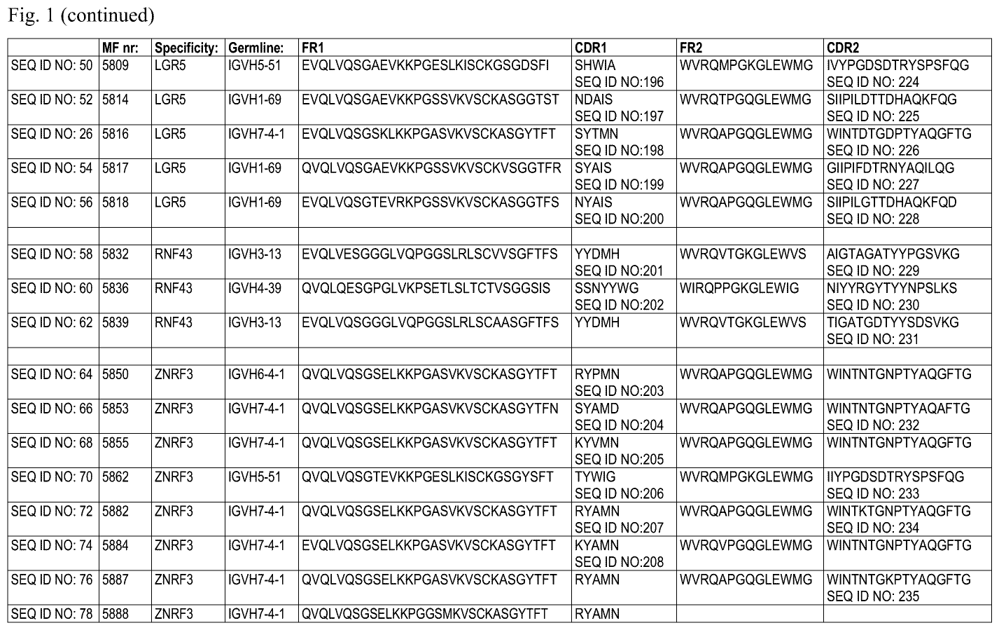

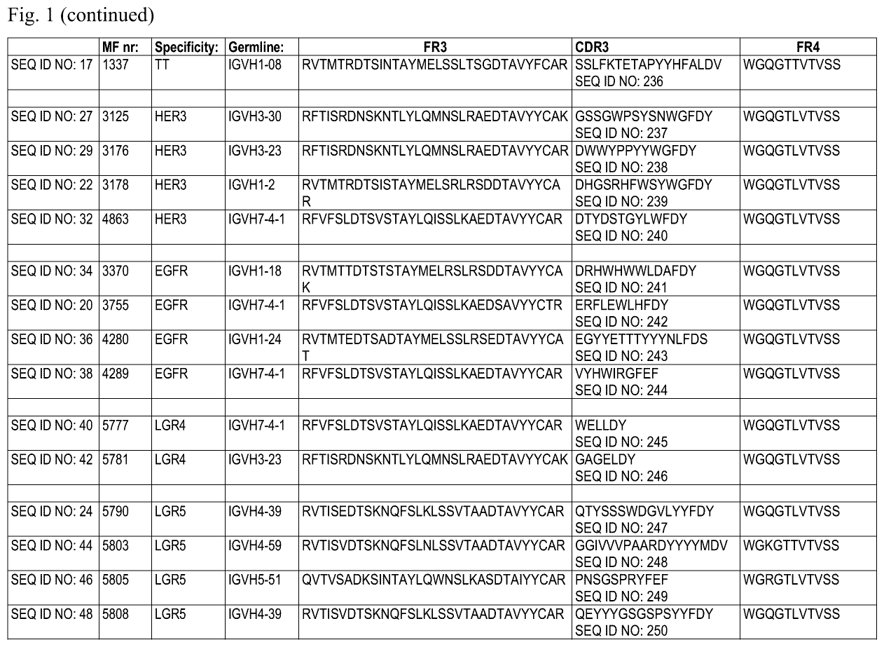

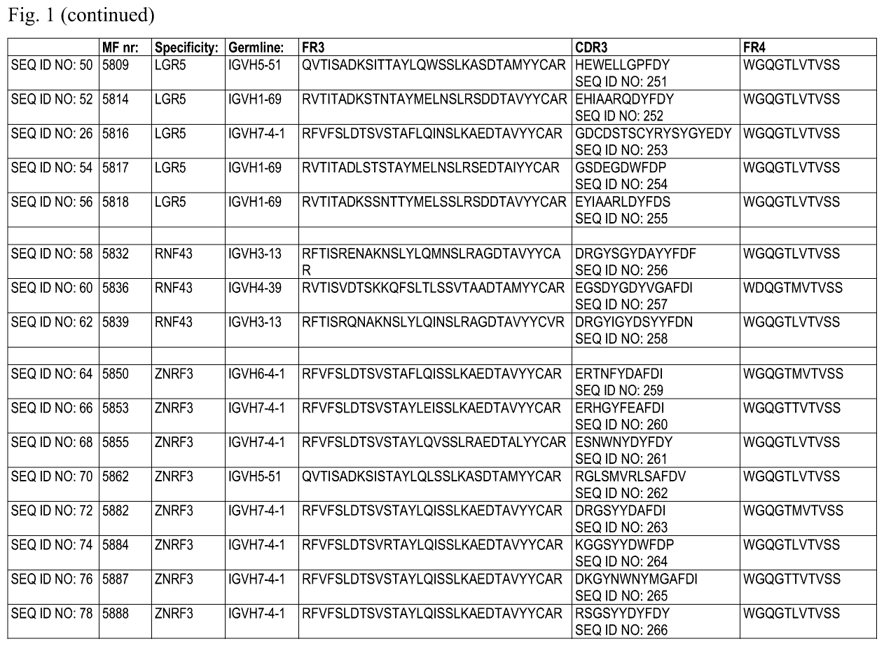

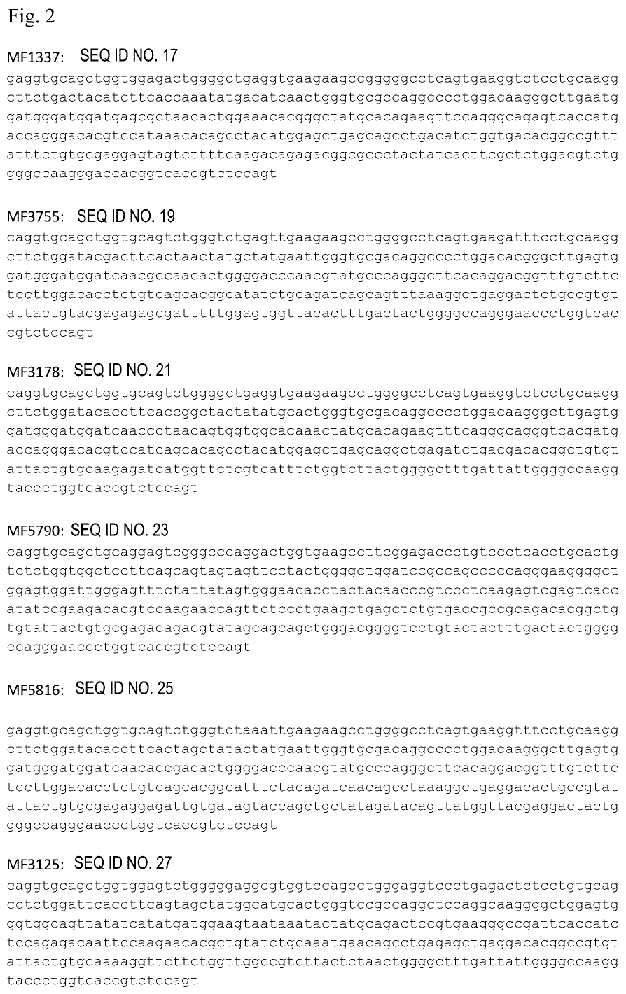

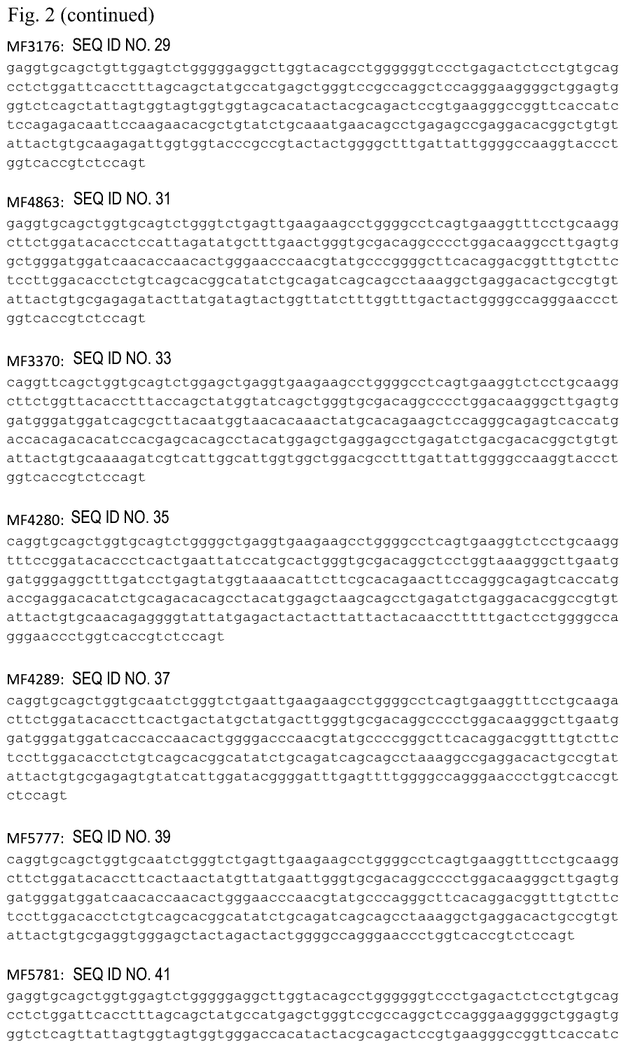

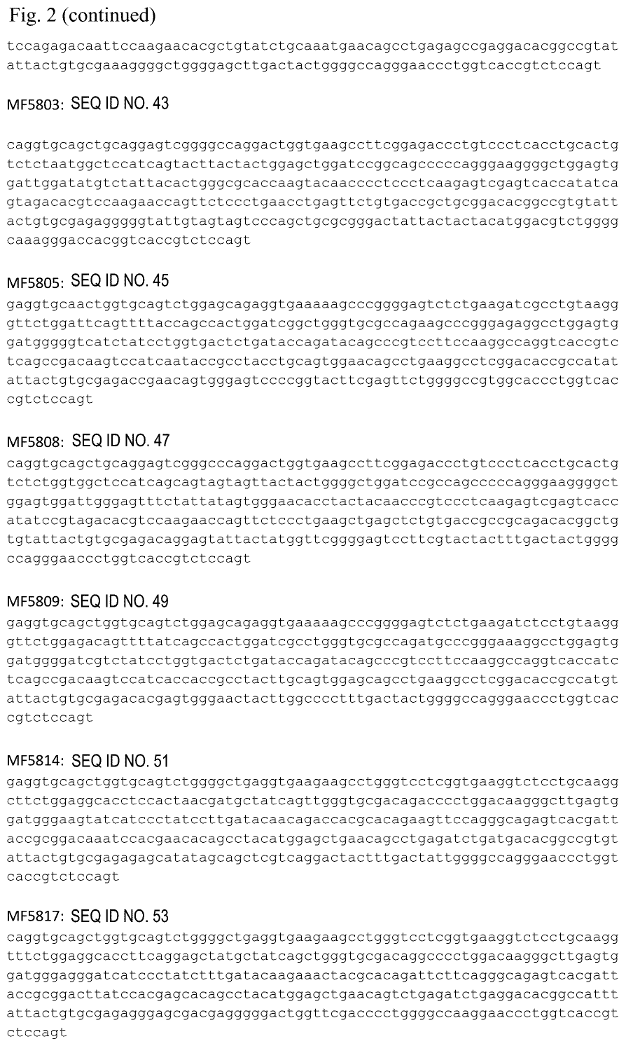

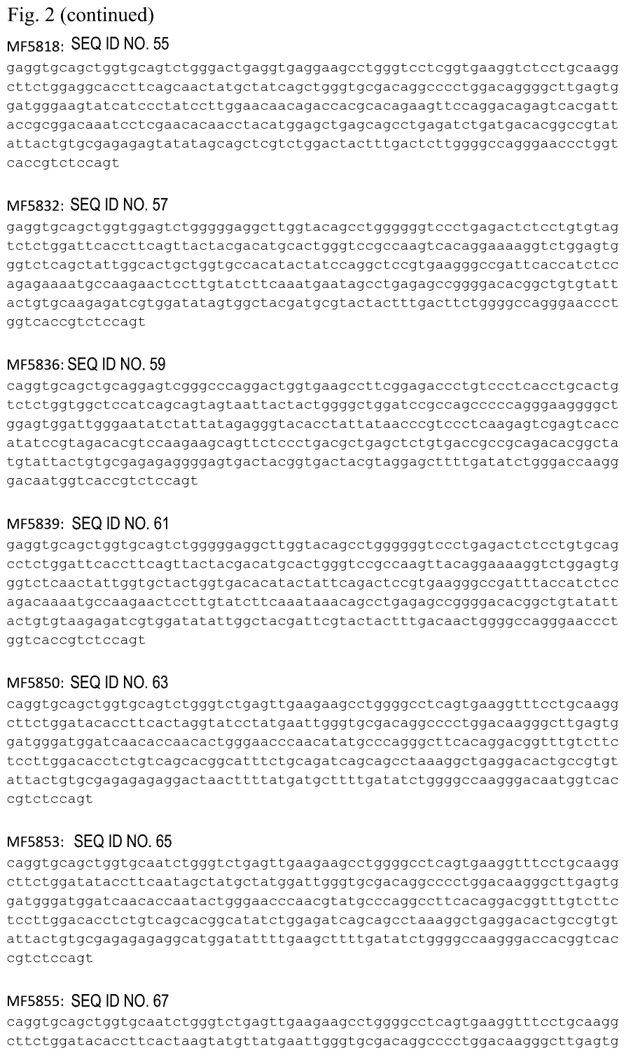

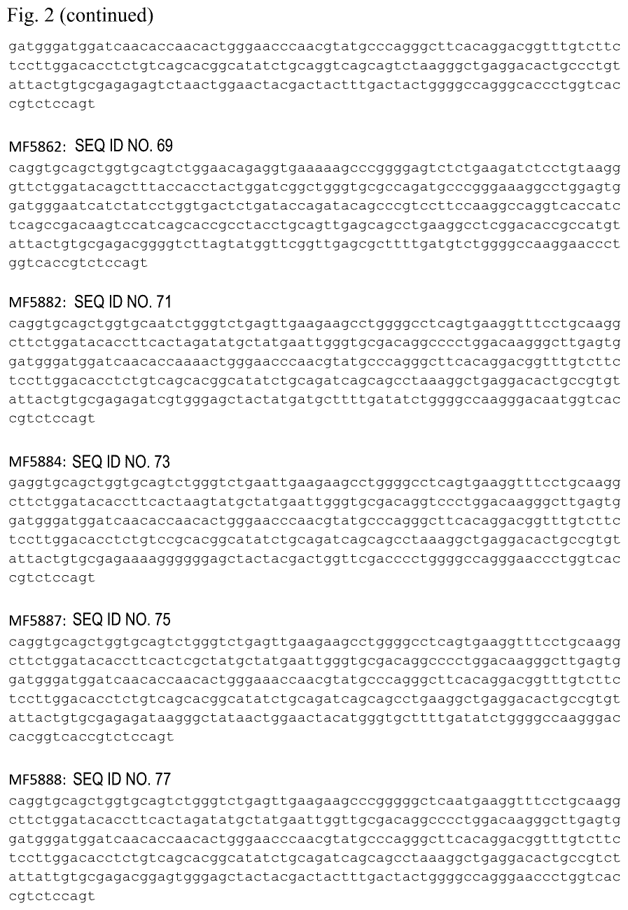

Aspect 11. The antibody of any one of aspects 1-10, wherein the variable domain that binds human EGFR, wherein a heavy chain variable region of said variable domain comprises at least the CDR3 sequence of the VH of MF3755 as depicted in FIG. 1 or wherein a heavy chain variable region of said variable domain comprises a heavy chain CDR3 sequence that differs in at most three, preferably in at most two, preferably in no more than one amino acid from a CDR3 sequence of the VH of MF3755 as depicted in FIG. 1.

Aspect 12. The antibody of aspect 10 or aspect 11, wherein said variable domain comprises a heavy chain variable region comprising at least the CDR1, CDR2 and CDR3 sequences of the VH of MF3755 as depicted in FIG. 1; or the CDR1, CDR2 and CDR3 sequences of the VH of MF3755 as depicted in FIG. 1 with at most three, preferably at most two, preferably at most one amino acid substitutions.

Aspect 13. The antibody of aspect 11 or aspect 12, wherein the variable domain that binds EGFR, comprises the sequence of the VH chain of MF3755 as depicted in FIG. 1; or the amino acid sequence of the VH chain of MF3755 depicted in FIG. 1 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3755.

Aspect 14. The antibody of any one of aspects 7-13, wherein the further protein is LGR4; LGR5; LGR6; RNF43 or ZNRF3.

Aspect 15. The antibody of aspect 14, wherein the further protein is LGR5. Aspect 16. A bispecific antibody comprising a variable domain that can bind an epitope on an extracellular part of EGFR and a variable domain that can bind a further protein, wherein the EGFR epitope is located within amino acid residues 420-480 of SEQ ID NO: 2 depicted in FIG. 40, which amino acid residues 1462; G465; K489; I491; N493; and C499 are involved in binding of the antibody to the epitope.

Aspect 17. The bispecific antibody of aspect 16, wherein the binding of the variable domain to EGFR is reduced with one or more of the following amino acid residue substitutions I462A; G465A; K489A; I491A; N493A; and C499A.

Aspect 18. The bispecific antibody of aspect 16 or aspect 17, wherein the further protein is membrane protein comprising an extracellular part.

Aspect 19. The bispecific antibody of any one of aspects 16-18, wherein the further protein is a membrane associated member of the WNT-pathway, preferably LGR5.

It has been shown that antibodies comprising one or more variable domains that bind EGFR with the mentioned epitope have a better effectivity when used to inhibit growth of an EGFR ligand responsive cancer or cell. In the context of bispecific antibodies, an arm of the antibody comprising an EGFR binding variable domain with the mentioned epitope combines better with a variety of other arms comprising variable domains that bind extra-cellular parts of other cell surface proteins.

1-28. (canceled)

29. A method for the treatment of an individual that has a cancer, the method comprising administering a bispecific antibody to said individual, wherein said bispecific antibody comprises a variable domain that binds an extracellular part of EGFR and a variable domain that binds an extracellular part of LGR5.

30. The method of claim 29, wherein said bispecific antibody comprises a variable domain that binds an extracellular part of EGFR and a variable domain that binds an extracellular part of LGR5, wherein
the VH chain of the variable domain that binds EGFR comprises the CDR1, CDR2 and CDR3 amino acid sequences NYAMN (SEQ ID NO:188), WINANTGDPTYAQGFTG (SEQ ID NO: 215), and ERFLEWLHFDY (SEQ ID NO: 242); and the VH chain of the variable domain that binds LGR5 comprises the CDR1, CDR2 and CDR3 amino acid sequences SSSSYWG (SEQ ID NO: 192), SFYYSGNTYYNPSLKS (SEQ ID NO: 220), and TYSSSWDGVLYYFDY (SEQ ID NO:247);
the VH chain of the variable domain that binds EGFR comprises the CDR1, CDR2 and CDR3 amino acid sequences NYAMN (SEQ ID NO: 188), WINANTGDPTYAQGFTG (SEQ ID NO: 215) and ERFLEWLHFDY (SEQ ID NO: 242); and the VH chain of the variable domain that binds LGR5 comprises the CDR1, CDR2 and CDR3 amino acid sequences TYYWS (SEQ ID NO: 193), YVYYTGRTKYNPSLKS (SEQ ID NO: 221) and GGIVVVPAARDYYYYMDV (SEQ ID NO: 248);
the VH chain of the variable domain that binds EGFR comprises the CDR1, CDR2 and CDR3 amino acid sequences NYAMN (SEQ ID NO: 188), WINANTGDPTYAQGFTG (SEQ ID NO: 215) and ERFLEWLHFDY (SEQ ID NO: 242), and the VH chain of the variable domain that binds LGR5 comprises the CDR1, CDR2 and CDR3 amino acid sequences SHWIG (SEQ ID NO: 194), VIYPGDSDTRYSPSFQG (SEQ ID NO: 222) and PNSGSPRYFEF (SEQ ID NO:249);
the VH chain of the variable domain that binds EGFR comprises the CDR1, CDR2 and CDR3 amino acid sequences NYAMN (SEQ ID NO: 188), WINANTGDPTYAQGFTG (SEQ ID NO: 215), and ERFLEWLHFDY (SEQ ID NO: 242), and the VH chain of the variable domain that binds LGR5 comprises the CDR1, CDR2 and CDR3 amino acid sequences SHWIA (SEQ ID NO: 196), IVYPGDSDTRYSPSFQG (SEQ ID NO: 224), and HEWELLGPFDY (SEQ ID NO: 251);
the VH chain of the variable domain that binds EGFR comprises the CDR1, CDR2 and CDR3 amino acid sequences NYAMN (SEQ ID NO: 188), WINANTGDPTYAQGFTG (SEQ ID NO: 215), and ERFLEWLHFDY (SEQ ID NO: 242) and the VH chain of the variable domain that binds LGR5 comprises the CDR1, CDR2 and CDR3 amino acid sequences NDAIS (SEQ ID NO: 197), SIIPILDTTDHAQKFQG (SEQ ID NO: 225), and EHIAARQDYFDY (SEQ ID NO: 252);
the VH chain of the variable domain that binds EGFR comprises the CDR1, CDR2 and CDR3 amino acid sequences NYAMN (SEQ ID NO: 188), WINANTGDPTYAQGFTG (SEQ ID NO: 215), and ERFLEWLHFDY (SEQ ID NO: 242); and the VH chain of the variable domain that binds LGR5 comprises the CDR1, CDR2 and CDR3 amino acid sequences SYTMN (SEQ ID NO: 198), WINTDTGDPTYAQGFTG (SEQ ID NO: 226), and GDCDSTSCYRYSYGYEDY (SEQ ID NO: 253);
the VH chain of the variable domain that binds EGFR comprises the CDR1, CDR2 and CDR3 amino acid sequences NYAMN (SEQ ID NO:188), WINANTGDPTYAQGFTG (SEQ ID NO: 215), and ERFLEWLHFDY (SEQ ID NO: 242); and the VH chain of the variable domain that binds LGR5 comprises the CDR1, CDR2 and CDR3 amino acid sequences SYAIS (SEQ ID NO: 199), GIIPIFDTRNYAQILQG (SEQ ID NO: 227), and GSDEGDWFDP (SEQ ID NO: 254);
the VH chain of the variable domain that binds EGFR comprises the CDR1, CDR2 and CDR3 amino acid sequences NYAMN (SEQ ID NO:188), WINANTGDPTYAQGFTG (SEQ ID NO: 215), and ERFLEWLHFDY (SEQ ID NO: 242); and the VH chain of the variable domain that binds LGR5 comprises the CDR1, CDR2 and CDR3 amino acid sequences NYAIS (SEQ ID NO: 200), SIIPILGTTDHAQKFQD (SEQ ID NO: 228), and EYIAARLDYFDS (SEQ ID NO: 255);
the VH chain of the variable domain that binds EGFR comprises the CDR1, CDR2 and CDR3 amino acid sequences ELSMH (SEQ ID NO: 189), GFDPEYGKTFFAQNFQG (SEQ ID NO: 216), and EGYYETTTYYYNLFDS (SEQ ID NO: 243); and the VH chain of the variable domain that binds LGR5 comprises the CDR1, CDR2 and CDR3 amino acid sequences SHWIG (SEQ ID NO: 194), VIYPGDSDTRYSPSFQG (SEQ ID NO: 222) and PNSGSPRYFEF (SEQ ID NO: 249);
the VH chain of the variable domain that binds EGFR comprises the CDR1, CDR2 and CDR3 amino acid sequences ELSMH (SEQ ID NO: 189), GFDPEYGKTFFAQNFQG (SEQ ID NO: 216), and EGYYETTTYYYNLFDS (SEQ ID NO: 243); and the VH chain of the variable domain that binds LGR5 comprises the CDR1, CDR2 and CDR3 amino acid sequences SYTMN (SEQ ID NO: 198), WINTDTGDPTYAQGFTG (SEQ ID NO:226), and GDCDSTSCYRYSYGYEDY (SEQ ID NO: 253);
the VH chain of the variable domain that binds EGFR comprises the CDR1, CDR2 and CDR3 amino acid sequences SYGIS (SEQ ID NO: 187), WISAYNGNTNYAQKLQG (SEQ ID NO: 214), and DRHWHWWLDAFDY (SEQ ID NO: 241); and the VH chain of the variable domain that binds LGR5 comprises the CDR1, CDR2 and CDR3 amino acid sequences SHWIG (SEQ ID NO: 194), VIYPGDSDTRYSPSFQG (SEQ ID NO: 222) and PNSGSPRYFEF (SEQ ID NO: 249); or
the VH chain of the variable domain that binds EGFR comprises the CDR1, CDR2 and CDR3 amino acid sequences SYGIS (SEQ ID NO: 187), WISAYNGNTNYAQKLQG (SEQ ID NO: 214), and DRHWHWWLDAFDY (SEQ ID NO: 241); and the VH chain of the variable domain that binds LGR5 comprises the CDR1, CDR2 and CDR3 amino acid sequences SYTMN (SEQ ID NO: 198), WINTDTGDPTYAQGFTG (SEQ ID NO: 226), and GDCDSTSCYRYSYGYEDY (SEQ ID NO: 253);
and wherein the first and second variable domains further comprise the light chain variable domain comprising a CDR1 sequence QSISSY (SEQ ID NO: 299), a CDR2 sequence AAS, and a CDR3 sequence QQSYSTPPT (SEQ ID NO: 300).

31. The method of claim 29, wherein the first and second variable domains further comprise a light chain variable domain comprising the amino acid sequence
(SEQ ID NO: 80)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

32. The method of claim 30, wherein the VH chain of the variable domain that binds EGFR comprises the amino acid sequence of SED ID NO: 20 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO:24 having at most 15 amino acid insertions, deletions, substitutions or a combination thereof which are not in the CDR1, CDR2 or CDR3 region.

33. The method of claim 30, wherein the VH chain of the variable domain that binds EGFR comprises the amino acid sequence of SEQ ID NO: 20 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO: 44 having at most 15 amino acid insertions, deletions, substitutions or a combination thereof which are not in the CDR1, CDR2 or CDR3 region.

34. The method of claim 30, wherein the VH chain of the variable domain that binds EGFR comprises the amino acid sequence of SEQ ID NO: 20, having at most 15 amino acid insertions, deletions, substitutions or a combination thereof which are not in the CDR1, CDR2 or CDR3 region and the VH chain of the variable domain that binds LGR5 comprises the amino acid sequence of SEQ ID NO: 46 having at most 15 amino acid insertions, deletions, substitutions or a combination thereof which are not in the CDR1, CDR2 or CDR3 region.

35. The method of claim 30, wherein the VH chain of the variable domain that binds EGFR comprises the amino acid sequence of SEQ ID NO: 20, having at most 15 amino acid insertions, deletions, substitutions or a combination thereof which are not in the CDR1, CDR2 or CDR3 region and the VH chain of the variable domain that binds LGR5 comprises the amino acid sequence of SEQ ID NO: 50 having at most 15 amino acid insertions, deletions, substitutions or a combination thereof which are not in the CDR1, CDR2 or CDR3 region.

36. The method of claim 30, wherein the VH chain of the variable domain that binds EGFR comprises the amino acid sequence of SEQ ID NO: 20, having at most 15 amino acid insertions, deletions, substitutions or a combination thereof which are not in the CDR1, CDR2 or CDR3 region and the VH chain of the variable domain that binds LGR5 comprises the amino acid sequence of SEQ ID NO: 52 having at most 15 amino acid insertions, deletions, substitutions or a combination thereof which are not in the CDR1, CDR2 or CDR3 region.

37. The method of claim 30, wherein the VH chain of the variable domain that binds EGFR comprises the amino acid sequence of SEQ ID NO: 20, having at most 15 amino acid insertions, deletions, substitutions or a combination thereof which are not in the CDR1, CDR2 or CDR3 region and the VH chain of the variable domain that binds LGR5 comprises the amino acid sequence of SEQ ID NO: 26 having at most 15 amino acid insertions, deletions, substitutions or a combination thereof which are not in the CDR1, CDR2 or CDR3 region.

38. The method of claim 30, wherein the VH chain of the variable domain that binds EGFR comprises the amino acid sequence of SEQ ID NO: 20 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO: 54 having at most 15 amino acid insertions, deletions, substitutions or a combination thereof which are not in the CDR1, CDR2 or CDR3 region.

39. The method of claim 30, wherein the VH chain of the variable domain that binds EGFR comprises the amino acid sequence of SEQ ID NO: 20 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO: 56 having at most 15 amino acid insertions, deletions, substitutions or a combination thereof which are not in the CDR1, CDR2 or CDR3 region.

40. The method of claim 30, wherein the VH chain of the variable domain that binds EGFR comprises the amino acid sequence of SEQ ID NO: 36 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO: 46 having at most 15 amino acid insertions, deletions, substitutions or a combination thereof which are not in the CDR1, CDR2 or CDR3 region.

41. The method of claim 30, wherein the VH chain of the variable domain that binds EGFR comprises the amino acid sequence of SEQ ID NO: 36 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO: 26 having at most 15 amino acid insertions, deletions, substitutions or a combination thereof which are not in the CDR1, CDR2 or CDR3 region.

42. The method of claim 30, wherein the VH chain of the variable domain that binds EGFR comprises the amino acid sequence of SEQ ID NO: 34 having at most 15 amino acid insertions, deletions, substitutions or a combination thereof which are not in the CDR1, CDR2 or CDR3 region, and the VH chain of the variable domain that binds LGR5 comprises the amino acid sequence of SEQ ID NO: 46 having at most 15 amino acid insertions, deletions, substitutions or a combination thereof which are not in the CDR1, CDR2 or CDR3 region.

43. The method of claim 30, wherein the VH chain of the variable domain that binds EGFR comprises the amino acid sequence SEQ ID NO: 34 having at most 15 amino acid insertions, deletions, substitutions or a combination thereof which are not in the CDR1, CDR2 or CDR3 region and the VH chain of the variable domain that binds LGR5 comprises the amino acid sequence of SEQ ID NO: 26 having at most 15 amino acid insertions, deletions, substitutions or a combination thereof which are not in the CDR1, CDR2 or CDR3 region.

44. The method of claim 30, wherein the VH chain of the variable domain that binds EGFR comprises the amino acid sequence of SED ID NO: 20 and the VH chain of the variable domain that binds LGR5 comprises the amino acid sequence of SEQ ID NO:24.

45. The method of claim 30, wherein the VH chain of the variable domain that binds EGFR comprises the amino acid sequence of SEQ ID NO: 20 and the VH chain of the variable domain that binds LGR5 comprises the amino acid sequence of SEQ ID NO: 44.

46. The method of claim 30, wherein the VH chain of the variable domain that binds EGFR comprises the amino acid sequence of SEQ ID NO: 20 and the VH chain of the variable domain that binds LGR5 comprises the amino acid sequence of SEQ ID NO: 46.

47. The method of claim 30, wherein the VH chain of the variable domain that binds EGFR comprises the amino acid sequence of SEQ ID NO: 20 and the VH chain of the variable domain that binds LGR5 comprises the amino acid sequence of SEQ ID NO: 50.

48. The method of claim 30, wherein the VH chain of the variable domain that binds EGFR comprises the amino acid sequence of SEQ ID NO: 20 and the VH chain of the variable domain that binds LGR5 comprises the amino acid sequence of SEQ ID NO: 52.

49. The method of claim 30, wherein the VH chain of the variable domain that binds EGFR comprises the amino acid sequence of SEQ ID NO: 20 and the VH chain of the variable domain that binds LGR5 comprises the amino acid sequence of SEQ ID NO: 26.

50. The method of claim 30, wherein the VH chain of the variable domain that binds EGFR comprises the amino acid sequence of SEQ ID NO: 20 and the VH chain of the variable domain that binds LGR5 comprises the amino acid sequence of SEQ ID NO: 54.

51. The method of claim 30, wherein the VH chain of the variable domain that binds EGFR comprises the amino acid sequence of SEQ ID NO:20 and the VH chain of the variable domain that binds LGR5 comprises the amino acid sequence of SEQ ID NO: 56.

52. The method of claim 30, wherein the VH chain of the variable domain that binds EGFR comprises the amino acid sequence of SEQ ID NO: 36 and the VH chain of the variable domain that binds LGR5 comprises the amino acid sequence of SEQ ID NO: 46.

53. The method of claim 30, wherein the VH chain of the variable domain that binds EGFR comprises the amino acid sequence of SEQ ID NO: 36 and the VH chain of the variable domain that binds LGR5 comprises the amino acid sequence of SEQ ID NO: 26.

54. The method of claim 30, wherein the VH chain of the variable domain that binds EGFR comprises the amino acid sequence of SEQ ID NO: 34 and the VH chain of the variable domain that binds LGR5 comprises the amino acid sequence of SEQ ID NO: 46.

55. The method of claim 30, wherein the VH chain of the variable domain that binds EGFR comprises the amino acid sequence SEQ ID NO: 34 and the VH chain of the variable domain that binds LGR5 comprises the amino acid sequence of SEQ ID NO: 26.

56. The method of claim 29, wherein the cancer is an adenocarcinoma.

57. The method of claim 29, wherein the cancer is colorectal cancer; pancreatic cancer; lung cancer; breast cancer; liver cancer; prostate cancer; ovarian cancer; cervical cancer; endometrial cancer; head and neck cancer; melanoma; testis cancer; urothelial cancer; renal cancer; stomach cancer; or carcinoid cancer.

58. The method of claim 29, wherein the cancer is colorectal cancer.

59. The method of claim 29, wherein the cancer is head and neck cancer.


SEQUENCE LISTING (300 sequences)

SEQ ID NO: 1 - protein, 907 aa, Homo sapiens
  SIGNAL 1..20
  DOMAIN 21..69: note = N-region
  DOMAIN 70..93: note = LRR1
  DOMAIN 94..117: note = LRR2
  DOMAIN 118..141: note = LRR3
  DOMAIN 142..165: note = LRR4
  DOMAIN 166..189: note = LRR5
  DOMAIN 190..213: note = LRR6
  DOMAIN 214..238: note = LRR7
  DOMAIN 239..257: note = LRR8
  DOMAIN 258..284: note = LRR9
  DOMAIN 285..308: note = LRR10
  DOMAIN 309..331: note = LRR11
  DOMAIN 332..355: note = LRR12
  DOMAIN 356..377: note = LRR13
  DOMAIN 378..400: note = LRR14
  DOMAIN 401..425: note = LRR15
  DOMAIN 426..448: note = LRR16
  DOMAIN 449..472: note = LRR17
  DOMAIN 473..560: note = CRL region
  DOMAIN 561..582: note = predicted TM1
  DOMAIN 592..617: note = predicted TM2
  DOMAIN 639..661: note = predicted TM3
  DOMAIN 680..704: note = predicted TM4
  DOMAIN 723..747: note = predicted TM5
  DOMAIN 766..789: note = predicted TM6
  DOMAIN 801..824: note = predicted TM7
  DOMAIN 825..907: note = C-terminal tail
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 2 - protein, 1210 aa, Homo sapiens [sequence omitted]

SEQ ID NO: 3 - protein, 997 aa, synthetic construct
  REGION 1..997: note = Amino acid sequence full length hLGR4-FLAG-HA insert
  SIGNAL 1..24
  REGION 952..953: note = MISC_FEATURE - linker
  REGION 954..961: note = MISC_FEATURE - FLG tag
  REGION 962..964: note = MISC_FEATURE - linker
  REGION 965..972: note = MISC_FEATURE - FLAG tag
  REGION 973..976: note = MISC_FEATURE - linker
  REGION 977..985: note = MISC_FEATURE - HA tag
  REGION 986..988: note = MISC_FEATURE - linker
  REGION 989..997: note = MISC_FEATURE - HA tag
MAGPLGLLCFLALGLLGSAGPSGAAPPLCAAPCSCDGDRRVDCSGKGLTAVPEGLSAFTQALDISMNNITQLPEDAFKNFPFLEELQLAGNDLSFIHPKALSGLKELKVLTLQNNQLKTVPSEAIRGLSALQSLRLDANHITSVPEDSFEGLVQLRHLWLDDNSLTEVPVHPLSNLPTLQALTLALNKISSIPDFAFTNLSSLVVLHLHNNKIRSLSQHCFDGLDNLETLDLNYNNLGEFPQAIKALPSLKELGFHSNSISVIPDGAFDGNPLLRTIHLYDNPLSFVGNSAFHNLSDLHSLVIRGASMVQQFPNLTGTVHLESLTLTGTKISSIPNNLCQEQKMLRTLDLSYNNIRDLPSFNGCHALEEISLQRNQIYQIKEGTFQGLISLRILDLSRNLIHEIHSRAFATLGPITNLDVSFNELTSFPTEGLNGLNQLKLVGNFKLKEALAAKDFVNLRSLSVPYAYQCCAFWGCDSYANLNTEDNSLQDHSVAQEKGTADAANVTSTLENEEHSQIIIHCTPSTGAFKPCEYLLGSWMIRLTVWFIFLVALFFNLLVILTTFASCTSLPSSKLFIGLISVSNLFMGIYTGILTFLDAVSWGRFAEFGIWWETGSGCKVAGFLAVFSSESAIFLLMLATVERSLSAKDIMKNGKSNHLKQFRVAALLAFLGATVAGCFPLFHRGEYSASPLCLPFPTGETPSLGFTVTLVLLNSLAFLLMAVIYTKLYCNLEKEDLSENSQSSMIKHVAWLIFTNCIFFCPVAFFSFAPLITAISISPEIMKSVTLIFFPLPACLNPVLYVFFNPKFKEDWKLLKRRVTKKSGSVSVSISSQGGCLEQDFYYDCGMYSHLQGNLTVCDCCESFLLTKPVSCKHLIKSHSCPALAVASCQRPEGYWSDCGTQSAHSDYADEEDSFVSDSSDQVQACGRACFYQSRGFPLVRYAYNLPRVKDSRDYKDDDDKAGADYKDDDDKLDGGYPYDVPDYAAGAYPYDVPDYA

SEQ ID NO: 4 - protein, 635 aa, synthetic construct
  REGION 1..635: note = Amino acid sequence hLGR4(ECD)-GPA33-FLAG insert in pVax1
  SIGNAL 1..24
  REGION 543..627: note = MISC_FEATURE - GPA33
  REGION 628..635: note = MISC_FEATURE - FLAG tag
MPGPLGLLCFLALGLLGSAGPSGAAPPLCAAPCSCDGDRRVDCSGKGLTAVPEGLSAFTQALDISMNNITQLPEDAFKNFPFLEELQLAGNDLSFIHPKALSGLKELKVLTLQNNQLKTVPSEAIRGLSALQSLRLDANHITSVPEDSFEGLVQLRHLWLDDNSLTEVPVHPLSNLPTLQALTLALNKISSIPDFAFTNLSSLVVLHLHNNKIRSLSQHCFDGLDNLETLDLNYNNLGEFPQAIKALPSLKELGFHSNSISVIPDGAFDGNPLLRTIHLYDNPLSFVGNSAFHNLSDLHSLVIRGASMVQQFPNLTGTVHLESLTLTGTKISSIPNNLCQEQKMLRTLDLSYNNIRDLPSFNGCHALEEISLQRNQIYQIKEGTFQGLISLRILDLSRNLIHEIHSRAFATLGPITNLDVSFNELTSFPTEGLNGLNQLKLVGNFKLKEALAAKDFVNLRSLSVPYAYQCCAFWGCDSYANLNTEDNSLQDHSVAQEKGTADAANVTSTLENEEHSQIIIHCTPSTGAFKPCEYLLGSWMIRVALYVGIAVGVVAALIIIGIIIYCCCCRGKDDNTEDKEDARPNREAYEEPPEQLRELSREREEEDDYRQEEQRSTGRESPDHLDQDYKDDDDK

SEQ ID NO: 5 - protein, 953 aa, synthetic construct
  REGION 1..953: note = Amino acid sequence full length hLGR5-FLAG-HA insert (
  SIGNAL 1..21
  REGION 908..909: note = MISC_FEATURE - linker
  REGION 910..918: note = MISC_FEATURE - FLAG tag
  REGION 919..921: note = MISC_FEATURE - linker
  REGION 922..929: note = MISC_FEATURE - FLAG tag
  REGION 930..933: note = MISC_FEATURE - linker
  REGION 934..942: note = MISC_FEATURE - HA tag
  REGION 943..945: note = MISC_FEATURE - linker
  REGION 946..953: note = MISC_FEATURE - HA tag
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSAKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCLARDYKDDDDKAGADYKDDDDKLDGGYPYDVPDYAAGAYPYDVPDYA

SEQ ID NO: 6 - protein, 654 aa, synthetic construct
  REGION 1..654: note = Amino acid sequence hLGR5(ECD)-GPA33(TM)-FLAG insert in pVax1
  SIGNAL 1..21
  REGION 563..646: note = MISC_FEATURE - GPA33
  REGION 647..654: note = MISC_FEATURE - FLAG tag
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRVALYVGIAVGVVAALIIIGIIIYCCCCRGKDDNTEDKEDARPNREAYEEPPEQLRELSREREEEDDYRQEEQRSTGRESPDHLDQDYKDDDDK

SEQ ID NO: 7 - protein, 936 aa, synthetic construct
  REGION 1..936: note = Amino acid sequence full length mouse (m)Lgr5-Myc-HIS insert in pEF1_Myc/His
  SIGNAL 1..21
  REGION 908..915: note = MISC_FEATURE - linker
  REGION 916..925: note = MISC_FEATURE - cMyc-tag
  REGION 926..930: note = MISC_FEATURE - linker
  REGION 931..936: note = MISC_FEATURE - His tag
MDTSCVHMLLSLLALLQLVAAGSSPGPDAIPRGCPSHCHCELDGRMLLRVDCSDLGLSELPSNLSVFTSYLDLSMNNISQLPASLLHRLCFLEELRLAGNALTHIPKGAFTGLHSLKVLMLQNNQLRQVPEEALQNLRSLQSLRLDANHISYVPPSCFSGLHSLRHLWLDDNALTDVPVQAFRSLSALQAMTLALNKIHHIADYAFGNLSSLVVLHLHNNRIHSLGKKCFDGLHSLETLDLNYNNLDEFPTAIKTLSNLKELGFHSNNIRSIPERAFVGNPSLITIHFYDNPIQFVGVSAFQHLPELRTLTLNGASHITEFPHLTGTATLESLTLTGAKISSLPQAVCDQLPNLQVLDLSYNLLEDLPSLSGCQKLQKIDLRHNEIYEIKGSTFQQLFNLRSLNLAWNKIAIIHPNAFSTLPSLIKLDLSSNLLSSFPVTGLHGLTHLKLTGNRALQSLIPSANFPELKIIEMPSAYQCCAFGGCENVYKISNQWNKDDGNSVDDLHKKDAGLFQVQDERDLEDFLLDFEEDLKALHSVQCSPSPGPFKPCEHLFGSWLIRIGVWTTAVLALSCNALVALTVFRTPLYISSIKLLIGVIAVVDILMGVSSAVLAAVDAFTFGRFAQHGAWWEDGIGCQIVGFLSIFASESSIFLLTLAALERGFSVKCSSKFEVKAPLFSLRAIVLLCVLLALTIATIPLLGGSKYNASPLCLPLPFGEPSTTGYMVALVLLNSLCFLIMTIAYTKLYCSLEKGELENLWDCSMVKHIALLLFANCILYCPVAFLSFSSLLNLTFISPDVIKFILLVIVPLPSCLNPLLYIVFNPHFKEDMGSLGKHTRFWMRSKHASLLSINSDDVEKRSCESTQALVSFTHASIAYDLPSTSGASPAYPMTESCHLSSVAFVPCLAAARGHPFEQKLISEEDLNMHTGHHHHHH

SEQ ID NO: 8 - protein, 281 aa, synthetic construct
  REGION 1..281: note = Human ZNRF3(ECD) in pDisplay
  SIGNAL 1..55
  REGION 222..231: note = MISC_FEATURE - cMyc-tag
  REGION 232..281: note = MISC_FEATURE - PDGFR
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMVDEQKLISEEDLNAVGQDTQEVIVVPHSLPFKVVVISAILALVVLTIISLIILIMLWQKKPR

SEQ ID NO: 9 - protein, 256 aa, synthetic construct
  REGION 1..256: note = Human ZNRF3 truncation mutant
  SIGNAL 1..55
  REGION 218..232: note = MISC_FEATURE - predicted TM region
  REGION 233..256: note = MISC_FEATURE - intracellular tail
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRPAV

SEQ ID NO: 10 - protein, 164 aa, synthetic construct
  REGION 1..164: note = Mouse ZNRF3 ECD-His in pUPE
  REGION 1..2: note = MISC_FEATURE - amino acids encoded by cloning sites
  REGION 155..157: note = MISC_FEATURE - amino acids encoded by cloning sites
  REGION 158..164: note = MISC_FEATURE - His tag
GSKETAFVEVVLFESSPSGDYTTHTTGLTGRFSRAGAMLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHLAAAHHHHHH

SEQ ID NO: 11 - protein, 278 aa, synthetic construct
  REGION 1..278: note = Mouse (m)ZNRF3 sequence in pDisplay
  SIGNAL 1..52
  REGION 217..218: note = MISC_FEATURE - amino acids that are encoded by cloning sites
  REGION 219..228: note = MISC_FEATURE - cMyc-tag
  REGION 229..278: note = MISC_FEATURE - PDGFR
MRPRSGGRPGAPGRRRRRLRRGPRGRRLPPPPPLPLLLGLLLAAAGPGAARAKETAFVEVVLFESSPSGDYTTHTTGLTGRFSRAGAMLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHLPPRQPTEYFDMVDEQKLISEEDLNAVGQDTQEVIVVPHSLPFKVVVISAILALVVLTIISLIILIMLWQKKPR

SEQ ID NO: 12 - protein, 261 aa, synthetic construct
  REGION 1..261: note = Human RNF43 in pDisplay
  SIGNAL 1..42
  REGION 200..201: note = MISC_FEATURE - amino acids encoded by cloning sites
  REGION 202..211: note = MISC_FEATURE - cMyc-tag
  REGION 212..261: note = MISC_FEATURE - PDGFR
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVVDEQKLISEEDLNAVGQDTQEVIVVPHSLPFKVVVISAILALVVLTIISLIILIMLWQKKPR

SEQ ID NO: 13 - protein, 261 aa, synthetic construct
  REGION 1..261: note = Mouse RNF43 in pDisplay
  REGION 200..201: note = MISC_FEATURE - amino acids encoded by cloning site
  REGION 202..211: note = MISC_FEATURE - cMyc-tag
  REGION 212..261: note = MISC_FEATURE - PDGFR
MSGGHQLQLAVLWPWLLMATLHAGFGHTGRVLAAAVESERSAEQKAVIRVIPLKMDPTGKLNLTLEGVFAGVAEVTPAEGKLMQSHPLYLCNASDDDNLEPGFISIVKLESPRRAPRPCLSLASKARMAGERGANAVLFDITEDRSAAEQLQQPLGLTKPVVLIWGSDAAKLMEFVYKNRKAYVWIELKEPPAGANYDVVDEQKLISEEDLNAVGQDTQEVIVVPHSLPFKVVVISAILALVVLTIISLIILIMLWQKKPR

SEQ ID NO: 14 - DNA, 43 bp, synthetic construct
  misc_feature 1..43: note = primer
  source 1..43: mol_type = other DNA
ccagcaggatccgccgccaccatggacacctcccggctcggtg

SEQ ID NO: 15 - DNA, 38 bp, synthetic construct
  misc_feature 1..38: note = primer
  source 1..38: mol_type = other DNA
ccagcagcggccgcttagagacatgggacaaatgccac

SEQ ID NO: 16 - protein, 907 aa, Macaca fascicularis
  SIGNAL 1..21
MDTSRLGVLLSLPVLLQLAAGSSSPRSGALLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRQVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPVTGLHGLTHLKLTGNHALQSLISSENFPELKIIEMPYAYQCCAFGVCENAYKISNQWNKGDNSSMDDLHKKDAGMFQVQDERDLEDFLLDFEEDLKALHSVQCSPSPGPFKPCEHLLDGWLIRIGVWTIAVLALTCNALVTSTVFRSPLYISPIKLLIGVIAVVNMLTGVSSAVLAGVDAFTFGSFARHGAWWENGVGCQVIGFLSIFASESSVFLLTLAALERGFSVKCSAKFETKAPFSSLKVIILLCALLALTMAAVPLLGGSEYGASPLCLPLPFGEPSTTGYMVALILLNSLCFLMMTIAYTKLYCNLDKGDLENIWDCSMVKHIALLLFTNCILYCPVAFLSFSSLLNLTFISPEVIKFILLVIVPLPACLNPLLYILFNPHFKEDLVSLGKQTYFWTRSKHPSLMSINSDDVEKQSCDSTQALVTFTSSSIAYDLPPSSVPSPAYPVTESCHLSSVAFVPCL

SEQ ID NO: 17 - DNA, 381 bp, synthetic construct
  misc_feature 1..381: note = MF1337
  source 1..381: mol_type = other DNA
  CDS 1..381
gaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagt

SEQ ID NO: 18 - protein, 127 aa, synthetic construct
  REGION 1..127: note = Synthetic Construct
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 19 - DNA, 360 bp, synthetic construct
  misc_feature 1..360: note = MF3755
  source 1..360: mol_type = other DNA
  CDS 1..360
caggtgcagctggtgcagtctgggtctgagttgaagaagcctggggcctcagtgaagatttcctgcaaggcttctggatacgacttcactaactatgctatgaattgggtgcgacaggcccctggacacgggcttgagtggatgggatggatcaacgccaacactggggacccaacgtatgcccagggcttcacaggacggtttgtcttctccttggacacctctgtcagcacggcatatctgcagatcagcagtttaaaggctgaggactctgccgtgtattactgtacgagagagcgatttttggagtggttacactttgactactggggccagggaaccctggtcaccgtctccagt

SEQ ID NO: 20 - protein, 120 aa, synthetic construct
  REGION 1..120: note = Synthetic Construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 21 - DNA, 372 bp, synthetic construct
  misc_feature 1..372: note = MF3178
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 22 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 23 - DNA, 378 bp, synthetic construct
  misc_feature 1..378: note = MF5790
  source 1..378: mol_type = other DNA
  CDS 1..378
caggtgcagctgcaggagtcgggcccaggactggtgaagccttcggagaccctgtccctcacctgcactgtctctggtggctccttcagcagtagtagttcctactggggctggatccgccagcccccagggaaggggctggagtggattgggagtttctattatagtgggaacacctactacaacccgtccctcaagagtcgagtcaccatatccgaagacacgtccaagaaccagttctccctgaagctgagctctgtgaccgccgcagacacggctgtgtattactgtgcgagacagacgtatagcagcagctgggacggggtcctgtactactttgactactggggccagggaaccctggtcaccgtctccagt

SEQ ID NO: 24 - protein, 126 aa, synthetic construct
  REGION 1..126: note = Synthetic Construct
QVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSS

SEQ ID NO: 25 - DNA, 381 bp, synthetic construct
  misc_feature 1..381: note = MF5816
  source 1..381: mol_type = other DNA
  CDS 1..381
gaggtgcagctggtgcagtctgggtctaaattgaagaagcctggggcctcagtgaaggtttcctgcaaggcttctggatacaccttcactagctatactatgaattgggtgcgacaggcccctggacaagggcttgagtggatgggatggatcaacaccgacactggggacccaacgtatgcccagggcttcacaggacggtttgtcttctccttggacacctctgtcagcacggcatttctacagatcaacagcctaaaggctgaggacactgccgtatattactgtgcgagaggagattgtgatagtaccagctgctatagatacagttatggttacgaggactactggggccagggaaccctggtcaccgtctccagt

SEQ ID NO: 26 - protein, 127 aa, synthetic construct
  REGION 1..127: note = Synthetic Construct
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 27 - DNA, 372 bp, synthetic construct
  misc_feature 1..372: note = MF3125
  source 1..372: mol_type = other DNA
  CDS 1..372
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaaaaggttcttctggttggccgtcttactctaactggggctttgattattggggccaaggtaccctggtcaccgtctccagt

SEQ ID NO: 28 - protein, 124 aa, synthetic construct
  REGION 1..124: note = Synthetic Construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGSSGWPSYSNWGFDYWGQGTLVTVSS

SEQ ID NO: 29 - DNA, 366 bp, synthetic construct
  misc_feature 1..366: note = MF3176
  source 1..366: mol_type = other DNA
  CDS 1..366
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggctgtgtattactgtgcaagagattggtggtacccgccgtactactggggctttgattattggggccaaggtaccctggtcaccgtctccagt

SEQ ID NO: 30 - protein, 122 aa, synthetic construct
  REGION 1..122: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWWYPPYYWGFDYWGQGTLVTVSS

SEQ ID NO: 31 - DNA, 366 bp, synthetic construct
  misc_feature 1..366: note = MF4863
  source 1..366: mol_type = other DNA
  CDS 1..366
gaggtgcagctggtgcagtctgggtctgagttgaagaagcctggggcctcagtgaaggtttcctgcaaggcttctggatacacctccattagatatgctttgaactgggtgcgacaggcccctggacaaggccttgagtggctgggatggatcaacaccaacactgggaacccaacgtatgcccggggcttcacaggacggtttgtcttctccttggacacctctgtcagcacggcatatctgcagatcagcagcctaaaggctgaggacactgccgtgtattactgtgcgagagatacttatgatagtactggttatctttggtttgactactggggccagggaaccctggtcaccgtctccagt

SEQ ID NO: 32 - protein, 122 aa, synthetic construct
  REGION 1..122: note = Synthetic Construct
EVQLVQSGSELKKPGASVKVSCKASGYTSIRYALNWVRQAPGQGLEWLGWINTNTGNPTYARGFTGRFVFSLDTSVSTAYLQISSLKAEDTAVYYCARDTYDSTGYLWFDYWGQGTLVTVSS

SEQ ID NO: 33 - DNA, 366 bp, synthetic construct
  misc_feature 1..366: note = MF3370
  source 1..366: mol_type = other DNA
  CDS 1..366
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggctgtgtattactgtgcaaaagatcgtcattggcattggtggctggacgcctttgattattggggccaaggtaccctggtcaccgtctccagt

SEQ ID NO: 34 - protein, 122 aa, synthetic construct
  REGION 1..122: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 35 - DNA, 375 bp, synthetic construct
  misc_feature 1..375: note = MF4280
  source 1..375: mol_type = other DNA
  CDS 1..375
caggtgcagctggtgcagtctggggctgaggtgaagaagcctggggcctcagtgaaggtctcctgcaaggtttccggatacaccctcactgaattatccatgcactgggtgcgacaggctcctggtaaagggcttgaatggatgggaggctttgatcctgagtatggtaaaacattcttcgcacagaacttccagggcagagtcaccatgaccgaggacacatctgcagacacagcctacatggagctaagcagcctgagatctgaggacacggccgtgtattactgtgcaacagaggggtattatgagactactacttattactacaacctttttgactcctggggccagggaaccctggtcaccgtctccagt

SEQ ID NO: 36 - protein, 125 aa, synthetic construct
  REGION 1..125: note = Synthetic Construct
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 37 - DNA, 357 bp, synthetic construct
  misc_feature 1..357: note = MF4289
  source 1..357: mol_type = other DNA
  CDS 1..357
caggtgcagctggtgcaatctgggtctgaattgaagaagcctggggcctcagtgaaggtttcctgcaagacttctggatacaccttcactgactatgctatgacttgggtgcgacaggcccctggacaagggcttgaatggatgggatggatcaccaccaacactggggacccaacgtatgccccgggcttcacaggacggtttgtcttctccttggacacctctgtcagcacggcatatctgcagatcagcagcctaaaggccgaggacactgccgtatattactgtgcgagagtgtatcattggatacggggatttgagttttggggccagggaaccctggtcaccgtctccagt

SEQ ID NO: 38 - protein, 119 aa, synthetic construct
  REGION 1..119: note = Synthetic Construct
QVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSS

SEQ ID NO: 39 - DNA, 345 bp, synthetic construct
  misc_feature 1..345: note = MF5777
  source 1..345: mol_type = other DNA
  CDS 1..345
caggtgcagctggtgcaatctgggtctgagttgaagaagcctggggcctcagtgaaggtttcctgcaaggcttctggatacaccttcactaactatgttatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgaggtgggagctactagactactggggccagggaaccctggtcaccgtctccagt

SEQ ID NO: 40 - protein, 115 aa, synthetic construct
  REGION 1..115: note = Synthetic Construct
QVQLVQSGSELKKPGASVKVSCKASGYTFTNYVMNWVRQAPGQGLEWMGWINTNTGNPTYAQGFTGRFVFSLDTSVSTAYLQISSLKAEDTAVYYCARWELLDYWGQGTLVTVSS

SEQ ID NO: 41 - DNA, 348 bp, synthetic construct
  misc_feature 1..348: note = MF5781
  source 1..348: mol_type = other DNA
  CDS 1..348
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttattagtggtagtggtgggaccacatactacgcagactccgtgaagggccggttcaccatctccagagacaattccaagaacacgctgtatctgcaaatgaacagcctgagagccgaggacacggccgtatattactgtgcgaaaggggctggggagcttgactactggggccagggaaccctggtcaccgtctccagt

SEQ ID NO: 42 - protein, 116 aa, synthetic construct
  REGION 1..116: note = Synthetic Construct
EVQLVESGGGLVQPGGSLRLSCAASGFTFSSYAMSWVRQAPGKGLEWVSVISGSGGTTYYADSVKGRFTISRDNSKNTLYLQMNSLRAEDTAVYYCAKGAGELDYWGQGTLVTVSS

SEQ ID NO: 43 - DNA, 378 bp, synthetic construct
  misc_feature 1..378: note = MF5803
  source 1..378: mol_type = other DNA
  CDS 1..378
caggtgcagctgcaggagtcggggccaggactggtgaagccttcggagaccctgtccctcacctgcactgtctctaatggctccatcagtacttactactggagctggatccggcagcccccagggaaggggctggagtggattggatatgtctattacactgggcgcaccaagtacaacccctccctcaagagtcgagtcaccatatcagtagacacgtccaagaaccagttctccctgaacctgagttctgtgaccgctgcggacacggccgtgtattactgtgcgagagggggtattgtagtagtcccagctgcgcgggactattactactacatggacgtctggggcaaagggaccacggtcaccgtctccagt

SEQ ID NO: 44 - protein, 126 aa, synthetic construct
  REGION 1..126: note = Synthetic Construct
QVQLQESGPGLVKPSETLSLTCTVSNGSISTYYWSWIRQPPGKGLEWIGYVYYTGRTKYNPSLKSRVTISVDTSKNQFSLNLSSVTAADTAVYYCARGGIVVVPAARDYYYYMDVWGKGTTVTVSS

SEQ ID NO: 45 - DNA, 360 bp, synthetic construct
  misc_feature 1..360: note = MF5805
  source 1..360: mol_type = other DNA
  CDS 1..360
gaggtgcaactggtgcagtctggagcagaggtgaaaaagcccggggagtctctgaagatcgcctgtaagggttctggattcagttttaccagccactggatcggctgggtgcgccagaagcccgggagaggcctggagtggatgggggtcatctatcctggtgactctgataccagatacagcccgtccttccaaggccaggtcaccgtctcagccgacaagtccatcaataccgcctacctgcagtggaacagcctgaaggcctcggacaccgccatatattactgtgcgagaccgaacagtgggagtccccggtacttcgagttctggggccgtggcaccctggtcaccgtctccagt

SEQ ID NO: 46 - protein, 120 aa, synthetic construct
  REGION 1..120: note = Synthetic Construct
EVQLVQSGAEVKKPGESLKIACKGSGFSFTSHWIGWVRQKPGRGLEWMGVIYPGDSDTRYSPSFQGQVTVSADKSINTAYLQWNSLKASDTAIYYCARPNSGSPRYFEFWGRGTLVTVSS

SEQ ID NO: 47 - DNA, 378 bp, synthetic construct
  misc_feature 1..378: note = MF5808
  source 1..378: mol_type = other DNA
  CDS 1..378
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtttctattatagtgggaacacctactacaacccgtccctcaagagtcgagtcaccatatccgtagacacgtccaagaaccagttctccctgaagctgagctctgtgaccgccgcagacacggctgtgtattactgtgcgagacaggagtattactatggttcggggagtccttcgtactactttgactactggggccagggaaccctggtcaccgtctccagt

SEQ ID NO: 48 - protein, 126 aa, synthetic construct
  REGION 1..126: note = Synthetic Construct
QVQLQESGPGLVKPSETLSLTCTVSGGSISSSSYYWGWIRQPPGKGLEWIGSFYYSGNTYYNPSLKSRVTISVDTSKNQFSLKLSSVTAADTAVYYCARQEYYYGSGSPSYYFDYWGQGTLVTVSS

SEQ ID NO: 49 - DNA, 360 bp, synthetic construct
  misc_feature 1..360: note = MF5809
  source 1..360: mol_type = other DNA
  CDS 1..360
gaggtgcagctggtgcagtctggagcagaggtgaaaaagcccggggagtctctgaagatctcctgtaagggttctggagacagttttatcagccactggatcgcctgggtgcgccagatgcccgggaaaggcctggagtggatggggatcgtctatcctggtgactctgataccagatacagcccgtccttccaaggccaggtcaccatctcagccgacaagtccatcaccaccgcctacttgcagtggagcagcctgaaggcctcggacaccgccatgtattactgtgcgagacacgagtgggaactacttggcccctttgactactggggccagggaaccctggtcaccgtctccagt

SEQ ID NO: 50 - protein, 120 aa, synthetic construct
  REGION 1..120: note = Synthetic Construct
EVQLVQSGAEVKKPGESLKISCKGSGDSFISHWIAWVRQMPGKGLEWMGIVYPGDSDTRYSPSFQGQVTISADKSITTAYLQWSSLKASDTAMYYCARHEWELLGPFDYWGQGTLVTVSS

SEQ ID NO: 51 - DNA, 363 bp, synthetic construct
  misc_feature 1..363: note = MF5814
  source 1..363: mol_type = other DNA
  CDS 1..363
gaggtgcagctggtgcagtctggggctgaggtgaagaagcctgggtcctcggtgaaggtctcctgcaaggcttctggaggcacctccactaacgatgctatcagttgggtgcgacagacccctggacaagggcttgagtggatgggaagtatcatccctatccttgatacaacagaccacgcacagaagttccagggcagagtcacgattaccgcggacaaatccacgaacacagcctacatggagctgaacagcctgagatctgatgacacggccgtgtattactgtgcgagagagcatatagcagctcgtcaggactactttgactattggggccagggaaccctggtcaccgtctccagt

SEQ ID NO: 52 - protein, 121 aa, synthetic construct
  REGION 1..121: note = Synthetic Construct
EVQLVQSGAEVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 53 - DNA, 357 bp, synthetic construct
  misc_feature 1..357: note = MF5817
  source 1..357: mol_type = other DNA
  CDS 1..357
caggtgcagctggtgcagtctggggctgaggtgaagaagcctgggtcctcggtgaaggtctcctgcaaggtttctggaggcaccttcaggagctatgctatcagctgggtgcgacaggcccctggacaagggcttgagtggatgggagggatcatccctatctttgatacaagaaactacgcacagattcttcagggcagagtcacgattaccgcggacttatccacgagcacagcctacatggagctgaacagtctgagatctgaggacacggccatttattactgtgcgagagggagcgacgagggggactggttcgacccctggggccaaggaaccctggtcaccgtctccagt

SEQ ID NO: 54 - protein, 119 aa, synthetic construct
  REGION 1..119: note = Synthetic Construct
QVQLVQSGAEVKKPGSSVKVSCKVSGGTFRSYAISWVRQAPGQGLEWMGGIIPIFDTRNYAQILQGRVTITADLSTSTAYMELNSLRSEDTAIYYCARGSDEGDWFDPWGQGTLVTVSS

SEQ ID NO: 55 - DNA, 363 bp, synthetic construct
  misc_feature 1..363: note = MF5818
  source 1..363: mol_type = other DNA
  CDS 1..363
gaggtgcagctggtgcagtctgggactgaggtgaggaagcctgggtcctcggtgaaggtctcctgcaaggcttctggaggcaccttcagcaactatgctatcagctgggtgcgacaggcccctggacaggggcttgagtggatgggaagtatcatccctatccttggaacaacagaccacgcacagaagttccaggacagagtcacgattaccgcggacaaatcctcgaacacaacctacatggagctgagcagcctgagatctgatgacacggccgtatattactgtgcgagagagtatatagcagctcgtctggactactttgactcttggggccagggaaccctggtcaccgtctccagt

SEQ ID NO: 56 - protein, 121 aa, synthetic construct
  REGION 1..121: note = Synthetic Construct
EVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSS

SEQ ID NO: 57 - DNA, 366 bp, synthetic construct
  misc_feature 1..366: note = MF5832
  source 1..366: mol_type = other DNA
  CDS 1..366
gaggtgcagctggtggagtctgggggaggcttggtacagcctggggggtccctgagactctcctgtgtagtctctggattcaccttcagttactacgacatgcactgggtccgccaagtcacaggaaaaggtctggagtgggtctcagctattggcactgctggtgccacatactatccaggctccgtgaagggccgattcaccatctccagagaaaatgccaagaactccttgtatcttcaaatgaatagcctgagagccggggacacggctgtgtattactgtgcaagagatcgtggatatagtggctacgatgcgtactactttgacttctggggccagggaaccctggtcaccgtctccagt

SEQ ID NO: 58 - protein, 122 aa, synthetic construct
  REGION 1..122: note = Synthetic Construct
EVQLVESGGGLVQPGGSLRLSCVVSGFTFSYYDMHWVRQVTGKGLEWVSAIGTAGATYYPGSVKGRFTISRENAKNSLYLQMNSLRAGDTAVYYCARDRGYSGYDAYYFDFWGQGTLVTVSS

SEQ ID NO: 59 - DNA, 372 bp, synthetic construct
  misc_feature 1..372: note = MF5836
  source 1..372: mol_type = other DNA
  CDS 1..372
caggtgcagctgcaggagtcgggcccaggactggtgaagccttcggagaccctgtccctcacctgcactgtctctggtggctccatcagcagtagtaattactactggggctggatccgccagcccccagggaaggggctggagtggattgggaatatctattatagagggtacacctattataacccgtccctcaagagtcgagtcaccatatccgtagacacgtccaagaagcagttctccctgacgctgagctctgtgaccgccgcagacacggctatgtattactgtgcgagagaggggagtgactacggtgactacgtaggagcttttgatatctgggaccaagggacaatggtcaccgtctccagt

SEQ ID NO: 60 - protein, 124 aa, synthetic construct
  REGION 1..124: note = Synthetic Construct
QVQLQESGPGLVKPSETLSLTCTVSGGSISSSNYYWGWIRQPPGKGLEWIGNIYYRGYTYYNPSLKSRVTISVDTSKKQFSLTLSSVTAADTAMYYCAREGSDYGDYVGAFDIWDQGTMVTVSS

SEQ ID NO: 61 - DNA, 366 bp, synthetic construct
  misc_feature 1..366: note = MF5839
  source 1..366: mol_type = other DNA
  CDS 1..366
gaggtgcagctggtgcagtctgggggaggcttggtacagcctggggggtccctgagactctcctgtgcagcctctggattcaccttcagttactacgacatgcactgggtccgccaagttacaggaaaaggtctggagtgggtctcaactattggtgctactggtgacacatactattcagactccgtgaagggccgatttaccatctccagacaaaatgccaagaactccttgtatcttcaaataaacagcctgagagccggggacacggctgtatattactgtgtaagagatcgtggatatattggctacgattcgtactactttgacaactggggccagggaaccctggtcaccgtctccagt

SEQ ID NO: 62 - protein, 122 aa, synthetic construct
  REGION 1..122: note = Synthetic Construct
EVQLVQSGGGLVQPGGSLRLSCAASGFTFSYYDMHWVRQVTGKGLEWVSTIGATGDTYYSDSVKGRFTISRQNAKNSLYLQINSLRAGDTAVYYCVRDRGYIGYDSYYFDNWGQGTLVTVSS

SEQ ID NO: 63 - DNA, 360 bp, synthetic construct
  misc_feature 1..360: note = M5850
  source 1..360: mol_type = other DNA
  CDS 1..360
caggtgcagctggtgcagtctgggtctgagttgaagaagcctggggcctcagtgaaggtttcctgcaaggcttctggatacaccttcactaggtatcctatgaattgggtgcgacaggcccctggacaagggcttgagtggatgggatggatcaacaccaacactgggaacccaacatatgcccagggcttcacaggacggtttgtcttctccttggacacctctgtcagcacggcatttctgcagatcagcagcctaaaggctgaggacactgccgtgtattactgtgcgagagagaggactaacttttatgatgcttttgatatctggggccaagggacaatggtcaccgtctccagt

SEQ ID NO: 64 - protein, 120 aa, synthetic construct
  REGION 1..120: note = Synthetic Construct
QVQLVQSGSELKKPGASVKVSCKASGYTFTRYPMNWVRQAPGQGLEWMGWINTNTGNPTYAQGFTGRFVFSLDTSVSTAFLQISSLKAEDTAVYYCARERTNFYDAFDIWGQGTMVTVSS

SEQ ID NO: 65 - DNA, 360 bp, synthetic construct
  misc_feature 1..360: note = MF5853
  source 1..360: mol_type = other DNA
  CDS 1..360
caggtgcagctggtgcaatctgggtctgagttgaagaagcctggggcctcagtgaaggtttcctgcaaggcttctggatataccttcaatagctatgctatggattgggtgcgacaggcccctggacaagggcttgagtggatgggatggatcaacaccaatactgggaacccaacgtatgcccaggccttcacaggacggtttgtcttctccttggacacctctgtcagcacggcatatctggagatcagcagcctaaaggctgaggacactgccgtgtattactgtgcgagagagaggcatggatattttgaagcttttgatatctggggccaagggaccacggtcaccgtctccagt

SEQ ID NO: 66 - protein, 120 aa, synthetic construct
  REGION 1..120: note = Synthetic Construct
QVQLVQSGSELKKPGASVKVSCKASGYTFNSYAMDWVRQAPGQGLEWMGWINTNTGNPTYAQAFTGRFVFSLDTSVSTAYLEISSLKAEDTAVYYCARERHGYFEAFDIWGQGTTVTVSS

SEQ ID NO: 67 - DNA, 360 bp, synthetic construct
  misc_feature 1..360: note = MF5855
  source 1..360: mol_type = other DNA
  CDS 1..360
caggtgcagctggtgcaatctgggtctgagttgaagaagcctggggcctcagtgaaggtttcctgcaaggcttctggatacaccttcactaagtatgttatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcaggtcagcagtctaagggctgaggacactgccctgtattactgtgcgagagagtctaactggaactacgactactttgactactggggccagggcaccctggtcaccgtctccagt

SEQ ID NO: 68 - protein, 120 aa, synthetic construct
  REGION 1..120: note = Synthetic Construct
QVQLVQSGSELKKPGASVKVSCKASGYTFTKYVMNWVRQAPGQGLEWMGWINTNTGNPTYAQGFTGRFVFSLDTSVSTAYLQVSSLRAEDTALYYCARESNWNYDYFDYWGQGTLVTVSS

SEQ ID NO: 69 - DNA, 366 bp, synthetic construct
  misc_feature 1..366: note = MF5862
  source 1..366: mol_type = other DNA
  CDS 1..366
caggtgcagctggtgcagtctggaacagaggtgaaaaagcccggggagtctctgaagatctcctgtaagggttctggatacagctttaccacctactggatcggctgggtgcgccagatgcccgggaaaggcctggagtggatgggaatcatctatcctggtgactctgataccagatacagcccgtccttccaaggccaggtcaccatctcagccgacaagtccatcagcaccgcctacctgcagttgagcagcctgaaggcctcggacaccgccatgtattactgtgcgagacggggtcttagtatggttcggttgagcgcttttgatgtctggggccaaggaaccctggtcaccgtctccagt

SEQ ID NO: 70 - protein, 122 aa, synthetic construct
  REGION 1..122: note = Synthetic Construct
QVQLVQSGTEVKKPGESLKISCKGSGYSFTTYWIGWVRQMPGKGLEWMGIIYPGDSDTRYSPSFQGQVTISADKSISTAYLQLSSLKASDTAMYYCARRGLSMVRLSAFDVWGQGTLVTVSS

SEQ ID NO: 71 - DNA, 360 bp, synthetic construct
  misc_feature 1..360: note = MF5882
  source 1..360: mol_type = other DNA
  CDS 1..360
caggtgcagctggtgcaatctgggtctgagttgaagaagcctggggcctcagtgaaggtttcctgcaaggcttctggatacaccttcactagatatgctatgaattgggtgcgacaggcccctggacaagggcttgagtggatgggatggatcaacaccaaaactgggaacccaacgtatgcccagggcttcacaggacggtttgtcttctccttggacacctctgtcagcacggcatatctgcagatcagcagcctaaaggctgaggacactgccgtgtattactgtgcgagagatcgtgggagctactatgatgcttttgatatctggggccaagggacaatggtcaccgtctccagt

SEQ ID NO: 72 - protein, 120 aa, synthetic construct
  REGION 1..120: note = Synthetic Construct
QVQLVQSGSELKKPGASVKVSCKASGYTFTRYAMNWVRQAPGQGLEWMGWINTKTGNPTYAQGFTGRFVFSLDTSVSTAYLQISSLKAEDTAVYYCARDRGSYYDAFDIWGQGTMVTVSS

SEQ ID NO: 73 - DNA, 360 bp, synthetic construct
  misc_feature 1..360: note = MF5884
  source 1..360: mol_type = other DNA
  CDS 1..360
gaggtgcagctggtgcagtctgggtctgaattgaagaagcctggggcctcagtgaaggtttcctgcaaggcttctggatacaccttcactaagtatgctatgaattgggtgcgacaggtccctggacaagggcttgagtggatgggatggatcaacaccaacactgggaacccaacgtatgcccagggcttcacaggacggtttgtcttctccttggacacctctgtccgcacggcatatctgcagatcagcagcctaaaggctgaggacactgccgtgtattactgtgcgagaaaaggggggagctactacgactggttcgacccctggggccagggaaccctggtcaccgtctccagt

SEQ ID NO: 74 - protein, 120 aa, synthetic construct
  REGION 1..120: note = Synthetic Construct
EVQLVQSGSELKKPGASVKVSCKASGYTFTKYAMNWVRQVPGQGLEWMGWINTNTGNPTYAQGFTGRFVFSLDTSVRTAYLQISSLKAEDTAVYYCARKGGSYYDWFDPWGQGTLVTVSS

SEQ ID NO: 75 - DNA, 369 bp, synthetic construct
  misc_feature 1..369: note = MF5887
  source 1..369: mol_type = other DNA
  CDS 1..369
caggtgcagctggtgcagtctgggtctgagttgaagaagcctggggcctcagtgaaggtttcctgcaaggcttctggatacaccttcactcgctatgctatgaattgggtgcgacaggcccctggacaagggcttgagtggatgggatggatcaacaccaacactgggaaaccaacgtatgcccagggcttcacaggacggtttgtcttctccttggacacctctgtcagcacggcatatctgcagatcagcagcctgaaggctgaggacactgccgtgtattactgtgcgagagataagggctataactggaactacatgggtgcttttgatatctggggccaagggaccacggtcaccgtctccagt

SEQ ID NO: 76 - protein, 123 aa, synthetic construct
  REGION 1..123: note = Synthetic Construct
QVQLVQSGSELKKPGASVKVSCKASGYTFTRYAMNWVRQAPGQGLEWMGWINTNTGKPTYAQGFTGRFVFSLDTSVSTAYLQISSLKAEDTAVYYCARDKGYNWNYMGAFDIWGQGTTVTVSS

SEQ ID NO: 77 - DNA, 360 bp, synthetic construct
  misc_feature 1..360: note = MF5888
  source 1..360: mol_type = other DNA
  CDS 1..360
caggtgcagctggtgcagtctgggtctgagttgaagaagcccgggggctcaatgaaggtttcctgcaaggcttctggatacaccttcactagatatgctatgaattggttgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtctattattgtgcgagacggagtgggagctactacgactactttgactactggggccagggaaccctggtcaccgtctccagt

SEQ ID NO: 78 - protein, 120 aa, synthetic construct
  REGION 1..120: note = Synthetic Construct
QVQLVQSGSELKKPGGSMKVSCKASGYTFTRYAMNWLRQAPGQGLEWMGWINTNTGNPTYAQGFTGRFVFSLDTSVSTAYLQISSLKAEDTAVYYCARRSGSYYDYFDYWGQGTLVTVSS

SEQ ID NO: 79 - DNA, 321 bp, synthetic construct
  misc_feature 1..321: note = common light chain - 1
  source 1..321: mol_type = other DNA
  CDS 1..321
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 80 - protein, 107 aa, synthetic construct
  REGION 1..107: note = Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 81 - DNA, 324 bp, synthetic construct
  misc_feature 1..324: note = common light chain - 2
  source 1..324: mol_type = other DNA
  CDS 1..324
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 82 - protein, 107 aa, synthetic construct [sequence omitted]

SEQ ID NO: 83 - DNA, 294 bp, synthetic construct
  misc_feature 1..294: note = VH-CH1
  source 1..294: mol_type = other DNA
  CDS 1..294
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 84 - protein, 98 aa, synthetic construct
  REGION 1..98: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 85 - DNA, 45 bp, synthetic construct
  misc_feature 1..45: note = VH-hinge
  source 1..45: mol_type = other DNA
  CDS 1..45
gagcccaaatcttgtgacaaaactcacacatgcccaccgtgccca

SEQ ID NO: 86 - protein, 15 aa, synthetic construct
  REGION 1..15: note = Synthetic Construct
EPKSCDKTHTCPPCP

SEQ ID NO: 87 - DNA, 330 bp, synthetic construct
  misc_feature 1..330: note = VH-CH2
  source 1..330: mol_type = other DNA
  CDS 1..330
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 88 - protein, 110 aa, synthetic construct
  REGION 1..110: note = Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 89 - DNA, 321 bp, synthetic construct
  misc_feature 1..321: note = VH-CH3
  source 1..321: mol_type = other DNA
  CDS 1..321
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 90 - protein, 106 aa, synthetic construct
  REGION 1..106: note = Synthetic Construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - DNA, 321 bp, synthetic construct
  misc_feature 1..321: note = VH-CH3
  source 1..321: mol_type = other DNA
  CDS 1..321
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 92 - protein, 106 aa, synthetic construct
  REGION 1..106: note = Synthetic Construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 110 aa, synthetic construct
  REGION 1..110: note = VH1-69 J00256-AEHJ2
QVQLVQSGAEVKKPGSSVKVSCKASGGTFSSYAISWVRQAPGQGLEWMGGIIPIFGTANYAQKKFQGRVTITADKSTSTAYMELSSLRSEDTAVYYCARWGRGTLVTVSS

SEQ ID NO: 94 - protein, 121 aa, synthetic construct
  REGION 1..121: note = member of supercluster 1
EVQLVQSGAEVKKPGSSVKVSCKAFGGTFNNYAINWVRQAPGQGLEWMGSIIPILGTVDHAQKFQGRVTITADKSTNTVYMELSSLRSDDTAVYYCAREHIAARLDYFDYWDLGTLVTVSS

SEQ ID NO: 95 - protein, 121 aa, synthetic construct; identical to SEQ ID NO: 52
  REGION 1..121: note = member of supercluster 1

SEQ ID NO: 96 - protein, 121 aa, synthetic construct
  REGION 1..121: note = member of supercluster 1
EVQLVQSGAEVKKPGSSVKVSCRASGDSVSNYAISWVRQAPGQGLEWMGSIIPILGTTDHAQQFQDRVTITADKSTSTAYMELGSLRSDDTAVYYCARERIAARQDYFDYWGQGTLVTVSS

SEQ ID NO: 97 - protein, 121 aa, synthetic construct
  REGION 1..121: note = member of supercluster 1
EVQLVQSGAEVKKPGSSVKVSCKASGGTFSNYAISWVRQAPGQGLEWMGSIIPILGTTDHAQKFQDRVTITADRSSNTTYMDLNSLRSDDTAVYYCARELIAARLDYFDSWGQGTLVTVSS

SEQ ID NO: 98 - protein, 121 aa, synthetic construct
  REGION 1..121: note = member of supercluster 1
EVQLVQSGAEVKKPGSSVKVSCKASGGTFSNYAISWVRQAPGQGLEWMGSIIPILGTTDHAQKFQDRVTITADKSSNTTYMDLSSLRSDDTAVYYCARERIAARLDYFDSWGQGTLVTVSS

SEQ ID NO: 99 - protein, 121 aa, synthetic construct
  REGION 1..121: note = member of supercluster 1
EVQLVQSGAEVKKPGSSVKVSCKSSGGTFKNYAISWVRQAPGQGLEWMGSIIPILGTTDHAQNFQDRVTITADKLSNTTYMELSSLRSDDTAVYYCAIELIAARLDYFHSWGQGTLVTVSS

SEQ ID NO: 100 - protein, 121 aa, synthetic construct
  REGION 1..121: note = member of supercluster 1
EVQLVQSGAEVKKPGSSVKVSCKASGGTVSNYAISWVRQAPGQGLDWMGSIIPILGTTDHAQKFQGRVTFTADKSTNTSYMELNSLRSDDTAVYYCARELIAARQDYFDYWGQGTLVTVSS

SEQ ID NO: 101 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQDRVTITADRSSNTTYMDLNSLRSDDTAVYYCARELIAARLDYFDSWGQGTLVTVSS

SEQ ID NO: 102 - protein, 121 aa, synthetic construct
  REGION 1..121: note = member of supercluster 1
QVQLVQSGAEVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 103 - protein, 121 aa, synthetic construct
  REGION 1..121: note = member of supercluster 1
QVQLVQSGAEVKKPGSSVKVSCKSSGGTFKNYAISWVRQAPGQGLEWMGSIIPILGTTDHAQNFQDRVTITADKLSNTTYMELSSLRSDDTAVYYCAIELIAARLDYFHSWGQGTLVTVSS

SEQ ID NO: 104 - protein, 121 aa, synthetic construct
  REGION 1..121: note = member of supercluster 1
EVQLVQSGAEVKKPGSSVKVSCKASGGTSTNYAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 105 - protein, 121 aa, synthetic construct
  REGION 1..121: note = member of supercluster 1
EVQLVQSGAEVKKPGSSVKVSCKASGGTSTNDAISWVRQTPGQGLEWMGSIIPILDTTDHAQKFQGRVTITADRSTNTAYMELNSLKSDDTAVYYCAREHIAARQDYFDYWGQGTLVTVSS

SEQ ID NO: 106 - protein, 121 aa, synthetic construct
  REGION 1..121: note = member of supercluster 1
EVQLVQSGAEVKKPGSSVKVSCKASGGTLSNYAISWVRQAPGQGLEWMGSIIPILGTTDHAQNFQDRVTITADKSSNTTYMELSSLRSDDTAVYYCARERIAARLDYFDSWGQGTLVTVSS

SEQ ID NO: 107 - protein, 121 aa, synthetic construct
  REGION 1..121: note = member of supercluster 1
EVQLVQSGAEVKKPGSSVKVSCKASGGTLSNYAISWVRQAPGQGLEWMGSIIPILGTIDHAQNFQDRVTITADKSSNTTYMELSSLRSDDTAVYYCARERIAARLDYFDSWGQGTLVTVSS

SEQ ID NO: 108 - protein, 121 aa, synthetic construct
  REGION 1..121: note = member of supercluster 1
QVQLVQSGAEVKKPGSSVKVSCRASGDSVSNYAISWVRQAPGQGLEWMGSIIPILGTTDHAQQFQDRVTITADKSTSTAYMELGSLRSDDTAVYYCARERIAARQDYFDYWGQGTLVTVSS

SEQ ID NO: 109 - protein, 128 aa, synthetic construct
  REGION 1..128: note = member of supercluster 1
EVQLVQSGAEVKKPGSSVKVSCKASGGTFSNYAISWVRQAPGQGLEWMGSIIPILGTTDHAEKFQGRVTITADKSTSTAYMDLSTAYMELSSLRSDDTAVYYCAREFIAARQDYFDYWGQGTLVTVSS

SEQ ID NO: 110 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQDRVTITADKSSNTTYMDLSSLRSDDTAVYYCARERIAARLDYFDSWGQGTLVTVSS

SEQ ID NO: 111 - protein, 121 aa, synthetic construct
  REGION 1..121: note = member of supercluster 1
EVQLVQSGAEVKKPGSSVKVSCKASGGTSTNYAISWVRQTPGQGLEWMGSIIPILDTTDHAQKFQGRVTITADKSTNTTYMELNSLRSDDTAVYYCAREHIAARLDYFDYWGQGTLVTVSS

SEQ ID NO: 112 - protein, 121 aa, synthetic construct
  REGION 1..121: note = member of supercluster 1
EVQLVQSGAEVKKPGSSVKVSCKAFGGTFNNYAINWVRQAPGQGLEWMGSIIPILGTVDHAQKFQGRVTITADKSTNTVYMELSSLRSDDTAVYYCAREHIAARLDYFDYWDLGILVTVSS

SEQ ID NO: 113 - protein, 121 aa, synthetic construct
  REGION 1..121: note = member of supercluster 1
EVQLVQSGAEVKKPGSSVKVSCKASGGTFSNYAISWVRQAPGQGLEWMGSIIPILGTTDHAQNFQDRVTITADKSSNTTYMELIGLKSDDTAVYYCARERIAARLDYFDSWGQGTLVTVSS

SEQ ID NO: 114 - protein, 121 aa, synthetic construct
  REGION 1..121: note = member of supercluster 1
RVQLVQSGAEVKKPGSSVKVSCKAFGGTFNNYAINWVRQAPGQGLEWMGSIIPILGTVDHAQKFQGRVTITADKSSNTVYMELSSLRSDDTAVYYCAREHIAARLDYFDSWGLGTLVTVSS

SEQ ID NO: 115 - protein, 121 aa, synthetic construct
  REGION 1..121: note = member of supercluster 1
RCSMVQSGSELKKPGASVKVSCRASGDSVSNYAISWVRQAPGQGLEWMGSIIPILGTTDHAQQFQDRVTITADKSTSTAYMELGSLRSDDTAVYYCARERIAARQDYFDYWGQGTLVTVSS

SEQ ID NO: 116 - protein, 121 aa, synthetic construct
  REGION 1..121: note = member of supercluster 1
RCSMVQSGAEVKKPGSSVKVSCKASGGTFSNYAISWVRQAPGQGLEWMGSIIPILGTTDHAQKFQDRVTITADRSSNTTYMDLNSLRSDDTAVYYCARELIAARLDYFDSWGQGTLVTVSS

SEQ ID NO: 117 - protein, 121 aa, synthetic construct
  REGION 1..121: note = member of supercluster 1
EVQLVQSGTEVKKPGSSVKVSCKASGGTFSNYAISWVRQAPGQGLEWMGSIIPILGTTDHTQKFQGRITITADKSTNTSYMDLNSLRSDDTAVYYCARERIAARQDYFDSWGQGTLVTVSS

SEQ ID NO: 118 - protein, 121 aa, synthetic construct
  REGION 1..121: note = member of supercluster 1
RLQLVQSGAEVKKPGSSVKVSCKASGGTSTNDAISWVRQAPGQGLEWMGSIIPILGTTDHAQNFQDRVTITADKSSNTTYMELIGLKSDDTAVYYCARERIAARLDYFDSWGQGTLVTVSS

SEQ ID NO: 119 - protein, 121 aa, synthetic construct
  REGION 1..121: note = member of supercluster 1
QVQLVQSGAEVKKPGSSVKVSCKASGGTSTNDAISWVRQTPEQGLEWMGSIIPILDTTDHAQKFQGRVTITADRSTNTAYMELNSLRSDDTAVYYCARELIAARLDYFDSWGQGTLVTVSS

SEQ ID NO: 120 - protein, 121 aa, synthetic construct
  REGION 1..121: note = member of supercluster 1
EVQLVQSGAEVKKPGSSVKVSCKASGGTFTNYAISWVRQAPGQGLEWMGSIIPILGTTDHAQNFQGRVTITADRSANTAYMELNSLRSDDTAVYYCAREHIAARQDYFDYWGQETLVTVSS

SEQ ID NO: 121 - protein, 121 aa, synthetic construct
  REGION 1..121: note = member of supercluster 1
EVQLVQSGAEVKKPGSSVKVSCKASGGTFNNYAISWVRQAPGQGLEWMGSIIPILGTTDHAQKFQGRVTITADISTITAYMELSSLRSDDTAIYYCARERIAARQDYFDFWGQGTLVTVSS

SEQ ID NO: 122 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQDRVTITAGKSSNTTYMDLSSLRSDDTAVYYCARERIAARLDYFDSWGQGTLVTVSS

SEQ ID NO: 123 - protein, 121 aa, synthetic construct
  REGION 1..121: note = member of supercluster 1
QVQLVQSGAAVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 124 - protein, 121 aa, synthetic construct
  REGION 1..121: note = member of supercluster 1
EVQLVQSGAEVKKPGSSVKVSCKASGGTSTNDAISWVRQTPEQGLEWMGSIIPILGTVDHAQKFQGRVTITADKSTNTVYMELSSLRSDDTAVYYCAREHIAARLDYFDYWDLGTLVTVSS

SEQ ID NO: 125 - protein, 121 aa, synthetic construct
  REGION 1..121: note = member of supercluster 1
EVQLVQSGAEVKKPGSSVKVSCKASGDTSTNYAISWVRQTPGQGLEWMGSIIPILGTTDHTQKFQDRVTITADKSTNTAYMELNSLRSDDTAVYYCAREHIASRQDFFDYWGQGTLVTVSS

SEQ ID NO: 126 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQDRVTITADKSSNTTYMELSSLRSDDTAVYYCARERIAARLDYFDSWGQGTLVTVSS

SEQ ID NO: 127 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQDRVTITADKSSNTTYMDLSSLRSDDTAVYYCARERIAARLDYFDYWGQGTLVTVSS

SEQ ID NO: 128 - protein, 128 aa, synthetic construct
  REGION 1..128: note = member of supercluster 1
EVQLVQSGAEVKKPGSSVKVSCKASGGTFSNYAISWVRQAPGQGLEWMGSIIPILGTTDHAEKFQGKVTITADKSTSTAYMDLSTAYMELSSLRSDDTAVYYCAREFIAARQDYFDYWGQGTLVTVSS

SEQ ID NO: 129 - protein, 121 aa, synthetic construct
  REGION 1..121: note = member of supercluster 1
EVQLVQSGAEVKKPGSSVKVSCKASGGTFSNYAISWVRQAPGQGLEWMGSIIPIFGTADNAQKFQGRVTITADKSTSTAYMELSSLRSDDTAVYYCARERIAARQDYFDYWGQGTLVTVSS

SEQ ID NO: 130 - protein, 121 aa, synthetic construct
  REGION 1..121: note = member of supercluster 1
EVQLVQSGSELKKPGSSVKVSCKASGGTSTNDAISWVRQTPGQGLEWMGSIIPILDTTDHAQKFQGRVTITADRSTNTAYMELNSLKSDDTAVYYCAREHIAARQDYFDYWGQGTLVTVSS

SEQ ID NO: 131 - protein, 121 aa, synthetic construct
  REGION 1..121: note = member of supercluster 1
QVQLVQSGAEVKKPGSSVKVSCKASGGTSTNDAISWVRQTPGQGLEWMGSIIPILDTTDHAQKFQGRVTITADRSTNTAYMELNSLKSDDTAVYYCAREHIAARQDYFDYWGQGTLVTVSS

SEQ ID NO: 132 - protein, 121 aa, synthetic construct
  REGION 1..121: note = member of supercluster 1
QVQLVQSGAEVKKPGSSVKVSCKASGGTLSNYAISWVRQAPGQGLEWMGSIIPILGTTDHAQNFQDRVTITADKSSNTTYMELSSLRSDDTAVYYCARERIAARLDYFDSWGQGTLVTVSS

SEQ ID NO: 133 - protein, 121 aa, synthetic construct
  REGION 1..121: note = member of supercluster 1
EVQLVQSGAEVKMPGSSVKVSCKASGGTSTNDAISWVRQTPGQGLEWMGSIIPILDTTDHAQKFQGRVTITADRSTNTAYMELNSLKSDDTAVYYCAREHIAARQDYFDYWGQGTLVTVSS

SEQ ID NO: 134 - protein, 121 aa, synthetic construct
  REGION 1..121: note = member of supercluster 1
RVQLVQSGAEVKKPGSSVKVSCKASGGTFNNYAINWVRQAPGQGLEWMGSIIPILGTVDHAQKFQGRVTITADKSTNTVYMELSSLRSDDTAVYYCAREHIAARLDYFDYWDLGTLVTVSS

SEQ ID NO: 135 - protein, 121 aa, synthetic construct
  REGION 1..121: note = member of supercluster 1
EVQLVQSGAEVKKPGSSVKVSCKASGGTFSSYAISWVRQAPGQGLEWMGSIIPILGTTDHAQKFQGRVTFTADKSTNTSYMELNSLRSDDSAVYYCARERIAARQDYFDSWGQGTLVTVSS

SEQ ID NO: 136 - protein, 121 aa, synthetic construct
  REGION 1..121: note = member of supercluster 1
RVQLVQSGSELKKPGASVKVSCKASGGTFSNYAISWVRQAPGQGLEWMGSIIPILGTTDHAQKFQDRVTITADKSSNTTYMDLSSLRSDDTAVYYCARERIAARLDYFDSWGQGTLVTVSS

SEQ ID NO: 137 - protein, 121 aa, synthetic construct
  REGION 1..121: note = member of supercluster 1
RVQLVQSGAEVKKPGSSVKVSCKAFGGTFNNYAINWVRQAPGQGLEWMGSIIPILGTVDHAQKFQGRVTITADKSTNTVYMELSSLRSDDTAVYYCAREHIAARLDYFDYWDLGTLVTVSS

SEQ ID NO: 138 - protein, 121 aa, synthetic construct
  REGION 1..121: note = member of supercluster 1
EVQLVQSGAEVKKPGSSVKVSCKASGGTLSNYAISWVRQAPGQGLEWMGSIIPILGTADHAQKFQGRVTITADKSTSTAYMELSSLRSDDTAVYYCARERIAARQDYFDYWGQGTLVTVSS

SEQ ID NO: 139 - protein, 128 aa, synthetic construct
  REGION 1..128: note = member of supercluster 1
EVQLVQSGTEVKKPGSSVKVSCKASGGTFSNYAISWVRQAPGQGLEWMGSIIPILGTTDHAEKFQGRVTITADKSTSTAYMDLSTAYMELSSLRSDDTAVYYCAREFIAARQDYFDYWGQGTLVTVSS

SEQ ID NO: 140 - protein, 121 aa, synthetic construct
  REGION 1..121: note = member of supercluster 1
EVQLVQSGAEVKKPGSSVKVSCKASGGTFSSYAISWVRQAPGQGLEWMGSIIPILGTVDHAQKFQGRVTITADKSTNTVYMELSSLRSDDTAVYYCAREHIAARLDYFDYWDLGTLVTVSS

SEQ ID NO: 141 - protein, 121 aa, synthetic construct
  REGION 1..121: note = member of supercluster 1
EVQLVQSGAEVKKPESSVKVSCKASGGTFSNYAISWVRQAPGQGLEWMGSIIPILGTTDHAQKFQDRVTITADKSSNTTYMELSSLRSDDTAVYYCAREHIAARLDYFDSWGQGTLVTVSS

SEQ ID NO: 142 - protein, 121 aa, synthetic construct; identical to SEQ ID NO: 56
  REGION 1..121: note = member of supercluster 1

SEQ ID NO: 143 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAEKFQGRVTITADKSTSTAYMELSSLRSDDTAVYYCAREFIAARQDYFDYWGQGTLVTVSS

SEQ ID NO: 144 - protein, 121 aa, synthetic construct
  REGION 1..121: note = member of supercluster 1
EVQLVQSGAEVKKPGSSVKVSCKSSGGTFKNYAISWVRQAPGQGLEWMGSIIPILGTTDHAQNFQDRVTITADKLSNTTYMELSSLRSDDTAVYYCARERIAARLDYFDSWGQGTLVTVSS

SEQ ID NO: 145 - protein, 121 aa, synthetic construct
  REGION 1..121: note = member of supercluster 1
EVQLVQSGAEVKKPGSSVKVSCEASGGTFTNYAISWVRQAPGQGLEWMGSIIPVLGTTDHAQKFQDRVTITADKSSNITYMELSSLRSDDTAVYYCARERIAARLDYFDSWGQGTLVTVSS

SEQ ID NO: 146 - protein, 121 aa, synthetic construct
  REGION 1..121: note = member of supercluster 1
QVQLVQSGAEVKKPGSSVKVSCKASGGTSTNYAISWVRQTPGQGLEWMGSIIPILDTTDHAQKFQGRVTITADKSTNTTYMELNSLRSDDTAVYYCAREHIAARLDYFDYWGQGTLVTVSS

SEQ ID NO: 147 - protein, 121 aa, synthetic construct
  REGION 1..121: note = member of supercluster 1
EVQLVQSGSELKKPGSSVKVSCKASGGTFSNYAISWVRQAPGQGLEWMGSIIPILGTTDHAQKFQDRVTITADKSSNTTYMDLSSLRSDDTAVYYCARERIAARLDYFDSWGQGTLVTVSS

SEQ ID NO: 148 - protein, 121 aa, synthetic construct
  REGION 1..121: note = member of supercluster 1
EVQLVQSGAEVKKPGSSVKVSCKASGGTFSNYAISWVRQAPGQGLEWMGSIIPILGTTDHVEKFQDRVTITADKSSNTTYMELSSLRSDDTAVYYCARELIAARLDYFDSWGQGTLVTVSS

SEQ ID NO: 149 - protein, 121 aa, synthetic construct
  REGION 1..121: note = member of supercluster 1
EVQLVQSGAEVKKPGSSVKVSCKASGGTFSNYAISWVRQAPGQGLEWMGSIIPILGTTDHAQKFQGRVTFTADKSTNTSYMELNSLRSDDSAVYYCARERIAARQDYFDSWGQGTLVTVSS

SEQ ID NO: 150 - protein, 121 aa, synthetic construct
  REGION 1..121: note = member of supercluster 1
QVQLVQSGAEVKKPGSSVKVSCKASGGTSTNDAISWVRQTPEQGLEWMGSIIPILDTTDHAQKFQGRVTITADRSTNTAYMELNSLRSDDTAVYYCAREHIAARQDYFDYWGQGTLVTVSS

SEQ ID NO: 151 - protein, 121 aa, synthetic construct
  REGION 1..121: note = member of supercluster 1
EVQLVQSGAEVKKPGSSVKVSCKASGGTFSSYAISWVRQAPGQGLEWMGSIIPILDTTDHAQKFQGRVTITADKSTNTAYMELNSLRSDDTAVYYCAREHIAARQDYFDYWGQGTLVTVSS

SEQ ID NO: 152 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQGRVTFTADKSTNTSYMELNSLRSDDSAVYYCARERIAARQDYFDSWGQGTLVTVSS

SEQ ID NO: 153 - protein, 121 aa, synthetic construct
  REGION 1..121: note = member of supercluster 1
EVQLVQSGAEVKKPGSSVKVSCKASGGTFSNYAISWVRQAPGQGLEWMGSIIPILGTTDHAQKFQDRVTITADKSSKTTYMELNSLRSDDTAVYYCARELIAARLDYFDSWGQGTLVTVSS

SEQ ID NO: 154 - protein, 121 aa, synthetic construct
  REGION 1..121: note = member of supercluster 1
EVQLVQSGAEVKKPGSSVKVSCKASGGTFSSYAISWVRQTPEQGLEWMGSIIPILDTTDHAQKFQGRVTITADRSTNTAYMELNSLRSDDTAVYYCAREHIAARQDYFDYWGQGTLVTVSS

SEQ ID NO: 155 - protein, 121 aa, synthetic construct
  REGION 1..121: note = member of supercluster 1
QVQLVQSGAEVKKPGSSVKVSCKASGGTFSNYAISWVRQAPGQGLEWMGSIIPILGTTDHAQKFQDRVTITADKSSNTTYMELSSLRSDDTAVYYCAREHIAARLDYFDSWGQGTLVTVSS

SEQ ID NO: 156 - protein, 121 aa, synthetic construct
  REGION 1..121: note = member of supercluster 1
RVQLVQSGAEVKKPGSSVKVSCRASGDSVSNYAISWVRQAPGQGLEWMGSIIPILGTTDHAQQFQDRVTITADKSTSTAYMELGSLRSDDTAVYYCARERIAARQDYFDYWGQGTLVTVSS

SEQ ID NO: 157 - protein, 99 aa, synthetic construct
  REGION 1..99: note = VH4-39|J00256-AEHJ6
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 158 - protein, 126 aa, synthetic construct; identical to SEQ ID NO: 24
  REGION 1..126: note = member of supercluster 8

SEQ ID NO: 159 - protein, 126 aa, synthetic construct
  REGION 1..126: note = member of supercluster 8
QVQLQESGPGLVKPSETLSLTCTVSGGSFSSSSSYWGWIRQPPGKGLEWIGSFYYSGNTYYNPSLKSRVTISVDTSKNQFSLKLSSVTAADTAVYYCARQTYSSSWDGVLYYFDYWGQGTLVTVSS

SEQ ID NO: 160 - protein, 124 aa, synthetic construct
  REGION 1..124: note = member of supercluster 8
QVQLQESGPGLVKPSETLSLTCTVSGGSISSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSS

SEQ ID NO: 161 - protein, 109 aa, synthetic construct
  REGION 1..109: note = member of supercluster10
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARWGQGTTVTVSS

SEQ ID NO: 162 - protein, 127 aa, synthetic construct; identical to SEQ ID NO: 26
  REGION 1..127: note = member of supercluster10

SEQ ID NO: 163 - protein, 127 aa, synthetic construct
  REGION 1..127: note = member of supercluster10
EVQLVQSGSELKKPGASVKVSCKASGYTFTSYAMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 164 - protein, 127 aa, synthetic construct
  REGION 1..127: note = member of supercluster10
EVQLVQSGSALKKPGASVKVSCKASGYTFTSYTMNWVRAQPGQGLEWMGWINTQTGNPTYAQGFTGRFVFSLDTSVSTAYLQISSLKAEDTAVYYCARGDCSSTSCYRYSYGYEDYWGQGTLVTVSS

SEQ ID NO: 165 - protein, 127 aa, synthetic construct
  REGION 1..127: note = member of supercluster10
EVQLVQSGSTLKKPGASVKVSCKASGYTFTSYTMNWVRQAPGQGLEWMGWIDTNTGDPTYAQGFTGRFVFSLDTSVTTAFLQINSLKAEDTAVYYCARGDCDSTSCYRYSYGYEDYWGQGTLVTVSS

SEQ ID NO: 166 - protein, 127 aa, synthetic construct
  REGION 1..127: note = member of supercluster10
GCSMVQSGSESKKPGASVKVSCKASGYTFTSYAMNWVRQAPGQGLEWMGWINTNTGNPTYAQGFTGRFVFSLDTSVSTAFLQINSLKAEDTAVYYCARGDCDSTSCYRYSYGYEDYWGQGTLVTVSS

SEQ ID NO: 167 - protein, 127 aa, synthetic construct
  REGION 1..127: note = member of supercluster10
EVQLVQSGSELKKPGASVKVSCKASGYTFTSYAMNWVRQAPGQGLEWMGWINTNTGNPTYAQGFTGRFVFSLDTSVTTAFLQINSLKAEDTAVYYCARGDCDSTSCYRYSYGYEDYWGQGTLVTVSS

SEQ ID NO: 168 - protein, 127 aa, synthetic construct
  REGION 1..127: note = member of supercluster10
EVQLVQSGSELKKPGASVKVSCKASGYTFTSYAMNWVRQAPGQGLEWMGWINTNTGNPTYAQGFTGRFVFSLDTSVSTAFLQINSLKAEDTAVYYCARGDCDSTSCYRYSYGYEDYWGQGTLVTVSS

SEQ ID NO: 169 - protein, 127 aa, synthetic construct
  REGION 1..127: note = member of supercluster10
QVQLVQSGSELKKPGASVKVSCKASGYTFTSYAMNWVRQAPGQGLEWMGWINTYTGDPTYAQGFTGRFVFSLDTSVSTAYLQINNLKAEDTAVYYCARGDCSSTSCYRYSYGYEDYWGQGTLVTVSS

SEQ ID NO: 170 - protein, 127 aa, synthetic construct
  REGION 1..127: note = member of supercluster10
QVQLVQSGSELKKPGASVKVSCKASGYTFTSYAMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 171 - protein, 127 aa, synthetic construct
  REGION 1..127: note = member of supercluster10
QVQLVQSGSELKKPGASVKVSCKASGYTFTSYTMNWVRQAPGQGLEWMGWIDTNTGDPTYAQGFTGRFVFSLDTSVTTAFLQINSLKAEDTAVYYCARGDCDSTSCYRYSYGYEDYWGQGTLVTVSS

SEQ ID NO: 172 - protein, 127 aa, synthetic construct
  REGION 1..127: note = member of supercluster10
EVQLVQSGSELKKPGASVKVSCKASGYTFTNYALNWVRQAPGQGLEWMGWINTNTGDPTYAQGFTGRFVFSLDTSVSSAFLQISSLKAEDTAVYYCARGDCDSTDCYRYSYGYEDYWGQGTLVTVSS

SEQ ID NO: 173 - protein, 127 aa, synthetic construct
  REGION 1..127: note = member of supercluster10
EVQLVQSGSKLKKPGASVKVSCKASGYTFTSYTMNWVRQAPGRGLEWMGWINTNTGDPTYAQGFTGRFVFSLDTSVSTAFLQINSLKAEDTAVYYCARGDCDSTSCYRYSYGYEDYWGQGTLVTVSS

SEQ ID NO: 174 - protein, 127 aa, synthetic construct; identical to SEQ ID NO: 26
  REGION 1..127: note = member of supercluster10

SEQ ID NO: 175 - protein, 127 aa, synthetic construct
  REGION 1..127: note = member of supercluster10
EVQLVQSGSELKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 176 - protein, 127 aa, synthetic construct
  REGION 1..127: note = member of supercluster10
EVQLVQSGSELKKPGASVKVSCKASGYTFTSYAMNWVRQAPGQGLEWMGWINTYTGDPTYAQGFTGRFVFSLDTSVSTAYLQINNLKAEDTAVYYCARGDCSSTSCYRYSYGYEDYWGQGTLVTVSS

SEQ ID NO: 177 - protein, 127 aa, synthetic construct
  REGION 1..127: note = member of supercluster10
EVQLVQSGSELKKPGASVKVSCKASGYTFTNYAMHWVRQAPGQGLEWMGWIDTNTGNPTYAQGFTGRFVFSLDTSVSTAYLQISSLKAEDTAVYYCARGDCSSTSCYRYSYGYEDYWGQGTLVTVSS

SEQ ID NO: 178 - protein, 127 aa, synthetic construct
  REGION 1..127: note = member of supercluster10
EVQLVQSGSKLEKPGASVKVSCKASGYTFTSYTMNWVRQAPGQGLEWMGWINTNTGDPTYAQGFTGRFVFSLDTSVSTAFLQINSLKAEDTAVYFCARGDCDSTSCYRYSYGYEDYWGQGTLVTVSS

SEQ ID NO: 179 - protein, 127 aa, synthetic construct
  REGION 1..127: note = member of supercluster10
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYFCARGDCDSTSCYRYSYGYEDYWGQGTLVTVSS

SEQ ID NO: 180 - protein, 127 aa, synthetic construct
  REGION 1..127: note = member of supercluster10
QVQLVQSGSELKKPGASVKVSCKASGYTFTSYAMNWVRQAPGQGLEWMGWINTNTGDPTYAQGFTGRFVFSLDTSVSTAFLQINSLKAEDTAVYFCARGDCDSTNCYRYSYGYEDYWGQGTLVTVSS

SEQ ID NO: 181 - protein, 127 aa, synthetic construct
  REGION 1..127: note = member of supercluster10
QVQLVQSGSTLKKPGASVKVSCKASGYTFTSYTMNWVRQAPGQGLEWMGWIDTNTGDPTYAQGFTGRFVFSLDTSVTTAFLQINSLKAEDTAVYFCARGDCDSTSCYRYSYGYEDYWGQGTLVTVSS

SEQ ID NO: 182 - protein, 5 aa, synthetic construct
  REGION 1..5: note = Synthetic sequence
KYDIN

SEQ ID NO: 183 - protein, 5 aa, synthetic construct
  REGION 1..5: note = Synthetic sequence
SYGMH

SEQ ID NO: 184 - protein, 5 aa, synthetic construct
  REGION 1..5: note = Synthetic sequence
SYAMS

SEQ ID NO: 185 - protein, 5 aa, synthetic construct
  REGION 1..5: note = Synthetic sequence
GYYMH

SEQ ID NO: 186 - protein, 5 aa, synthetic construct
  REGION 1..5: note = Synthetic sequence
RYALN

SEQ ID NO: 187 - protein, 5 aa, synthetic construct
  REGION 1..5: note = Synthetic sequence
SYGIS

SEQ ID NO: 188 - protein, 5 aa, synthetic construct
  REGION 1..5: note = Synthetic sequence
NYAMN

SEQ ID NO: 189 - protein, 5 aa, synthetic construct
  REGION 1..5: note = Synthetic sequence
ELSMH

SEQ ID NO: 190 - protein, 5 aa, synthetic construct
  REGION 1..5: note = Synthetic sequence
DYAMT

SEQ ID NO: 191 - protein, 5 aa, synthetic construct
  REGION 1..5: note = Synthetic sequence
NYVMN

SEQ ID NO: 192 - protein, 7 aa, synthetic construct
  REGION 1..7: note = Synthetic sequence
SSSSYWG

SEQ ID NO: 193 - protein, 5 aa, synthetic construct
  REGION 1..5: note = Synthetic sequence
TYYWS

SEQ ID NO: 194 - protein, 5 aa, synthetic construct
  REGION 1..5: note = Synthetic sequence
SHWIG

SEQ ID NO: 195 - protein, 7 aa, synthetic construct
  REGION 1..7: note = Synthetic sequence
SSSYYWG

SEQ ID NO: 196 - protein, 5 aa, synthetic construct
  REGION 1..5: note = Synthetic sequence
SHWIA

SEQ ID NO: 197 - protein, 5 aa, synthetic construct
  REGION 1..5: note = Synthetic sequence
NDAIS

SEQ ID NO: 198 - protein, 5 aa, synthetic construct
  REGION 1..5: note = Synthetic sequence
SYTMN

SEQ ID NO: 199 - protein, 5 aa, synthetic construct
  REGION 1..5: note = Synthetic sequence
SYAIS

SEQ ID NO: 200 - protein, 5 aa, synthetic construct
  REGION 1..5: note = Synthetic sequence
NYAIS

SEQ ID NO: 201 - protein, 5 aa, synthetic construct
  REGION 1..5: note = Synthetic sequence
YYDMH

SEQ ID NO: 202 - protein, 7 aa, synthetic construct
  REGION 1..7: note = Synthetic sequence
SSNYYWG

SEQ ID NO: 203 - protein, 5 aa, synthetic construct
  REGION 1..5: note = Synthetic sequence
RYPMN

SEQ ID NO: 204 - protein, 5 aa, synthetic construct
  REGION 1..5: note = Synthetic sequence
SYAMD

SEQ ID NO: 205 - protein, 5 aa, synthetic construct
  REGION 1..5: note = Synthetic sequence
KYVMN

SEQ ID NO: 206 - protein, 5 aa, synthetic construct
  REGION 1..5: note = Synthetic sequence
TYWIG

SEQ ID NO: 207 - protein, 5 aa, synthetic construct
  REGION 1..5: note = Synthetic sequence
RYAMN

SEQ ID NO: 208 - protein, 5 aa, synthetic construct
  REGION 1..5: note = Synthetic sequence
KYAMN

SEQ ID NO: 209 - protein, 17 aa, synthetic construct
  REGION 1..17: note = Synthetic sequence
WMSANTGNTGYAQKFQG

SEQ ID NO: 210 - protein, 17 aa, synthetic construct
  REGION 1..17: note = Synthetic sequence
VISYDGSNKYYADSVKG

SEQ ID NO: 211 - protein, 17 aa, synthetic construct
  REGION 1..17: note = Synthetic sequence
AISGSGGSTYYADSVKG

SEQ ID NO: 212 - protein, 17 aa, synthetic construct
  REGION 1..17: note = Synthetic sequence
WINPNSGGTNYAQKFQG

SEQ ID NO: 213 - protein, 17 aa, synthetic construct
  REGION 1..17: note = Synthetic sequence
WINTNTGNPTYARGFTG

SEQ ID NO: 214 - protein, 17 aa, synthetic construct
  REGION 1..17: note = Synthetic sequence
WISAYNGNTNYAQKLQG

SEQ ID NO: 215 - protein, 17 aa, synthetic construct
  REGION 1..17: note = Synthetic sequence
WINANTGDPTYAQGFTG

SEQ ID NO: 216 - protein, 17 aa, synthetic construct
  REGION 1..17: note = Synthetic sequence
GFDPEYGKTFFAQNFQG

SEQ ID NO: 217 - protein, 17 aa, synthetic construct
  REGION 1..17: note = Synthetic sequence
WITTNTGDPTYAPGFTG

SEQ ID NO: 218 - protein, 17 aa, synthetic construct
  REGION 1..17: note = Synthetic sequence
WINTNTGNPTYAQGFTG

SEQ ID NO: 219 - protein, 17 aa, synthetic construct
  REGION 1..17: note = Synthetic sequence
VISGSGGTTYYADSVKG

SEQ ID NO: 220 - protein, 16 aa, synthetic construct
  REGION 1..16: note = Synthetic sequence
SFYYSGNTYYNPSLKS

SEQ ID NO: 221 - protein, 16 aa, synthetic construct
  REGION 1..16: note = Synthetic sequence
YVYYTGRTKYNPSLKS

SEQ ID NO: 222 - protein, 17 aa, synthetic construct
  REGION 1..17: note = Synthetic sequence
VIYPGDSDTRYSPSFQG

SEQ ID NO: 223 - skipped

SEQ ID NO: 224 - protein, 17 aa, synthetic construct
  REGION 1..17: note = Synthetic sequence
IVYPGDSDTRYSPSFQG

SEQ ID NO: 225 - protein, 17 aa, synthetic construct
  REGION 1..17: note = Synthetic sequence
SIIPILDTTDHAQKFQG

SEQ ID NO: 226 - protein, 17 aa, synthetic construct
  REGION 1..17: note = Synthetic sequence
WINTDTGDPTYAQGFTG

SEQ ID NO: 227 - protein, 17 aa, synthetic construct
  REGION 1..17: note = Synthetic sequence
GIIPIFDTRNYAQILQG

SEQ ID NO: 228 - protein, 17 aa, synthetic construct
  REGION 1..17: note = Synthetic sequence
SIIPILGTTDHAQKFQD

SEQ ID NO: 229 - protein, 16 aa, synthetic construct
  REGION 1..16: note = Synthetic sequence
AIGTAGATYYPGSVKG

SEQ ID NO: 230 - protein, 16 aa, synthetic construct
  REGION 1..16: note = Synthetic sequence
NIYYRGYTYYNPSLKS

SEQ ID NO: 231 - protein, 16 aa, synthetic construct
  REGION 1..16: note = Synthetic sequence
TIGATGDTYYSDSVKG

SEQ ID NO: 232 - protein, 17 aa, synthetic construct
  REGION 1..17: note = Synthetic sequence
WINTNTGNPTYAQAFTG

SEQ ID NO: 233 - protein, 17 aa, synthetic construct
  REGION 1..17: note = Synthetic sequence
IIYPGDSDTRYSPSFQG

SEQ ID NO: 234 - protein, 17 aa, synthetic construct
  REGION 1..17: note = Synthetic sequence
WINTKTGNPTYAQGFTG

SEQ ID NO: 235 - protein, 17 aa, synthetic construct
  REGION 1..17: note = Synthetic sequence
WINTNTGKPTYAQGFTG

SEQ ID NO: 236 - protein, 18 aa, synthetic construct
  REGION 1..18: note = Synthetic sequence
SSLFKTETAPYYHFALDV

SEQ ID NO: 237 - protein, 15 aa, synthetic construct
  REGION 1..15: note = Synthetic sequence
GSSGWPSYSNWGFDY

SEQ ID NO: 238 - protein, 13 aa, synthetic construct
  REGION 1..13: note = Synthetic sequence
DWWYPPYYWGFDY

SEQ ID NO: 239 - protein, 15 aa, synthetic construct
  REGION 1..15: note = Synthetic sequence
DHGSRHFWSYWGFDY

SEQ ID NO: 240 - protein, 13 aa, synthetic construct
  REGION 1..13: note = Synthetic sequence
DTYDSTGYLWFDY

SEQ ID NO: 241 - protein, 13 aa, synthetic construct
  REGION 1..13: note = Synthetic sequence
DRHWHWWLDAFDY

SEQ ID NO: 242 - protein, 11 aa, synthetic construct
  REGION 1..11: note = Synthetic sequence
ERFLEWLHFDY

SEQ ID NO: 243 - protein, 16 aa, synthetic construct
  REGION 1..16: note = Synthetic sequence
EGYYETTTYYYNLFDS

SEQ ID NO: 244 - protein, 10 aa, synthetic construct
  REGION 1..10: note = Synthetic sequence
VYHWIRGFEF

SEQ ID NO: 245 - protein, 6 aa, synthetic construct
  REGION 1..6: note = Synthetic sequence
WELLDY

SEQ ID NO: 246 - protein, 7 aa, synthetic construct
  REGION 1..7: note = Synthetic sequence
GAGELDY

SEQ ID NO: 247 - protein, 16 aa, synthetic construct
  REGION 1..16: note = Synthetic sequence
QTYSSSWDGVLYYFDY

SEQ ID NO: 248 - protein, 18 aa, synthetic construct
  REGION 1..18: note = Synthetic sequence
GGIVVVPAARDYYYYMDV

SEQ ID NO: 249 - protein, 11 aa, synthetic construct
  REGION 1..11: note = Synthetic sequence
PNSGSPRYFEF

SEQ ID NO: 250 - protein, 16 aa, synthetic construct
  REGION 1..16: note = Synthetic sequence
QEYYYGSGSPSYYFDY

SEQ ID NO: 251 - protein, 11 aa, synthetic construct
  REGION 1..11: note = Synthetic sequence
HEWELLGPFDY

SEQ ID NO: 252 - protein, 12 aa, synthetic construct
  REGION 1..12: note = Synthetic sequence
EHIAARQDYFDY

SEQ ID NO: 253 - protein, 18 aa, synthetic construct
  REGION 1..18: note = Synthetic sequence
GDCDSTSCYRYSYGYEDY

SEQ ID NO: 254 - protein, 10 aa, synthetic construct
  REGION 1..10: note = Synthetic sequence
GSDEGDWFDP

SEQ ID NO: 255 - protein, 12 aa, synthetic construct
  REGION 1..12: note = Synthetic sequence
EYIAARLDYFDS

SEQ ID NO: 256 - protein, 14 aa, synthetic construct
  REGION 1..14: note = Synthetic sequence
DRGYSGYDAYYFDF

SEQ ID NO: 257 - protein, 14 aa, synthetic construct
  REGION 1..14: note = Synthetic sequence
EGSDYGDYVGAFDI

SEQ ID NO: 258 - protein, 14 aa, synthetic construct
  REGION 1..14: note = Synthetic sequence
DRGYIGYDSYYFDN

SEQ ID NO: 259 - protein, 11 aa, synthetic construct
  REGION 1..11: note = Synthetic sequence
ERTNFYDAFDI

SEQ ID NO: 260 - protein, 11 aa, synthetic construct
  REGION 1..11: note = Synthetic sequence
ERHGYFEAFDI

SEQ ID NO: 261 - protein, 11 aa, synthetic construct
  REGION 1..11: note = Synthetic sequence
ESNWNYDYFDY

SEQ ID NO: 262 - protein, 13 aa, synthetic construct
  REGION 1..13: note = Synthetic sequence
RGLSMVRLSAFDV

SEQ ID NO: 263 - protein, 11 aa, synthetic construct
  REGION 1..11: note = Synthetic sequence
DRGSYYDAFDI

SEQ ID NO: 264 - protein, 11 aa, synthetic construct
  REGION 1..11: note = Synthetic sequence
KGGSYYDWFDP

SEQ ID NO: 265 - protein, 14 aa, synthetic construct
  REGION 1..14: note = Synthetic sequence
DKGYNWNYMGAFDI

SEQ ID NO: 266 - protein, 11 aa, synthetic construct
  REGION 1..11: note = Synthetic sequence
RSGSYYDYFDY

SEQ ID NO: 267 - protein, 24 aa, synthetic construct
  REGION 1..24: note = Signal Peptide
MAGPLGLLCFLALGLLGSAGPSGA

SEQ ID NO: 268 - protein, 927 aa, synthetic construct
  REGION 1..927: note = hLGR4 (FL)
APPLCAAPCSCDGDRRVDCSGKGLTAVPEGLSAFTQALDISMNNITQLPEDAFKNFPFLEELQLAGNDLSFIHPKALSGLKELKVLTLQNNQLKTVPSEAIRGLSALQSLRLDANHITSVPEDSFEGLVQLRHLWLDDNSLTEVPVHPLSNLPTLQALTLALNKISSIPDFAFTNLSSLVVLHLHNNKIRSLSQHCFDGLDNLETLDLNYNNLGEFPQAIKALPSLKELGFHSNSISVIPDGAFDGNPLLRTIHLYDNPLSFVGNSAFHNLSDLHSLVIRGASMVQQFPNLTGTVHLESLTLTGTKISSIPNNLCQEQKMLRTLDLSYNNIRDLPSFNGCHALEEISLQRNQIYQIKEGTFQGLISLRILDLSRNLIHEIHSRAFATLGPITNLDVSFNELTSFPTEGLNGLNQLKLVGNFKLKEALAAKDFVNLRSLSVPYAYQCCAFWGCDSYANLNTEDNSLQDHSVAQEKGTADAANVTSTLENEEHSQIIIHCTPSTGAFKPCEYLLGSWMIRLTVWFIFLVALFFNLLVILTTFASCTSLPSSKLFIGLISVSNLFMGIYTGILTFLDAVSWGRFAEFGIWWETGSGCKVAGFLAVFSSESAIFLLMLATVERSLSAKDIMKNGKSNHLKQFRVAALLAFLGATVAGCFPLFHRGEYSASPLCLPFPTGETPSLGFTVTLVLLNSLAFLLMAVIYTKLYCNLEKEDLSENSQSSMIKHVAWLIFTNCIFFCPVAFFSFAPLITAISISPEIMKSVTLIFFPLPACLNPVLYVFFNPKFKEDWKLLKRRVTKKSGSVSVSISSQGGCLEQDFYYDCGMYSHLQGNLTVCDCCESFLLTKPVSCKHLIKSHSCPALAVASCQRPEGYWSDCGTQSAHSDYADEEDSFVSDSSDQVQACGRACFYQSRGFPLVRYAYNLPRVKD

SEQ ID NO: 269 - protein, 8 aa, synthetic construct
  REGION 1..8: note = FLAG TAG
DYKDDDDK

SEQ ID NO: 270 - protein, 4 aa, synthetic construct
  REGION 1..4: note = Linker REgion
LDGG

SEQ ID NO: 271 - protein, 9 aa, synthetic construct
  REGION 1..9: note = HA tag
YPYDVPDYA

SEQ ID NO: 272 - protein, 24 aa, synthetic construct
  REGION 1..24: note = Signal Peptide
MPGPLGLLCFLALGLLGSAGPSGA

SEQ ID NO: 273 - protein, 518 aa, synthetic construct
  REGION 1..518: note = hLGR4 (ECD)
APPLCAAPCSCDGDRRVDCSGKGLTAVPEGLSAFTQALDISMNNITQLPEDAFKNFPFLEELQLAGNDLSFIHPKALSGLKELKVLTLQNNQLKTVPSEAIRGLSALQSLRLDANHITSVPEDSFEGLVQLRHLWLDDNSLTEVPVHPLSNLPTLQALTLALNKISSIPDFAFTNLSSLVVLHLHNNKIRSLSQHCFDGLDNLETLDLNYNNLGEFPQAIKALPSLKELGFHSNSISVIPDGAFDGNPLLRTIHLYDNPLSFVGNSAFHNLSDLHSLVIRGASMVQQFPNLTGTVHLESLTLTGTKISSIPNNLCQEQKMLRTLDLSYNNIRDLPSFNGCHALEEISLQRNQIYQIKEGTFQGLISLRILDLSRNLIHEIHSRAFATLGPITNLDVSFNELTSFPTEGLNGLNQLKLVGNFKLKEALAAKDFVNLRSLSVPYAYQCCAFWGCDSYANLNTEDNSLQDHSVAQEKGTADAANVTSTLENEEHSQIIIHCTPSTGAFKPCEYLLGSWMIR

SEQ ID NO: 274 - protein, 85 aa, synthetic construct
  REGION 1..85: note = GPA33 Sequence containing the TM Region
VALYVGIAVGVVAALIIIGIIIYCCCCRGKDDNTEDKEDARPNREAYEEPPEQLRELSREREEEDDYRQEEQRSTGRESPDHLDQ

SEQ ID NO: 275 - protein, 21 aa, synthetic construct
  REGION 1..21: note = Signal Peptide
MDTSRLGVLLSLPVLLQLATG

SEQ ID NO: 276 - protein, 541 aa, synthetic construct
  REGION 1..541: note = hLGR5 (ECD)
GGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIR

SEQ ID NO: 277 - protein, 85 aa, synthetic construct; identical to SEQ ID NO: 274
  REGION 1..85: note = GPA33 Sequence Containing the TM Region

SEQ ID NO: 278 - protein, 21 aa, synthetic construct
  REGION 1..21: note = Signal Peptide
MDTSCVHMLLSLLALLQLVAA

SEQ ID NO: 279 - protein, 886 aa, synthetic construct
  REGION 1..886: note = mLGR5 (FL)
GSSPGPDAIPRGCPSHCHCELDGRMLLRVDCSDLGLSELPSNLSVFTSYLDLSMNNISQLPASLLHRLCFLEELRLAGNALTHIPKGAFTGLHSLKVLMLQNNQLRQVPEEALQNLRSLQSLRLDANHISYVPPSCFSGLHSLRHLWLDDNALTDVPVQAFRSLSALQAMTLALNKIHHIADYAFGNLSSLVVLHLHNNRIHSLGKKCFDGLHSLETLDLNYNNLDEFPTAIKTLSNLKELGFHSNNIRSIPERAFVGNPSLITIHFYDNPIQFVGVSAFQHLPELRTLTLNGASHITEFPHLTGTATLESLTLTGAKISSLPQAVCDQLPNLQVLDLSYNLLEDLPSLSGCQKLQKIDLRHNEIYEIKGSTFQQLFNLRSLNLAWNKIAIIHPNAFSTLPSLIKLDLSSNLLSSFPVTGLHGLTHLKLTGNRALQSLIPSANFPELKIIEMPSAYQCCAFGGCENVYKISNQWNKDDGNSVDDLHKKDAGLFQVQDERDLEDFLLDFEEDLKALHSVQCSPSPGPFKPCEHLFGSWLIRIGVWTTAVLALSCNALVALTVFRTPLYISSIKLLIGVIAVVDILMGVSSAVLAAVDAFTFGRFAQHGAWWEDGIGCQIVGFLSIFASESSIFLLTLAALERGFSVKCSSKFEVKAPLFSLRAIVLLCVLLALTIATIPLLGGSKYNASPLCLPLPFGEPSTTGYMVALVLLNSLCFLIMTIAYTKLYCSLEKGELENLWDCSMVKHIALLLFANCILYCPVAFLSFSSLLNLTFISPDVIKFILLVIVPLPSCLNPLLYIVFNPHFKEDMGSLGKHTRFWMRSKHASLLSINSDDVEKRSCESTQALVSFTHASIAYDLPSTSGASPAYPMTESCHLSSVAFVPCL

SEQ ID NO: 280 - protein, 8 aa, synthetic construct
  REGION 1..8: note = Linker
AAARGHPF

SEQ ID NO: 281 - protein, 10 aa, synthetic construct
  REGION 1..10: note = cMyc-Derived Epitope Tag
EQKLISEEDL

SEQ ID NO: 282 - protein, 5 aa, synthetic construct
  REGION 1..5: note = Linker
NMHTG

SEQ ID NO: 283 - protein, 6 aa, synthetic construct
  REGION 1..6: note = Hexa His tag
HHHHHH

SEQ ID NO: 284 - protein, 55 aa, synthetic construct
  REGION 1..55: note = Signal Peptide
MRPRSGGRPGATGRRRRRLRRRPRGLRCSRLPPPPPLPLLLGLLLAAAGPGAARA

SEQ ID NO: 285 - protein, 163 aa, synthetic construct
  REGION 1..163: note = ECD of human ZNRF3 VD
KETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFD

SEQ ID NO: 286 - protein, 886 aa, synthetic construct
  REGION 1..886: note = Cynomolgus LGR5
SSSPRSGALLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRQVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPVTGLHGLTHLKLTGNHALQSLISSENFPELKIIEMPYAYQCCAFGVCENAYKISNQWNKGDNSSMDDLHKKDAGMFQVQDERDLEDFLLDFEEDLKALHSVQCSPSPGPFKPCEHLLDGWLIRIGVWTIAVLALTCNALVTSTVFRSPLYISPIKLLIGVIAVVNMLTGVSSAVLAGVDAFTFGSFARHGAWWENGVGCQVIGFLSIFASESSVFLLTLAALERGFSVKCSAKFETKAPFSSLKVIILLCALLALTMAAVPLLGGSEYGASPLCLPLPFGEPSTTGYMVALILLNSLCFLMMTIAYTKLYCNLDKGDLENIWDCSMVKHIALLLFTNCILYCPVAFLSFSSLLNLTFISPEVIKFILLVIVPLPACLNPLLYILFNPHFKEDLVSLGKQTYFWTRSKHPSLMSINSDDVEKQSCDSTQALVTFTSSSIAYDLPPSSVPSPAYPVTESCHLSSVAFVPCL

SEQ ID NO: 287 - protein, 50 aa, synthetic construct
  REGION 1..50: note = PDGFR sequence containing the TM region
NAVGQDTQEVIVVPHSLPFKVVVISAILALVVLTIISLIILIMLWQKKPR

SEQ ID NO: 288 - protein, 163 aa, synthetic construct; identical to SEQ ID NO: 285
  REGION 1..163: note = ECD of human ZNRF3

SEQ ID NO: 289 - protein, 14 aa, synthetic construct
  REGION 1..14: note = Predicted TM region
MGIFLAFFVVVSLV

SEQ ID NO: 290 - protein, 24 aa, synthetic construct
  REGION 1..24: note = Intra-cellular tail
CLILLVKIKLKQRRSQNSMNRPAV

SEQ ID NO: 291 - protein, 153 aa, synthetic construct
  REGION 1..153: note = ECD of mouse ZNRF3
KETAFVEVVLFESSPSGDYTTHTTGLTGRFSRAGAMLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHL

SEQ ID NO: 292 - protein, 52 aa, synthetic construct
  REGION 1..52: note = Signal Peptide
MRPRSGGRPGAPGRRRRRLRRGPRGRRLPPPPPLPLLLGLLLAAAGPGAARA

SEQ ID NO: 293 - protein, 164 aa, synthetic construct
  REGION 1..164: note = ECD mZNRF3
KETAFVEVVLFESSPSGDYTTHTTGLTGRFSRAGAMLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHLPPRQPTEYFDM

SEQ ID NO: 294 - protein, 42 aa, synthetic construct
  REGION 1..42: note = Signal Peptide
MSGGHQLQLAALWPWLLMATLQAGFGRTGLVLAAAVESERSA

SEQ ID NO: 295 - protein, 157 aa, synthetic construct
  REGION 1..157: note = ECD of human RNF43
EQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDV

SEQ ID NO: 296 - protein, 42 aa, synthetic construct
  REGION 1..42: note = Signal Peptide
MSGGHQLQLAVLWPWLLMATLHAGFGHTGRVLAAAVESERSA

SEQ ID NO: 297 - protein, 157 aa, synthetic construct
  REGION 1..157: note = ECD of mouse RNF43
EQKAVIRVIPLKMDPTGKLNLTLEGVFAGVAEVTPAEGKLMQSHPLYLCNASDDDNLEPGFISIVKLESPRRAPRPCLSLASKARMAGERGANAVLFDITEDRSAAEQLQQPLGLTKPVVLIWGSDAAKLMEFVYKNRKAYVWIELKEPPAGANYDV

SEQ ID NO: 298 - protein, 21 aa, synthetic construct
  REGION 1..21: note = Signal Peptide
MDTSRLGVLLSLPVLLQLAAG

SEQ ID NO: 299 - protein, 6 aa, synthetic construct
  REGION 1..6: note = CDR 1
QSISSY

SEQ ID NO: 300 - protein, 9 aa, synthetic construct
  REGION 1..9: note = CDR3
QQSYSTPPT